(19) United States
(12) Patent Application Publication
(10) Pub. No.: US 2022/0340711 A1
(43) Pub. Date: Oct. 27, 2022

(54) CATIONIC POLYMER WITH ALKYL SIDE CHAINS

(71) Applicant: GenEdit, Inc., South San Francisco, CA (US)

(72) Inventors: Kunwoo Lee, South San Francisco, CA (US); Santanu Maity, South San Francisco, CA (US); Franco Duarte, South San Francisco, CA (US)

(21) Appl. No.: 17/605,981

(22) PCT Filed: Apr. 23, 2020
(86) PCT No.: PCT/US20/29660

§ 371 Date: Oct. 22, 2021

Related U.S. Application Data
(60) Provisional application No. 62/837,658, filed on Apr. 23, 2019.
(60) Provisional application No. 62/853,658, filed on May 28, 2019.

(51) Int. Cl. C08G 69/10; C08G 69/40; A61K 47/54; A61K 47/59; A61K 9/51

(52) U.S. Cl. CPC C08G 69/10; C08G 69/40; A61K 47/549; A61K 47/595; A61K 9/5146

(57) ABSTRACT

Provided is a polymer comprising a hydrolysable polymer backbone, the polymer backbone comprising (i) monomer units with a side chain comprising a hydrophobic group; (ii) monomer units with a side chain comprising an oligoamine or polyamine; and optionally (iii) monomer units with a side chain comprising an ionizable group, as well as a method of preparing said polymer, and a method of delivering a nucleic acid and/or polypeptide to a cell using the polymer.

### CROSS REFERENCE TO RELATED APPLICATIONS

This patent application claims priority to U.S. provisional patent application 62/837,658 filed Apr. 23, 2019, and U.S. provisional patent application 62/853,658 filed May 28, 2019, the entire disclosures of which are hereby incorporated by reference.

### BACKGROUND OF THE INVENTION

Peptide, protein, and nucleic based technologies have countless applications to prevent, cure and treat diseases. However, the safe and effective delivery of large molecules (e.g., polypeptides and nucleic acids) to their target tissues remains problematic. Accordingly, there continues to be a need for new compositions and methods useful for delivering therapeutic molecules.

### BRIEF SUMMARY OF THE INVENTION

Provided herein is a polymer comprising a hydrolysable polymer backbone, the polymer backbone comprising (i) monomer units with a side chain comprising a hydrophobic group; (ii) monomer units with a side chain comprising an oligoamine or polyamine; and optionally (iii) monomer units with a side chain comprising an ionizable group, optionally with a pKa less than 7.

Also provided herein is a polymer comprising a structure of Formula 1:

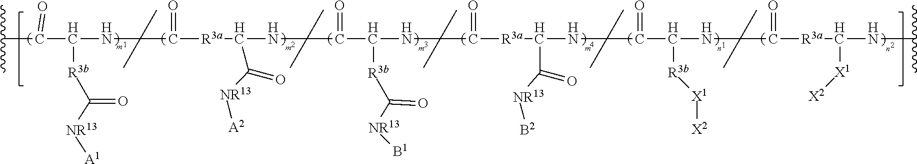

wherein:

each of m¹, m², m³, and m⁴ is an integer from 0 to 1000, provided that the sum of m¹+m²+m³+m⁴ is greater than 5;

each of n¹ and n² is an integer from 0 to 1000, provided that the sum of n¹+n² is greater than 2;

the symbol “/” indicates that the units separated thereby are linked randomly or in any order;

each instance of R^{3a} is independently a methylene or ethylene group;

each instance of R^{3b} is independently a methylene or ethylene group; each X¹ independently is —C(O)O—, —C(O)NR¹³—, —C(O)—, —S(O)(O)—, or a bond;

each instance of R¹³ is independently hydrogen, an aryl group, a heterocyclic group, a C₁-C₁₂ alkyl group, alkenyl group, cycloalkyl group, or cycloalkenyl group, any of which can be optionally substituted with one or more substituents;

each instance of X² is independently a C₁-C₁₂ alkyl group, cycloalkyl group, alkenyl group, cycloalkenyl group, aryl group, heteroalkyl group, heterocyclic group, or combination thereof optionally comprising one or more primary, secondary, or tertiary amines; any of which can be substituted with one or more substituents;

A¹ and A² are each independently a group of formula

—(CH₂)ₚ₁—[NR²—(CH₂)_{q1}—]ᵣ₁NR²₂;

—(CH₂)ₚ₂—N[—(CH₂)_{q2}—NR²₂]₂;

—(CH₂)ₚ₃—N{[—(CH₂)_{q3}—NR²₂][—(CH₂)_{q4}—NR²—]r₂R²}; or

—(CH₂)ₚ₄—N{—(CH₂)_{q5}—N[—(CH₂)_{q6}—NR²₂]₂}₂,

B¹ and B² are each independently

—(CH₂)ₚ₁—[NR²—(CH₂)_{q1}—]ᵣ₁NR²—(CH₂)ₛ₁—R⁴—R⁵;

—(CH₂)ₚ₂—N[—(CH₂)_{q2}—NR²—(CH₂)ₛ₂—R⁴—R⁵]₂:

—(CH₂)ₚ₃—N{[—(CH₂)_{q3}—NR²][—(CH₂)_{q4}—NR²—]ᵣ₂(CH₂)ₛ₃—R⁴—R⁵};

—(CH₂)ₚ₄—N{—(CH₂)_{q5}—N[—(CH₂)_{q6}—NR²—(CH₂)ₛ₄—R⁴—R⁵]₂}₂;

—(CH₂)ₚ₁—[NR²—(CH₂)_{q1}—]ᵣ₁NR²—CH₂—CHOH—R⁵;

—(CH₂)ₚ₂—N[—(CH₂)_{q2}—NR²—]ᵣ₂CH₂—CHOH—R⁵;

—(CH₂)ₚ₃—N{[—(CH₂)₃—NR²₂][—(CH₂)₄—NR²—]ᵣ₂—CH₂—CHOH—R⁵;

—(CH₂)ₚ₄—N{—(CH₂)_{q5}—N[—(CH₂)_{q6}—NR²—CH₂—CHOH—R⁵]₂}₂;

—(CH₂)ₚ₁—[NR²—(CH₂)_{q1}—]ᵣ₁NR²—(CH₂)ₛ₁—R⁵;

—(CH₂)ₚ₂—N[—(CH₂)_{q2}—NR²—(CH₂)ₛ₂—R⁵]₂;

—(CH₂)ₚ₃—N{[—(CH₂)_{q3}—NR²₂][—(CH₂)_{q4}—NR²—]ᵣ₂(CH₂)ₛ₃—R⁵ };

—(CH₂)ₚ₄—N{—(CH₂)_{q5}—N[—(CH₂)_{q6}—NR²—(CH₂)ₛ₄—R⁵]₂}₂;

—(CH₂)ₚ₁—[N{(CH₂)ₛ₁—R⁴—R⁵}—(CH₂)_{q1}—]ᵣ₁NR²₂;

—(CH₂)ₚ₁—[N{(CH₂)ₛ₁—R⁵}—(CH₂)_{q1}—]ᵣ₁NR²₂,

—(CH₂)ₚ₁—[NR²—(CH₂)_{q1}—]ᵣ₁NR²—CH(CONH₂)—(CH₂)ₛ₁—R⁵; or

—(CH₂)ₚ₁—[NR²—(CH₂)_{q1}—]ᵣ₁NR²—CH(CONH₂)—(CH₂)ₛ₁—R⁴—R⁵,

wherein p1 to p4, q1 to q6, r1 and r2, and s1 to s4 are each independently an integer of 1 to 5; each instance of R² is independently hydrogen or a C₁-C₁₂ alkyl group, alkenyl group, cycloalkyl group, or cycloalkenyl group, or R² is combined with a second R² so as to form a heterocyclic group; each instance of R⁴ is independently —C(O)O—, —C(O)NH—, —C—O—C(O)—O—C—, —O—, or —S(O)(O)—; and each instance of R⁵ is independently an alkyl group, cycloalkyl group, alkenyl group, cycloalkenyl group, aryl group, heteroalkyl group, heterocyclic group, or combination thereof optionally comprising from 2 to 8 tertiary amines or a substituent comprising a tissue-specific or cell-specific targeting moiety.

The foregoing polymer of Formula 1 can further comprise an ionizable group. In some embodiments, the ionizable group is provided by R⁵ of Formula 1. In other embodiments, the polymer further comprises a monomer with a side chain comprising an ionizable group.

The disclosure also provides a composition comprising a polymer comprising the structure of Formula 1 and a nucleic acid and/or polypeptide. Further provided is a method of preparing a polymer comprising a structure of Formula 1, as well as methods of using the polymers and compositions comprising same, for example, to deliver a nucleic acid or protein to a cell.

### BRIEF DESCRIPTION OF THE DRAWINGS

FIG. 1 provides the amino acid sequence of Cas9 from Streptococcus pyogene (SEQ ID NO:1).

FIG. 2 provides the amino acid sequence of Cpf1 from Francisella tularensis subsp. Novicida U112 (SEQ ID NO:2).

FIG. 3 is a graph of the degree of substitution of the hydrophobic moiety of Polymer A as a result of the number of equivalents of hydrophobic moiety added in the reaction mixture.

FIG. 4 is a graph of the degree of substitution of the hydrophobic moiety of Polymer B as a result of the number of equivalents of hydrophobic moiety added in the reaction mixture.

FIG. 5 is a graph illustrating transfection efficiency of Polymer A nanoparticles in HEK293T cells as a function of RFP fluorescence, as described in Example 3.

FIG. 6 is a graph illustrating transfection efficiency of Polymer A nanoparticles in HEK293T cells as a function of RFP fluorescence, as described in Example 4.

FIG. 7 is a graph illustrating transfection efficiency of Polymer A nanoparticles in HepG2 cells as a function of RFP fluorescence, as described in Example 4.

FIG. 8 is a graph illustrating transfection efficiency of Polymer A nanoparticles in primary myoblasts as a function of RFP fluorescence, as described in Example 4.

FIG. 9 is a graph illustrating transfection efficiency of Polymer A nanoparticles and Polymer B nanoparticles in HEK293T cells as a function of RFP fluorescence, as described in Example 4.

FIG. 10 is a graph is a graph illustrating transfection efficiency of Polymer A and Polymer B nanoparticles containing Cas9 in HEK293T cells as a function of GFP knock-out, as described in Example 5.

FIG. 11 is a graph is a graph illustrating transfection efficiency of Polymer A and Polymer B nanoparticles containing Cpf1 in HEK293T cells as a function of GFP knock-out, as described in Example 5.

FIG. 12 is a graph illustrating transfection efficiency of Polymer A nanoparticles in HEK293T cells as a function of RFP fluorescence, as described in Example 6.

FIG. 13 is a graph illustrating cell viability of Hep3B cells after treatment of cells with Polymer A nanoparticles, as described in Example 7.

FIG. 14 provides the sequence of AsCpf1 (SEQ ID NO: 19).

FIG. 15 provides the sequence of LbCpf1 (SEQ ID NO: 20).

FIG. 16 shows the dynamic light scattering of particles containing mCherry mRNA and polymer H27N, as described in Example 9.

FIG. 17 shows the dynamic light scattering of particles containing Cas9 RNP and polymer H27N, as described in Example 10.

FIG. 18 is a graph illustrating transfection efficiency of Polymer H27N nanoparticles in HEK293T cells as a function of RFP fluorescence, as described in Example 12.

FIG. 19 is a graph illustrating transfection efficiency of Polymer H27N nanoparticles in Hep3B cells as a function of nonhomologous end joining (NHEJ) efficiency, as described in Example 13.

FIG. 20 is a graph illustrating transfection efficiency of Polymer H27N nanoparticles in Hep3B cells as a function of nonhomologous end joining (NHEJ) efficiency, as described in Example 14.

FIG. 21 is a schematic illustration of a mouse Loxp-luciferase reporter function.

FIGS. 22A-22C show the bioluminescence imaging of luciferase expressing mice treated with compositions as described in Example 15.

FIG. 23 is a schematic illustration of a mouse ai9 reporter function.

FIG. 24 shows the bioluminescence imaging of delivery of Cre mRNA to the rostral and caudal sections of the brain of luciferase expressing mice treated with compositions as described in Example 17.

FIG. 25 is a graph illustrating transfection efficiency of polymer nanoparticles as a function of RFP fluorescence, as described in Example 23.

### DETAILED DESCRIPTION OF THE INVENTION

The invention provides a polymer comprising a hydrolysable polymer backbone, the polymer backbone comprising (i) monomer units with a side chain comprising a hydrophobic group; (ii) monomer units with a side chain comprising an oligoamine or polyamine; and optionally (iii) monomer units with a side chain comprising an ionizable group, optionally with a pKa less than 7.

As used herein, the phrase hydrolysable polymer backbone refers to a polymer backbone having bonds that are susceptible to cleavage under physiological conditions (e.g., physiological pH, physiological temperature, or in a given in vivo tissue such as blood, serum, etc. due to naturally occurring factors (e.g., enzymes). Generally, the hydrolysable polymer backbone comprises a polyamide, poly-N-alkylamide, polyester, polycarbonate, polycarbamate, or a combination thereof. In certain embodiments, the hydrolysable polymer backbone comprises a polyamide.

The monomer units with a side chain comprising a hydrophobic group, can comprise any hydrophobic group. Examples of hydrophobic groups include, for instance, a C₁-C₁₂ (e.g., C₂-C₁₂, C₂-C₁₀, C₂-C₈, C₂-C₆, C₃-C₁₂, C₃-C₁₀, C₃-C₈, C₃-C₆, C₄-C₁₂, C₄-C₁₀, C₄C₈, C₄-C₆, C₆-C₁₂, C₆-C₈, C₈-C₁₂, C₈-C_{10,}) alkyl group, a C₂-C₁₂ (e.g., C₂-C₆, C₃-C₁₂, C₃-Cl₀, C₃-C₈, C₃-C₆, C₄-C₁₂, C₄-C₁₀, C₄-C₈, C₄-C₆, C₆-C₁₂, C₆-C₈, C₈-C₁₂, C₈-C_{10,}) alkenyl group, or a C₃-C₁₂ (C₃-Cl₀, C₃-C₈, C₃-C₆, C₄-C₁₂, C₄-C₁₀, C₄-C₈, C₄-C₆, C₆-C₁₂, C₆-C₈, C₈- C₁₂, C₈-C₁₀,) cycloalkyl group or cycloalkenyl group. In certain embodiments, the hydrophobic group comprises a C₄-C₁₂ alkyl group, alkenyl group, cycloalkyl group, or cycloalkenyl group. In some embodiments, the hydrophobic group comprises fewer than 8 carbons or fewer than 6 carbons. For example, the hydrophobic group can comprise a C₂-C₈ or C₂-C₆ (e.g., C₃-C₈ or C₃-C₆) alkyl group. The alkyl or alkenyl groups can be branched or straight-chain. In any of the foregoing embodiments, the hydrophobic group can be linked to the polymer backbone direclty or via a linkage comprising, for instance, an ester, an amide, or an ether group, optionally further comprising an alkylene linker (e.g., a methylene or ethylene linker).

The polymer also comprises monomer units with a side chain comprising an oligoamine or polyamine. As used herein, the term “oligoamine” refers to any chemical moiety having two or three amine groups, and the term “polyamine” refers to any chemical moiety having four or more (e.g., 4, 5, 6, 7, 8, 9, 10, 11, 12, 13, 14, 15, 16, etc.) amine groups. The amine groups can be primary amine groups, secondary amine groups, tertiary amine groups, or any combination thereof. In certain embodiments, the oligoamine or polyamine is of the formula:

—(CH₂)ₚ₁—[NR²—(CH₂)_{q1}—]ᵣ₁NR²₂;

—(CH₂)ₚ₂—N[—(CH₂)_{q2}—NR²₂]₂;

—(CH₂)ₚ₃—N{[—(CH₂)_{q3}—NR²₂][—(CH₂)_{q4}—NR²—]r2R²};

—(CH₂)ₚ₄—N{—(CH₂)_{q5}—N[—(CH₂)_{q6}—NR²₂]₂}₂,

—(CH₂)ₚ₁—[NR²—(CH₂)_{q1}—]ᵣ₁NR²—(CH₂)ₛ₁—R⁴—R⁵;

—(CH₂)ₚ₂—N[—(CH₂)_{q2}—NR²—(CH₂)ₛ₂—R⁴—R⁵]₂;

—(CH₂)ₚ₃—N{[—(CH₂)_{q3}—NR²₂][—(CH₂)_{q4}—NR²—]ᵣ₂(CH₂)ₛ₃—R⁴—R⁵};

—(CH₂)ₚ₄—N{—(CH₂)_{q5}—N[—(CH₂)_{q6}—NR²—(CH₂)ₛ₄—R⁴—R⁵]₂}₂;

—(CH₂)ₚ₁—[NR²—(CH₂)_{q1}—]ᵣ₁NR²—CH₂—CHOH—R⁵;

—(CH₂)ₚ₂—N[—(CH₂)_{q2}—NR²—CH₂—CHOH—R^{5]}₂;

—(CH₂)ₚ₃—N{[—(CH₂)_{q3}—NR²₂][—(CH₂)_{q4}—NR²—]ᵣ₂—CH₂—CHOH—R⁵};

—(CH₂)ₚ₄—N{—(CH₂)_{q5}—N[—(CH₂)_{q6}—NR²—CH₂—CHOH—R⁵]₂}₂;

—(CH₂)ₚ₁—[NR²—(CH₂)_{q1}—]ᵣ₁NR²—(CH₂)ₛ₁—R⁵;

—(CH₂)ₚ₂—N[—(CH₂)_{q2}—NR²—(CH₂)ₛ₂—R⁵]₂;

—(CH₂)ₚ₃—N{[—(CH₂)_{q3}—NR²₂][—(CH₂)_{q4}—NR²—]ᵣ₂(CH₂)ₛ₃—R⁵};

—(CH₂)ₚ₄—N{—(CH₂)_{q5}—N[—(CH₂)_{q6}—NR²—(CH₂)ₛ₄—R⁵]₂}₂;

—(CH₂)ₚ₁—[N{(CH₂)ₛ₁—R⁴—R⁵}—(CH₂)_{q1}—]ᵣ₁NR²₂;

—(CH₂)ₚ₁—[N{(CH₂)ₛ₁—R⁵}—(CH₂)_{q1}—]ᵣ₁NR²₂,

—(CH₂)ₚ₁—[NR²—(CH₂)_{q1}—]ᵣ₁NR²—CH(CONH₂)—(CH₂)ₛ₁—R⁵; or

—(CH₂)ₚ₁—[NR²—(CH₂)_{q1}—]ᵣ₁NR²—CH(CONH₂)—(CH₂)ₛ₁—R⁴—R⁵,

wherein p1 to p4, q1 to q6, r1 and r2, and s1 to s4 are each independently an integer of 1 to 5; each instance of R² is independently hydrogen or a C₁-C₁₂ alkyl group, alkenyl group, cycloalkyl group, or cycloalkenyl group, or R² is combined with a second R² so as to form a heterocyclic group; each instance of R⁴ is independently —C(O)O—, —C(O)NH—, —O—, —C—O—C(O)—C—O—, or —S(O)(O)—; and each instance of R⁵ is independently an alkyl group, cycloalkyl group, alkenyl group, cycloalkenyl group, aryl group, heteroalkyl group, heterocyclic group, or combination thereof optionally comprising from 2 to 8 tertiary amines or a substituent comprising a tissue-specific or cell-specific targeting moiety.

In some embodiments, the oligoamine or polyamine is of the formula:

—(CH₂)ₚ₁—[NR²—(CH₂)_{q1}—]ᵣ₁NR²₂;

—(CH₂)ₚ₂—N[—(CH₂)_{q2}—NR²₂]₂;

—(CH₂)ₚ₃—N{[—(CH₂)_{q3}—NR²₂][—(CH₂)_{q4}—NR²—]r₂R²}; or

—(CH₂)ₚ₄—N{—(CH₂)_{q5}—N[—(CH₂)_{q6}—NR²₂]₂}₂,

wherein p1 to p4, q1 to q6, and r1 and r2 are each independently an integer of 1 to 5 (e.g., 1, 2, or 3); and each instance of R² is independently hydrogen or a C₁-C₁₂ (e.g., C₁-C₆, C₁-C₃, C₂, or CO alkyl group, alkenyl group, cycloalkyl group, or cycloalkenyl group, or R² is combined with a second R² so as to form a heterocyclic group. It is understood that the alkenyl groups must have at least 2 carbons (e.g., C₂-C₁₂, C₂-C₆, etc.) and the cycloalkyl and cycloalkenyl groups must have at least 3 carbons (e.g., C₃-C₁₂, C₃-C₆, etc.). In some embodiments, the polyamine is —(CH₂)ₚ₁—[NR²—(CH₂)_{q1}—]ᵣ₁NR²₂, optionally wherein R² is independently hydrogen or a C₁-C₃ alkyl (e.g., methyl or ethyl).

In some embodiments, the polymer further comprises monomer units with a side chain comprising an ionizable group. As used herein, the phrase “ionizable group” refers to any chemical moiety with a substituent that can readily be converted into a charged species. For example, the ionizable group can be a group that is a proton-donor or proton-acceptor. The group can be protonated or deprotonated under physiological conditions. In certain embodiments, the ionizable group has a pKa less than 7 (in water at 25° C.). For example, the ionizable group described herein can have a pKa of less than 6, a pKa of less than 5, a pKa of less than 4, a pKa of less than 3, a pKa of less than 2, or a pKa of less than 1. Alternatively, or additionally, the ionizable group described herein can have a pKa of greater than −2, a pKa of greater than −1, a pKa of greater than 0, a pKa of greater than 1, a pKa of greater than 2, a pKa of greater than 3, a pKa of greater than 4, a pKa of greater than 5, or a pKa of greater than 6. Accordingly, the ionizable group described herein can have a pKa from −2 to 7, for example, a pKa from −1 to 7, a pKa from 0 to 7, a pKa from 1 to 7, a pKa from 2 to 7, a pKa from 3 to 7, a pKa from 4 to 7, a pKa from 5 to 7, a pKa from 6 to 7, a pKa from 0 to 6, a pKa from 2 to 6, a pKa from 4 to 6, a pKa from 0 to 5, a pKa from 2 to 5, or a pKa from 4 to 5. Examples of ionizable groups include, for instance, sulfonic acid, sulfonamide, carboxylic acid, thiol, phenol, amine salt, imide, and amide groups.

In some embodiments, the polymer has an overall pKa of less than 7 (in water at 25° C.). For example, the polymer described herein can have a pKa of less than 6, a pKa of less than 5, a pKa of less than 4, a pKa of less than 3, a pKa of less than 2, or a pKa of less than 1. Alternatively, or additionally, the polymer described herein can have a pKa of greater than −2, a pKa of greater than −1, a pKa of greater than 0, a pKa of greater than 1, a pKa of greater than 2, a pKa of greater than 3, a pKa of greater than 4, a pKa of greater than 5, or a pKa of greater than 6. Accordingly, the polymer described herein can have a pKa from −2 to 7, for example, a pKa from −1 to 7, a pKa from 0 to 7, a pKa from 1 to 7, a pKa from 2 to 7, a pKa from 3 to 7, a pKa from 4 to 7, a pKa from 5 to 7, a pKa from 6 to 7, a pKa from 0 to 6, a pKa from 2 to 6, a pKa from 4 to 6, a pKa from 0 to 5, a pKa from 2 to 5, ora pKa from 4 to 5.

The polymer can comprise any suitable number or amount (e.g., weight or number percent composition) of the monomer units with a side chain comprising a hydrophobic group, the monomer units with a side chain comprising an oligoamine or polyamine, and, when present, the monomer units with a side chain comprising an ionizable group. In some embodiments, the polymer comprises about 1 to about 80 mol % (e.g., about 5 to about 80 mol %, about 10 to about 80 mol %, about 20 to about 80 mol %, about 40 to about 80 mol %, about 1 to about 60 mol %, about 1 to about 40 mol %, about 1 to about 20 mol %, or about 1 to about 10 mol %) of the monomer units having a hydrophobic group, about 1 to about 80 mol % (e.g., about 5 to about 80 mol %, about 10 to about 80 mol %, about 20 to about 80 mol %, about 40 to about 80 mol %, about 1 to about 60 mol %, about 1 to about 40 mol %, about 1 to about 20 mol %, or about 1 to about 10 mol %) of the monomer units having an oligoamine or polyamine, and 0 to about 80 mol % (e.g., about 5 to about 80 mol %, about 10 to about 80 mol %, about 20 to about 80 mol %, about 40 to about 80 mol %, about 1 to about 60 mol %, about 1 to about 40 mol %, about 1 to about 20 mol %, or about 1 to about 10 mol %) of the monomer units having an ionizable group.

Provided herein is a polymer comprising a structure of Formula 1:

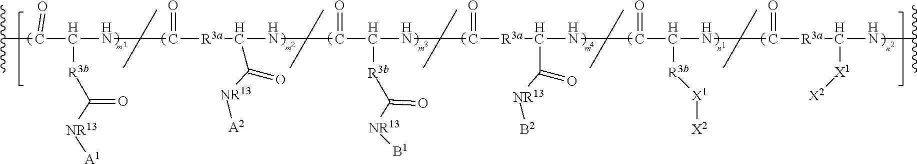

wherein:

each of m¹, m², m³, and m⁴ is an integer from 0 to 1000, provided that the sum of m¹+m²+m³+m⁴ is greater than 5;

each of n¹ and n² is an integer from 0 to 1000, provided that the sum of n¹+n² is greater than 2;

the symbol “/” indicates that the units separated thereby are linked randomly or in any order;

each instance of R^{3a} is independently a methylene or ethylene group;

each instance of R^{3b} is independently a methylene or ethylene group;

each X¹ independently is —C(O)O—, —C(O)NR¹³—, —C(O)—, —S(O)(O)—, or a bond;

each instance of R¹³ is independently hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which can be optionally substituted with one or more substituents;

each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof optionally comprising one or more primary, secondary, or tertiary amines; any of which are optionally substituted with one or more substituents;

A¹ and A² are each independently a group of formula

—(CH₂)ₚ₁—[NR²—(CH₂)_{q1}—]ᵣ₁NR²₂;

—(CH₂)ₚ₂—N[—(CH₂)_{q2}—NR²₂]₂;

—(CH₂)ₚ₃—N{[—(CH₂)_{q3}—NR²₂][—(CH₂)_{q4}—NR²—]r₂R²}; or

—(CH₂)ₚ₄—N{—(CH₂)_{q5}—N[—(CH₂)_{q6}—NR²₂]₂}₂,

B¹ and B² are each independently

—(CH₂)ₚ₁—[NR²—(CH₂)_{q1}—]ᵣ₁NR²—(CH₂)ₛ₁—R⁴—R⁵;

—(CH₂)ₚ₂—N[—(CH₂)_{q2}—NR²—(CH₂)ₛ₂—R⁴—R⁵]₂;

—(CH₂)ₚ₃—N{[—(CH₂)_{q3}—NR²₂][—(CH₂)_{q4}—NR²—]ᵣ₂(CH₂)ₛ₃—R⁴—R⁵};

—(CH₂)ₚ₄—N{—(CH₂)_{q5}—N[—(CH₂)_{q6}—NR²—(CH₂)ₛ₄—R⁴—R⁵]₂}₂;

—(CH₂)ₚ₁—[NR²—(CH₂)_{q1}—]ᵣ₁NR²—CH₂—CHOH—R⁵;

—(CH₂)ₚ₂—N[—(CH₂)_{q2}—NR²—CH₂—CHOH—R⁵;

—(CH₂)ₚ₃—N{[—(CH₂)_{q3}—NR²₂][—(CH₂)_{q4}—NR²—]ᵣ₂—CH₂—CHOH—R⁵;

—(CH₂)ₚ₄—N{—(CH₂)_{q5}—N[—(CH₂)_{q6}—NR²—CH₂—CHOH—R⁵]₂}₂;

—(CH₂)ₚ₁—[NR²—(CH₂)_{q1}—]ᵣ₁NR²—(CH₂)ₛ₁—R⁵;

—(CH₂)ₚ₂—N[—(CH₂)_{q2}—NR²—(CH₂)ₛ₂—R⁵]₂;

—(CH₂)ₚ₃—N{[—(CH₂)_{q3}—NR²₂][—(CH₂)_{q4}—NR²—]ᵣ₂(CH₂)ₛ₃—R⁵};

—(CH₂)ₚ₄—N{—(CH₂)_{q5}—N[—(CH₂)_{q6}—NR²—(CH₂)ₛ₄—R⁵]₂}₂;

—(CH₂)ₚ₁—[N{(CH₂)ₛ₁—R⁴—R⁵}—(CH₂)_{q1}—]ᵣ₁NR²₂;

—(CH₂)ₚ₁—[N{(CH₂)ₛ₁—R⁵}—(CH₂)_{q1}—]ᵣ₁NR²₂,

—(CH₂)ₚ₁—[NR²—(CH₂)_{q1}—]ᵣ₁NR²—CH(CONH₂)—(CH₂)ₛ₁—R⁵; or

—(CH₂)ₚ₁—[NR²—(CH₂)_{q1}—]ᵣ₁NR²—CH(CONH₂)—(CH₂)ₛ₁—R⁴—R⁵,

wherein p1 to p4, q1 to q6, r1 and r2, and s1 to s4 are each independently an integer of 1 to 5; each instance of R² is independently hydrogen or a C₁-C₁₂ alkyl group, alkenyl group, cycloalkyl group, or cycloalkenyl group, or R² is combined with a second R² so as to form a heterocyclic group; each instance of R⁴ is independently —C(O)O—, —C(O)NH—, —O—, —C—O—C(O)—C—O—, or S(O)(O)—; and each instance of R⁵ is independently an alkyl group, cycloalkyl group, alkenyl group, cycloalkenyl group, aryl group, heteroalkyl group, heterocyclic group, or combination thereof optionally comprising from 2 to 8 tertiary amines or a substituent comprising a tissue-specific or cell-specific targeting moiety.

As used herein, “alkyl” or “alkylene” refers to a substituted or unsubstituted hydrocarbon chain. The alkyl group can have any number of carbon atoms (e.g., C₁-C₁₀₀ alkyl, C₁-C₅₀ alkyl, C₁-C₁₂ alkyl, C₁-C₈ alkyl, C₁-C₆ alkyl, C₁-C₄ alkyl, C₁-C₂ alkyl, etc.). The alkyl or alkylene can be saturated, or can be unsaturated (e.g., to provide an alkenyl or alkynyl), and can be linear, branched, straight-chained, cyclic (e.g., cycloalkyl or cycloalkenyl), or a combination thereof. Cyclic groups can be monocyclic, fused to form bicyclic or tricyclic groups, linked by a bond, or spirocyclic. In some embodiments, the alkyl substituent can be interrupted by one or more heteroatoms (e.g., oxygen, nitrogen, and sulfur), thereby providing a heteroalkyl, heteroalkylene, or heterocyclyl (i.e., a heterocyclic group). In some embodiments, the alkyl is substituted with one or more substituents.

The term “aryl” refers to an aromatic ring system having any suitable number of ring atoms and any suitable number of rings. Aryl groups can include, for instance, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. In some embodiments, the aryl group comprises an alkylene linking group so as to form an arylalkyl group (e.g., a benzyl group). Some aryl groups have from 6 to 12 ring members, such as phenyl, naphthyl or biphenyl. Other aryl groups have from 6 to 10 ring members, such as phenyl or naphthyl. In some embodiments, the aryl substituent can be interrupted by one or more heteroatoms (e.g., oxygen, nitrogen, and sulfur), thereby proving a heterocyclyl (i.e., a heterocyclic or heteroaryl group). In some embodiments, the aryl is substituted with one or more substituents.

The term “heterocyclyl,” or “heterocyclic group” refers to a cyclic group, e.g., aromatic (e.g., heteroaryl) or non-aromatic where the cyclic group has one or more heteroatoms (e.g., oxygen, nitrogen, and sulfur). In some embodiments, the heterocyclyl or heterocyclic group (i.e., cyclic group, e.g., aromatic (e.g., heteroaryl) or non-aromatic where the cyclic group has one or more heteroatoms) is substituted with one or more substituents.

As used herein, the term “substituted” can mean that one or more hydrogens on the designated atom or group (e.g., substituted alkyl group) are replaced with another group provided that the designated atom's normal valence is not exceeded. For example, when the substituent is oxo (i.e., ═O), then two hydrogens on the atom are replaced. Substituent groups can include one or more of a hydroxyl, an amino (e.g., primary, secondary, or tertiary), an aldehyde, a carboxylic acid, an ester, an amide, a ketone, nitro, an urea, a guanidine, cyano, fluoroalkyl (e.g., trifluoromethane), halo (e.g., fluoro), aryl (e.g., phenyl), heterocyclyl or heterocyclic group (i.e., cyclic group, e.g., aromatic (e.g., heteroaryl) or non-aromatic where the cyclic group has one or more heteroatoms), oxo, or combinations thereof. Combinations of substituents and/or variables are permissible provided that the substitutions do not significantly adversely affect synthesis or use of the compound.

According to Formula 1, each of m¹, m², m³, and m⁴ is an integer from 0 to 1000 (e.g., 0 to 500, 0 to 200, 0 to 100, or 0 to 50), provided that the sum of m¹+m²+m³+m⁴ is greater than 5, such as 5-5000, 5-2000, 5-1000, 5-500, 5-100, or 5-50. In some embodiments, the sum of m¹+m²+m³+m⁴ is greater than 10 or greater than 20 (e.g., 10-5000, 10-2000, 10-1000, 10-500, 10-100, or 10-50; or 20-5000, 20-2000, 20-1000, 20-500, 20-100, or 20-50). Furthermore, each of n¹ and n² is an integer from 0 to 1000 (e.g., 0 to 500, 0 to 200, 0 to 100, 0 to 50, or 0 to 25), provided that the sum of n¹+n² is greater than 2 (e.g., 2-2000, 2-1000, 2-500, 2-200, 2-100, 2-50, or 2-25). In some embodiments, the sum of n¹+n² is greater than 5 or greater than 10 (e.g., 5-2000, 5-1000, 5-500, 5-200, 5-100, 5-50, or 5-25; or 10-2000, 10-1000, 10-500, 10-200, 10-100, 10-50, or 10-25). In other words, the polymer comprises at least some monomeric units comprising groups A¹, A², B¹, and/or B², herein referred to collectively as the “A monomers” and “B monomers,” respectively. Similarly, the polymer comprises at least some monomeric units comprising groups X¹ and/or X², herein referred to collectively as the “X monomers.” In some embodiments, m¹ and m² are zero, such that the polymer comprises no A¹ or A² groups. In some embodiments, m³ and m⁴ are zero, such that the polymer comprises no B¹ or B² groups.

The polymer can comprise any suitable ratio of A and B monomers to X monomers. In some embodiments, the polymer comprises a ratio of A and B monomers to X monomers (e.g., the ratio of (m¹+m²+m³+m⁴)/(n¹+n²)) of about 25 or less, and, optionally, about 1 or more. For example, the ratio of A and B monomers to X monomers can be about 1 to about 25, from about 1 to about 20, from about 1 to about 10, from about 1 to about 5, from about 5 to about 25, from about 10 to about 25, or from about 15 to about 25.

In embodiments where the polymer comprises both A monomers and B monomers, the polymer can comprise any suitable ratio of A monomers to B monomers. In some embodiments, the ratio of A monomers to B monomers (e.g., (m¹+m²)/(m³+m⁴)) can be about 20 or less (e.g., about 10 or less, about 5 or less, about 2 or less, or even about 1 or less). In some embodiments, the ratio of (m¹+m²)/(m³+m⁴) is about 0.2 or more, such as about 0.5 or more.

The polymer can exist as any suitable structure type. For example, the polymer can exist as an alternating polymer, random polymer, block polymer, graft polymer, linear polymer, branched polymer, cyclic polymer, or a combination thereof. In certain embodiments, the polymer is a random polymer, block polymer, graft polymer, or a combination thereof.

Thus, in the structure of Formula 1, the monomers (which can be referred to by their respective side chains A¹, A², B¹, B², X¹, and X²) can be arranged randomly or in any order. The integers m¹, m², m³, m⁴, n¹, and n² merely denote the number of the respective monomers that appear in the chain overall, and do not necessarily imply or represent any particular order or blocks of those monomers, although blocks or stretches of a given monomer might be present in some embodiments. For instance, the structure of Formula 1 can comprise the monomers in the order -A¹-A²-B¹-B²-, -A²-A¹-B²-B¹-, -A¹-B¹-A²-B²-, etc.

Furthermore, the polymer can comprise blocks of A and/or B polymers (e.g., [A monomers]ₘ₁₊ₘ2-[B monomers]ₘ₃₊ₘ₄) in any order). The polymer can comprise individual X monomers interspersed with the A and B monomers (e.g., -A-X-B-, -A-B-X-, -B-X-A, etc.), or the polymer can be “capped” with one or more X monomers (e.g., a block of X monomers) at one or both ends of the polymer. Likewise, when the polymer comprises blocks of A and/or B monomers, the polymer can comprise blocks of X monomers interspersed between blocks of A and/or B monomers, or the polymer can be “capped” with one or more X monomers (e.g., a block of X monomers) at one or both ends of the polymer. In some embodiments, the polypeptide (e.g., polyaspartamide) backbone will be arranged in an alpha/beta configuration, such that the “1” and “2” monomers will alternate (e.g., -A¹-A²-B¹-B²-, -A²-A¹-B²-B¹-, -A¹-B²-B¹-A²-, -A²-B¹-B²-A¹-, -B¹-A²-B¹-A²-, etc.), wherein the polymer is capped with X monomers or the X monomers are interspersed throughout. However, the “A” and “B” sidechains (e.g., A¹/A² and B¹/B²) can be dispersed randomly throughout the polymer backbone.

In the polymer structures, R^{3a} and R^{3b} are each independently a methylene or ethylene group. In some embodiments, R^{3a} is an ethylene group and R^{3b} is a methylene group; or R^{3a} is a methylene group and R^{3b} is an ethylene group. In certain embodiments, R^{3a} and R^{3b} are each an ethylene group. In some embodiments, R^{3a} and R^{3b} are each a methylene group.

In the polymers described herein, each X¹ group independently is —C(O)O—, —C(O)NR¹³—, —C(O)—, —S(O)(O)—, or a bond. Each X¹ group can be the same or different from one another. In some embodiments, X¹ is —C(O)NR¹³—. In some embodiments, X¹ is —C(O)O—.

Each instance of R¹³ is independently hydrogen or a C₁-C₁₂ (e.g., C₁-C₈, C₁-C_{6,} or C₁-C₃) alkyl group, C₂-C₁₂ (e.g., C₂-C₈, C₂-C_{6,} or C₂-C₃) alkenyl group, C₃-C₁₂ (e.g., C₃-C₈, C₃-C_{6,} or C₃-C₅) cycloalkyl group, C₃-C₁₂ (e.g., C₃-C₈, C₃-C_{6,} or C₃-C₅) cycloalkenyl group, aryl group, or heterocyclic group (e.g., 3-12, 3-10, 3-8, or 3-6 membered heterocyclic group comprising one, two, or three heteroatoms), any of which can be substituted with one or more substituents. In some embodiments, R¹³ is a C₁-C₁₂ alkyl group (e.g., a C₁-C₁₀ alkyl group; a C₁-C₈ alkyl group; a C₁-C₆ alkyl group; a C₁-C₄ alkyl group, a C₁-C₃ alkyl group, or a C₁ or C₂ alkyl group) which can be linear or branched. In certain embodiments, each R¹³ is methyl or hydrogen. In some embodiments, R¹³ is methyl; in other embodiments, R¹³ is hydrogen. Each R¹³ is independently chosen and can be the same or different; however, in some embodiments, each R¹³ is the same (e.g., all methyl or all hydrogen).

Each instance of X² is independently C₁-C₁₂ (e.g., C₁-C₈, C₁-C₆, or C₁-C₃) alkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, C₃-C₁₂ (e.g., C₃-C₈, C₃-C₆, or C₃-C₅) cycloalkenyl group, aryl group, or heterocyclic group (e.g., 3-12, 3-10, 3-8, or 3-6 membered heterocyclic group comprising one, two, or three heteroatoms)or combination thereof, any of which can be substituted with one or more substituents. In some embodiments, X² optionally can comprise one or more primary, secondary, or tertiary amines. Accordingly, each X² is independently selected and, therefore, can be the same or different from one another. In certain embodiments, each instance of X² is independently a C₁-C₁₂ (e.g., C₁-C₈, C₁-C₆, or C₁-C₃) alkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, C₃-C₁₂ (e.g., C₃-C₈, C₃-C₆, or C₃-C₅) cycloalkenyl group, or combination thereof optionally comprising one or more primary, secondary, or tertiary amines. In some embodiments, one or more (or all) X² groups can be independently a C₂-C₁₂ (e.g., C₃-C₁₂, C₃-C₈, C₃-C₆, C₄-C₁₂, C₄-C₆, C₆-C_{12,} or C₈-C₁₂) alkyl group or alkenyl group, or C₃-C₁₂ (e.g., C₃-C₈, C₃-C₆, C₄-C₁₂, C₄-C₆, C₆-C_{12,} or C₈-C₁₂) cycloalkenyl group. In other embodiments, one or more (or all) X² groups can be independently a C₁-C₈ (e.g., C₁-C₆, C₁-C₄, C₁-C₃, C₂-C_{8,} or C₂-C₆) alkyl groups. Any of the foregoing alkyl or alkenyl groups can be linear or branched.

Groups A¹ and A² are independently selected and, therefore, can be the same or different from one another. Similarly, groups B¹ and B² are independently selected and can be the same or different from one another. However, in some embodiments, A¹ and A² are the same and/or B¹ and B² are the same.

In groups A¹, A², B¹, and B², integers p1to p4 (i.e., p1, p2, p3, and p4), q1 to q6 (i.e., q1, q2, q3, q4, q5, and q6), r1, r2, and s1 to s4 (i.e., s1, s2, s3, and s4) are each independently an integer of 1 to 5 (e.g., 1, 2, 3, 4, or 5). In some embodiments, p1 to p4 (i.e., p1, p2, p3, and p4), q1 to q6 (i.e., q1, q2, q3, q4, q5, and q6), r1, r2, and/or s1 to s4 are each independently an integer of 1 to 3 (e.g., 1, 2, or 3). In certain embodiments, p1 to p4 (i.e., p1, p2, p3, and p4), q1 to q6 (i.e., q1, q2, q3, q4, q5, and q6), and/or s1 to s4 (i.e., s1, s2, s3, and s4) are each 2. In some embodiments, p1 to p4 (i.e., p1, p2, p3, and p4) and/or q1 to q6 (i.e., q1, q2, q3, q4, q5, and q6) are each 2, and r1, r2, and s1 to s4 (i.e., s1, s2, s3, and s4) are each 1.

Each instance of R² can be hydrogen or a C₁-C₁₂ (e.g., C₁-C₈, C₁-C₆, or C₁-C₃) alkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, C₃-C₁₂ (e.g., C₃-C₈, C₃-C₆, or C₃-C₅) cycloalkenyl group, or R² is combined with a second R² so as to form a heterocyclic group. In some embodiments, R² is hydrogen or a C₁-C₁₂ alkyl (e.g., a C₁-C₁₀ alkyl group; a C₁-C₈ alkyl group; a C₁-C₆ alkyl group; a C₁-C₄ alkyl group, a C₁-C₃ alkyl group, or a C₁ or C₂ alkyl group) that can be linear or branched. In certain embodiments, R² is methyl. In other embodiments, R² can be hydrogen. Each R² is independently chosen and can be the same or different. In some embodiments, each IV in a given is the same (e.g., all methyl or all hydrogen).

Each instance of R⁴ is independently —C(O)O—, —C(O)NH—, or —S(O)(O)—. In some embodiments, each instance of R⁴ is independently —C(O)O— or —C(O)NH—. In certain embodiments, each instance of R⁴ is —C(O)O—. In certain embodiments, each instance of R⁴ is —C(O)NH—.

Each instance of R⁵ is independently an alkyl group, cycloalkyl group, alkenyl group, cycloalkenyl group, aryl group, heteroalkyl group, heterocyclic group, or combination thereof, optionally comprising from 2 to 8 tertiary amines or a substituent comprising a tissue-specific or cell-specific targeting moiety. R⁵ can comprise from about 2 to about 50 carbon atoms (e.g., from about 2 to about 40 carbon atoms, from about 2 to about 30 carbon atoms, from about 2 to about 20 carbon atoms, from about 2 to about 16 carbon atoms, from about 2 to about 12 carbon atoms, from about 2 to about 10 carbon atoms, or from about 2 to about 8 carbon atoms). In some embodiments, R⁵ is a heteroalkyl group comprising from 2 to 8 (i.e., 2, 3, 4, 5, 6, 7, or 8) tertiary amines. The tertiary amines can be part of the heteroalkyl backbone (i.e., the longest continuous chain of atoms in the heteroalkyl group, or a pendant substituent. Thus, for instance, the heteroalkyl group comprising the tertiary amines can provide an alkylamino group, amino alkyl group, alkylaminoalkyl group, aminoalkylamino group, or the like comprising 2 to 8 tertiary amines.

In some embodiments, each R⁵ is independently selected from:

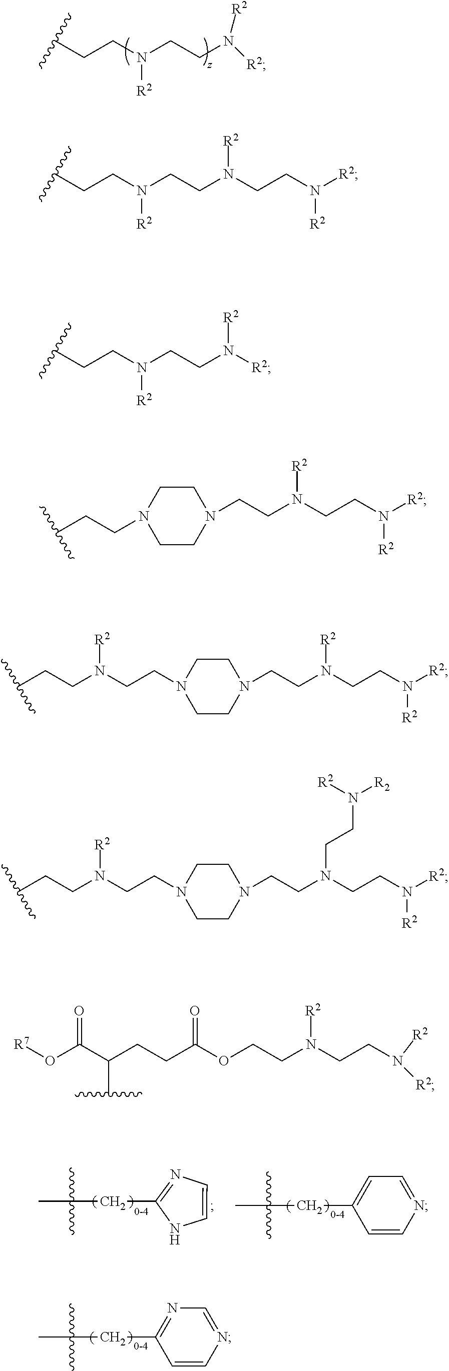

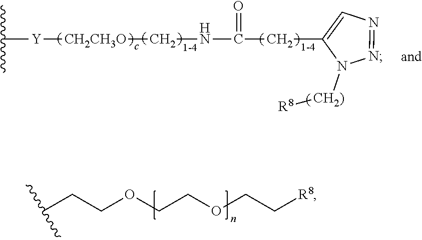

wherein

each instance of R² is as described above;

R⁷ is a C₁-C₅₀ alkyl group, alkenyl group, cycloalkyl group, or cycloalkenyl group optionally substituted with one or more amines;

z is an integer from 1 to 5;

c is an integer from 0 to 50;

Y is optionally present and is a cleavable linker;

n is an integer from 0 to 50; and

R⁸ is a tissue-specific or cell-specific targeting moiety. C₁-C₁₂ alkyl group, alkenyl group, cycloalkyl group, or cycloalkenyl group.

R⁷ can be a C₁-C₅₀ (e.g., C₁-C₄₀, C₁-C₃₀, C₁-C₂₀, C₁-C₁₀, C₄-C_{12,} or C₆-C₈) alkyl group, alkenyl group, cycloalkyl group, or cycloalkenyl group optionally substituted with one or more amines. In some embodiments, R⁷ is a C₄-C₁₂, such as a C₆-C_{8,} alkyl group, alkenyl group, cycloalkyl group, or cycloalkenyl group optionally substituted with one or more amines. In some embodiments, R⁷ is substituted with one or more amines. In certain embodiments, R⁷ is substituted with 2 to 8 (i.e., 2, 3, 4, 5, 6, 7, or 8) tertiary amines. The tertiary amines can be a part of the alkyl group (i.e., encompassed in the alkyl group backbone) or a pendant substituent.

Each instance of Y is optionally present. As used herein, the phrase “optionally present” means that a substituent designated as optionally present can be present or not present, and when that substituent is not present, the adjoining substituents are bound directly to each other. When Y is present, Y is a cleavable linker. As used herein, the phrase “cleavable linker” refers to any chemical element that connects two species that can be cleaved as to separate the two species. For example, the cleavable linker can be cleaved by a hydrolytic process, photochemical process, radical process, enzymatic process, electrochemical process, or a combination thereof. Exemplary cleavable linkers include, but are not limited to:

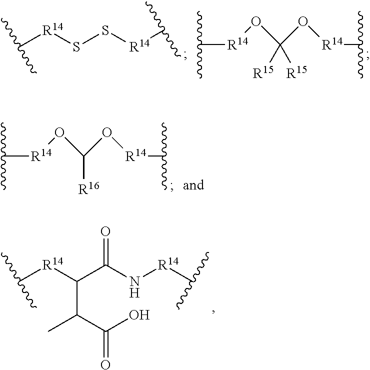

wherein each occasion of R¹⁴ independently is a C₁-C₄ alkyl group, each occasion of R¹⁵ independently is hydrogen, an aryl group, a heterocyclic group (e.g., aromatic or non-aromatic), a C₁-C₁₂ alkyl group, alkenyl group, cycloalkyl group, or cycloalkenyl group, and R¹⁶ is a six-membered aromatic or heteroaromatic group optionally substituted with one or more —OCH₃, —NHCH₃, —N(CH₃)₂, —SCH₃, —OH, or a combination thereof.

In some embodiments, each of A¹ and A² is independently a group of formula —(CH₂)ₚ₁—[NH—(CH₂)ₐ₁—]ᵣ₁NH₂ or —(CH₂)ₚ₁—[NH—(CH₂)_{q1}—]ᵣ₁NHCH₃, or a group —(CH₂)₂—NH—(CH₂)₂—NH₂ or —(CH₂)₂—NH—(CH₂)₂—NHCH₃ or —(CH₂)₂—NH—(CH₂)₂—NH₂. In some embodiments, each of A¹ and A² is independently a group of formula —(CH₂)ₚ₁—[N(R²))—(CH₂)_{q1}—]ᵣ₁N(R²)₂ or —(CH₂)ₚ₁—[N(R²)—(CH₂)_{q1}—]ᵣ₁NH(R²), wherein R² is a methyl or ethyl; or a group —(CH₂)₂—N(CH₃)—(CH₂)₂—NH₂, or —(CH₂)₂—N(CH₃)—(CH₂)₂—NHCH₃, or —(CH₂)₂—N(CH₃)—(CH₂)₂—N(CH₃)_{2.}

In addition, or alternatively, each of B¹ and B² is a group of formula —(CH₂)ₚ₁—[NH—(CH₂)_{q1}—]ᵣ₁NH—(CH₂)₂—R⁴—R⁵, such as a group —(CH₂)₂—NH—(CH₂)₂—NH—(CH₂)₂—R⁴—R⁵, or a group —(CH₂)₂—NH—(CH₂)₂—NH—(CH₂)₂—C(O)—O—R⁵, wherein R⁴ and R⁵ are as described above.

In some embodiments, the polymer of Formula 1 does not have any B monomers (e.g., m³ and m⁴ are both 0). Thus, also provided is a polymer of having the structure of Formula 4:

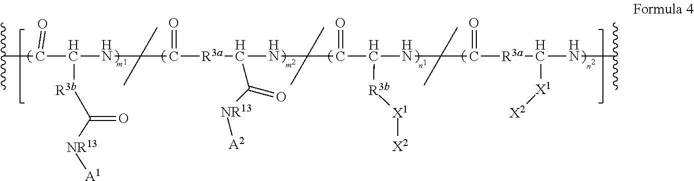

wherein:

each of m¹ and m² is an integer from 0 to 1000 (e.g., 0 to 500, 0 to 200, 0 to 100, or 0 to 50), provided that the sum of m¹+m² is greater than 5 (e.g., 5-2000, 5-1000, 5-500, 5-100, or 5-50). In some embodiments, the sum of m¹+m² is greater than 10 or greater than 20 (e.g., 10-5000, 10-2000, 10-1000, 10-500, 10-100, or 10-50; or 20-5000, 20-2000, 20-1000, 20-500, 20-100, or 20-50). Furthermore, each of n¹ and n² is an integer from 0 to 1000 (e.g., 0 to 500, 0 to 200, 0 to 100, 0 to 50, or 0-25), provided that the sum of n¹+n² is greater than 2 (e.g., 2-2000, 2-1000, 2-500, 2-200, 2-100, 2-50, or 2-25). In some embodiments, the sum of n¹+n² is greater than 5 or greater than 10 (e.g., 5-2000, 5-1000, 5-500, 5-200, 5-100, 5-50, or 5-25; or 10-2000, 10-1000, 10-500, 10-200, 10-100, 10-50, or 10-25).

In some embodiments of the polymer of Formula 4, A¹ and A² are each independently a group of formula

—(CH₂)ₚ₁—[NR²—(CH₂)_{q1}—]ᵣ₁NR²₂;

—(CH₂)ₚ₂—N[—(CH₂)_{q2}—NR²₂]₂;

—(CH₂)ₚ₃—N{[—(CH₂)_{q3}—NR²₂][—(CH₂)_{q4}—NR²—]ᵣ₂R²}; or

—(CH₂)ₚ₄—N{—(CH₂)_{q5}—N[—(CH₂)_{q6}—NR²₂]₂}₂,

wherein p1 to p4, q1 to q6, and r1 and r2 are each independently an integer of 1 to 5 (e.g., an integer of 1-3); and each instance of R² is independently hydrogen or a C₁-C₁₂ (e.g., C₁-C₈, C₁-C₆, or C₁-C₃) alkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, C₃-C₁₂ (e.g., C₃-C₈, C₃-C₆, or C₃-C₅) cycloalkenyl group. In some embodiments, each nitrogen in group A¹ and A² containing R² substituents is a tertiary amine, with the exception that the terminal amine can be a primary, secondary, or tertiary amine or, in some embodiments, a secondary or tertiary amine. By way of further illustration, each of A¹ and A² can be —(CH₂)₂—NR²—(CH₂)₂—NR²₂, wherein each instance of R² is independently a hydrogen, alkyl group, alkenyl group, cycloalkyl group, or cycloalkenyl group as described above, particularly an aklyl such as methyl or ethyl, optionally wherein each amine is a tertiary amine with the exception that the terminal amine is a secondary or tertiary amine.

Specific non-limiting examples of groups A¹ and A² include, for instance, —NH—CH₂—CH₂—N(CH₃)—CH₂—CH₂—N(CH₃)₂; —N(CH₃)—CH₂—CH₂—N(CH₃)—CH₂—CH₂—N(CH₃)₂; —NH—CH₂—CH₂—N(CH₃)—CH₂—CH₂—N(CH₃)—CH₂—CH₂—N(CH₃)₂; —N(CH₃)—CH₂—CH₂—N(CH₃)—CH₂—CH₂—N(CH₃)—CH₂—CH₂—N(CH₃)₂; —NH—CH₂—CH₂—N(CH₃)—CH₂—CH₂—NH(CH₃); —N(CH₃)—CH₂—CH₂—N(CH₃)—CH₂—CH₂—NH(CH₃); —NH—CH₂—CH₂—N(CH₃)—CH₂—CH₂—N(CH₃)—CH₂—CH₂—NH(CH₃); —N(CH₃)—CH₂—CH₂—N(CH₃)—CH₂—CH₂—N(CH₃)—CH₂—CH₂—NH(CH₃).

All other aspects of the polymer of Formula 4 are as described with respect to Formula 1, including all embodiments thereof with respect to substituents of Formula 4. Thus, for instance, in some embodiments of Formula 4, each instance of R¹³ can be any group as described with respect to Formula 1, including specific embodiments in which R¹³ is hydrogen or methyl, and each instance of R^{3a} and R^{3b} can be any group as described with respect to Formula 1, including embodiments wherein R^{3a} and R³ are methylene or ethylene. Similarly, X¹ and X² can be any group as described with respect to Formula 1, including embodiments wherein X¹ is —C(O)NR¹³— or —C(O)O— and/or one or more (or all) X² groups can be independently a C₁-C₈ (e.g., C₁-C₆, C₁-C₄, C₁-C₃, C₂-C₈, or C₂-C₆) alkyl group.

In some embodiments, the polymer has structure of Formula 1A:

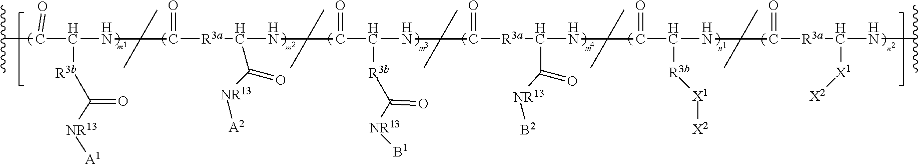

wherein

Q is of formula:

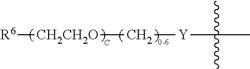

c is an integer from 0 to 50;

Y is optionally present and is a cleavable linker;

each of m¹, m², m³, and m⁴ is an integer from 0 to 1000, provided that the sum of m¹+m²+m³+m⁴ is greater than 5;

each of n¹ and n² is an integer from 0 to 1000, provided that the sum of n¹+n² is greater than 2;

the symbol “/” indicates that the units separated thereby are linked randomly or in any order;

R¹ is hydrogen, an aryl group, a heterocyclic group, a C₁-C₁₂ (e.g., C₁-C₈, C₁-C₆, or C₁-C₃) alkyl or heteroalkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, or C₃-C₁₂ (e.g., C₃-C₈, C₃-C₆, or C₃-C₅) cycloalkenyl group, optionally substituted with one or more substituents; and

R⁶ is hydrogen, an amino group, an aryl group, a heterocyclic group, a C₁-C₁₂ (e.g., C₁-C₈, C₁-C₆, or C₁-C₃) alkyl or heteroalkyl group, a C₂-C₁₂ (e.g., C₂-C₈, C₂-C₆, or C₂-C₃) alkenyl group, a C₃-C₁₂ (e.g., C₃-C₈, C₃-C₆, or C₃-C₅) cycloalkyl group, or a C₃-C₁₂ (e.g., C₃-C₈, C₃-C₆, or C₃-C₅) cycloalkenyl group, optionally substituted with one or more amines; or a tissue-specific or cell-specific targeting moiety.

All other aspects of Formula 1A are as described with respect to Formula 1, above, including any and all embodiments thereof

In some embodiments, the polymer has structure of Formula 1B:

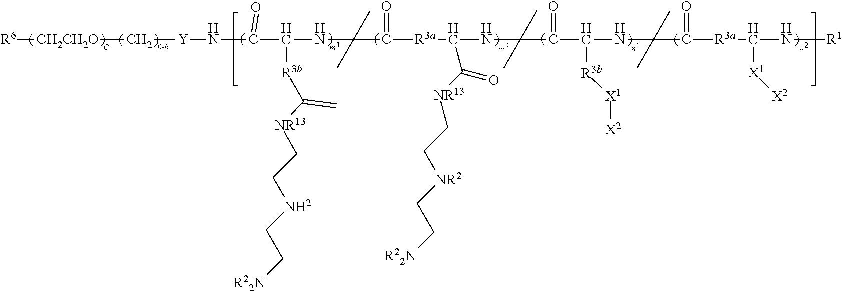

wherein

c is an integer from 0 to 50;

Y is optionally present and is a cleavable linker;

each of m¹ and m² is an integer from 0 to 1000, provided that the sum of m¹+m² is greater than 5;

each of n¹ and n² is an integer from 0 to 1000, provided that the sum of n¹+n² is greater than 2;

the symbol “/” indicates that the units separated thereby are linked randomly or in any order;

R¹ is hydrogen, an aryl group, a heterocyclic group, a C₁-C₁₂ (e.g., C₁-C₈, C₁-C₆, or C₁-C₃) alkyl or heteroalkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, or C₃-C₁₂ (e.g., C₃-C₈, C₃-C₆, or C₃-C₅) cycloalkenyl group optionally substituted with one or more substituents; and

R⁶ is hydrogen, an amino group, an aryl group, a heterocyclic group, a C₁-C₁₂ (e.g., C₁-C₈, C₁-C₆, or C₁-C₃) alkyl or heteroalkyl group, a C₂-C₁₂ (e.g., C₂-C₈, C₂-C₆, or C₂-C₃) alkenyl group, a C₃-C₁₂ (e.g., C₃-C₈, C₃-C₆, or C₃-C₅) cycloalkyl group, or a C₃-C₁₂ (e.g., C₃-C₈, C₃-C₆, or C₃-C₅) cycloalkenyl group optionally substituted with one or more amines; or a tissue-specific or cell-specific targeting moiety.

All other aspects of Formula 1B are as described with respect to Formula 1 and Formula 4, including any and all embodiments thereof.

In some embodiments, the polymer has structure of Formula 1C:

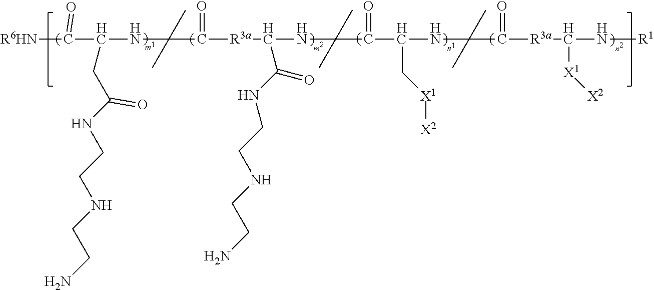

wherein

each of m¹ and m² is an integer from 0 to 1000, provided that the sum of m¹+m² is greater than 5;

each of n¹ and n² is an integer from 0 to 1000, provided that the sum of n¹+n² is greater than 2;

the symbol “/” indicates that the units separated thereby are linked randomly or in any order;

R¹ is hydrogen, an aryl group, a heterocyclic group, a C₁-C₁₂ (e.g., C₁-C₈, C₁-C₆, or C₁-C₃) alkyl or heteroalkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, or C₃-C₁₂ (e.g., C₃-C₈, C₃-C₆, or C₃-C₅) cycloalkenyl group optionally substituted with one or more substituents; and

R⁶ is hydrogen, an amino group, an aryl group, a heterocyclic group, a C₁-C₁₂ (e.g., C₁-C₈, C₁-C₆, or C₁-C₃) alkyl or heteroalkyl group, a C₂-C₁₂ (e.g., C₂-C₈, C₂-C₆, or C₂-C₃) alkenyl group, a C₃-C₁₂ (e.g., C₃-C₈, C₃-C₆, or C₃-C₅) cycloalkyl group, or a C₃-C₁₂ (e.g., C₃-C₈, C₃-C₆, or C₃-C₅) cycloalkenyl group optionally substituted with one or more amines; or a tissue-specific or cell-specific targeting moiety. All other aspects of Formula 1C are as described with respect to Formula 1 and Formula 4 including any and all embodiments thereof.

In some embodiments, R¹ and/or R⁶ is a C₁-C₁₂ alkyl (e.g., a C₁-C₁₀ alkyl group; a C₁-C₈ alkyl group; a C₁-C₆ alkyl group; a C₁-C₄ alkyl group, a C₁-C₃ alkyl group, or a C₁ or C₂ alkyl group), which can be linear or branched, optionally substituted with one or more substituents. In certain embodiments, the heteroalkyl or alkyl group comprises or is substituted with one or more amines, for instance, from 2 to 8 (i.e., 2, 3, 4, 5, 6, 7, or 8) tertiary amines. The tertiary amines can be a part of the heteroalkyl backbone chain or pendant substituents.

The polymer can be any suitable polymer, provided the polymer comprises the foregoing polymer structure. In some embodiments, the polymer is a block copolymer comprising a polymer block having the structure of Formula 1 and one or more other polymer blocks (e.g., an ethylene oxide subunit, or a propylene oxide subunit). In other embodiments, the structure of Formula 1 is the only polymeric unit of the polymer, which can comprise any suitable end groups. In certain embodiments, the polymer further comprises a substituent comprising a tissue-specific or cell-specific targeting moiety.

In some embodiments, the polymer has structure of Formula 5A:

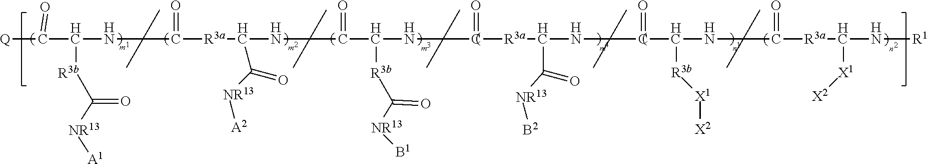

wherein

Q is of formula:

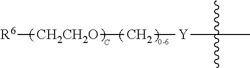

c is an integer from 2 to 200 (e.g., 2 to 150, 2 to 100, 2 to 50, 10 to 200, 10 to 150, 10 to 100, 10 to 50, 25 to 200, 25 to 150, 25 to 100, 25 to 50, 50 to 200, 50 to 150, or 50 to 100);

Y is optionally present and is a cleavable linker;

and all other substituents are as described with respect to Formulas 1, 1A-1C, and 4, including any and all embodiments thereof.

In some embodiments, the polymer has structure of Formula 5B:

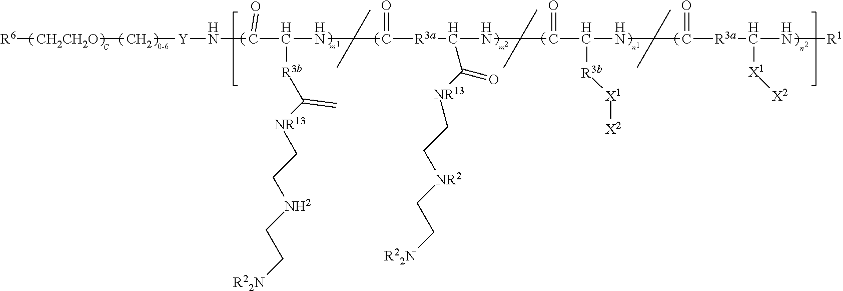

wherein

c is an integer from 2 to 200 (e.g., 2 to 150, 2 to 100, 2 to 50, 10 to 200, 10 to 150, 10 to 100, 10 to 50, 25 to 200, 25 to 150, 25 to 100, 25 to 50, 50 to 200, 50 to 150, or 50 to 100);

Y is optionally present and is a cleavable linker;

and all other substituents are as described with respect to Formulas 1, 1A-1C, and 4, including any and all embodiments thereof

Non-limiting examples of the polymers provided herein include, for instance:

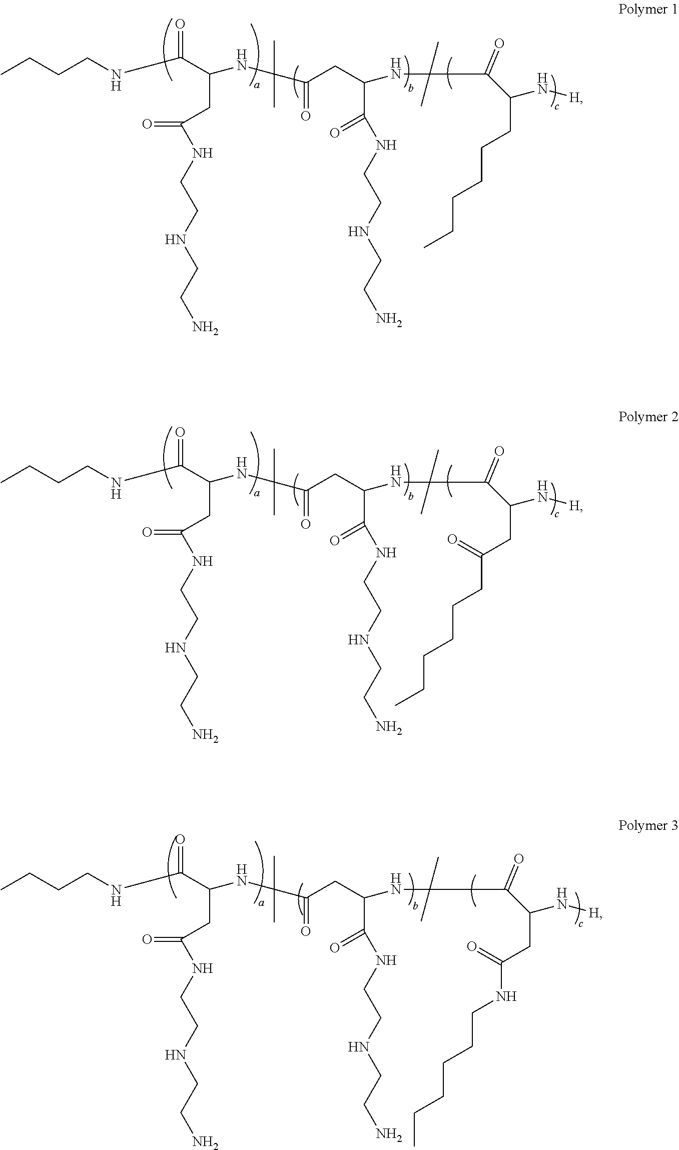

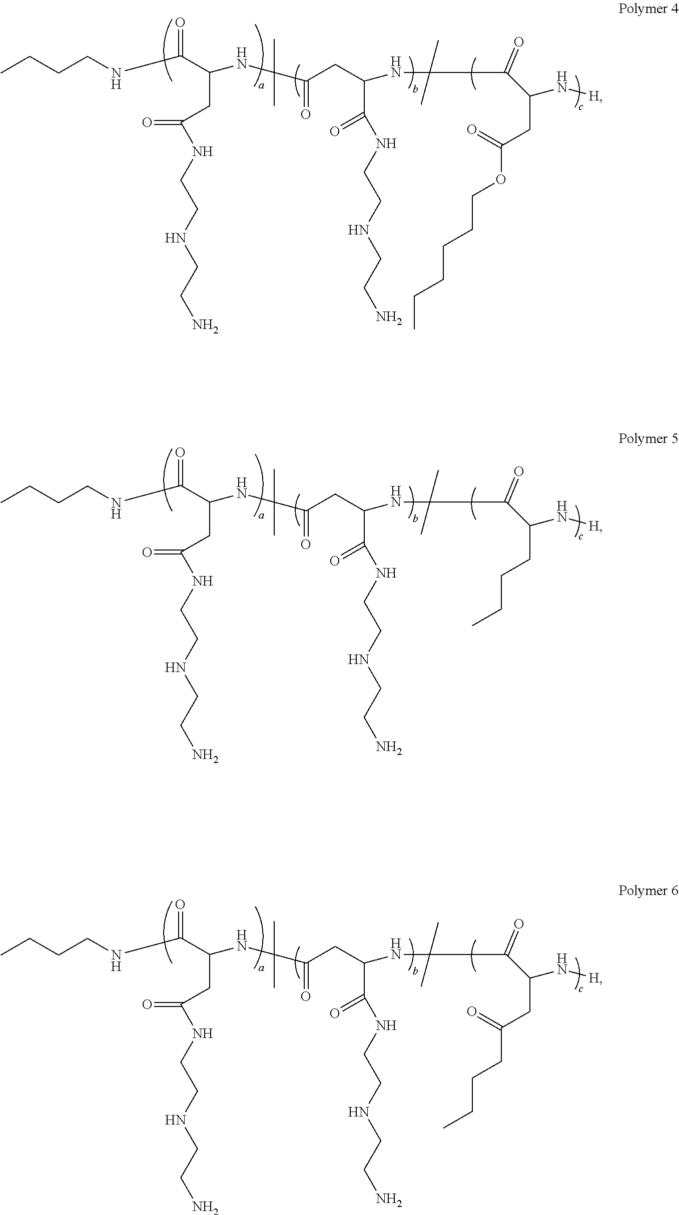

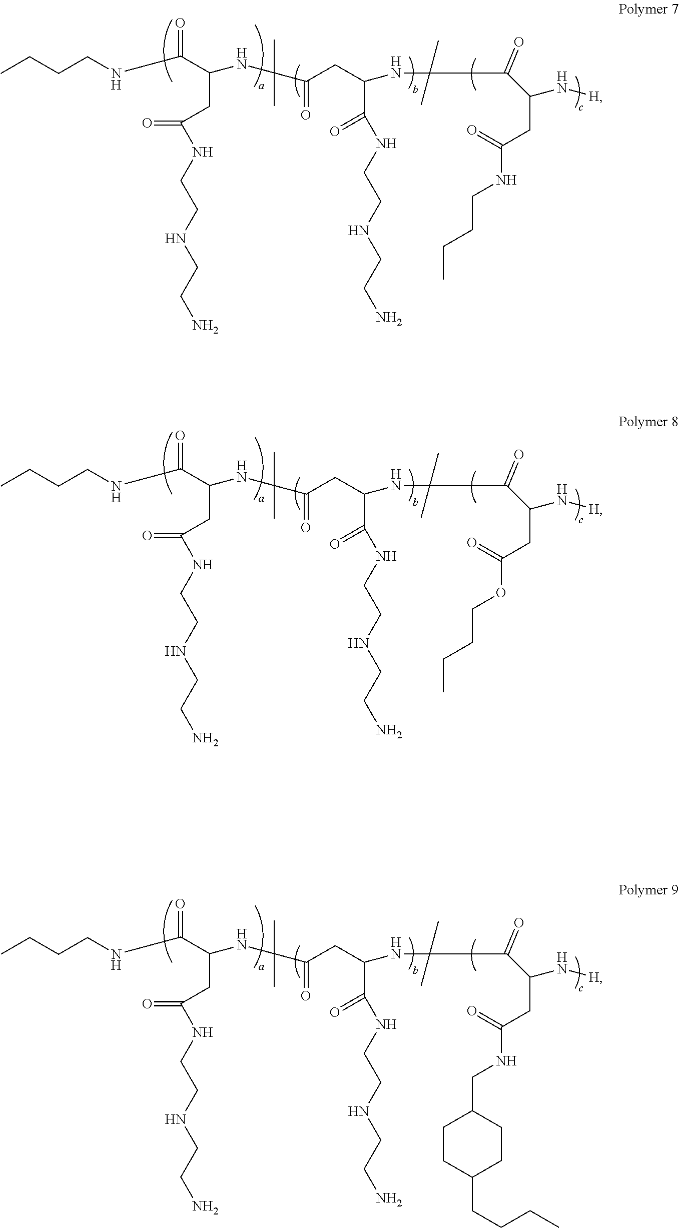

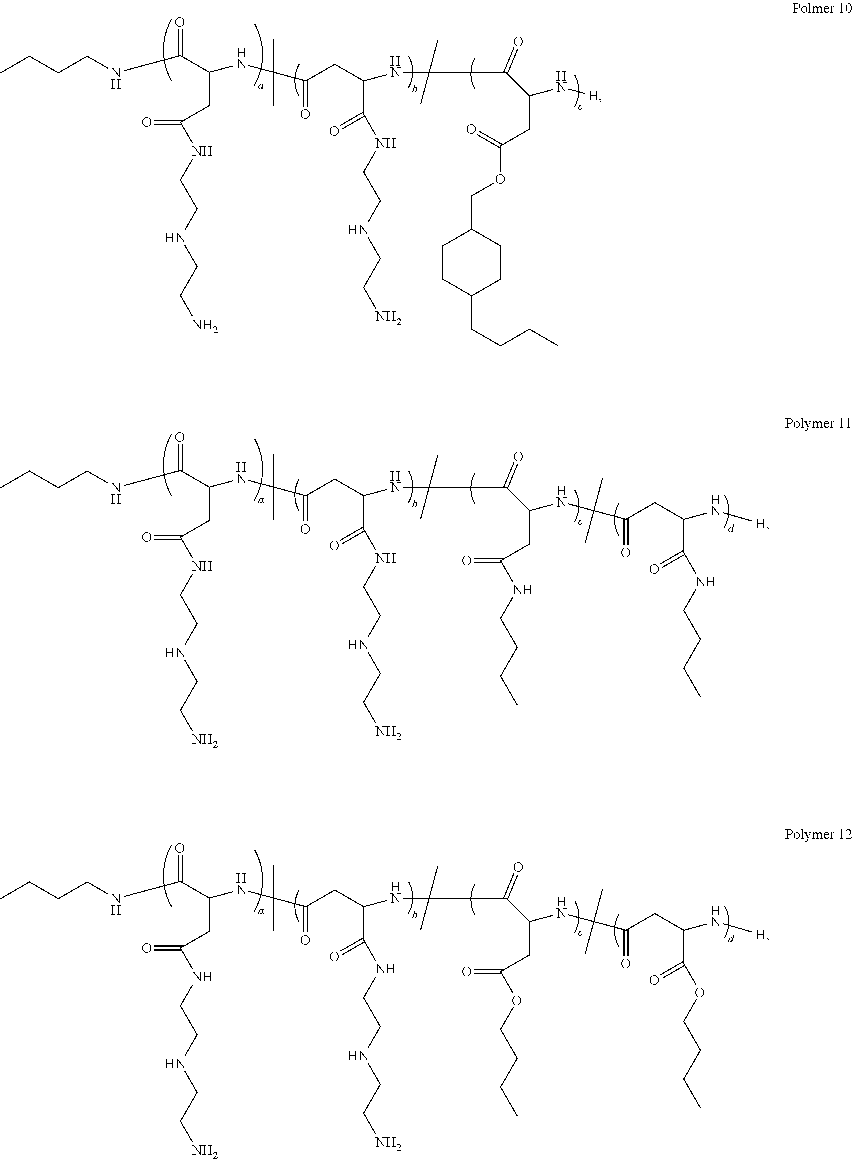

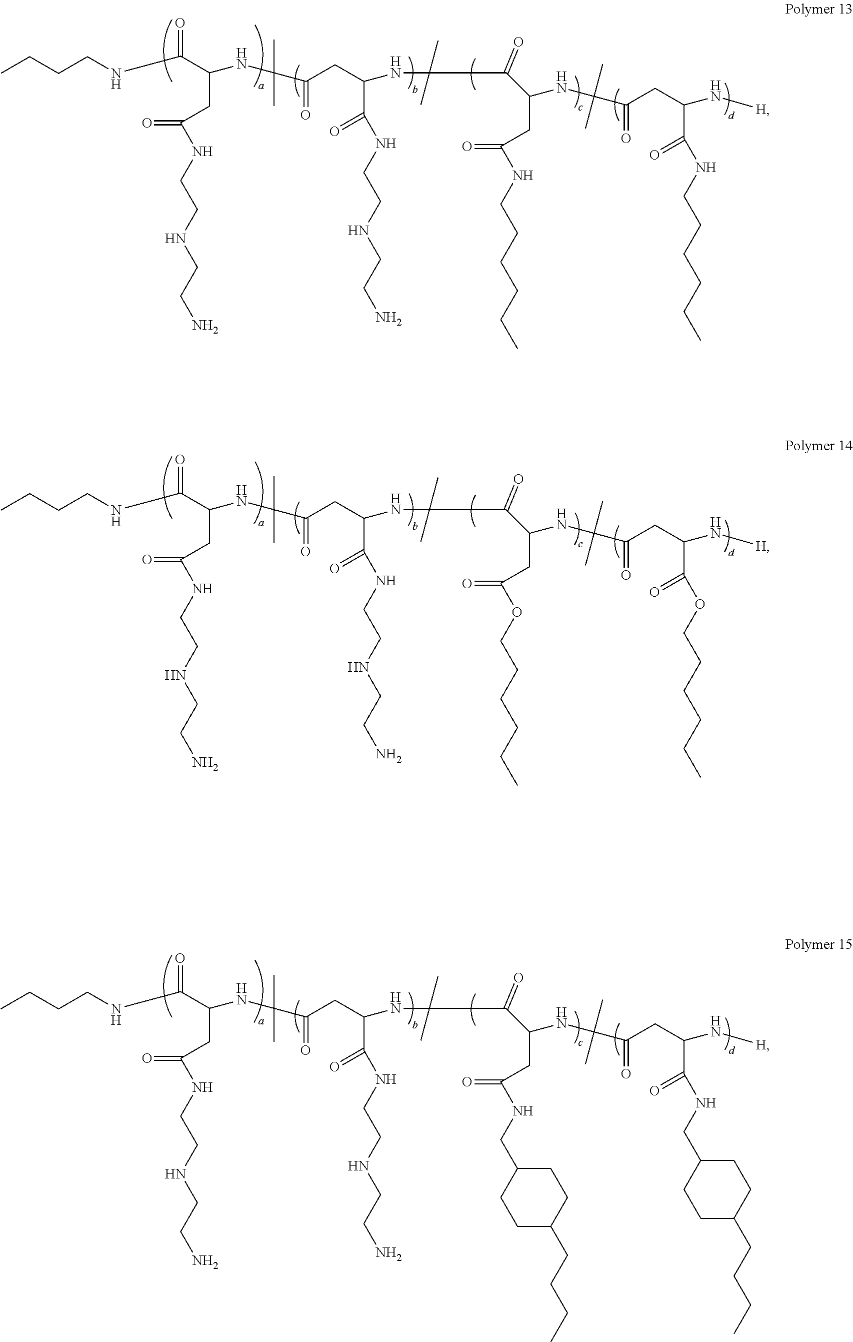

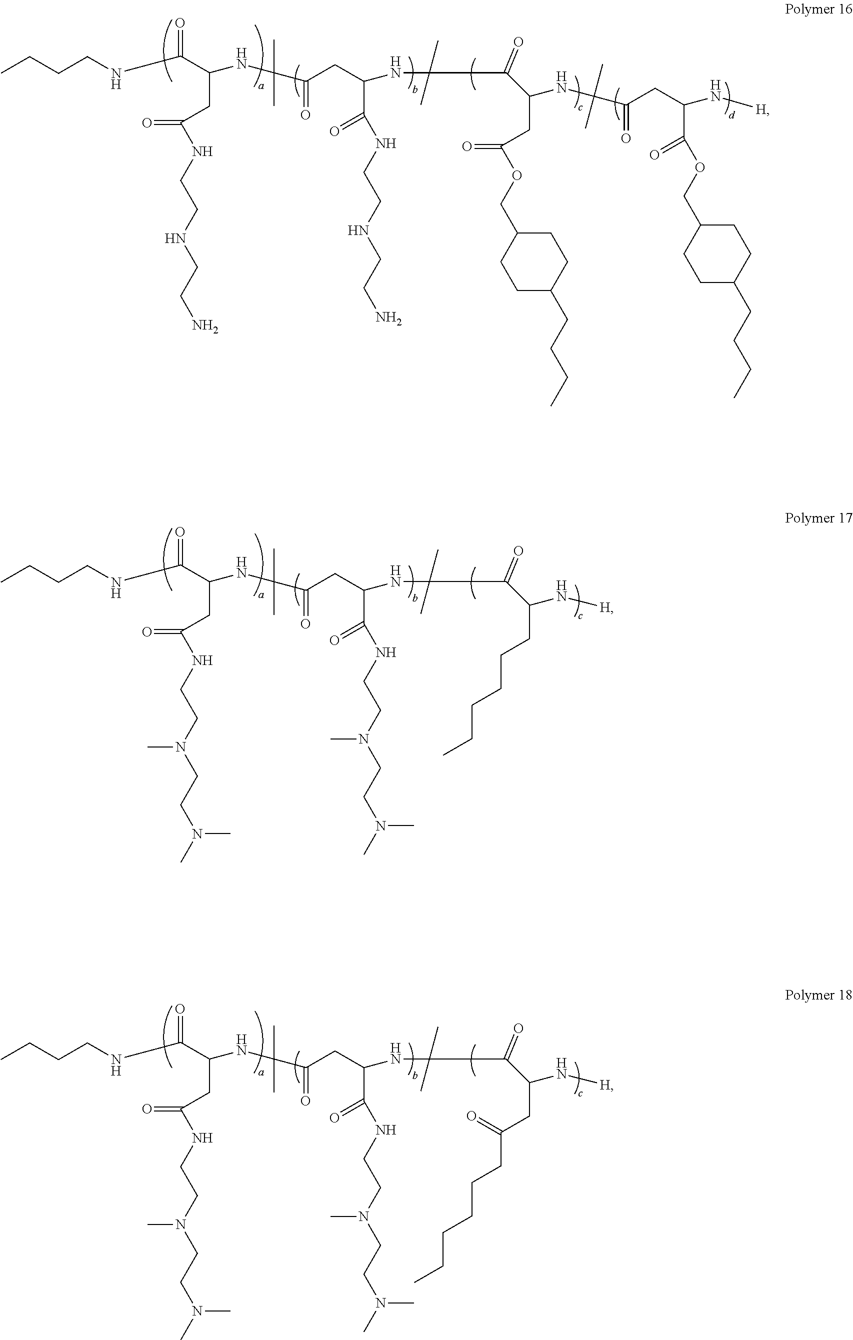

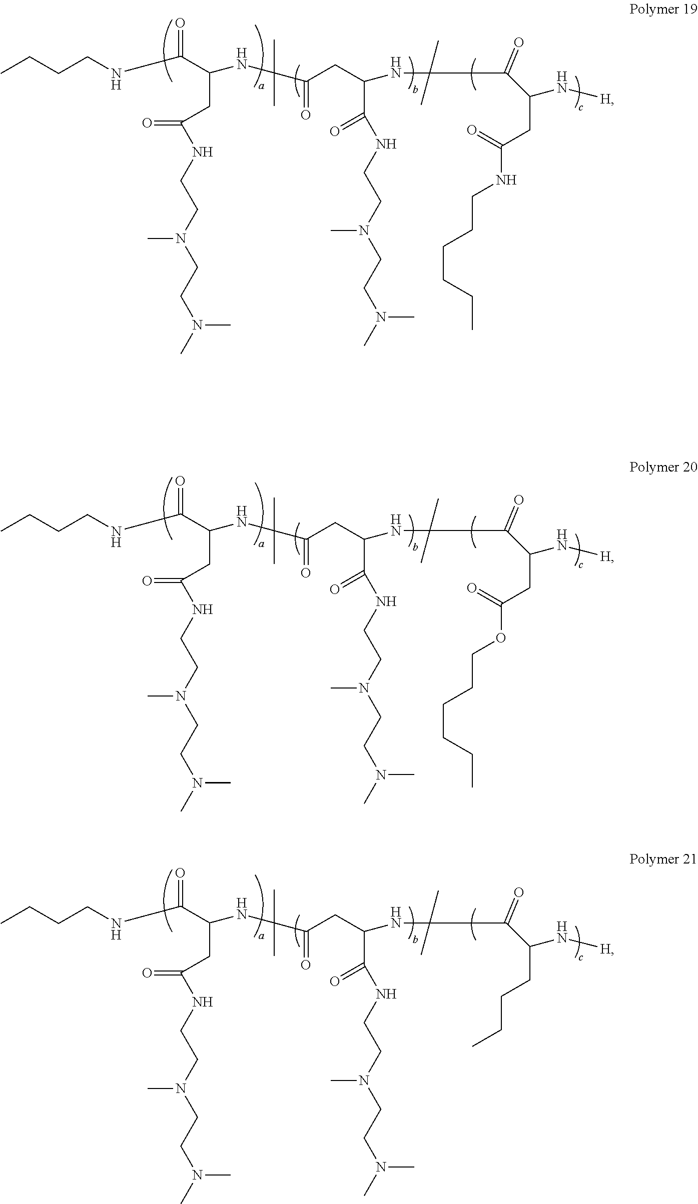

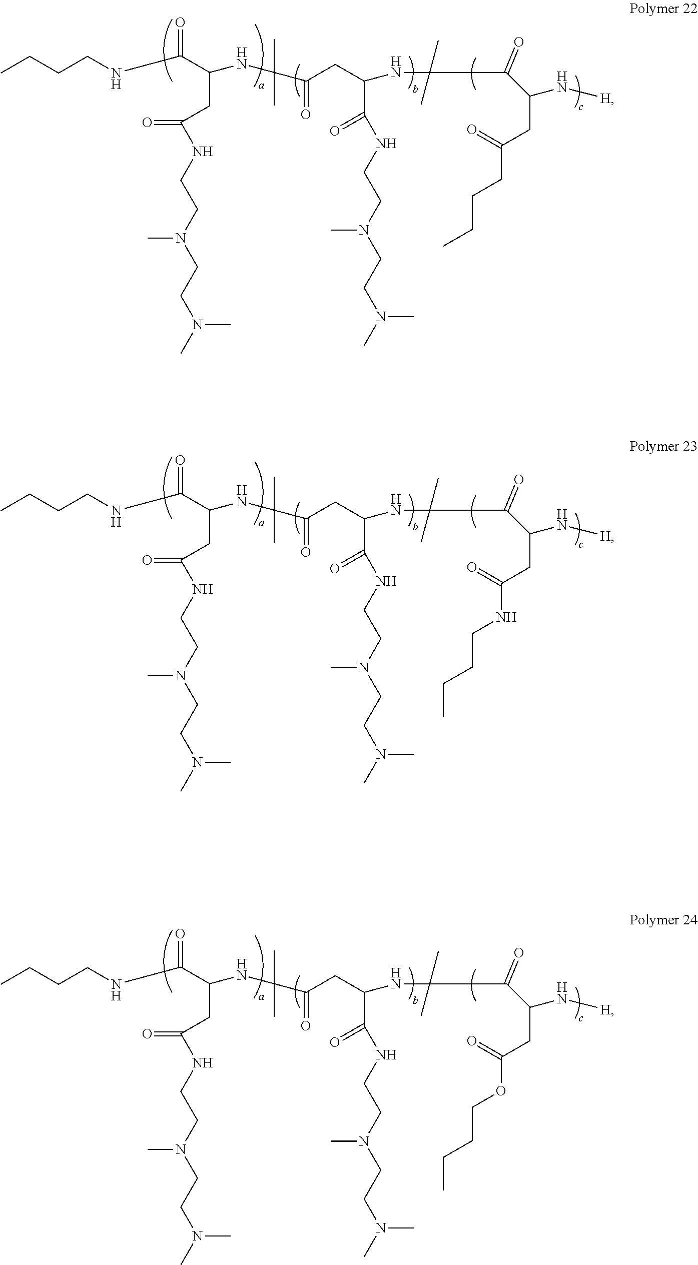

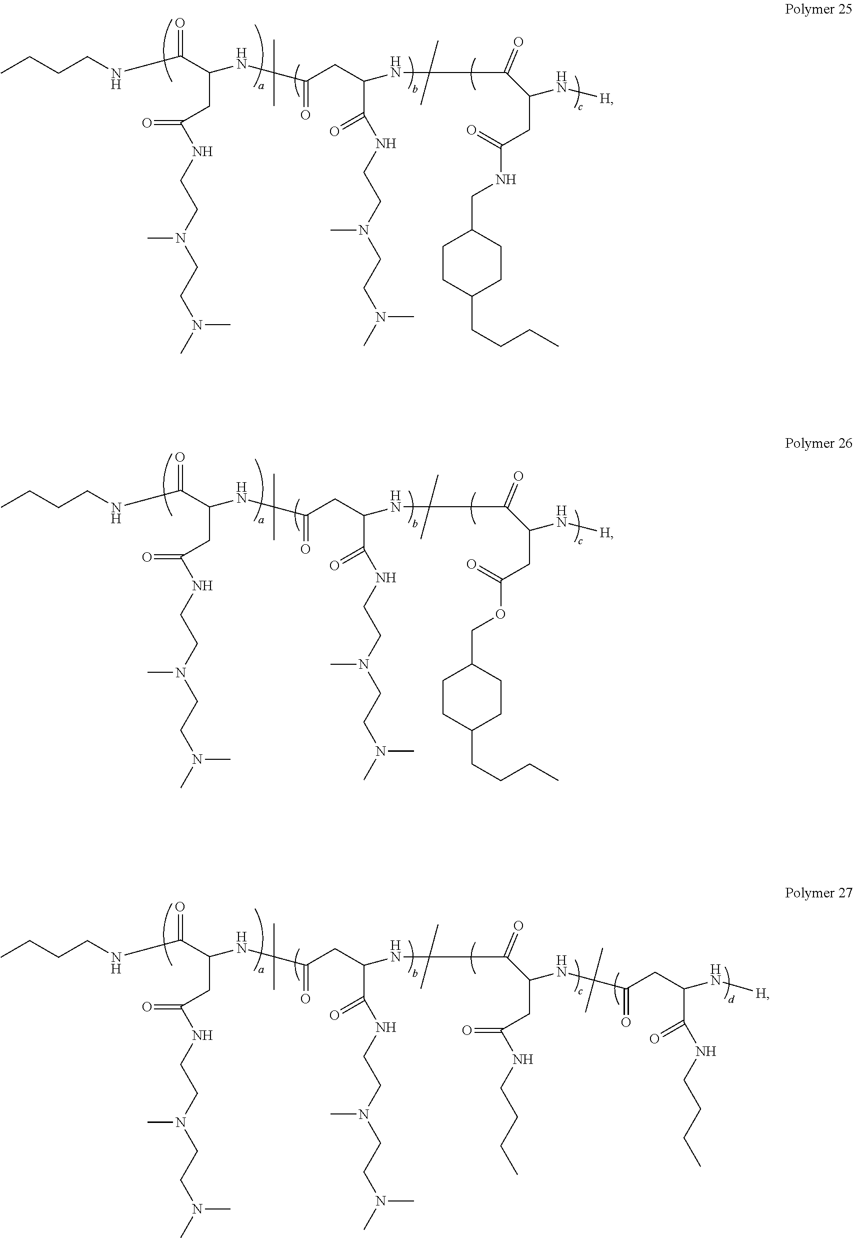

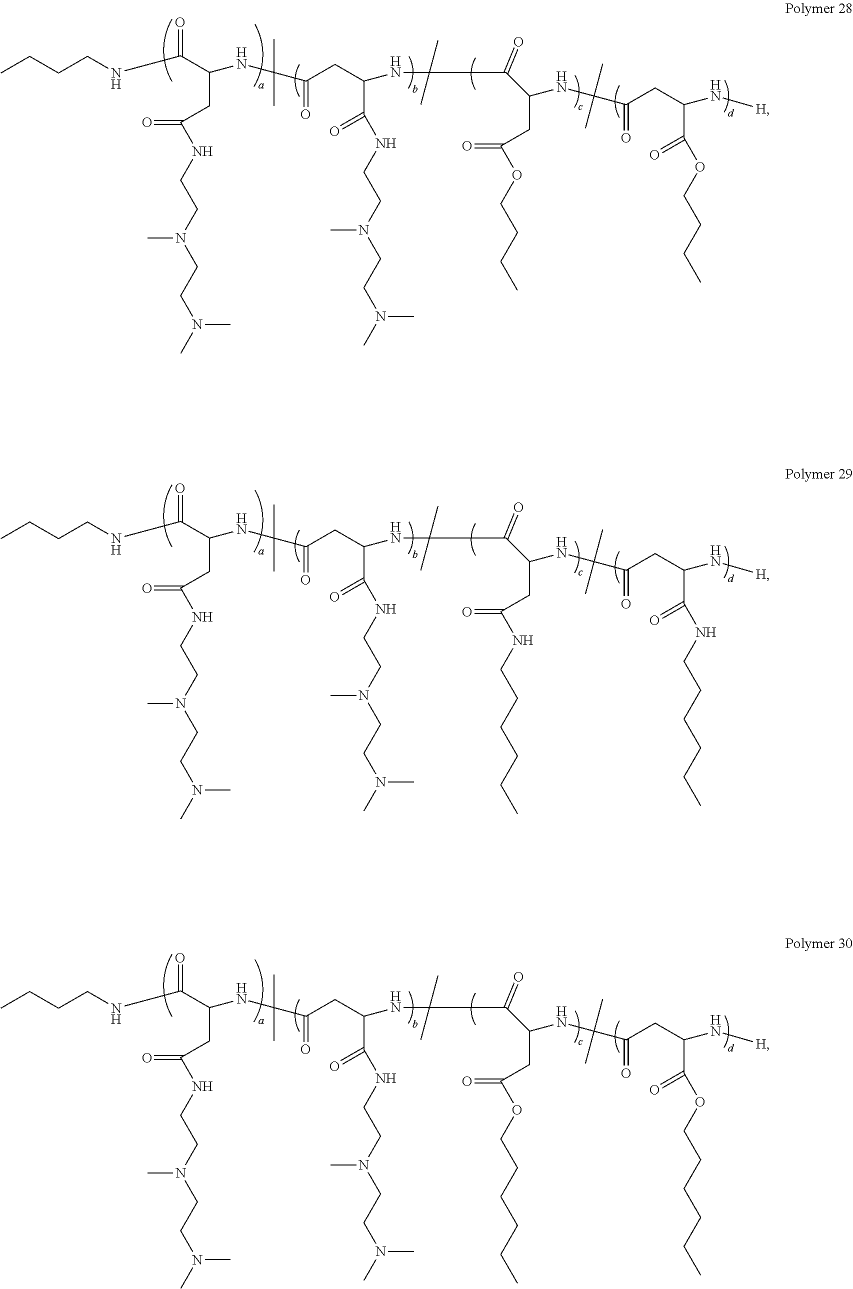

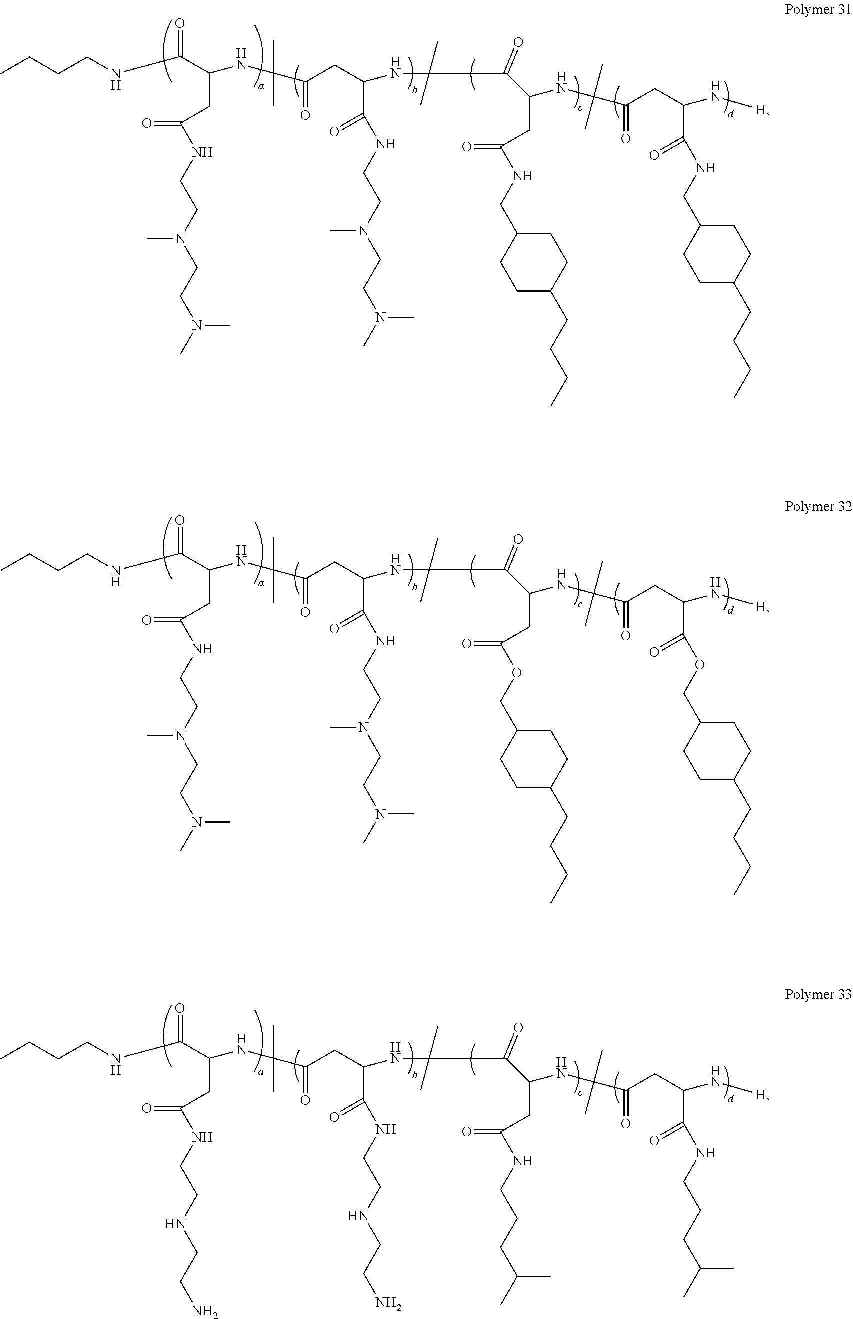

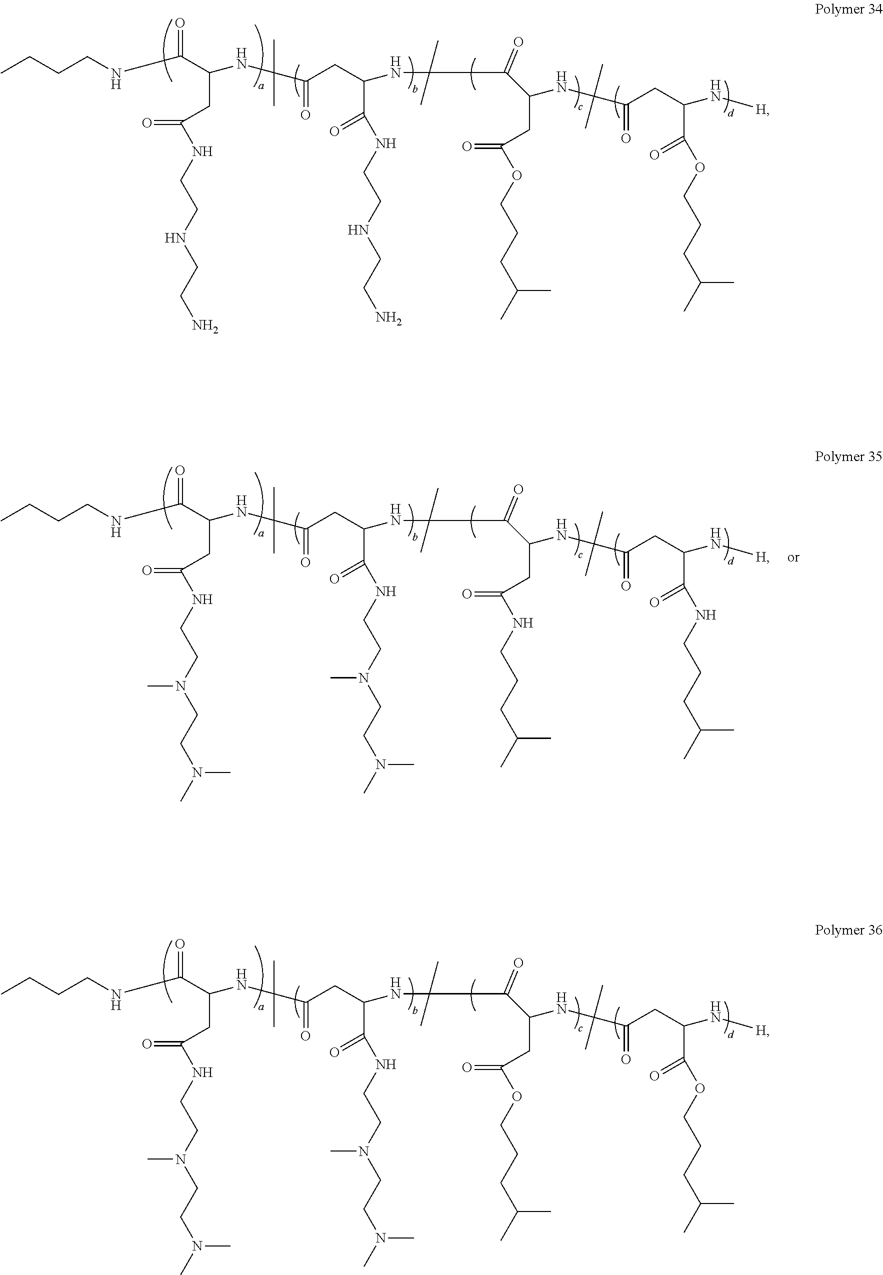

wherein (a+b) is from about 5 to about 65 (e.g., about 5 to about 50, about 5 to about 40, about 5 to about 30, about 5 to about 20, or about 5 to about 10) and (c+d) is from about 2 to about 60 (e.g., about 2 to about 50, about 2 to about 40, about 2 to about 30, about 2 to about 20, or about 2 to about 10). In other embodiments, (a+b) is about 45 and (c+d) is about 20. Again, the indication of the number of units (“a”, “b”, “c”, and “d”) in these exemplary polymers does not imply a block co-polymer structure; rather, these numbers indicate the number of units overall, which units can be randomly arranged as indicated by the “/” symbols in the formulas.

Additional specific examples of polymers provided by the present disclosure (e.g., polymers with ionic or ionizable groups) further include the following:

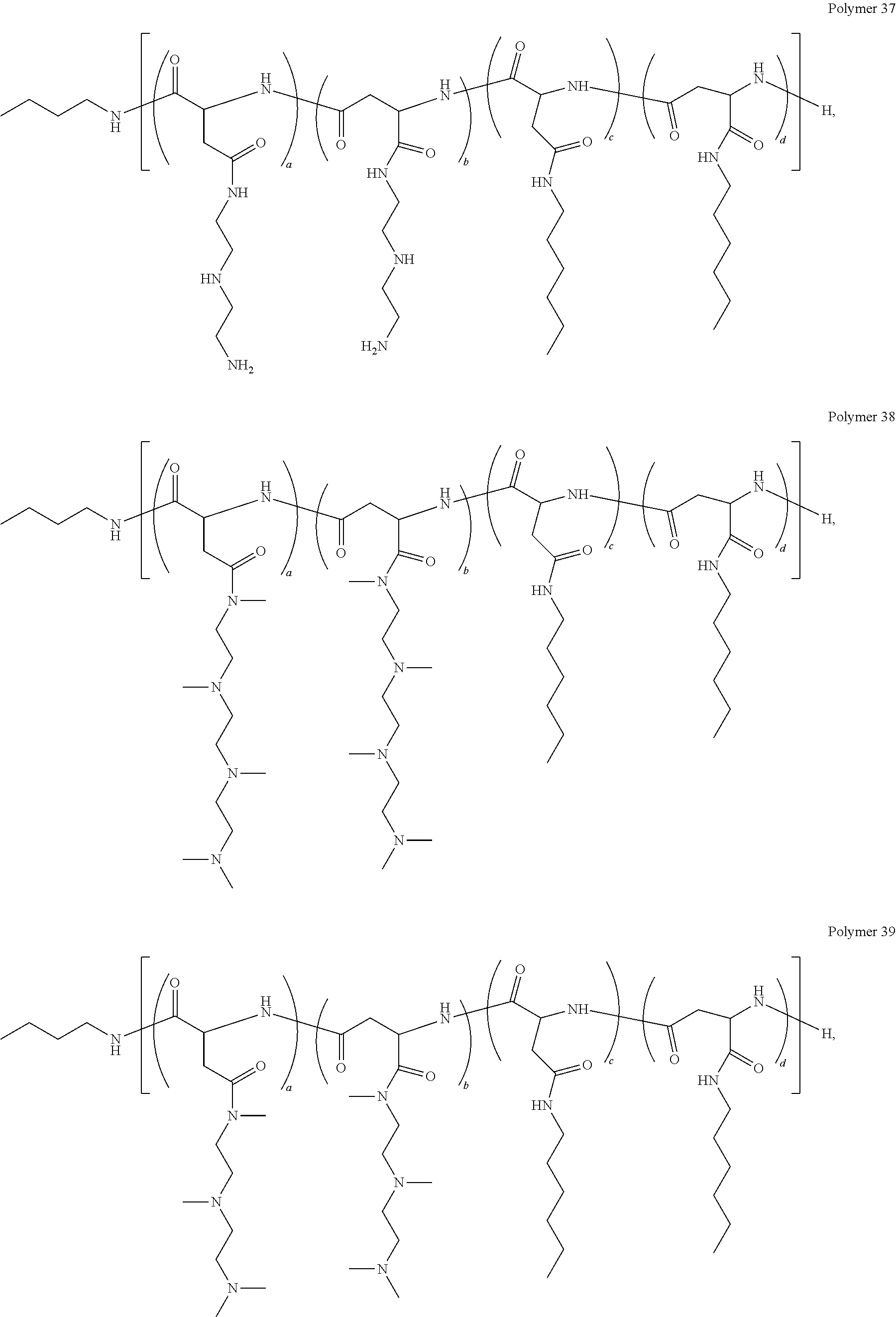

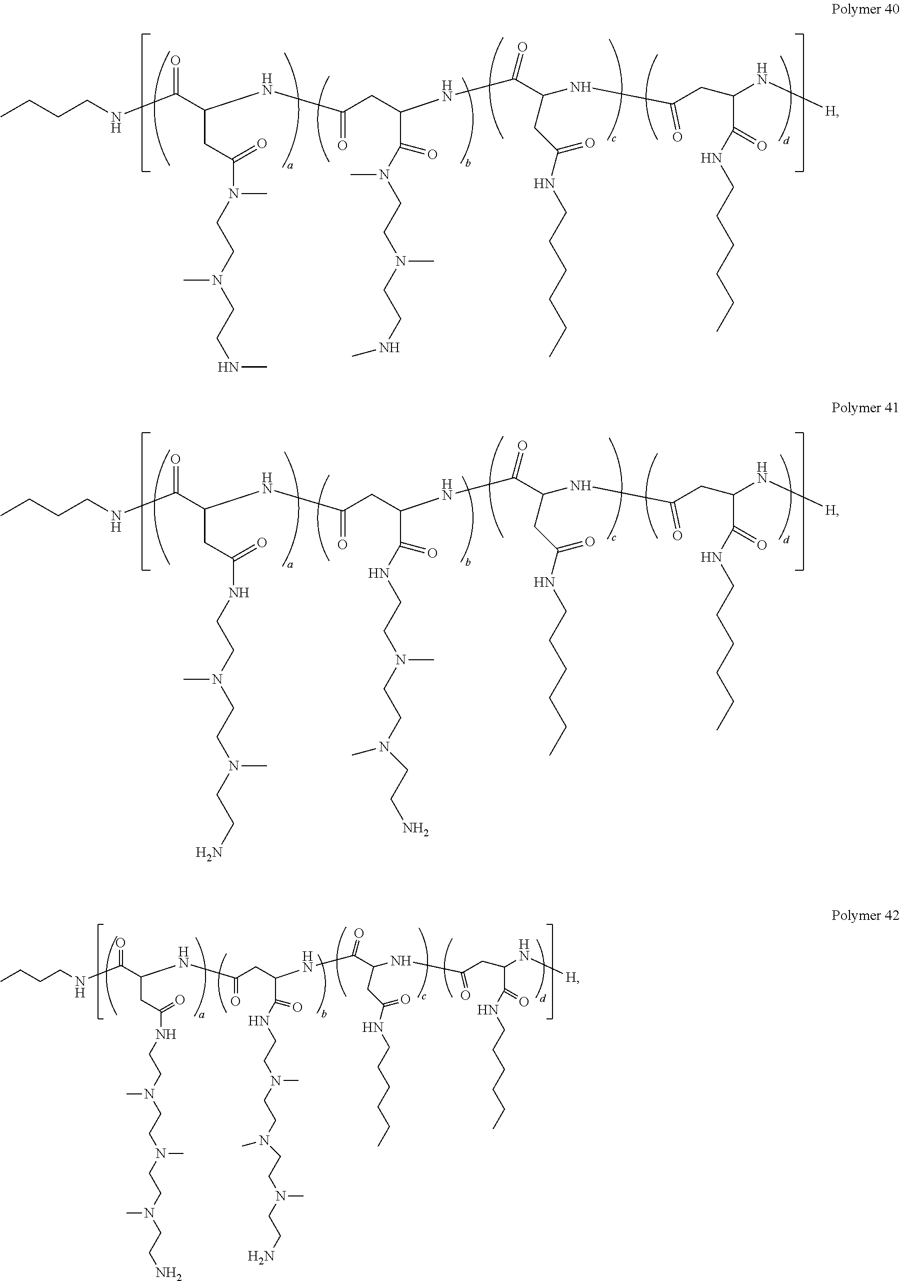

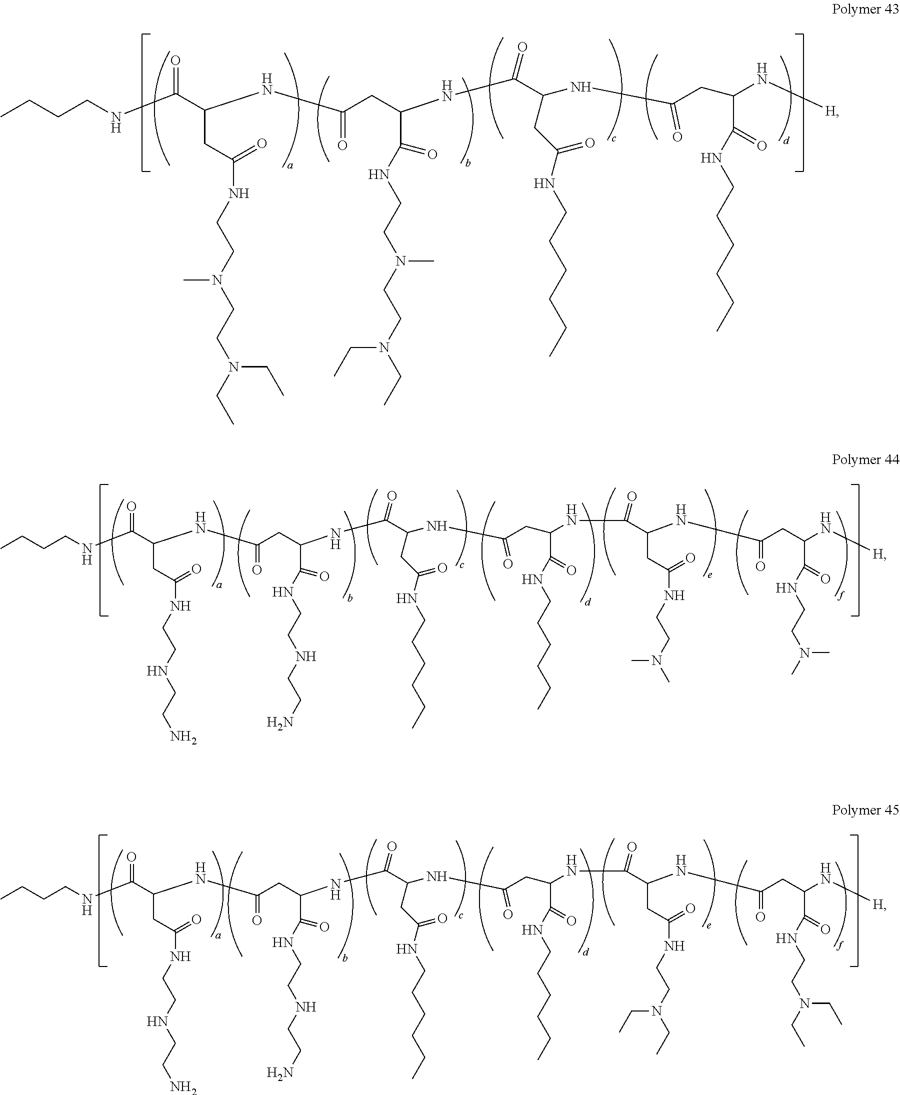

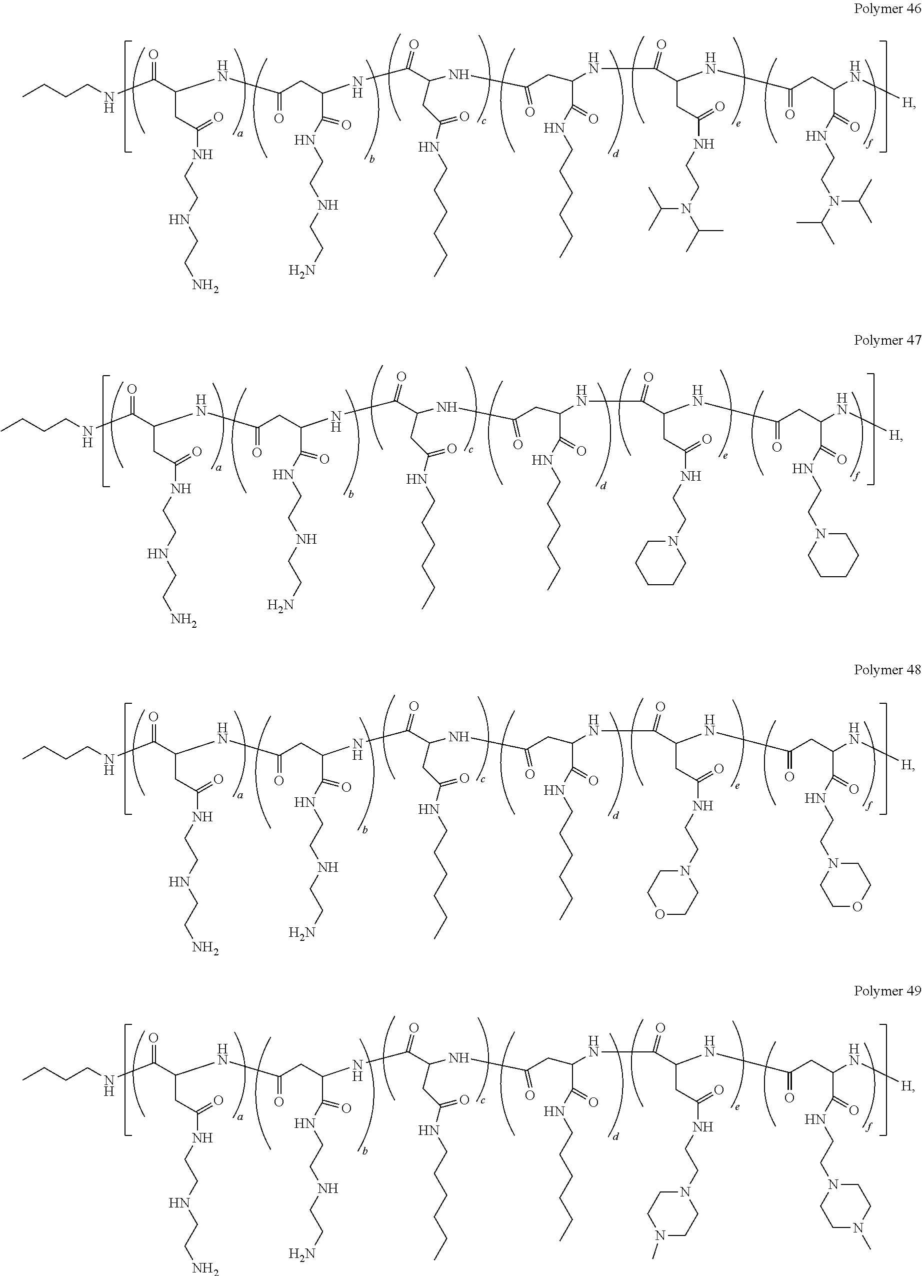

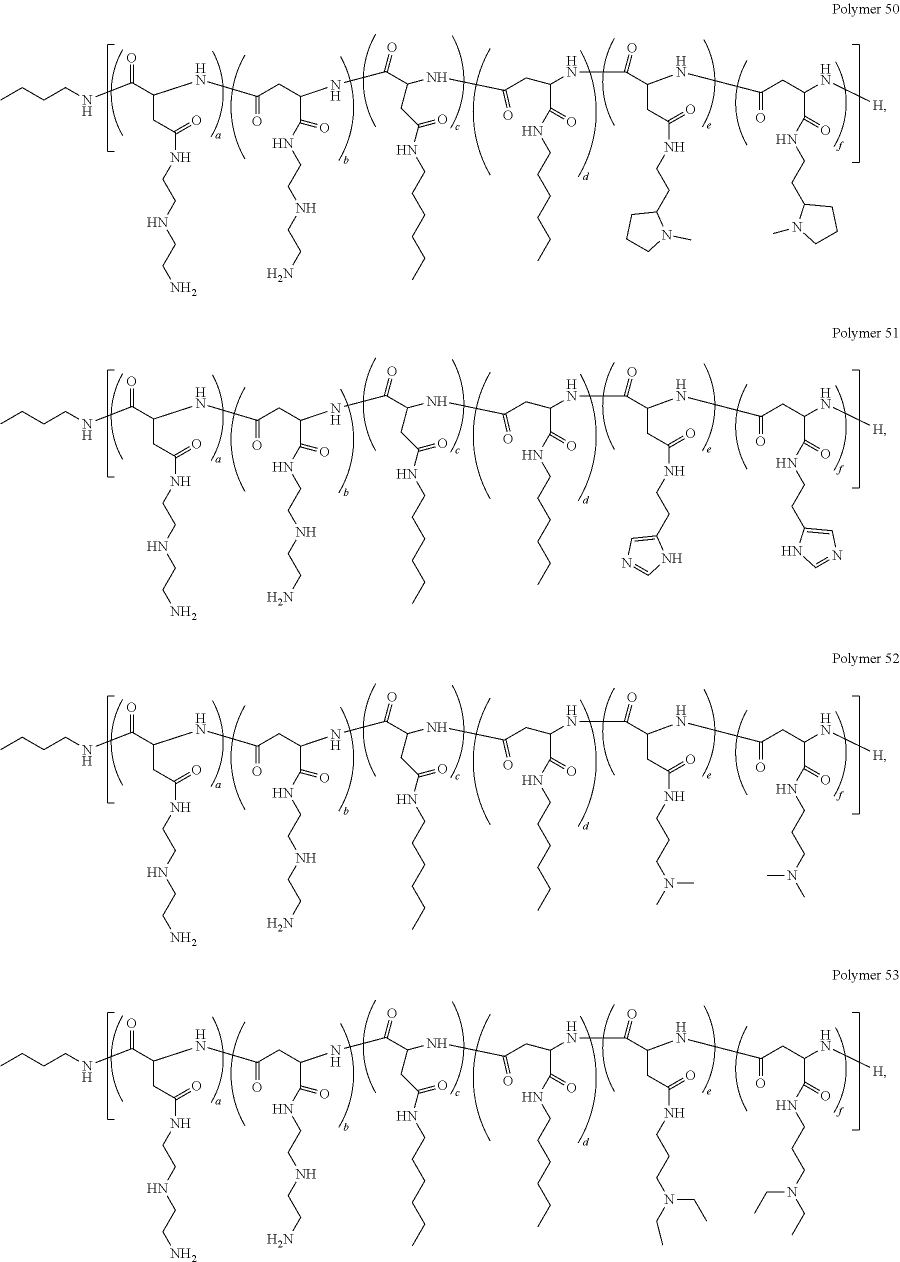

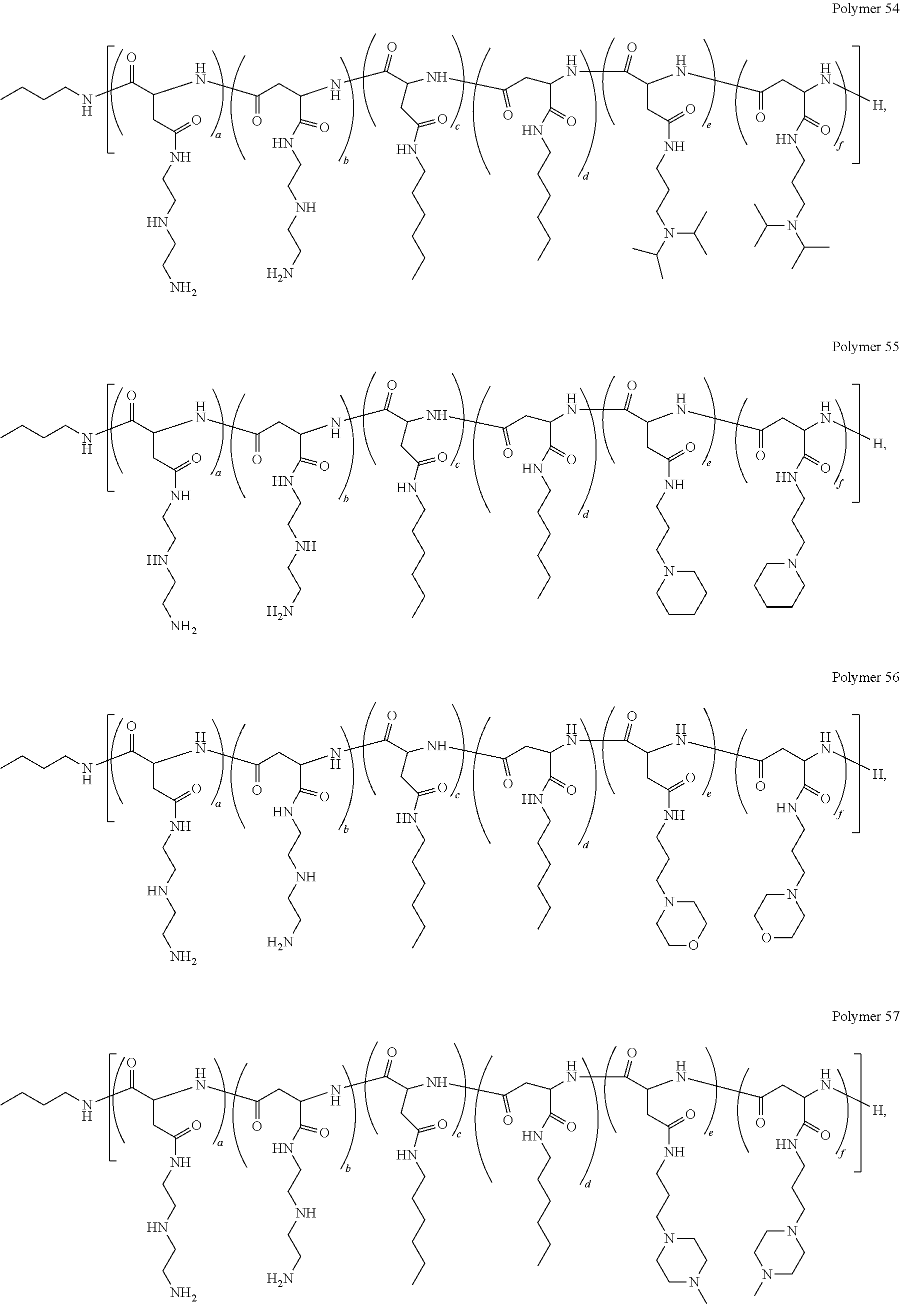

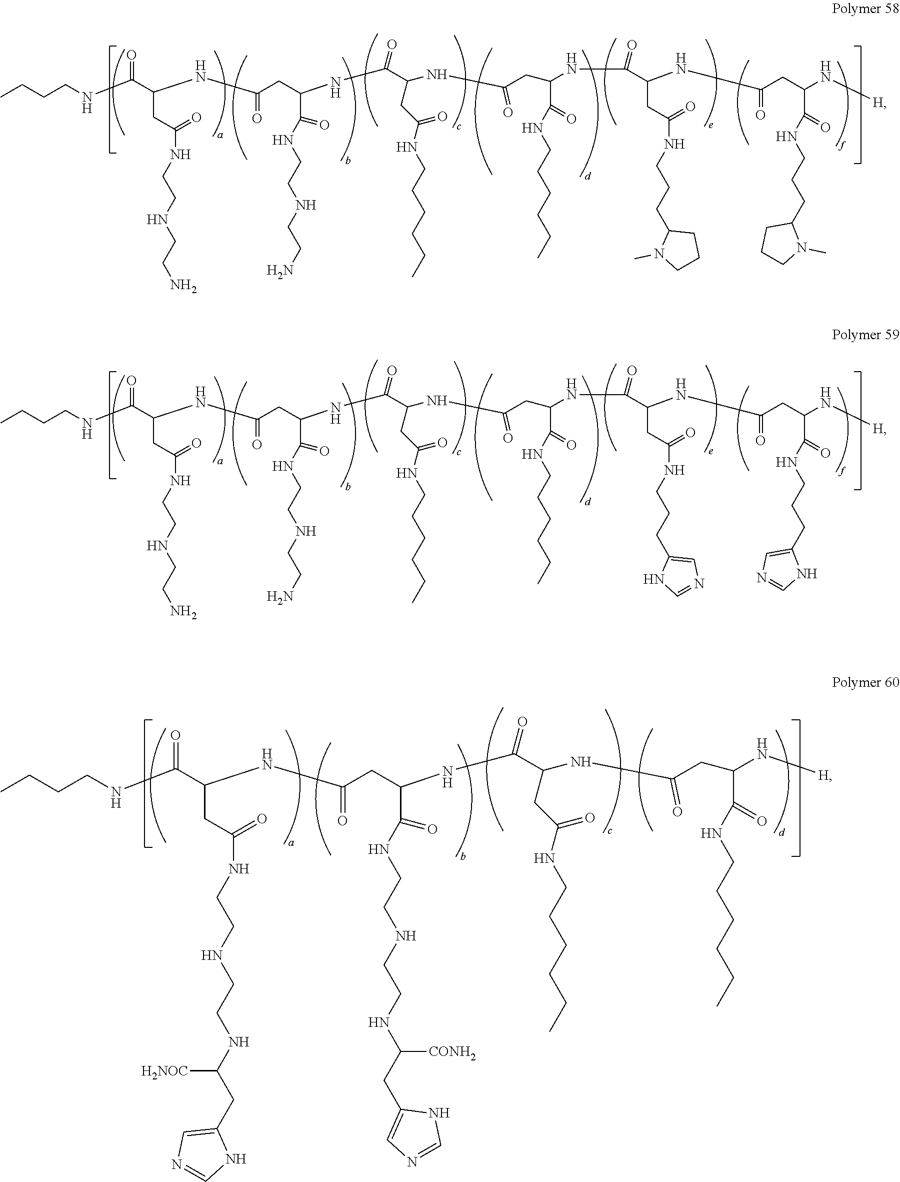

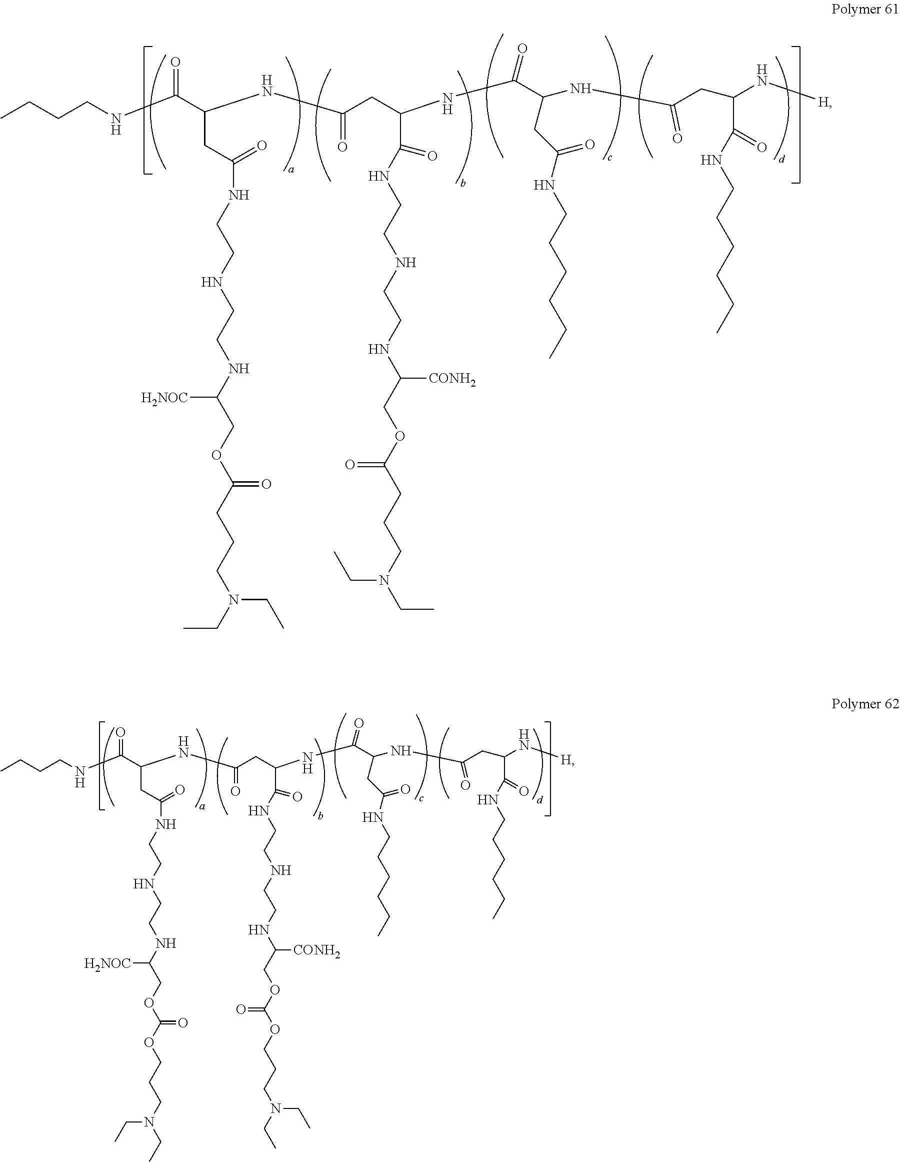

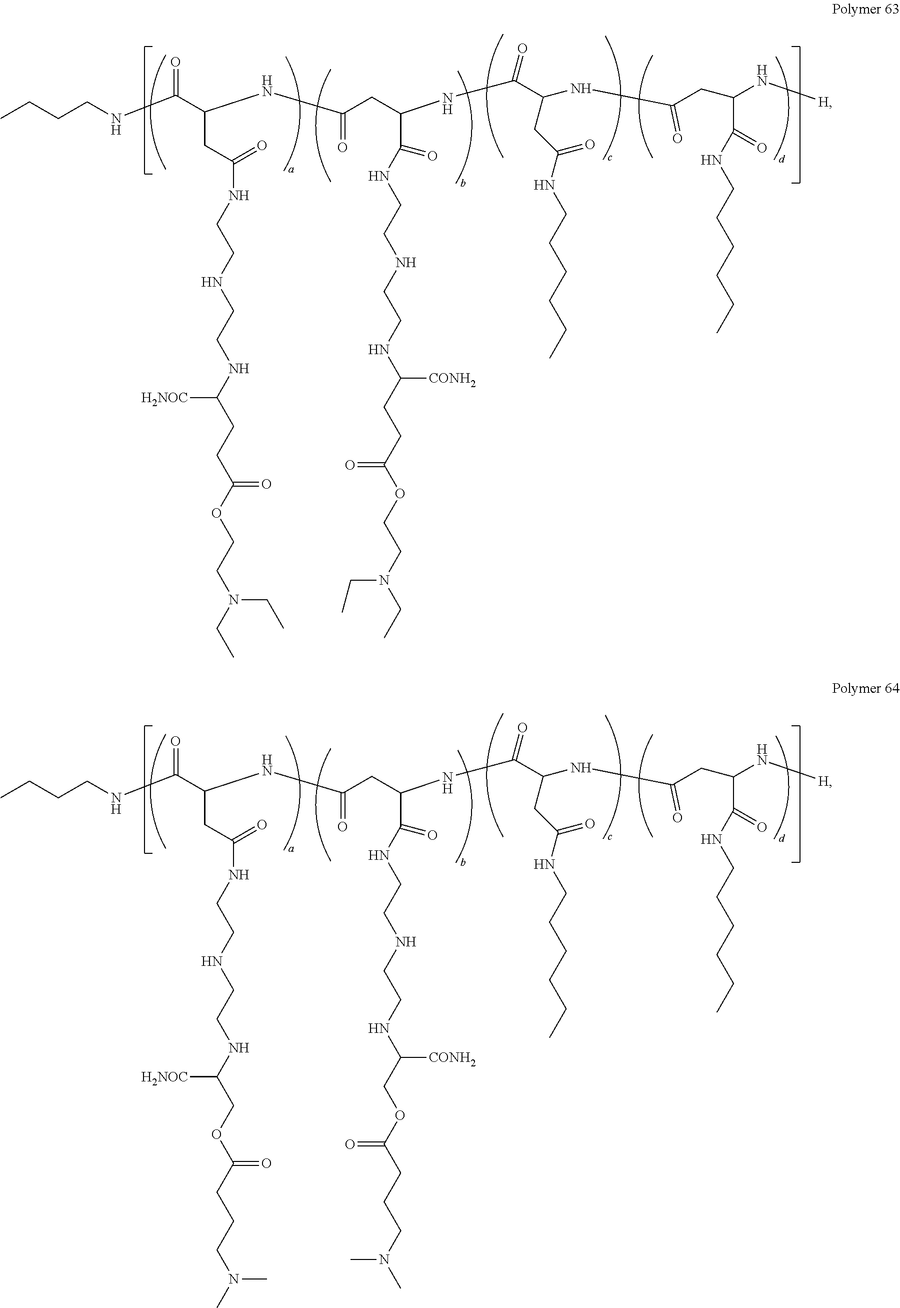

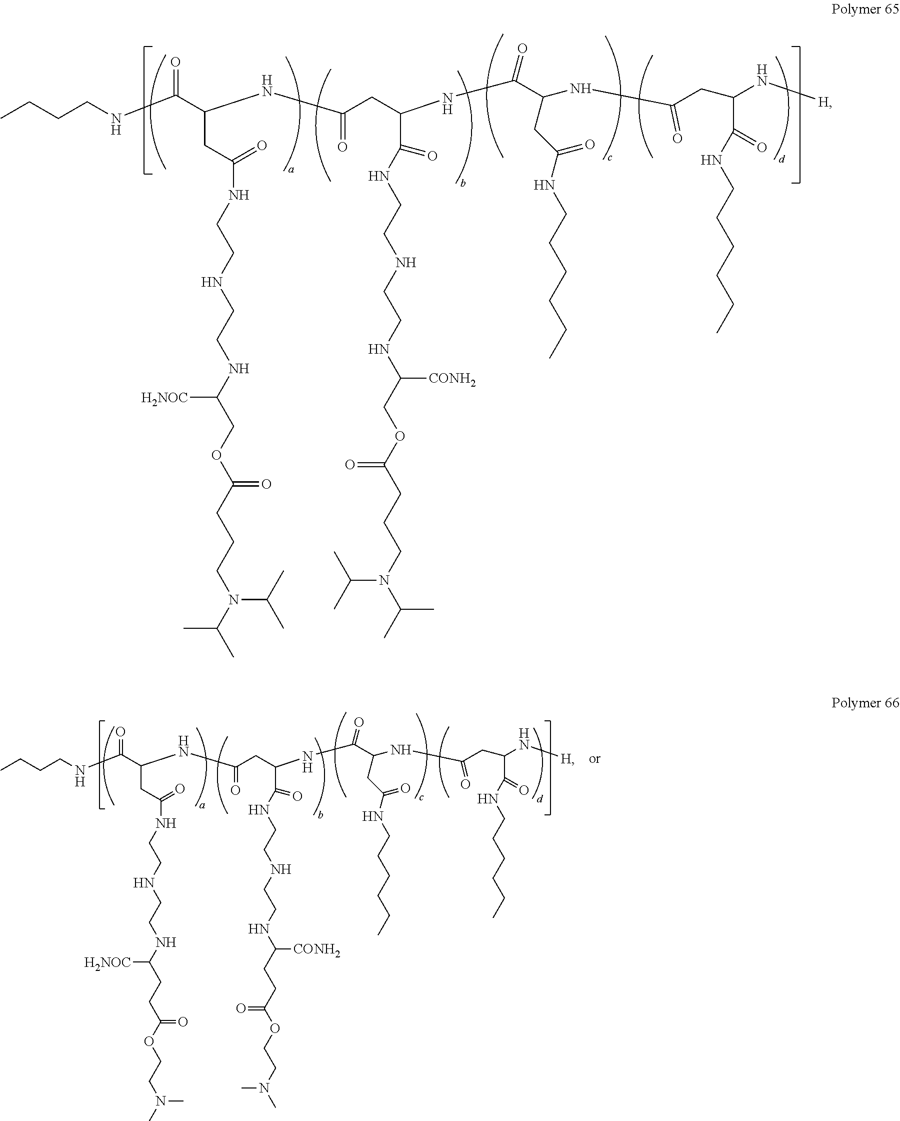

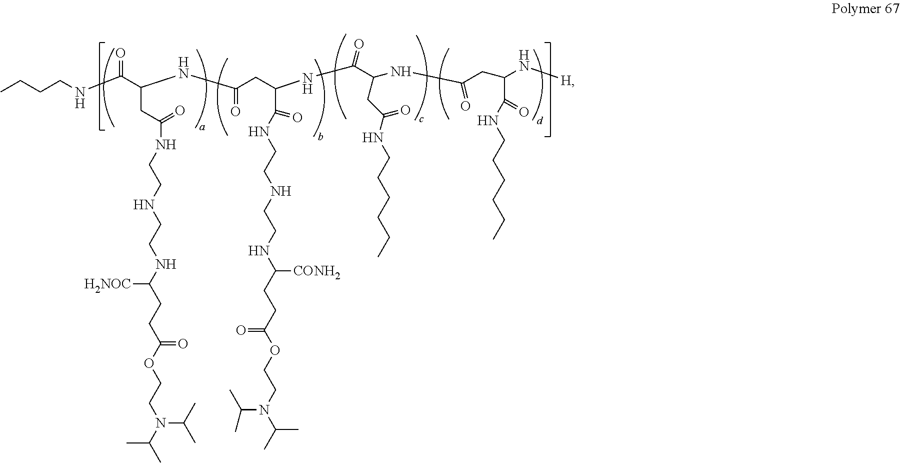

Further examples of polymers provided herein comprising PEG terminal groups are as follows:

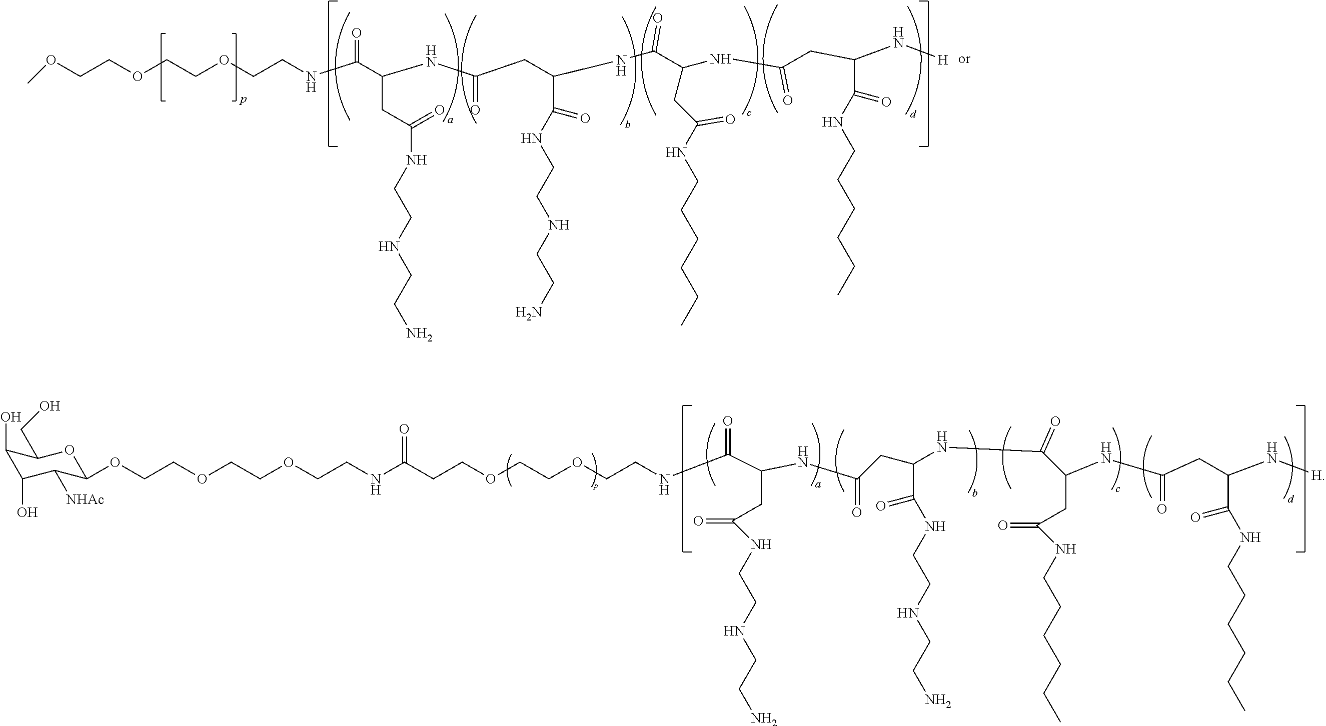

The indication of the number of units (“a”, “b”, “c”, and “d”) in these exemplary polymers does not imply a block co-polymer structure; rather, these numbers indicate the number of particular monomer units overall, which units can be arranged in any order, including blocks of monomers or monomers randomly arranged throughout the polymer. In some instances, but not all, this is additionally indicated by the “/” symbols in the formulas; however, the absence of a “/” should not be taken to mean that the polymers are joined in a particular order. In some embodiments of the foregoing Polymers 1-69, the monomers designated by parenthesis and an integer (“a”, “b”, “c”, or “d”) are randomly arranged or dispersed throughout the polymer.

In any of the foregoing polymers, (a+b) is from about 5 to about 65 (e.g., about 5 to about 50, about 5 to about 40, about 5 to about 30, about 5 to about 20, or about 5 to about 10) and (c+d) is from about 2 to about 60 (e.g., about 2 to about 50, about 2 to about 40, about 2 to about 30, about 2 to about 20, or about 2 to about 10). In certain embodiments, (a+b) is about 55 and (c+d) is about 10. In other embodiments, (a+b) is about 45 and (c+d) is about 20. In certain embodiments, (a+b+c+d) is about 10-500, such as about 10-400, about 10-200, or about 10-100 (e.g., about 25-100 or about 50-75).

The polymer can contain any suitable proportion of (a+b) to (c+d). In other embodiments, (a+b) ranges from 10-95% (e.g., 10-75%, 10-65%, 10-50%, 20-95%, 20-75%, 20-65%, 20-50%, 30-95%, 30-75%, 30-65%, or 30-50%) of the total number of polymer units (a+b+c+d). In other embodiments, (c+d) ranges from 5-90% of the total number of polymer units (a+b+c+d) (e.g., 5-75%, 5-65%, 5-50%, 5-40%, 5-30%, 10-90%, 10-75%, 10-65%, 10-50%, 10-40%, or 10-30%). In still other embodiments, the ratio of (a+b):(c+d) can be about 1 to about 25, from about 1 to about 20, from about 1 to about 10, from about 1 to about 5, from about 5 to about 25, from about 10 to about 25, or from about 15 to about 25.

Certain of the above polymers comprise monomers with ionizable side chains “e” and “f,” in which case a, b, c, and d are as described above, and (e+f) is from about 2 to about 60 (e.g., about 2 to about 50, about 2 to about 40, about 2 to about 30, about 2 to about 20, or about 2 to about 10). In addition, each instance of p is independently an integer from 2 to 200 (e.g., 2 to 150, 2 to 100, 2 to 50, 10 to 200, 10 to 150, 10 to 100, 10 to 50, 25 to 200, 25 to 150, 25 to 100, 25 to 50, 50 to 200, 50 to 150, or 50 to 100). Furthermore, (a+b+c+d+e+f) is about 10-500, such as about 10-400, about 10-200, or about 10-100 (e.g., about 25-100 or about 50-75). Again, the indication of the number of units (“a”, “b”, “c”, “d,” “e,” and “f”) in these exemplary polymers does not imply a block co-polymer structure; rather, these numbers indicate the number of units overall, which units can be randomly arranged.

Some of the above particular examples of polymers provided by the disclosure are depicted with specific terminal groups (e.g., alkylamino, hydrogen, or PEG); however, any of the foregoing particular structures can comprise different terminal groups. For example, any of the foregoing structures comprise a group of R¹, R⁶, or Q as described herein at either or both termini of the polymer backbone.

Any of the forgoing polymers can comprise a tissue-specific or cell-specific targeting moiety at a position indicated in the described formulas, or the polymers can be otherwise modified to include a tissue-specific or cell-specific targeting moiety. For example, the moiety can be added to a terminus of the polymer, or a terminal amine of groups A¹, A², B¹, and/or B² can be modified (e.g., by a Michael addition reaction, an epoxide opening, a displacement reaction, or any other suitable technique) to attach the tissue-specific or cell-specific targeting moiety. The tissue-specific or cell-specific targeting moiety can be any small molecule, protein (e.g., antibody or antigen), amino acid sequence, sugar, oligonucleotide, metal-based nanoparticle, or combination thereof, capable of recognizing (e.g., specifically binding) a given target tissue or cell (e.g., specifically binding a particular ligand, receptor, or other protein or molecule that allows the targeting moiety to discriminate between the target tissue or cell and other non-target tissues or cells). In some embodiments, the tissue-specific or cell-specific targeting moiety is a receptor for a ligand. In some embodiments, the tissue-specific or cell-specific targeting moiety is a ligand for a receptor.

The tissue-specific or cell-specific targeting moiety can be used to target any desired tissue or cell type. In some embodiments, the tissue-specific or cell-specific targeting moiety localizes the polymer to tissues of the peripheral nervous system, the central nervous system, liver, muscle (e.g., cardiac muscle), lung, bone (e.g., hematopoietic cells), or the eye of the subject. In certain embodiments, the tissue-specific or cell-specific targeting moiety localizes the polymer to tumor cells. For example, the tissue-specific or cell-specific targeting moiety can be a sugar that binds to a receptor on a specific tissue or cell.

In some embodiments, the tissue-specific or cell-specific targeting moiety is:

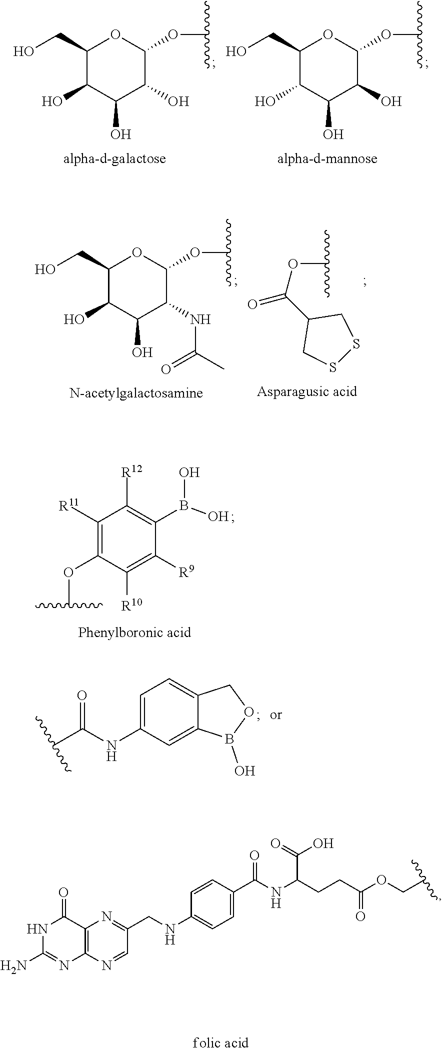

wherein each of R⁹, R¹⁰, R¹¹, and R¹² is independently hydrogen, halogen, C₁-C₄ alkyl, or C₁-C₄ alkoxy, optionally substituted with one or more amino groups. The specified tissue-specific or cell-specific targeting moieties can be chosen to localize the polymer to a tissue described herein. For example, alpha-d-mannose can be used to localize the polymer to the peripheral nervous system, the central nervous system, or immune cells, alpha-d-galactose and N-acetylgalactosamine can be used to localize the polymer to liver hepatocytes, and folic acid can be used to localize the polymer to tumor cells.

Typically, the polymer is cationic (i.e., positively charged at pH 7 and 23° C.). As used herein, “cationic” polymers refer to polymers having an overall net positive charge, whether the polymer comprises only cationic monomer units or a combination of cationic monomer units and non-ionic or anionic monomer units.

In certain embodiments, the polymer has a weight average molecular weight of from about 5 kDa to about 2,000 kDa. The polymer can have a weight average molecular weight of about 2,000 kDa or less, for example, about 1,800 kDa or less, about 1,600 kDa or less, about 1,400 kDa or less, about 1,200 kDa or less, about 1,000 kDa or less, about 900 kDa, or less, about 800 kDa, or less, about 700 kDa or less, about 600 kDa or less, about 500 kDa or less, about 100 kDa or less, or about 50 kDa or less. Alternatively, or in addition, the polymer can have a weight average molecular weight of about 10 kDa or more, for example, about 50 kDa or more, about 100 kDa or more, about 200 kDa or more, about 300 kDa or more, or about 400 kDa or more. Thus, the polymer can have a weight average molecular weight bounded by any two of the aforementioned endpoints. For example, the polymer can have a weight average molecular weight of from about 10 kDa to about 50 kDa, from about 10 kDa to about 100 kDa, from about 10 kDa to about 500 kDa, from about 50 kDa to about 500 kDa, from about 100 kDa to about 500 kDa, from about 200 kDa to about 500 kDa, from about 300 kDa to about 500 kDa, from about 400 kDa to about 500 kDa, from about 400 kDa to about 600 kDa, from about 400 kDa to about 700 kDa, from about 400 kDa to about 800 kDa, from about 400 kDa to about 900 kDa, from about 400 kDa to about 1,000 kDa, from about 400 kDa to about 1,200 kDa, from about 400 kDa to about 1,400 kDa, from about 400 kDa to about 1,600 kDa, from about 400 kDa to about 1,800 kDa, from about 400 kDa to about 2,000 kDa, from about 200 kDa to about 2,000 kDa, from about 500 kDa to about 2,000 kDa, or from about 800 kDa to about 2,000 kDa.

The weight average molecular weight can be determined by any suitable technique. Generally, the weight average molecular weight is determined using size exclusion chromatography equipped with a column, selected from TSKgel Guard, GMPW, GMPW, G1000PW, and a Waters 2414 (Waters Corporation, Milford, Mass.) refractive index detector. Moreover, the weight average molecular weight is determined from calibration with polyethylene oxide/polyethylene glycol standards ranging from 150-875,000 Daltons.

### Methods of Preparation

The invention also provides a method of preparing a polymer described herein. In some embodiments, the method comprises preparing a polymer of Formula 4:

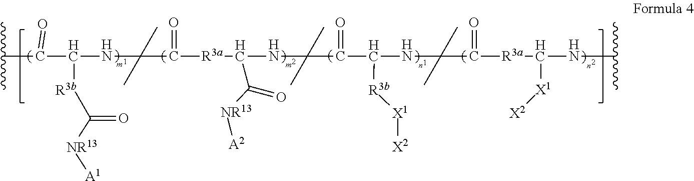

as described herein from a polymer comprising a structure of Formula 2 or Formula 3:

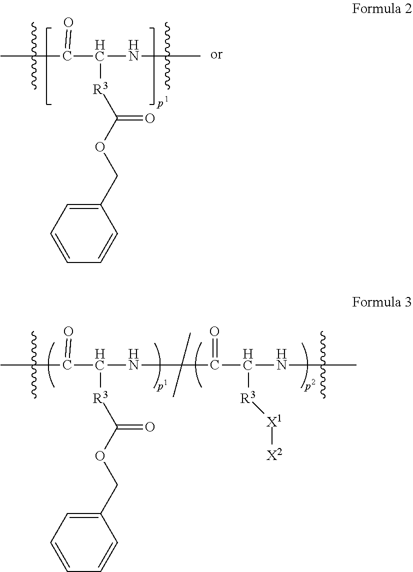

wherein,

p¹ is an integer from 1 to 2000 (e.g., from 1 to 1000, from 1 to 500, from 1 to 200, from 1 to 100, from 5 to 2000, from 5 to 1000, from 5 to 500, from 5 to 200, or from 5 to 100);

p² is an integer from 1 to 2000 (e.g., from 1 to 1000, from 1 to 500, from 1 to 200, from 1 to 100, from 2 to 2000, from 2 to 1000, from 2 to 500, from 2 to 200, or from 2 to 100);

each R³ is independently a methylene or ethylene group;

and X¹ and X² are as previously described with respect to Formulas 1, 1A-1C, and 4. Thus, for instance, each X¹ independently is —C(O)O—, —C(O)NR¹³—, —C(O)—, —S(O)(O)—, or a bond; and each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof optionally substituted with one or more substituents. All other embodiments of X¹ and X² as previously described with respect to Formulas 1, 1A-1C, and 4 also apply to X¹ and X² of Formulas 2 and 3.

The method comprises combining the structure of Formula 2 or Formula 3 with a compound of formula HNR¹³A¹ and/or HNR¹³A², and optionally a compound of formula H₂NX² or HOX². More specifically, the structure of Formula 2 can be combined (reacted) with (a) a compound of formula HNR¹³A¹ and/or HNR¹³A², and (b) a compound of formula H₂NX² or HOX², simultaneously or sequentially in any order, to provide the compound of Formula 4. Similarly, the compound of Formula 3, which already includes an X² group, can be combined (reacted) with a compound of formula HNR¹³A¹ and/or HNR¹³A² to provide the compound of Formula 4.

In the compound of HNR¹³A¹ and/or HNR¹³A², each instance of R¹³ is as previously described with respect to the polymers of Formulas 1, 1A-1C, and 4, including any and all embodiments thereof. Thus, for instance, each instance of R¹³ can be independently hydrogen, an aryl group, a heterocyclic group, an alkyl group, alkenyl group, cycloalkyl group, or cycloalkenyl group, any of which can be optionally substituted with one or more substituents.

Similarly, A¹ and A² are as previously described with respect to the polymers of Formulas 1, 1A-1C, and 4. Thus, for instance, A¹ and A² are each independently a group of formula

—(CH₂)ₚ₁—[NR²—(CH₂)_{q1}—]ᵣ₁NR²₂;

—(CH₂)ₚ₂—N[—(CH₂)_{q2}—NR²₂]₂;

—(CH₂)ₚ₃—N{[—(CH₂)_{q3}—NR²₂][—(CH₂)_{q4}—NR²—]r₂R²}; or

—(CH₂)ₚ₄—N{—(CH₂)_{q5}—N[—(CH₂)_{q6}—NR²₂]₂}₂,

wherein p1 to p4, q1 to q6, and r1 and r2 are each independently an integer of 1 to 5; and each instance of R² is independently hydrogen, an aryl group, a heterocyclic group, a C₁-C₁₂ alkyl group, alkenyl group, cycloalkyl group, or cycloalkenyl group, or a C₁-C₁₂ linear or branched alkyl group optionally substituted with one or more substituents, or R² is combined with a second R² so as to form a heterocyclic group. In some embodiments, A¹ and A² are the same.

Group X² of the compound of formula H₂NX² or HOX² is as described with respect to Formulas 1, 1A-1C, 3, and 4, including any and all embodiments thereof

All other substituents and aspects of Formulae 2, 3, and 4, are as described herein with respect to the polymers of the invention (e.g., Formulae 1, 1A, 1B, 1C, and 4), including any and all embodiments thereof.

The compounds of HNR¹³A¹ and/or HNR¹³A² and of formula H₂NX² or HOX² can be added to the compound of formula 2 or 3 in any suitable manner and amount depending upon the desired degree of substitution. In some embodiments, about 1-400 equivalents (e.g., about 1-350, 1-300, 1-250, 1-200, 1-150, 1-100, 1-50, 10-400, 10-350, 10-300, 10-250, 10-200, 10-150, 10-100, 10-50, 20-400, 20-350, 20-300, 20-250, 20-200, 20-150, 20-100, 20-50, 30-400, 30-350, 30-300, 30-250, 30-200, 30-150, 30-100, 30-50, 40-400, 40-350, 40-300, 40-250, 40-200, 40-150, 40-100, 40-50, 50-400, 50-350, 50-300, 50-250, 50-200, 50-150, or 50-100 equivalents) of the compound of formula H₂NX² or HOX² is added to polymer of Formula 2. Also, in some embodiments, about 1-400 equivalents (e.g., about 1-350, 1-300, 1-250, 1-200, 1-150, 1-100, 1-50, 10-400, 10-350, 10-300, 10-250, 10-200, 10-150, 10-100, 10-50, 20-400, 20-350, 20-300, 20-250, 20-200, 20-150, 20-100, 20-50, 30-400, 30-350, 30-300, 30-250, 30-200, 30-150, 30-100, 30-50, 40-400, 40-350, 40-300, 40-250, 40-200, 40-150, 40-100, 40-50, 50-400, 50-350, 50-300, 50-250, 50-200, 50-150, or 50-100 equivalents) of the compound of formula HNR¹³A¹ and/or HNR¹³A² is added to the polymer of Formula 2 or Formula 3.

In embodiments where the method comprises adding a compound of formula HNR¹³A¹ and/or HNR¹³A² and a compound of formula H₂NX² or HOX² to the polymer of Formula 2, the compound of formula HNR¹³A¹ and/or HNR¹³A² and the compound of formula H₂NX² or HOX² can be present in the reaction mixture in any suitable ratio. For example, the compound of formula HNR¹³A¹ and/or HNR¹³A² and the compound of formula H₂NX² or HOX² can be present in a molar ratio of about 150:1 to about 1:150. In some embodiments, a ratio of about 150:1 to about 1:1, such as about 50:1 to about 1:1 (e.g., about 25:1 to about 1:1, about 10:1 to about 1:1, about 5:1 to about 1:1, or about 2.5:1 to about 1:1) is used. In other embodiments, the ratio is about 1:150 to about 1:1, such as about 1:50 to about 1:1 (e.g., about 1:25 to about 1:1, about 1:10 to about 1:1, about 1:5 to about 1:1, or about 1:2.5 to about 1:1). In still other embodiments, the ratio is about 1:10 to about 1:150, about 1:40 to about 1:150, or about 1:80 to about 1:150.

In some embodiments, the polymer comprising a structure of Formula 2 or Formula 3 is a polymer of Formula 2A or Formula 3A, respectively:

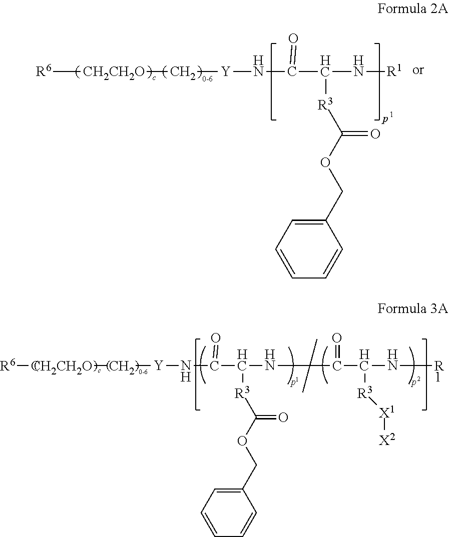

wherein c, Y, R¹, and R⁶ are as previously described with respect to the polymers of Formulae 1A and 1B, including any and all embodiments thereof; and p¹, p², R³, X¹, and X², are as described above with respect to Formulae 2 and 3. Thus, for instance:

p¹is an integer from 1 to 2000;

p² is an integer from 1 to 2000;

each R³ is independently a methylene or ethylene group;

each X¹ independently is —C(O)O—, —C(O)NR¹³—, —C(O)—, —S(O)(O)—, or a bond;

each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof optionally substituted with one or more substituents, or any other embodiments of X¹ and X² as previously described with respect to Formulas 1,1A-1C, 2, 3, and 4;

the symbol “/” indicates that the units separated thereby are linked randomly or in any order;

c is an integer from 0 to 50;

Y is optionally present and is a cleavable linker;

R¹ is hydrogen, an aryl group, a heterocyclic group, a C₁-C₁₂ alkyl group, alkenyl group, cycloalkyl group, or cycloalkenyl group, or a C₁-C₁₂ linear or branched alkyl group optionally substituted with one or more substituents; and

R⁶ is hydrogen, an amino group, an aryl group, a heterocyclic group, a C₁-C₁₂ alkyl group, a C₁-C₁₂ heteroalkyl group, alkenyl group, cycloalkyl group, or cycloalkenyl group, a C₁-C₁₂ linear or branched alkyl group optionally substituted with one or more amines; or a tissue-specific or cell-specific targeting moiety. All aspects of Formulae 2A and 3A are otherwise as described herein with respect to the polymers of the invention (e.g., Formulae 1, 2, 1A, 1B, 1C, 3, and 4).

In certain embodiments, the polymer comprising a structure of Formula 2 or Formula 3 is a polymer of Formula 2B or Formula 3B, respectively:

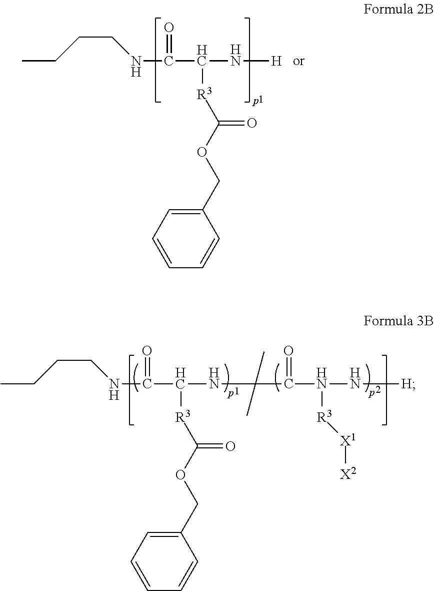

wherein p¹, p², R³, X¹, and X², are as described above with respect to Formulae 2, 2A, 3, and 3A. Thus, for instance,

p¹ is an integer from 1 to 2000;

p² is an integer from 1 to 2000;

each R³ is independently a methylene or ethylene group;

each X¹ independently is —C(O)O—, —C(O)NR¹³—, —C(O)—, —S(O)(O)—, or a bond;

each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof optionally substituted with one or more substituents, or any other embodiments of X¹ and X² as previously described with respect to Formulas 1, 1A-1C, 2, 3, and 4; and

the symbol “/” indicates that the units separated thereby are linked randomly or in any order. All aspects of Formulae 2B and 3B, are otherwise as described herein with respect to the polymers of the invention (e.g., Formulae 1, 1A, 1B, 1C, 2, 2A, 3, 3A, and 4).

In some embodiments, the method also provides a method of preparing a polymer of Formula 1, which comprises modifying at least a portion of groups A¹ and/or A² of a polymer comprising a structure of Formula 4:

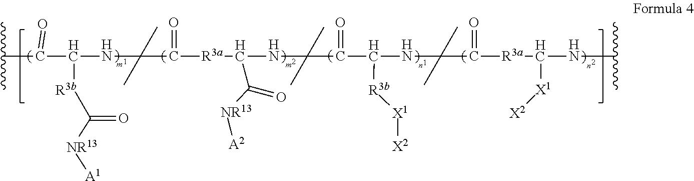

to produce a polymer comprising a structure of Formula 1:

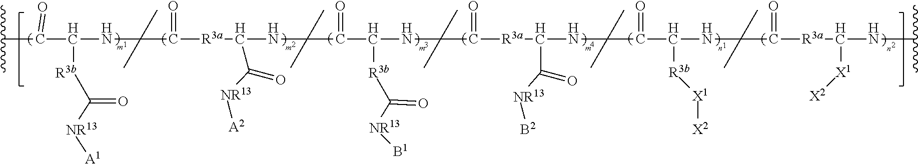

All aspects of the polymers of Formula 1 and 4 are as previously disclosed herein. Thus, for instance:

each of m¹, m², m³, and m⁴ is an integer from 0 to 1000, provided that the sum of m¹+m²+m³+m⁴ is greater than 5;

each of n¹ and n² is an integer from 0 to 1000, provided that the sum of n¹+n² is greater than 2;

the symbol “/” indicates that the units separated thereby are linked randomly or in any order;

each instance of R^{3a} is independently a methylene or ethylene group;

each instance of R^{3b} is independently a methylene or ethylene group;

each X¹ independently is —C(O)O—, —C(O)NR¹³—, —C(O)—, —S(O)(O)—, or a bond;

each instance of R¹³ is independently hydrogen, an aryl group, a heterocyclic group, a C₁-C₁₂ alkyl group, alkenyl group, cycloalkyl group, or cycloalkenyl group, any of which can be optionally substituted with one or more substituents;

each instance of X² is independently a C₁-C₁₂ alkyl group, cycloalkyl group, alkenyl group, cycloalkenyl group, aryl group, heteroalkyl group, heterocyclic group, or combination thereof optionally substituted with one or more substituents;

A¹ and A² are each independently a group of formula

—(CH₂)ₚ₁—[NR²—(CH₂)_{q1}—]ᵣ₁NR²₂;

—(CH₂)ₚ₂—N[—(CH₂)_{q2}—NR²₂]₂;

—(CH₂)ₚ₃—N{[—(CH₂)_{q3}—NR²₂][—(CH₂)_{q4}—NR²—]r₂R²}; or

—(CH₂)ₚ₄—N{—(CH₂)_{q5}—N[—(CH₂)_{q6}—NR²₂]₂}₂,

B¹ and B² are each independently

—(CH₂)ₚ₁—[NR²—(CH₂)_{q1}—]ᵣ₁NR²—(CH₂)ₛ₁—R⁴—R⁵;

—(CH₂)ₚ₂—N[—(CH₂)_{q2}—NR²—(CH₂)ₛ₂—R⁴—R⁵]₂;

—(CH₂)ₚ₃—N{[—(CH₂)_{q3}—NR²₂][—(CH₂)_{q4}—NR²—]ᵣ₂(CH₂)_{s3—R}⁴—R⁵ };

—(CH₂)ₚ₄—N{—(CH₂)_{q5}—N[—(CH₂)_{q6}—NR²—(CH₂)ₛ₄—R⁴—R⁵]₂}₂;

—(CH₂)ₚ₁—[NR²—(CH₂)_{q1}—]ᵣ₁NR²—CH₂—CHOH—R⁵;

—(CH₂)ₚ₂—N[—(CH₂)_{q2}—NR²—CH₂—CHOH—R⁵;

—(CH₂)ₚ₃—N{[—(CH₂)_{q3}—NR²₂][—(CH₂)_{q4}—NR²—]ᵣ₂—CH₂—CHOH—R⁵;

—(CH₂)ₚ₄—N{—(CH₂)_{q5}—N[—(CH₂)_{q6}—NR²—CH₂—CHOH—R⁵]₂}₂;

—(CH₂)ₚ₁—[NR²—(CH₂)_{q1}—]ᵣ₁NR²—(CH₂)ₛ₁—R⁵;

—(CH₂)ₚ₂—N[—(CH₂)_{q2}—NR²—(CH₂)ₛ₂—R⁵]₂;

—(CH₂)ₚ₃—N{[—(CH₂)_{q3}—NR²₂][—(CH₂)_{q4}—NR²—]ᵣ₂(CH₂)ₛ₃—R⁵};

—(CH₂)ₚ₄—N{—(CH₂)_{q5}—N[—(CH₂)_{q6}—NR²—(CH₂)ₛ₄—R⁵]₂}₂;

—(CH₂)ₚ₁—[N{(CH₂)ₛ₁—R⁴—R⁵}—(CH₂)_{q1}—]ᵣ₁NR²₂;

—(CH₂)ₚ₁—[N{(CH₂)ₛ₁—R⁵}—(CH₂)_{q1}—]ᵣ₁NR²₂,

—(CH₂)ₚ₁—[NR²—(CH₂)_{q1}—]ᵣ₁NR²—CH(CONH₂)—(CH₂)ₛ₁—R⁵; or

—(CH₂)ₚ₁—[NR²—(CH₂)_{q1}—]ᵣ₁NR²—CH(CONH₂)—(CH₂)ₛ₁—R⁴—R⁵,

wherein p1 to p4, q1 to q6, r1 and r2, and s1 to s4 are each independently an integer of 1 to 5; each instance of R² is independently hydrogen or a C₁-C₁₂ alkyl group, alkenyl group, cycloalkyl group, or cycloalkenyl group, or R² is combined with a second R² so as to form a heterocyclic group; each instance of R⁴ is independently —C(O)O—, —C(O)NH—, —O—C(O)O—, or —S(O)(O)—; and each instance of R⁵ is independently an alkyl group, cycloalkyl group, alkenyl group, cycloalkenyl group, aryl group, heteroalkyl group, heterocyclic group, or combination thereof optionally comprising from 2 to 8 tertiary amines or a substituent comprising a tissue-specific or cell-specific targeting moiety. All aspects of Formulae 1, 1A-1C, and 4, are otherwise as described herein with respect to the polymers of the invention, including any and all embodiments of the structures of Formulae 1, 1A-1C, and 4 described herein.

The polymer comprising a structure of Formula 1 or Formula 4 can be any polymer described herein, including Formulas 1A, 1B, and 1C, as well as any and all embodiments thereof as described with respect to the polymer of the invention.

The groups designated A¹ and/or A² of the polymer of Formula 4 can be modified by any suitable means to produce groups designated B¹ and/or B². For example, the groups designated A¹ and/or A² can be modified by a Michael addition reaction, an epoxide opening, or a displacement reaction. In preferred embodiments, the groups designated A¹ and/or A² are modified by a Michael addition reaction.

In one embodiment, groups A¹ and/or A² of the polymer comprising a structure of Formula 4 are modified by a Michael addition reaction between the polymer comprising the structure of Formula 4 and α,β-unsaturated carbonyl compound. As used herein, the term “Michael addition” refers to a nucleophilic addition of a nucleophile of the polymer (e.g., a carbanion, an oxygen anion, a nitrogen anion, an oxygen atom, a nitrogen atom, or a combination thereof) to an α,β-unsaturated carbonyl compound. Accordingly, the Michael addition reaction is between the polymer comprising the structure of Formula 4 and an α,β-unsaturated carbonyl compound. In some embodiments, the nucleophile of the polymer is a nitrogen anion, a nitrogen atom, or a combination thereof.

The α,β-unsaturated carbonyl compound can be any αaβ-unsaturated carbonyl compound capable of accepting a Michael addition from a nucleophile. In some embodiments, the α,β-unsaturated carbonyl compound is an acrylate, an acrylamide, a vinyl sulfone, or a combination thereof. Accordingly, the Michael addition reaction can be between the polymer comprising the structure of Formula 4 and an acrylate, an acrylamide, a vinyl sulfone, or a combination thereof. Thus, in some embodiments, the method comprises contacting the polymer comprising the structure of Formula 4 and an acrylate; contacting the polymer comprising the structure of Formula 4 and an acrylamide; or contacting the polymer comprising the structure of Formula 4 and a vinyl sulfone.

In embodiments where the groups designated A¹ and/or A² are modified by a Michael addition reaction, they produce groups designated B¹ and/or B² of the formula:

—(CH₂)ₚ₁—[NR²—(CH₂)_{q1}—]ᵣ₁NR²—(CH₂)ₛ₁—R⁴—R⁵;

—(CH₂)ₚ₂—N[—(CH₂)_{q2}—NR²—(CH₂)ₛ₂—R⁴—R⁵]₂;

—(CH₂)ₚ₃—N{[—(CH₂)_{q3}—NR²₂][—(CH₂)_{q4}—NR²—]ᵣ₂(CH₂)ₛ₃—R⁴—R⁵};

—(CH₂)ₚ₄—N{—(CH₂)_{q5}—N[—(CH₂)_{q6}—NR²—(CH₂)ₛ₄—R⁴—R⁵]₂}₂; or

—(CH₂)ₚ₁—[N{(CH₂)ₛ₁—R⁴—R⁵}—(CH₂)_{q1}—]ᵣ₁NR²₂;

wherein p1 to p4, q1 to q6, r1 and r2, and s1 to s4 are each independently an integer of 1 to 5; each instance of R² is independently hydrogen, an aryl group, a heterocyclic group, a C₁-C₁₂ alkyl group, alkenyl group, cycloalkyl group, or cycloalkenyl group, or a C₁-C₁₂ linear or branched alkyl group optionally substituted with one or more substituents, or R² is combined with a second R² so as to form a heterocyclic group; each instance of R⁴ is independently —C(O)O—, —C(O)NH—, —O—C(O)O—, or —S(O)(O)—; and each instance of R⁵ is independently an alkyl group, cycloalkyl group, alkenyl group, cycloalkenyl group, aryl group, heteroalkyl group, heterocyclic group, or combination thereof optionally comprising from 2 to 8 tertiary amines or a substituent comprising a tissue-specific or cell-specific targeting moiety.

Examples of acrylates, acrylamides, and vinyl sulfones suitable for use include an acrylate of the formula:

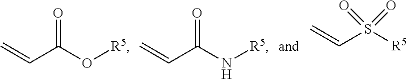

wherein R⁵ is as described with respect to any of Formulas 1, 1A, 1B, or 1C.

In some embodiments, the Michael addition reaction is facilitated by an acid and/or base. The acid and/or base can be any suitable acid and/or base with any suitable pKa. The acid and/or base can be an organic acid (e.g., p-toluenesulfonic acid), organic base (e.g., triethylamine), inorganic acid (e.g., titanium tetrachloride), inorganic base (e.g., potassium carbonate), or a combination thereof

In some embodiments, the Michael addition reaction is facilitated by an acid. The acid can be a Bronsted acid or a Lewis acid. In embodiments where the acid is a Bronsted acid, the acid can be a weak acid (i.e., a pKa of from about 4 to about 7) or a strong acid (i.e., a pKa of from about −2 to about 4). Typically, the acid is a weak acid. In certain embodiments, the acid is a Lewis acid. For example, the acid can be bis(trifluoromethanesulfon)imide or p-toluenesulfonic acid.

In some embodiments, the Michael addition reaction is facilitated by a base. The base can be a weak base (i.e., a pKa of from about 7 to about 12) or a strong base (i.e., a pKa of from about 12 to about 50). Typically, the base is a weak base. For example, the base can be triethylamine, diisopropylethylamine, pyridine, N-methyl morpholine, or N,N-dimethyl-piperazine, or derivatives thereof.

In some embodiments, the Michael addition reaction is performed in a solvent. The solvent can be any suitable solvent, or mixture of solvents, capable of solubilizing the polymer and theα,β-unsaturated carbonyl compound to be reacted. For example, the solvent can include water, protic organic solvents, and/or aprotic organic solvents. An exemplary list of solvents includes water, dichloromethane, diethyl ether, dimethyl sulfoxide, acetonitrile, methanol, and ethanol.

In one embodiment, groups A¹ and/or A² of the polymer are modified by an epoxide opening reaction between the polymer and an epoxide compound. As used herein, the term “epoxide opening” refers to a nucleophilic addition of a nucleophile of the polymer (e.g., a carbanion, an oxygen anion, a nitrogen anion, an oxygen atom, a nitrogen atom, or a combination thereof) to an epoxide compound, thereby opening the epoxide. Accordingly, the epoxide opening reaction is between the polymer and an epoxide compound. In some embodiments, the nucleophile of the polymer is a nitrogen anion, a nitrogen atom, or a combination thereof.

In embodiments where the groups designated A¹ and/or A² are modified by an epoxide opening reaction, they produce groups designated B¹ and/or B² of the formula:

—(CH₂)ₚ₁—[NR²—(CH₂)_{q1}—]ᵣ₁NR²—CH₂—CHOH—R⁵;

—(CH₂)ₚ₂—N[—(CH₂)_{q2}—NR²—CH₂—CHOH—R⁵;

—(CH₂)ₚ₃—N{[—(CH₂)_{q3}—NR²₂][—(CH₂)_{q4}—NR²—]ᵣ₂—CH₂—CHOH—R⁵;

—(CH₂)ₚ₄—N{—(CH₂)_{q5}—N[—(CH₂)_{q6}—NR²—CH₂—CHOH—R⁵]₂}₂;

wherein p1 to p4, q1 to q6, and r1 and r2 are each independently an integer of 1 to 5; each instance of R² is independently hydrogen, an aryl group, a heterocyclic group, a C₁-C₁₂ alkyl group, alkenyl group, cycloalkyl group, or cycloalkenyl group, or a C₁-C₁₂ linear or branched alkyl group optionally substituted with one or more substituents, or R² is combined with a second R² so as to form a heterocyclic group; and each instance of R⁵ is independently an alkyl group, cycloalkyl group, alkenyl group, cycloalkenyl group, aryl group, heteroalkyl group, heterocyclic group, or combination thereof optionally comprising from 2 to 8 tertiary amines or a substituent comprising a tissue-specific or cell-specific targeting moiety.

Examples of epoxides suitable for use include epoxides of the formula:

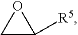

wherein R⁵ is as described with respect to any of Formulas 1, 1A, 1B, or 1C.

In some embodiments, the epoxide opening reaction is facilitated by an acid and/or base. The acid and/or base can be any suitable acid and/or base with any suitable pKa. The acid and/or base can be an organic acid (e.g., p-toluenesulfonic acid), organic base (e.g., triethylamine), inorganic acid (e.g., titanium tetrachloride), inorganic base (e.g., potassium carbonate), or a combination thereof.

In some embodiments, the epoxide opening reaction is facilitated by an acid. The acid can be a Bronsted acid or a Lewis acid. In embodiments where the acid is a Bronsted acid, the acid can be a weak acid (i.e., a pKa of from about 4 to about 7) or a strong acid (i.e., a pKa of from about −2 to about 4). Typically, the acid is a weak acid. In certain embodiments, the acid is a Lewis acid. For example, the acid can be bis(trifluoromethanesulfon)imide or p-toluenesulfonic acid.

In some embodiments, the epoxide opening reaction is facilitated by a base. The base can be a weak base (i.e., a pKa of from about 7 to about 12) or a strong base (i.e., a pKa of from about 12 to about 50). Typically, the base is a weak base. For example, the base can be triethylamine, diisopropylethylamine, pyridine, N-methyl morpholine, or N,N-dimethyl-piperazine, or derivatives thereof.

In some embodiments, the epoxide opening reaction is performed in a solvent. The solvent can be any suitable solvent, or mixture of solvents, capable of solubilizing the polymer and the epoxide compound to be reacted. For example, the solvent can include water, protic organic solvents, and/or aprotic organic solvents. An exemplary list of solvents includes water, dichloromethane, diethyl ether, dimethyl sulfoxide, acetonitrile, methanol, and ethanol.

In one embodiment, groups A¹ and/or A² of the polymer are modified by a displacement reaction between the polymer and a compound comprising a leaving group (e.g., chloride atom, bromide atom, iodide atom, tosylate, triflate, mesylate, etc.). As used herein, the term “displacement” refers to a nucleophilic addition of a nucleophile of the polymer (e.g., a carbanion, an oxygen anion, a nitrogen anion, an oxygen atom, a nitrogen atom, or a combination thereof) to a compound comprising a leaving group. Accordingly, the displacement reaction is between the polymer and a compound comprising a leaving group. In some embodiments, the nucleophile of the polymer is a nitrogen anion, a nitrogen atom, or a combination thereof.

In embodiments where the groups designated A¹ and/or A² are modified by a displacement reaction, they produce groups designated B¹ and/or B² of the formula:

—(CH₂)ₚ₁—[NR²—(CH₂)_{q1}—]ᵣ₁NR²—(CH₂)ₛ₁—R⁵;

—(CH₂)ₚ₂—N[—(CH₂)_{q2}—NR²—(CH₂)ₛ₂—R⁵]₂;

—(CH₂)ₚ₃—N{[—(CH₂)_{q3}—NR²₂][—(CH₂)_{q4}—NR²—]ᵣ₂(CH₂)ₛ₃—R⁵};

—(CH₂)ₚ₄—N{—(CH₂)_{q5}—N[—(CH₂)_{q6}—NR²—(CH₂)ₛ₄—R⁵]₂}₂; or

—(CH₂)ₚ₁—[N{(CH₂)ₛ₁—R⁵}—(CH₂)_{q1}—]ᵣ₁NR²₂,

wherein p1 to p4, q1 to q6, r1 and r2, and s1 to s4 are each independently an integer of 1 to 5; each instance of R² is independently hydrogen, an aryl group, a heterocyclic group, a C₁-C₁₂ alkyl group, alkenyl group, cycloalkyl group, or cycloalkenyl group, or a C₁-C₁₂ linear or branched alkyl group optionally substituted with one or more substituents, or R² is combined with a second R² so as to form a heterocyclic group; and each instance of R⁵ is independently an alkyl group, cycloalkyl group, alkenyl group, cycloalkenyl group, aryl group, heteroalkyl group, heterocyclic group, or combination thereof optionally comprising from 2 to 8 tertiary amines or a substituent comprising a tissue-specific or cell-specific targeting moiety.

Examples of compounds containing a leaving group suitable for use include compound of formula:

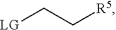

wherein LG is a leaving group (e.g., chloride atom, bromide atom, iodide atom, tosylate, triflate, mesylate, etc.) and R⁵ is as described with respect to any of Formulas 1, 1A, 1B, or 1C.

In some embodiments, the displacement reaction is facilitated by an acid and/or base. The acid and/or base can be any suitable acid and/or base with any suitable pKa. The acid and/or base can be an organic acid (e.g., p-toluenesulfonic acid), organic base (e.g., triethylamine), inorganic acid (e.g., titanium tetrachloride), inorganic base (e.g., potassium carbonate), or a combination thereof.

In some embodiments, the displacement reaction is facilitated by an acid. The acid can be a Bronsted acid or a Lewis acid. In embodiments where the acid is a Bronsted acid, the acid can be a weak acid (i.e., a pKa of from about 4 to about 7) or a strong acid (i.e., a pKa of from about −2 to about 4). Typically, the acid is a weak acid. In certain embodiments, the acid is a Lewis acid. For example, the acid can be bis(trifluoromethanesulfon)imide or p-toluenesulfonic acid.

In some embodiments, the displacement reaction is facilitated by a base. The base can be a weak base (i.e., a pKa of from about 7 to about 12) or a strong base (i.e., a pKa of from about 12 to about 50). Typically, the base is a weak base. For example, the base can be triethylamine, diisopropylethylamine, pyridine, N-methyl morpholine, or N,N-dimethyl-piperazine, or derivatives thereof.

In some embodiments, the displacement reaction is performed in a solvent. The solvent can be any suitable solvent, or mixture of solvents, capable of solubilizing the polymer and the compound comprising a leaving group to be reacted. For example, the solvent can include water, protic organic solvents, and/or aprotic organic solvents. An exemplary list of solvents includes water, dichloromethane, diethyl ether, dimethyl sulfoxide, acetonitrile, methanol, and ethanol.

In some embodiments, the method further comprises isolating the polymer comprising the structure of Formula 1. The polymer comprising the structure of Formula 1 can be isolated by any suitable means. For example, the polymer comprising the structure of Formula 1 can be isolated by extraction, crystallization, recrystallization, column chromatography, filtration, or any combination thereof.

### Compositions

The polymers provided herein can be used for any purpose. However, it is believed the polymers are particularly useful for delivering nucleic acids and/or polypeptides (e.g., protein) to cells. Thus, provided herein is a composition comprising a polymer as described herein and a nucleic acid and/or polypeptide (e.g., protein).

In some embodiments, the composition comprises a nucleic acid. Any nucleic acid can be used. An exemplary list of nucleic acids includes guide and/or donor nucleic acids of CRISPR systems, siRNA, microRNA, interfering RNA or RNAi, dsRNA, mRNA, DNA vector, ribozymes, antisense polynucleotides, and DNA expression cassettes encoding siRNA, microRNA, dsRNA, ribozymes or antisense nucleic acids. SiRNA comprises a double stranded structure typically containing 15-50 base pairs and preferably 19-25 base pairs and having a nucleotide sequence identical or nearly identical to an expressed target gene or RNA within the cell. An siRNA may be composed of two annealed polynucleotides or a single polynucleotide that forms a hairpin structure. MicroRNAs (miRNAs) are small noncoding polynucleotides, about 22 nucleotides long, that direct destruction or translational repression of their mRNA targets. Antisense polynucleotides comprise sequence that is complimentary to a gene or mRNA. Antisense polynucleotides include, but are not limited to: morpholinos, 2′-O-methyl polynucleotides, DNA, RNA and the like. The polynucleotide-based expression inhibitor may be polymerized in vitro, recombinant, contain chimeric sequences, or derivatives of these groups. The polynucleotide-based expression inhibitor may contain ribonucleotides, deoxyribonucleotides, synthetic nucleotides, or any suitable combination such that the target RNA and/or gene is inhibited.

The composition also can comprise any protein for delivery, in addition to or instead of a nucleic acid. The polypeptide can be any suitable polypeptide. For example, the polypeptide can be a zinc finger nuclease, a transcription activator-like effector nuclease (“TALEN”), a recombinase, a deaminase, an endonuclease, or a combination thereof. In some embodiments, the polypeptide is an RNA-guided endonuclease (e.g., a Cas9 polypeptide, a Cpf1 polypeptide, or variants thereof) or a DNA recombinase (e.g., a Cre polypeptide).

It is believed the polymers provided herein are particularly useful for delivering one or more components of a CRISPR system. Thus, in some embodiments, the composition comprises a guide RNA, an RNA-guided endonuclease or nucleic acid encoding same, and/or a donor nucleic acid. The composition can comprise one, two, or all three components together with the polymer described herein. Furthermore, the composition can comprise a plurality of guide RNAs, RNA-guided endonucleases or nucleic acids encoding same, and/or donor nucleic acids. For instance, multiple different guide RNAs for different target sites could be included, optionally with multiple different donor nucleic acids and even multiple different RNA guided endonucleases or nucleic acids encoding same.

Furthermore, the components of the CRISPR system can be combined with one another (when multiple components are present) and the polymer in any particular manner or order. In some embodiments, the guide RNA is complexed with an RNA endonuclease prior to combining with the polymer. In addition, or instead, the guide RNA can be linked (covalently or non-covalently) to a donor nucleic acid prior to combining with the polymer.

The compositions are not limited with respect to any particular CRISPR system (i.e., any particular guide RNA, RNA-guided endonuclease, or donor nucleic acid), many of which are known. Nevertheless, for the sake of further illustration, the components of some such systems are described below.

Donor Nucleic Acid

The donor nucleic acid (or “donor sequence” or “donor polynucleotide” or “donor DNA”) is a nucleic acid sequence to be inserted at the cleavage site induced by an RNA-directed endonuclease (e.g., a Cas9 polypeptide or a Cpf1 polypeptide). The donor polynucleotide will contain sufficient homology to a target genomic sequence at the cleavage site, e.g. 70%, 80%, 85%, 90%, 95%, or 100% homology with the nucleotide sequences flanking the cleavage site, e.g. within about 50 bases or less of the cleavage site, e.g. within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some embodiments may be used for other purposes (e.g., to signify expression at the targeted genomic locus). In some embodiments, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad Sci USA 84:4959-4963; Nehls et al. (1996) Science 272:886-889. Amplification procedures such as rolling circle amplification can also be advantageously employed, as exemplified herein. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or polymer, or can be delivered by viruses (e.g., adenovirus, AAV), as described herein for nucleic acids encoding a Cas9 guide RNA and/or a Cas9 fusion polypeptide and/or donor polynucleotide.

Guide Nucleic Acid

In some embodiments, the composition comprises guide nucleic acid. Guide nucleic acids suitable for inclusion in a composition of the present disclosure include single-molecule guide RNAs (“single-guide RNA”/“sgRNA”) and dual-molecule guide nucleic acids (“dual-guide RNA”/“dgRNA”).

A guide nucleic acid (e.g., guide RNA) suitable for inclusion in a complex of the present disclosure directs the activities of an RNA-guided endonuclease (e.g., a Cas9 or Cpf1 polypeptide) to a specific target sequence within a target nucleic acid. A guide nucleic acid (e.g., guide RNA) comprises: a first segment (also referred to herein as a “nucleic acid targeting segment”, or simply a “targeting segment”); and a second segment (also referred to herein as a “protein-binding segment”). The terms “first” and “second” do not imply the order in which the segments occur in the guide RNA. The order of the elements relative to one another depends upon the particular RNA-guided polypeptide to be used. For instance, guide RNA for Cas9 typically has the protein-binding segment located 3′ of the targeting segment, whereas guide RNA for Cpf1 typically has the protein-binding segment located 5′ of the targeting segment.

The guide RNA may be introduced into a cell in linear or circular form. If introduced in linear form, the ends of the guide RNA may be protected (e.g., from exonucleolytic degradation) by methods known to those of skill in the art. Amplification procedures such as rolling circle amplification can also be advantageously employed, as exemplified herein.

### First Segment: Targeting Segment

The first segment of a guide nucleic acid (e.g., guide RNA) includes a nucleotide sequence that is complementary to a sequence (a target site) in a target nucleic acid. In other words, the targeting segment of a guide nucleic acid (e.g., guide RNA) can interact with a target nucleic acid (e.g., an RNA, a DNA, a double-stranded DNA) in a sequence-specific manner via hybridization (i.e., base pairing). As such, the nucleotide sequence of the targeting segment may vary and can determine the location within the target nucleic acid that the guide nucleic acid (e.g., guide RNA) and the target nucleic acid will interact. The targeting segment of a guide nucleic acid (e.g., guide RNA) can be modified (e.g., by genetic engineering) to hybridize to any desired sequence (target site) within a target nucleic acid.

The targeting segment can have a length of from 12 nucleotides to 100 nucleotides. The nucleotide sequence (the targeting sequence, also referred to as a guide sequence) of the targeting segment that is complementary to a nucleotide sequence (target site) of the target nucleic acid can have a length of 12 nt or more. For example, the targeting sequence of the targeting segment that is complementary to a target site of the target nucleic acid can have a length of 12 nt or more, 15 nt or more, 17 nt or more, 18 nt or more, 19 nt or more, 20 nt or more, 25 nt or more, 30 nt or more, 35 nt or more or 40 nt.

The percent complementarity between the targeting sequence (i.e., guide sequence) of the targeting segment and the target site of the target nucleic acid can be 60% or more (e.g., 65% or more, 70% or more, 75% or more, 80% or more, 85% or more, 90% or more, 95% or more, 97% or more, 98% or more, 99% or more, or 100%). In some embodiments, the percent complementarity between the targeting sequence of the targeting segment and the target site of the target nucleic acid is 100% over the seven contiguous 5′-mose nucleotides of the target site of the target nucleic acid. In some embodiments, the percent complementarity between the targeting sequence of the targeting segment and the target site of the target nucleic acid is 60% or more over 20 contiguous nucleotides. In some embodiments, the percent complementarity between the targeting sequence of the targeting segment and the target site of the target nucleic acid is 100% over the seventeen, eighteen, nineteen or twenty contiguous 5′-most nucleotides of the target site of the target nucleic acid and as low as 0% or more over the remainder. In such a case, the targeting sequence can be considered to be 17, 18, 19 or 20 nucleotides in length, respectively.

### Second Segment: Protein-Binding Segment

The protein-binding segment of a guide nucleic acid (e.g., guide RNA) interacts with (binds) an RNA-guided endonuclease. The guide nucleic acid (e.g., guide RNA) guides the bound endonuclease to a specific nucleotide sequence within target nucleic acid (the target site) via the above mentioned targeting segment/targeting sequence/guide sequence. The protein-binding segment of a guide nucleic acid (e.g., guide RNA) comprises two stretches of nucleotides that are complementary to one another. The complementary nucleotides of the protein-binding segment hybridize to form a double stranded RNA duplex (dsRNA).

### Single and Dual Guide Nucleic A4cids

A dual guide nucleic acid (e.g., guide RNA) comprises two separate nucleic acid molecules. Each of the two molecules of a subject dual guide nucleic acid (e.g., guide RNA) comprises a stretch of nucleotides that are complementary to one another such that the complementary nucleotides of the two molecules hybridize to form the double stranded RNA duplex of the protein-binding segment.

In some embodiments, the duplex-forming segment of the activator is 60%, 65%, 70%, 75%, 80%, 85%, 90%, 95%, 98%, 99% or more identical or 100% identical to one of the activator (tracrRNA) molecules set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

In some embodiments, the duplex-forming segment of the targeter is 60%, 65%, 70%, 75%, 80%, 85%, 90%, 95%, 98%, 99% or more identical or 100% identical to one of the targeter (crRNA) sequences set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

A dual guide nucleic acid (e.g., guide RNA) can be designed to allow for controlled (i.e., conditional) binding of a targeter with an activator. Because a dual guide nucleic acid (e.g., guide RNA) is not functional unless both the activator and the targeter are bound in a functional complex with Cas9, a dual guide nucleic acid (e.g., guide RNA) can be inducible (e.g., drug inducible) by rendering the binding between the activator and the targeter to be inducible. As one non-limiting example, RNA aptamers can be used to regulate (i.e., control) the binding of the activator with the targeter. Accordingly, the activator and/or the targeter can include an RNA aptamer sequence.

Aptamers (e.g., RNA aptamers) are known in the art and are generally a synthetic version of a riboswitch. The terms “RNA aptamer” and “riboswitch” are used interchangeably herein to encompass both synthetic and natural nucleic acid sequences that provide for inducible regulation of the structure (and therefore the availability of specific sequences) of the nucleic acid molecule (e.g., RNA, DNA/RNA hybrid, etc.) of which they are part. RNA aptamers usually comprise a sequence that folds into a particular structure (e.g., a hairpin), which specifically binds a particular drug (e.g., a small molecule). Binding of the drug causes a structural change in the folding of the RNA, which changes a feature of the nucleic acid of which the aptamer is a part. As non-limiting examples: (i) an activator with an aptamer may not be able to bind to the cognate targeter unless the aptamer is bound by the appropriate drug; (ii) a targeter with an aptamer may not be able to bind to the cognate activator unless the aptamer is bound by the appropriate drug; and (iii) a targeter and an activator, each comprising a different aptamer that binds a different drug, may not be able to bind to each other unless both drugs are present. As illustrated by these examples, a dual guide nucleic acid (e.g., guide RNA) can be designed to be inducible.

Examples of aptamers and riboswitches can be found, for example, in: Nakamura et al., Genes Cells. 2012 May;17(5):344-64; Vavalle et al., Future Cardiol. 2012 May; 8(3):371-82; Citartan et al., Biosens Bioelectron. 2012 Apr. 15; 34(1):1-11; and Liberman et al., Wiley Interdiscip Rev RNA. 2012 May-June; 3(3):369-84; all of which are herein incorporated by reference in their entirety.

Non-limiting examples of nucleotide sequences that can be included in a dual guide nucleic acid (e.g., guide RNA) included in International Patent Application Nos. PCT/US2016/052690 and PCT/US2017/062617, or complements thereof that can hybridize to form a protein binding segment.

A subject single guide nucleic acid (e.g., guide RNA) comprises two stretches of nucleotides (much like a “targeter” and an “activator” of a dual guide nucleic acid) that are complementary to one another, hybridize to form the double stranded RNA duplex (dsRNA duplex) of the protein-binding segment (thus resulting in a stem-loop structure), and are covalently linked by intervening nucleotides (“linkers” or “linker nucleotides”). Thus, a subject single guide nucleic acid (e.g., a single guide RNA) can comprise a targeter and an activator, each having a duplex-forming segment, where the duplex-forming segments of the targeter and the activator hybridize with one another to form a dsRNA duplex. The targeter and the activator can be covalently linked via the 3′ end of the targeter and the 5′ end of the activator. Alternatively, targeter and the activator can be covalently linked via the 5′ end of the targeter and the 3′ end of the activator.

The linker of a single guide nucleic acid can have a length of from 3 nucleotides to 100 nucleotides. In some embodiments, the linker of a single guide nucleic acid (e.g., guide RNA) is 4 nt.

An exemplary single guide nucleic acid (e.g., guide RNA) comprises two complementary stretches of nucleotides that hybridize to form a dsRNA duplex. In some embodiments, one of the two complementary stretches of nucleotides of the single guide nucleic acid (e.g., guide RNA) (or the DNA encoding the stretch) is 60%, 65%, 70%, 75%, 80%, 85%, 90%, 95%, 98%, 99% or more identical or 100% identical to one of the activator (tracrRNA) molecules set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

In some embodiments, one of the two complementary stretches of nucleotides of the single guide nucleic acid (e.g., guide RNA) (or the DNA encoding the stretch) is 60%, 65%, 70%, 75%, 80%, 85%, 90%, 95%, 98%, 99% or more identical or 100% identical to one of the targeter (crRNA) sequences set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

In some embodiments, one of the two complementary stretches of nucleotides of the single guide nucleic acid (e.g., guide RNA) (or the DNA encoding the stretch) is 60%, 65%, 70%, 75%, 80%, 85%, 90%, 95%, 98%, 99% or more identical or 100% identical to one of the targeter (crRNA) sequences or activator (tracrRNA) sequences set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

Appropriate cognate pairs of targeters and activators can be routinely determined by taking into account the species name and base-pairing (for the dsRNA duplex of the protein-binding domain) Any activator/targeter pair can be used as part of dual guide nucleic acid (e.g., guide RNA) or as part of a single guide nucleic acid (e.g., guide RNA).

In some embodiments, an activator (e.g., a trRNA, trRNA-like molecule, etc.) of a dual guide nucleic acid (e.g., guide RNA) (e.g., a dual guide RNA) or a single guide nucleic acid (e.g., guide RNA) (e.g., a single guide RNA) includes a stretch of nucleotides with 60%, 65%, 70%, 75%, 80%, 85%, 90%, 95%, 98%, 99% or more, or 100% sequence identity with an activator (tracrRNA) molecule set forth in International Patent Application Nos. PCT/US2016/052690 and PCT/US2017/062617, or a complement thereof.

In some embodiments, an activator (e.g., a trRNA, trRNA-like molecule, etc.) of a dual guide nucleic acid (e.g., a dual guide RNA) or a single guide nucleic acid (e.g., a single guide RNA) includes 30 or more nucleotides (nt) (e.g., 40 or more, 50 or more, 60 or more, 70 or more, 75 or more nt). In some embodiments, an activator (e.g., a trRNA, trRNA-like molecule, etc.) of a dual guide nucleic acid (e.g., a dual guide RNA) or a single guide nucleic acid (e.g., a single guide RNA) has a length in a range of from 30 to 200 nucleotides (nt).

The protein-binding segment can have a length of from 10 nucleotides to 100 nucleotides.

Also with regard to both a subject single guide nucleic acid (e.g., single guide RNA) and to a subject dual guide nucleic acid (e.g., dual guide RNA), the dsRNA duplex of the protein-binding segment can have a length from 6 base pairs (bp) to 50 bp. The percent complementarity between the nucleotide sequences that hybridize to form the dsRNA duplex of the protein-binding segment can be 60% or more. For example, the percent complementarity between the nucleotide sequences that hybridize to form the dsRNA duplex of the protein-binding segment can be 65% or more, 70% or more, 75% or more, 80% or more, 85% or more, 90% or more, 95% or more, 98% or more, or 99% or more (e.g., in some embodiments, there are some nucleotides that do not hybridize and therefore create a bulge within the dsRNA duplex. In some embodiments, the percent complementarity between the nucleotide sequences that hybridize to form the dsRNA duplex of the protein-binding segment is 100%.

### Hybrid Guide Nucleic Acids

In some embodiments, a guide nucleic acid is two RNA molecules (dual guide RNA). In some embodiments, a guide nucleic acid is one RNA molecule (single guide RNA). In some embodiments, a guide nucleic acid is a DNA/RNA hybrid molecule. In such embodiments, the protein-binding segment of the guide nucleic acid is RNA and forms an RNA duplex. Thus, the duplex-forming segments of the activator and the targeter is RNA. However, the targeting segment of a guide nucleic acid can be DNA. Thus, if a DNA/RNA hybrid guide nucleic acid is a dual guide nucleic acid, the “targeter” molecule and be a hybrid molecule (e.g., the targeting segment can be DNA and the duplex-forming segment can be RNA). In such embodiments, the duplex-forming segment of the “activator” molecule can be RNA (e.g., in order to form an RNA-duplex with the duplex-forming segment of the targeter molecule), while nucleotides of the “activator” molecule that are outside of the duplex-forming segment can be DNA (in which case the activator molecule is a hybrid DNA/RNA molecule) or can be RNA (in which case the activator molecule is RNA). If a DNA/RNA hybrid guide nucleic acid is a single guide nucleic acid, then the targeting segment can be DNA, the duplex-forming segments (which make up the protein-binding segment of the single guide nucleic acid) can be RNA, and nucleotides outside of the targeting and duplex-forming segments can be RNA or DNA.

A DNA/RNA hybrid guide nucleic can be useful in some embodiments, for example, when a target nucleic acid is an RNA. Cas9 normally associates with a guide RNA that hybridizes with a target DNA, thus forming a DNA-RNA duplex at the target site. Therefore, when the target nucleic acid is an RNA, it is sometimes advantageous to recapitulate a DNA-RNA duplex at the target site by using a targeting segment (of the guide nucleic acid) that is DNA instead of RNA. However, because the protein-binding segment of a guide nucleic acid is an RNA-duplex, the targeter molecule is DNA in the targeting segment and RNA in the duplex-forming segment. Hybrid guide nucleic acids can bias Cas9 binding to single stranded target nucleic acids relative to double stranded target nucleic acids.

### Exemplary Guide Nucleic Acids

Any guide nucleic acid can be used. Many different types of guide nucleic acids are known in the art. The guide nucleic selected will be appropriately paired to the particular CRISPR system being used (e.g., the particular RNA guided endonuclease being used). Thus, the guide nucleic acid can be, for instance, a guide nucleic acid corresponding to any RNA guided endonuclease described herein or known in the art. Guide nucleic acids and RNA guided endonucleases are described, for example, in International Patent Application Nos. PCT/US2016/052690 and PCT/US2017/062617

In some embodiments, a suitable guide nucleic acid includes two separate RNA polynucleotide molecules. In some embodiments, the first of the two separate RNA polynucleotide molecules (the activator) comprises a nucleotide sequence having 60% or more (e.g., 65% or more, 70% or more, 75% or more, 80% or more, 85% or more, 90% or more, 95% or more, 98% or more, 99% or more, or 100%) nucleotide sequence identity over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides) to any one of the nucleotide sequences set forth in International Patent Application Nos. PCT/US2016/052690 and PCT/US2017/062617, or a complement thereof. In some embodiments, the second of the two separate RNA polynucleotide molecules (the targeter) comprises a nucleotide sequence having 60% or more (e.g., 65% or more, 70% or more, 75% or more, 80% or more, 85% or more, 90% or more, 95% or more, 98% or more, 99% or more, or 100%) nucleotide sequence identity over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides) to any one of the nucleotide sequences set forth in International Patent Application Nos. PCT/US2016/052690 and PCT/US2017/062617, or a complement thereof.

In some embodiments, a suitable guide nucleic acid is a single RNA polynucleotide and comprises first and second nucleotide sequence having 60% or more (e.g., 65% or more, 70% or more, 75% or more, 80% or more, 85% or more, 90% or more, 95% or more, 98% or more, 99% or more, or 100%) nucleotide sequence identity over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides) to any one of the nucleotide sequences set forth in International Patent Application Nos. PCT/US2016/052690 and PCT/US2017/062617, or complements thereof.

In some embodiments, the guide RNA is a Cpf1 and/or Cas9 guide RNA. A Cpf1 and/or Cas9 guide RNA can have a total length of from 30 nucleotides (nt) to 100 nt, e.g., from 30 nt to 40 nt, from 40 nt to 45 nt, from 45 nt to 50 nt, from 50 nt to 60 nt, from 60 nt to 70 nt, from 70 nt to 80 nt, from 80 nt to 90 nt, or from 90 nt to 100 nt. In some embodiments, a Cpf1 and/or Cas9 guide RNA has a total length of 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, or 50 nt. A Cpf1 and/or Cas9 guide RNA can include a target nucleic acid-binding segment and a duplex-forming segment.

The target nucleic acid-binding segment of a Cpf1 and/or Cas9 guide RNA can have a length of from 15 nt to 30 nt, e.g., 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt. In some embodiments, the target nucleic acid-binding segment has a length of 23 nt. In some embodiments, the target nucleic acid-binding segment has a length of 24 nt. In some embodiments, the target nucleic acid-binding segment has a length of 25 nt.

The target nucleic acid-binding segment of a Cpf1 and/or Cas9 guide RNA can have 100% complementarity with a corresponding length of target nucleic acid sequence. The targeting segment can have less than 100% complementarity with a corresponding length of target nucleic acid sequence. For example, the target nucleic acid binding segment of a Cpf1 and/or Cas9 guide RNA can have 1, 2, 3, 4, or 5 nucleotides that are not complementary to the target nucleic acid sequence. For example, in some embodiments, where a target nucleic acid-binding segment has a length of 25 nucleotides, and the target nucleic acid sequence has a length of 25 nucleotides, in some embodiments, the target nucleic acid-binding segment has 100% complementarity to the target nucleic acid sequence. As another example, in some embodiments, where a target nucleic acid-binding segment has a length of 25 nucleotides, and the target nucleic acid sequence has a length of 25 nucleotides, in some embodiments, the target nucleic acid-binding segment has 1 non-complementary nucleotide and 24 complementary nucleotides with the target nucleic acid sequence.

The duplex-forming segment of a Cpf1 and/or Cas9 guide RNA can have a length of from 15 nt to 25 nt, e.g., 15 nt, 16 nt, 17 nt, 18 nt, 19 nt, 20 nt, 21 nt, 22 nt, 23 nt, 24 nt, or 25 nt.

In some embodiments, the duplex-forming segment of a Cpf1 guide RNA can comprise the nucleotide sequence 5′-AAUUUCUACUGUUGUAGAU-3′.

### Additional Elements

In some embodiments, a guide nucleic acid (e.g., guide RNA) includes an additional segment or segments (in some embodiments at the 5′ end, in some embodiments the 3′ end, in some embodiments at either the 5′ or 3′ end, in some embodiments embedded within the sequence (i.e., not at the 5′ and/or 3′ end), in some embodiments at both the 5′ end and the 3′ end, in some embodiments embedded and at the 5′ end and/or the 3′ end, etc.). For example, a suitable additional segment can include a 5′ cap (e.g., a 7-methylguanylate cap (m⁷G)); a 3′ polyadenylated tail (i.e., a 3′ poly(A) tail); a ribozyme sequence (e.g. to allow for self-cleavage of a guide nucleic acid or component of a guide nucleic acid, e.g., a targeter, an activator, etc.); a riboswitch sequence (e.g., to allow for regulated stability and/or regulated accessibility by proteins and protein complexes); a sequence that forms a dsRNA duplex (i.e., a hairpin)); a sequence that targets an RNA to a subcellular location (e.g., nucleus, mitochondria, chloroplasts, and the like); a modification or sequence that provides for tracking (e.g., a label such as a fluorescent molecule (i.e., fluorescent dye), a sequence or other moiety that facilitates fluorescent detection; a sequence or other modification that provides a binding site for proteins (e.g., proteins that act on DNA, including transcriptional activators, transcriptional repressors, DNA methyltransferases, DNA demethylases, histone acetyltransferases, histone deacetylases, proteins that bind RNA (e.g., RNA aptamers), labeled proteins, fluorescently labeled proteins, and the like); a modification or sequence that provides for increased, decreased, and/or controllable stability; and combinations thereof.

RNA-Guided Endonuclease

In addition to, or instead of, a guide nucleic acid, the composition can comprise an RNA-guided endonuclease protein or nucleic acid (e.g., mRNA or vector) encoding same. Any RNA-guided endonuclease can be used. The selection of the RNA guided endonuclease used will depend, at least in part, to the intended end-use of the CRISPR system employed.

In some embodiments, the polypeptide is a Cas 9 polypeptide. Suitable Cas9 polypeptides for inclusion in a composition of the present disclosure include a naturally-occurring Cas9 polypeptide (e.g., naturally occurs in bacterial and/or archaeal cells), or a non-naturally-occurring Cas9 polypeptide (e.g., the Cas9 polypeptide is a variant Cas9 polypeptide, a chimeric polypeptide as discussed below, and the like), as described below. In some embodiments, one skilled in the art can appreciate that the Cas9 polypeptide disclosed herein can be any variant derived or isolated from any source. In other embodiments, the Cas9 peptide of the present disclosure can include one or more of the mutations described in the literature, including but not limited to the functional mutations described in: Fonfara et al. Nucleic Acids Res. 2014 February; 42(4):2577-90; Nishimasu H. et al. Cell. 2014 Feb. 27; 156(5):935-49; Jinek M. et al. Science. 2012 337:816-21; and Jinek M. et al. Science. 2014 Mar. 14; 343(6176); see also U.S. patent application Ser. No. 13/842,859, filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference. Thus, in some embodiments, the systems and methods disclosed herein can be used with the wild type Cas9 protein having double-stranded nuclease activity, Cas9 mutants that act as single stranded nickases, or other mutants with modified nuclease activity. As such, a Cas9 polypeptide that is suitable for inclusion in a composition of the present disclosure can be an enzymatically active Cas9 polypeptide, e.g., can make single- or double-stranded breaks in a target nucleic acid, or alternatively can have reduced enzymatic activity compared to a wild-type Cas9 polypeptide.

Naturally occurring Cas9 polypeptides bind a guide nucleic acid, are thereby directed to a specific sequence within a target nucleic acid (a target site), and cleave the target nucleic acid (e.g., cleave dsDNA to generate a double strand break, cleave ssDNA, cleave ssRNA, etc.). A subject Cas9 polypeptide comprises two portions, an RNA-binding portion and an activity portion. The RNA-binding portion interacts with a subject guide nucleic acid, and an activity portion exhibits site-directed enzymatic activity (e.g., nuclease activity, activity for DNA and/or RNA methylation, activity for DNA and/or RNA cleavage, activity for histone acetylation, activity for histone methylation, activity for RNA modification, activity for RNA-binding, activity for RNA splicing etc. In some embodiments the activity portion exhibits reduced nuclease activity relative to the corresponding portion of a wild type Cas9 polypeptide. In some embodiments, the activity portion is enzymatically inactive.

Assays to determine whether a protein has an RNA-binding portion that interacts with a subject guide nucleic acid can be any convenient binding assay that tests for binding between a protein and a nucleic acid. Exemplary binding assays include binding assays (e.g., gel shift assays) that involve adding a guide nucleic acid and a Cas9 polypeptide to a target nucleic acid.

Assays to determine whether a protein has an activity portion (e.g., to determine if the polypeptide has nuclease activity that cleave a target nucleic acid) can be any convenient nucleic acid cleavage assay that tests for nucleic acid cleavage. Exemplary cleavage assays that include adding a guide nucleic acid and a Cas9 polypeptide to a target nucleic acid.

In some embodiments, a suitable Cas9 polypeptide for inclusion in a composition of the present disclosure has enzymatic activity that modifies target nucleic acid (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity).

In other embodiments, a suitable Cas9 polypeptide for inclusion in a composition of the present disclosure has enzymatic activity that modifies a polypeptide (e.g., a histone) associated with target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

Many Cas9 orthologues from a wide variety of species have been identified and in some embodiments, the proteins share only a few identical amino acids. All identified Cas9 orthologues have the same domain architecture with a central HNH endonuclease domain and a split RuvC/RNaseH domain. Cas9 proteins share 4 key motifs with a conserved architecture. Motifs 1, 2, and 4 are RuvC like motifs while motif 3 is an HNH-motif.

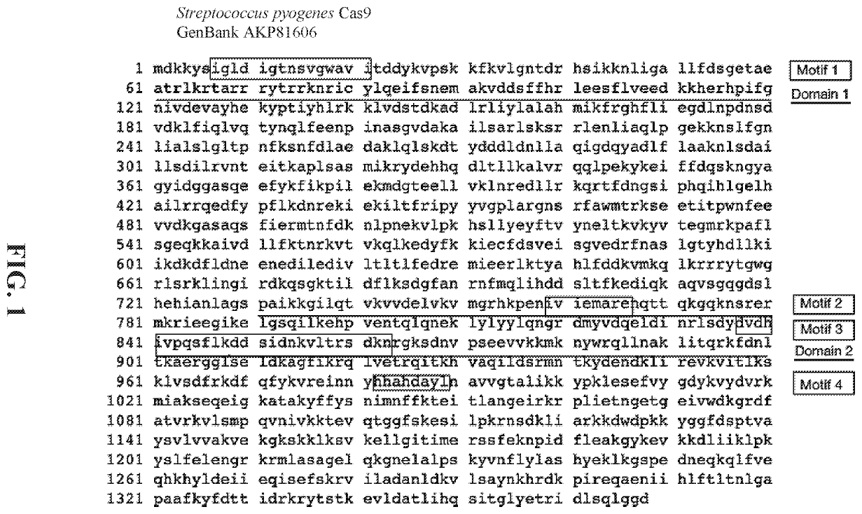

In some embodiments, a suitable Cas9 polypeptide comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity to the Cas9 amino acid sequence depicted in FIG. 1 (SEQ ID NO:1); or alternatively to motifs 1-4 of the Cas9 amino acid sequence depicted in Table 1 below; or alternatively to amino acids 7-166 or 731-1003 of the Cas9 amino acid sequence depicted in FIG. 1 (SEQ ID NO:1)

In some embodiments, a Cas9 polypeptide comprises an amino acid sequence having 75% or more, 80% or more, 85% or more, 90% or more, 95% or more, or 98%, amino acid sequence identity to the amino acid sequence depicted in FIG. 1 and set forth in SEQ ID NO:1; and comprises amino acid substitutions of N497, R661, Q695, and Q926 relative to the amino acid sequence set forth in SEQ ID NO:1; or comprises an amino acid substitution of K855 relative to the amino acid sequence set forth in SEQ ID NO:1; or comprises amino acid substitutions of K810, K1003, and R1060 relative to the amino acid sequence set forth in SEQ ID NO:1; or comprises amino acid substitutions of K848, K1003, and R1060 relative to the amino acid sequence set forth in SEQ ID NO:1.

As used herein, the term “Cas9 polypeptide” encompasses the term “variant Cas9 polypeptide”; and the term “variant Cas9 polypeptide” encompasses the term “chimeric Cas9 polypeptide.”

### Variant Cas9 Polypeptides

A suitable Cas9 polypeptides for inclusion in a composition of the present disclosure includes a variant Cas9 polypeptide. A variant Cas9 polypeptide has an amino acid sequence that is different by one amino acid (e.g., has a deletion, insertion, substitution, fusion) (i.e., different by at least one amino acid) when compared to the amino acid sequence of a wild type Cas9 polypeptide (e.g., a naturally occurring Cas9 polypeptide, as described above). In some instances, the variant Cas9 polypeptide has an amino acid change (e.g., deletion, insertion, or substitution) that reduces the nuclease activity of the Cas9 polypeptide. For example, in some instances, the variant Cas9 polypeptide has less than 50%, less than 40%, less than 30%, less than 20%, less than 10%, less than 5%, or less than 1% of the nuclease activity of the corresponding wild-type Cas9 polypeptide. In some embodiments, the variant Cas9 polypeptide has no substantial nuclease activity. When a Cas9 polypeptide is a variant Cas9 polypeptide that has no substantial nuclease activity, it can be referred to as “dCas9.”

In some embodiments, a variant Cas9 polypeptide has reduced nuclease activity. For example, a variant Cas9 polypeptide suitable for use in a binding method of the present disclosure exhibits less than about 20%, less than about 15%, less than about 10%, less than about 5%, less than about 1%, or less than about 0.1%, of the endonuclease activity of a wild-type Cas9 polypeptide, e.g., a wild-type Cas9 polypeptide comprising an amino acid sequence as depicted in FIG. 1 (SEQ ID NO:1).

In some embodiments, a variant Cas9 polypeptide can cleave the complementary strand of a target nucleic acid but has reduced ability to cleave the non-complementary strand of a double stranded target nucleic acid. For example, the variant Cas9 polypeptide can have a mutation (amino acid substitution) that reduces the function of the RuvC domain (e.g., “domain 1” of FIG. 1). As a non-limiting example, in some embodiments, a variant Cas9 polypeptide has a D10A mutation (e.g., aspartate to alanine at an amino acid position corresponding to position 10 of SEQ ID NO:1) and can therefore cleave the complementary strand of a double stranded target nucleic acid but has reduced ability to cleave the non-complementary strand of a double stranded target nucleic acid (thus resulting in a single strand break (SSB) instead of a double strand break (DSB) when the variant Cas9 polypeptide cleaves a double stranded target nucleic acid) (see, for example, Jinek et al., Science. 2012 Aug. 17; 337(6096):816-21).

In some embodiments, a variant Cas9 polypeptide can cleave the non-complementary strand of a double stranded target nucleic acid but has reduced ability to cleave the complementary strand of the target nucleic acid. For example, the variant Cas9 polypeptide can have a mutation (amino acid substitution) that reduces the function of the HNH domain (RuvC/HNH/RuvC domain motifs, “domain 2” of FIG. 1). As a non-limiting example, in some embodiments, the variant Cas9 polypeptide can have an H840A mutation (e.g., histidine to alanine at an amino acid position corresponding to position 840 of SEQ ID NO:1) (FIG. 1) and can therefore cleave the non-complementary strand of the target nucleic acid but has reduced ability to cleave the complementary strand of the target nucleic acid (thus resulting in a SSB instead of a DSB when the variant Cas9 polypeptide cleaves a double stranded target nucleic acid). Such a Cas9 polypeptide has a reduced ability to cleave a target nucleic acid (e.g., a single stranded target nucleic acid) but retains the ability to bind a target nucleic acid (e.g., a single-stranded or a double-stranded target nucleic acid).

In some embodiments, a variant Cas9 polypeptide has a reduced ability to cleave both the complementary and the non-complementary strands of a double stranded target nucleic acid. As a non-limiting example, in some embodiments, the variant Cas9 polypeptide harbors both the D10A and the H840A mutations (e.g., mutations in both the RuvC domain and the HNH domain) such that the polypeptide has a reduced ability to cleave both the complementary and the non-complementary strands of a double stranded target nucleic acid. Such a Cas9 polypeptide has a reduced ability to cleave a target nucleic acid (e.g., a single-stranded target nucleic acid or a double-stranded target nucleic acid) but retains the ability to bind a target nucleic acid (e.g., a single stranded target nucleic acid or a double-stranded target nucleic acid).

As another non-limiting example, in some embodiments, the variant Cas9 polypeptide harbors W476A and W1126A mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors P475A, W476A, N477A, D1125A, W1126A, and D1127A mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors H840A, W476A, and W1126A, mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors H840A, D10A, W476A, and W1126A, mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors, H840A, P475A, W476A, N477A, D1125A, W1126A, and D1127A mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors D10A, H840A, P475A, W476A, N477A, D1125A, W1126A, and D1127A mutations such that the polypeptide has a reduced ability to cleave a target nucleic acid. Such a Cas9 polypeptide has a reduced ability to cleave a target nucleic acid but retains the ability to bind a target nucleic acid.

Other residues can be mutated to achieve the above effects (i.e. inactivate one or the other nuclease portions). As non-limiting examples, residues D10, G12, G17, E762, H840, N854, N863, H982, H983, A984, D986, and/or A987 can be altered (i.e., substituted) (see Table 1 for more information regarding the conservation of Cas9 amino acid residues). Also, mutations other than alanine substitutions are suitable.

In some embodiments, a variant Cas9 polypeptide that has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the variant Cas9 polypeptide can still bind to target nucleic acid in a site-specific manner (because it is still guided to a target nucleic acid sequence by a guide nucleic acid) as long as it retains the ability to interact with the guide nucleic acid.

TABLE 1
Table 1 lists 4 motifs that are present in
Cas9 sequences from various species The
amino acids listed here are from the Cas9
from S. pyogenes (SEQ ID NO: 1).

| | | Amino acids | Highly |
|---|---|---|---|
| Motif | Motif | (residue #s) | conserved |
| 1 | RuvC | IGLDIGTNSVGWAVI (7-21) | D10, G12, |
| | | (SEQ ID NO: 3) | G17 |
| 2 | RuvC | IVIEMARE (759-766) | E762 |
| | | (SEQ ID NO: 4) | |
| 3 | HNH- | DVDHIVPQSFLKDDSIDNKVLTRSDKN | H840, |
| | motif | (837-863) (SEQ ID NO: 5) | N854, |
| | | | N863 |
| 4 | RuvC | HHAHDAYL (982-989) | H982, |
| | | (SEQ ID NO: 6) | H983, |
| | | | A984, |
| | | | D986, |
| | | | A987 |

In addition to the above, a variant Cas9 protein can have the same parameters for sequence identity as described above for Cas9 polypeptides. Thus, in some embodiments, a suitable variant Cas9 polypeptide comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity of the Cas9 amino acid sequence depicted in FIG. 1 (SEQ ID NO:1), or alternatively to motifs 1-4 (motifs 1-4 of SEQ ID NO:1 are SEQ ID NOs: 3-6, respectively, as depicted in Table 1); or alternatively to amino acids 7-166 or 731-1003 of the Cas9 amino acid sequence depicted in FIG. 1 (SEQ ID NO:1). Any Cas9 protein as defined above can be used as a Cas9 polypeptide, or as part of a chimeric Cas9 polypeptide, in a composition of the present disclosure, including those specifically referenced in International Patent Application Nos. PCT/U52016/052690 and PCT/US2017/062617.

In some embodiments, a suitable variant Cas9 polypeptide comprises an amino acid sequence having 60% or more, 70% or more, 75% or more, 80% or more, 85% or more, 90% or more, 95% or more, 99% or more, or 100% amino acid sequence identity to the Cas9 amino acid sequence depicted in FIG. 1 (SEQ ID NO:1). Any Cas9 protein as defined above can be used as a variant Cas9 polypeptide or as part of a chimeric variant Cas9 polypeptide in a composition of the present disclosure, including those specifically referenced in International Patent Application Nos. PCT/US2016/052690 and PCT/US2017/062617.

### Chimeric Polypeptides (Fusion Polypeptides)

In some embodiments, a variant Cas9 polypeptide is a chimeric Cas9 polypeptide (also referred to herein as a fusion polypeptide, e.g., a “Cas9 fusion polypeptide”). A Cas9 fusion polypeptide can bind and/or modify a target nucleic acid (e.g., cleave, methylate, demethylate, etc.) and/or a polypeptide associated with target nucleic acid (e.g., methylation, acetylation, etc., of, for example, a histone tail).

A Cas9 fusion polypeptide is a variant Cas9 polypeptide by virtue of differing in sequence from a wild type Cas9 polypeptide (e.g., a naturally occurring Cas9 polypeptide). A Cas9 fusion polypeptide is a Cas9 polypeptide (e.g., a wild type Cas9 polypeptide, a variant Cas9 polypeptide, a variant Cas9 polypeptide with reduced nuclease activity (as described above), and the like) fused to a covalently linked heterologous polypeptide (also referred to as a “fusion partner”). In some embodiments, a Cas9 fusion polypeptide is a variant Cas9 polypeptide with reduced nuclease activity (e.g., dCas9) fused to a covalently linked heterologous polypeptide. In some embodiments, the heterologous polypeptide exhibits (and therefore provides for) an activity (e.g., an enzymatic activity) that will also be exhibited by the Cas9 fusion polypeptide (e.g., methyltransferase activity, acetyltransferase activity, kinase activity, ubiquitinating activity, etc.). In some such embodiments, a method of binding, e.g., where the Cas9 polypeptide is a variant Cas9 polypeptide having a fusion partner (i.e., having a heterologous polypeptide) with an activity (e.g., an enzymatic activity) that modifies the target nucleic acid, the method can also be considered to be a method of modifying the target nucleic acid. In some embodiments, a method of binding a target nucleic acid (e.g., a single stranded target nucleic acid) can result in modification of the target nucleic acid. Thus, in some embodiments, a method of binding a target nucleic acid (e.g., a single stranded target nucleic acid) can be a method of modifying the target nucleic acid.

In some embodiments, the heterologous sequence provides for subcellular localization, i.e., the heterologous sequence is a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an endoplasmic reticulum (ER) retention signal, and the like). In some embodiments, a variant Cas9 does not include a NLS so that the protein is not targeted to the nucleus (which can be advantageous, e.g., when the target nucleic acid is an RNA that is present in the cytosol). In some embodiments, the heterologous sequence can provide a tag (i.e., the heterologous sequence is a detectable label) for ease of tracking and/or purification (e.g., a fluorescent protein, e.g., green fluorescent protein (GFP), YFP, RFP, CFP, mCherry, tdTomato, and the like; a histidine tag, e.g., a 6× His tag; a hemagglutinin (HA) tag; a FLAG tag; a Myc tag; and the like). In some embodiments, the heterologous sequence can provide for increased or decreased stability (i.e., the heterologous sequence is a stability control peptide, e.g., a degron, which in some embodiments is controllable (e.g., a temperature sensitive or drug controllable degron sequence, see below). In some embodiments, the heterologous sequence can provide for increased or decreased transcription from the target nucleic acid (i.e., the heterologous sequence is a transcription modulation sequence, e.g., a transcription factor/activator or a fragment thereof, a protein or fragment thereof that recruits a transcription factor/activator, a transcription repressor or a fragment thereof, a protein or fragment thereof that recruits a transcription repressor, a small molecule/drug-responsive transcription regulator, etc.). In some embodiments, the heterologous sequence can provide a binding domain (i.e., the heterologous sequence is a protein binding sequence, e.g., to provide the ability of a Cas9 fusion polypeptide to bind to another protein of interest, e.g., a DNA or histone modifying protein, a transcription factor or transcription repressor, a recruiting protein, an RNA modification enzyme, an RNA-binding protein, a translation initiation factor, an RNA splicing factor, etc.). A heterologous nucleic acid sequence may be linked to another nucleic acid sequence (e.g., by genetic engineering) to generate a chimeric nucleotide sequence encoding a chimeric polypeptide.

A subject Cas9 fusion polypeptide (Cas9 fusion protein) can have multiple (1 or more, 2 or more, 3 or more, etc.) fusion partners in any combination of the above. As an illustrative example, a Cas9 fusion protein can have a heterologous sequence that provides an activity (e.g., for transcription modulation, target modification, modification of a protein associated with a target nucleic acid, etc.) and can also have a subcellular localization sequence. In some embodiments, such a Cas9 fusion protein might also have a tag for ease of tracking and/or purification (e.g., green fluorescent protein (GFP), YFP, RFP, CFP, mCherry, tdTomato, and the like; a histidine tag, e.g., a 6XHis tag; a hemagglutinin (HA) tag; a FLAG tag; a Myc tag; and the like). As another illustrative example, a Cas9 protein can have one or more NLSs (e.g., two or more, three or more, four or more, five or more, 1, 2, 3, 4, or 5 NLSs). In some embodiments a fusion partner (or multiple fusion partners) (e.g., an NLS, a tag, a fusion partner providing an activity, etc.) is located at or near the C-terminus of Cas9. In some embodiments a fusion partner (or multiple fusion partners) (e.g., an NLS, a tag, a fusion partner providing an activity, etc.) is located at the N-terminus of Cas9. In some embodiments a Cas9 has a fusion partner (or multiple fusion partners)(e.g., an NLS, a tag, a fusion partner providing an activity, etc.) at both the N-terminus and C-terminus.

Suitable fusion partners that provide for increased or decreased stability include, but are not limited to degron sequences. Degrons are readily understood by one of ordinary skill in the art to be amino acid sequences that control the stability of the protein of which they are part. For example, the stability of a protein comprising a degron sequence is controlled in part by the degron sequence. In some embodiments, a suitable degron is constitutive such that the degron exerts its influence on protein stability independent of experimental control (i.e., the degron is not drug inducible, temperature inducible, etc.) In some embodiments, the degron provides the variant Cas9 polypeptide with controllable stability such that the variant Cas9 polypeptide can be turned “on” (i.e., stable) or “off” (i.e., unstable, degraded) depending on the desired conditions. For example, if the degron is a temperature sensitive degron, the variant Cas9 polypeptide may be functional (i.e., “on”, stable) below a threshold temperature (e.g., 42° C., 41° C., 40° C., 39° C., 38° C., 37° C., 36° C., 35° C., 34° C., 33° C., 32° C., 31° C., 30° C., etc.) but non-functional (i.e., “off”, degraded) above the threshold temperature. As another example, if the degron is a drug inducible degron, the presence or absence of drug can switch the protein from an “off” (i.e., unstable) state to an “on” (i.e., stable) state or vice versa. An exemplary drug inducible degron is derived from the FKBP12 protein. The stability of the degron is controlled by the presence or absence of a small molecule that binds to the degron.

Examples of suitable degrons include, but are not limited to those degrons controlled by Shield-1, DHFR, auxins, and/or temperature. Non-limiting examples of suitable degrons are known in the art (e.g., Dohmen et al., Science, 1994. 263(5151): p. 1273-1276: Heat-inducible degron: a method for constructing temperature-sensitive mutants; Schoeber et al., Am J Physiol Renal Physiol. 2009 January; 296(1):F204-11: Conditional fast expression and function of multimeric TRPV5 channels using Shield-1; Chu et al., Bioorg Med Chem Lett. 2008 Nov. 15; 18(22):5941-4: Recent progress with FKBP-derived destabilizing domains ; Kanemaki, Pflugers Arch. 2012 Dec. 28: Frontiers of protein expression control with conditional degrons; Yang et al., Mol Cell. 2012 Nov. 30; 48(4):487-8: Titivated for destruction: the methyl degron; Barbour et al., Biosci Rep. 2013 Jan. 18; 33(1): Characterization of the bipartite degron that regulates ubiquitin-independent degradation of thymidylate synthase; and Greussing et al., J Vis Exp. 2012 Nov. 10; (69): Monitoring of ubiquitin-proteasome activity in living cells using a Degron (dgn)-destabilized green fluorescent protein (GFP)-based reporter protein; all of which are hereby incorporated in their entirety by reference).

Exemplary degron sequences have been well-characterized and tested in both cells and animals. Thus, fusing Cas9 (e.g., wild type Cas9; variant Cas9; variant Cas9 with reduced nuclease activity, e.g., dCas9; and the like) to a degron sequence produces a “tunable” and “inducible” Cas9 polypeptide. Any of the fusion partners described herein can be used in any desirable combination. As one non-limiting example to illustrate this point, a Cas9 fusion protein (i.e., a chimeric Cas9 polypeptide) can comprise a YFP sequence for detection, a degron sequence for stability, and transcription activator sequence to increase transcription of the target nucleic acid. A suitable reporter protein for use as a fusion partner for a Cas9 polypeptide (e.g., wild type Cas9, variant Cas9, variant Cas9 with reduced nuclease function, etc.), includes, but is not limited to, the following exemplary proteins (or functional fragment thereof): his3, β-galactosidase, a fluorescent protein (e.g., GFP, RFP, YFP, cherry, tomato, etc., and various derivatives thereof), luciferase, β-glucuronidase, and alkaline phosphatase. Furthermore, the number of fusion partners that can be used in a Cas9 fusion protein is unlimited. In some embodiments, a Cas9 fusion protein comprises one or more (e.g. two or more, three or more, four or more, or five or more) heterologous sequences.

Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity, any of which can be directed at modifying nucleic acid directly (e.g., methylation of DNA or RNA) or at modifying a nucleic acid-associated polypeptide (e.g., a histone, a DNA binding protein, and RNA binding protein, and the like). Further suitable fusion partners include, but are not limited to boundary elements (e.g., CTCF), proteins and fragments thereof that provide periphery recruitment (e.g., Lamin A, Lamin B, etc.), and protein docking elements (e.g., FKBP/FRB, Pil1/Aby1, etc.).

Examples of various additional suitable fusion partners (or fragments thereof) for a subject variant Cas9 polypeptide include, but are not limited to those described in the PCT patent applications: WO2010/075303, WO2012/068627, and WO2013/155555 which are hereby incorporated by reference in their entirety.

Suitable fusion partners include, but are not limited to, a polypeptide that provides an activity that indirectly increases transcription by acting directly on the target nucleic acid or on a polypeptide (e.g., a histone, a DNA-binding protein, an RNA-binding protein, an RNA editing protein, etc.) associated with the target nucleic acid. Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity.

Additional suitable fusion partners include, but are not limited to, a polypeptide that directly provides for increased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.).

Non-limiting examples of fusion partners to accomplish increased or decreased transcription include transcription activator and transcription repressor domains (e.g., the Krüppel associated box (KRAB or SKD); the Mad mSIN3 interaction domain (SID); the ERF repressor domain (ERD), etc.). In some such embodiments, a Cas9 fusion protein is targeted by the guide nucleic acid to a specific location (i.e., sequence) in the target nucleic acid and exerts locus-specific regulation such as blocking RNA polymerase binding to a promoter (which selectively inhibits transcription activator function), and/or modifying the local chromatin status (e.g., when a fusion sequence is used that modifies the target nucleic acid or modifies a polypeptide associated with the target nucleic acid). In some embodiments, the changes are transient (e.g., transcription repression or activation). In some embodiments, the changes are inheritable (e.g., when epigenetic modifications are made to the target nucleic acid or to proteins associated with the target nucleic acid, e.g., nucleosomal histones).

Non-limiting examples of fusion partners for use when targeting ssRNA target nucleic acids include (but are not limited to): splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); heliembodiments; RNA-binding proteins; and the like. It is understood that a fusion partner can include the entire protein or in some embodiments can include a fragment of the protein (e.g., a functional domain).

In some embodiments, the heterologous sequence can be fused to the C-terminus of the Cas9 polypeptide. In some embodiments, the heterologous sequence can be fused to the N-terminus of the Cas9 polypeptide. In some embodiments, the heterologous sequence can be fused to an internal portion (i.e., a portion other than the N- or C-terminus) of the Cas9 polypeptide.

In addition the fusion partner of a chimeric Cas9 polypeptide can be any domain capable of interacting with ssRNA (which, for the purposes of this disclosure, includes intramolecular and/or intermolecular secondary structures, e.g., double-stranded RNA duplexes such as hairpins, stem-loops, etc.), whether transiently or irreversibly, directly or indirectly, including but not limited to an effector domain selected from the group comprising; Endonucleases (for example RNase I, the CRR22 DYW domain, Dicer, and PIN (PilT N-terminus) domains from proteins such as SMG5 and SMG6); proteins and protein domains responsible for stimulating RNA cleavage (for example CPSF, CstF, CFIm and CFIIm); Exonucleases (for example XRN-1 or Exonuclease T) ; Deadenylases (for example HNT3); proteins and protein domains responsible for nonsense mediated RNA decay (for example UPF1, UPF2, UPF3, UPF3b, RNP S1, Y14, DEK, REF2, and SRm160); proteins and protein domains responsible for stabilizing RNA (for example PABP) ; proteins and protein domains responsible for repressing translation (for example Ago2 and Ago4); proteins and protein domains responsible for stimulating translation (for example Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (for example PAP1, GLD-2, and Star-PAP) ; proteins and protein domains responsible for polyuridinylation of RNA (for example CI D1 and terminal uridylate transferase) ; proteins and protein domains responsible for RNA localization (for example from IMP1, ZBP1, She2p, She3p, and Bicaudal-D); proteins and protein domains responsible for nuclear retention of RNA (for example Rrp6); proteins and protein domains responsible for nuclear export of RNA (for example TAP, NXF1, THO, TREX, REF, and Aly) ; proteins and protein domains responsible for repression of RNA splicing (for example PTB, Sam68, and hnRNP A1) ; proteins and protein domains responsible for stimulation of RNA splicing (for example Serine/Arginine-rich (SR) domains) ; proteins and protein domains responsible for reducing the efficiency of transcription (for example FUS (TLS)); and proteins and protein domains responsible for stimulating transcription (for example CDK7 and HIV Tat). Alternatively, the effector domain may be selected from the group comprising Endonucleases; proteins and protein domains capable of stimulating RNA cleavage; Exonucleases; Deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription ; and proteins and protein domains capable of stimulating transcription. Another suitable fusion partner is a PUF RNA-binding domain, which is described in more detail in WO2012068627.

Some RNA splicing factors that can be used (in whole or as fragments thereof) as fusion partners for a Cas9 polypeptide have modular organization, with separate sequence-specific RNA binding modules and splicing effector domains. For example, members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP A1 binds to exonic splicing silencers (ESSs) through its RRM domains and inhibits exon inclusion through a C-terminal Glycine-rich domain. Some splicing factors can regulate alternative use of splice site (ss) by binding to regulatory sequences between the two alternative sites. For example, ASF/SF2 can recognize ESEs and promote the use of intron proximal sites, whereas hnRNP Al can bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple c\acute{ω}-elements that are located in either the core exon region or the exon extension region (i.e., between the two alternative 5′ splice sites). For more examples, see WO2010075303.

In some embodiments, a Cas9 polypeptide (e.g., a wild type Cas9, a variant Cas9, a variant Cas9 with reduced nuclease activity, etc.) can be linked to a fusion partner via a peptide spacer.

In some embodiments, a Cas9 polypeptide comprises a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD attached to another molecule facilitates entry of the molecule into the nucleus (e.g., in some embodiments, a PTD includes a nuclear localization signal (NLS)). In some embodiments, a Cas9 polypeptide comprises two or more NLSs, e.g., two or more NLSs in tandem. In some embodiments, a PTD is covalently linked to the amino terminus of a Cas9 polypeptide. In some embodiments, a PTD is covalently linked to the carboxyl terminus of a Cas9 polypeptide. In some embodiments, a PTD is covalently linked to the amino terminus and to the carboxyl terminus of a Cas9 polypeptide. In some embodiments, a PTD is covalently linked to a nucleic acid (e.g., a guide nucleic acid, a polynucleotide encoding a guide nucleic acid, a polynucleotide encoding a Cas9 polypeptide, etc.). Exemplary PTDs include but are not limited to a minimal undecapeptide protein transduction domain (corresponding to residues 47-57 of HIV-1 TAT comprising YGRKKRRQRRR; SEQ ID NO:7); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:8); Transportan GWTLNSAGYLLGKINLKALAALAKKIL (SEQ ID NO:9); KALAWEAKLAKALAKALAKHLAKALAKALKCEA (SEQ ID NO:10); and RQIKIWFQNRRMKWKK (SEQ ID NO:11). Exemplary PTDs include but are not limited to, YGRKKRRQRRR (SEQ ID NO:12), RKKRRQRRR (SEQ ID NO:13); an arginine homopolymer of from 3 arginine residues to 50 arginine residues; Exemplary PTD domain amino acid sequences include, but are not limited to, any of the following: YGRKKRRQRRR (SEQ ID NO:14); RKKRRQRR (SEQ ID NO:15); YARAAARQARA (SEQ ID NO:16); THRLPRRRRRR (SEQ ID NO:17); and GGRRARRRRRR (SEQ ID NO:18). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

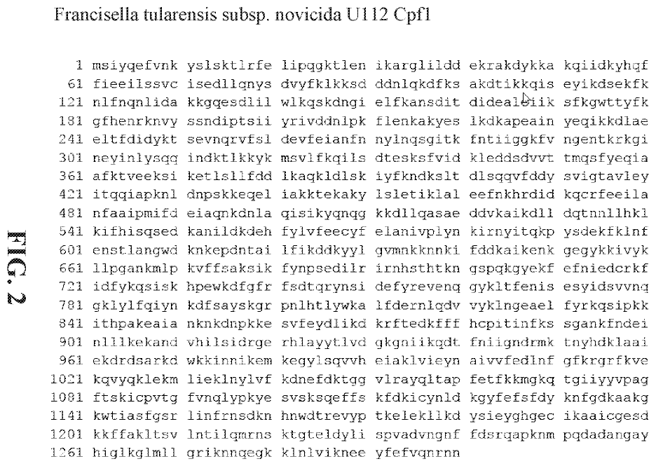

In some embodiments, the composition can comprise a Cpf1 RNA-guided endonuclease, an example of which is provided in FIG. 2, 16, or 17. Another name for the Cpf1 RNA-guided endonuclease is Cas12a. The Cpf1 CRISPR systems of the present disclosure comprise i) a single endonuclease protein, and ii) a crRNA, wherein a portion of the 3′ end of crRNA contains the guide sequence complementary to a target nucleic acid. In this system, the Cpf1 nuclease is directly recruited to the target DNA by the crRNA. In some embodiments, guide sequences for Cpf1 must be at least 12 nt, 13 nt, 14 nt, 15 nt, or 16 nt in order to achieve detectable DNA cleavage, and a minimum of 14 nt, 15 nt, 16 nt, 17 nt, or 18 nt to achieve efficient DNA cleavage.

The Cpf1 systems of the present disclosure differ from Cas9 in a variety of ways. First, unlike Cas9, Cpf1 does not require a separate tracrRNA for cleavage. In some embodiments, Cpf1 crRNAs can be as short as about 42-44 bases long—of which 23-25 nt is guide sequence and 19 nt is the constitutive direct repeat sequence. In contrast, the combined Cas9 tracrRNA and crRNA synthetic sequences can be about 100 bases long.

Second, Cpf1 prefers a “TTN” PAM motif that is located 5′ upstream of its target. This is in contrast to the “NGG” PAM motifs located on the 3′ of the target DNA for Cas9 systems. In some embodiments, the uracil base immediately preceding the guide sequence cannot be substituted (Zetsche, B. et al. 2015. “Cpf1 Is a Single RNA-Guided Endonuclease of a Class 2 CRISPR-Cas System” Cell 163, 759-771, which is hereby incorporated by reference in its entirety for all purposes).

Third, the cut sites for Cpf1 are staggered by about 3-5 bases, which create “sticky ends” (Kim et al., 2016. “Genome-wide analysis reveals specificities of Cpf1 endonucleases in human cells” published online Jun. 6, 2016). These sticky ends with 3-5 bp overhangs are thought to facilitate NHEJ-mediated-ligation, and improve gene editing of DNA fragments with matching ends. The cut sites are in the 3′ end of the target DNA, distal to the 5′ end where the PAM is. The cut positions usually follow the 18th base on the non-hybridized strand and the corresponding 23rd base on the complementary strand hybridized to the crRNA.

Fourth, in Cpf1 complexes, the “seed” region is located within the first 5 nt of the guide sequence. Cpf1 crRNA seed regions are highly sensitive to mutations, and even single base substitutions in this region can drastically reduce cleavage activity (see Zetsche B. et al. 2015 “Cpf1 Is a Single RNA-Guided Endonuclease of a Class 2 CRISPR-Cas System” Cell 163, 759-771). Critically, unlike the Cas9 CRISPR target, the cleavage sites and the seed region of Cpf1 systems do not overlap. Additional guidance on designing Cpf1 crRNA targeting oligos is available on (Zetsche B. et al. 2015. “Cpf1 Is a Single RNA-Guided Endonuclease of a Class 2 CRISPR-Cas System” Cell 163, 759-771).

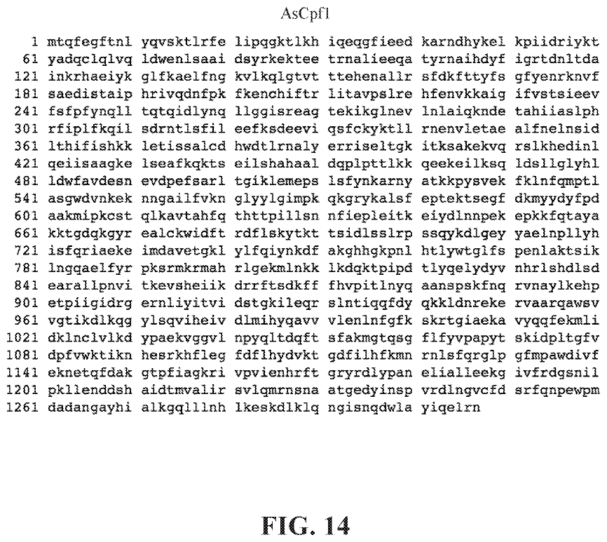

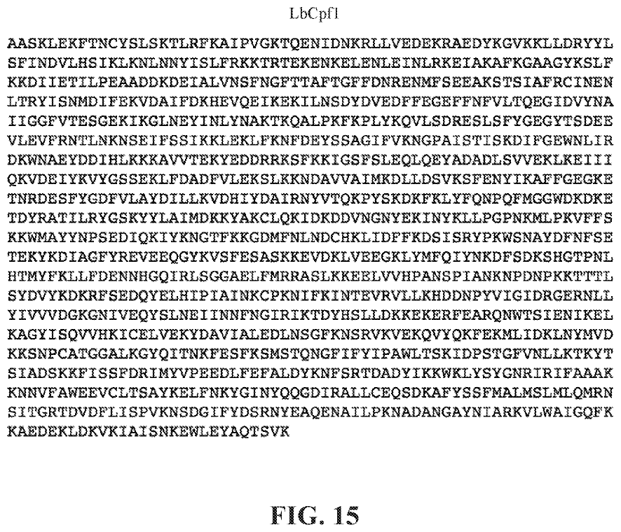

Persons skilled in the art will appreciate that the Cpf1 disclosed herein can be any variant derived or isolated from any source, many of which are known in the art. For example, in some embodiments, the Cpf1 peptide of the present disclosure can include FnCPF1 (e.g., SEQ ID NO: 2) set forth in FIG. 2, AsCpf1 (e.g., FIG. 14), LbCpf1 (e.g., FIG. 15) or any other of the many known Cpf1 proteins from various other microorganism species, and synthetic variants thereof.

In some embodiments, the composition comprises a Cpf1 polypeptide. In some embodiments, the Cpf1 polypeptide is enzymatically active, e.g., the Cpf1 polypeptide, when bound to a guide RNA, cleaves a target nucleic acid. In some embodiments, the Cpf1 polypeptide exhibits reduced enzymatic activity relative to a wild-type Cpf1 polypeptide (e.g., relative to a Cpf1 polypeptide comprising the amino acid sequence depicted in FIG. 2, 16, or 17), and retains DNA binding activity.

In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence depicted in FIG. 2, 16, or 17. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of the amino acid sequence depicted in FIG. 2, 16, or 17.

In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI domain of a Cpf1 polypeptide of the amino acid sequence depicted in FIG. 2, 16, or 17. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCII domain of a Cpf1 polypeptide of the amino acid sequence depicted in FIG. 2, 16, or 17. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCIII domain of a Cpf1 polypeptide of the amino acid sequence depicted in FIG. 2, 16, or 17.

In some embodiments, the Cpf1 polypeptide exhibits reduced enzymatic activity relative to a wild-type Cpf1 polypeptide (e.g., relative to a Cpf1 polypeptide comprising the amino acid sequence depicted in FIG. 2, 16, or 17), and retains DNA binding activity. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence depicted in FIG. 2, 16, or 17; and comprises an amino acid substitution (e.g., a D→A substitution) at an amino acid residue corresponding to amino acid 917 of the amino acid sequence depicted in FIG. 2, 16, or 17. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence depicted in FIG. 2, 16, or 17; and comprises an amino acid substitution (e.g., an E→A substitution) at an amino acid residue corresponding to amino acid 1006 of the amino acid sequence depicted in FIG. 2, 16, or 17. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence depicted in FIG. 2, 16, or 17; and comprises an amino acid substitution (e.g., a D→A substitution) at an amino acid residue corresponding to amino acid 1255 of the amino acid sequence depicted in FIG. 2, 16, or 17.

In some embodiments, the Cpf1 polypeptide is a fusion polypeptide, e.g., where a Cpf1 fusion polypeptide comprises: a) a Cpf1 polypeptide; and b) a heterologous fusion partner. In some embodiments, the heterologous fusion partner is fused to the N-terminus of the Cpf1 polypeptide. In some embodiments, the heterologous fusion partner is fused to the C-terminus of the Cpf1 polypeptide. In some embodiments, the heterologous fusion partner is fused to both the N-terminus and the C-terminus of the Cpf1 polypeptide. In some embodiments, the heterologous fusion partner is inserted internally within the Cpf1 polypeptide.

Suitable heterologous fusion partners include NLS, epitope tags, fluorescent polypeptides, and the like.

Linked Guide RNA and Donor Nucleic Acid

In one aspect, the invention provides a complex comprising a CRISPR system comprising an RNA-guided endonuclease (e.g. a Cas9 or Cpf1 polypeptide), a guide RNA and a donor polynucleotide, wherein the guide RNA and the donor polynucleotide are linked. As exemplified herein, the guide RNA and donor polynucleotide can be either covalently or non-covalently linked. In one embodiment, the guide RNA and donor polynucleotide are chemically ligated. In another embodiment, the guide RNA and donor polynucleotide are enzymatically ligated. In one embodiment, the guide RNA and donor polynucleotide hybridize to each other. In another embodiment, the guide RNA and donor polynucleotide both hybridize to a bridge sequence. Any number of such hybridization schemes are possible.

Deaminase

In some embodiments, the complex or composition further comprises a deaminase (e.g., an adenine base editor). As used herein, the term “deaminase” or “deaminase domain” refers to an enzyme that catalyzes the removal of an amine group from a molecule, or deamination. In some embodiments, the deaminase is a cytidine deaminase, catalyzing the hydrolytic deamination of cytidine or deoxycytidine to uridine or deoxyuridine, respectively. In some embodiments, the deaminase is a cytosine deaminase, catalyzing the hydrolytic deamination of cytosine to uracil (e.g., in RNA) or thymine (e.g., in DNA).

In some embodiments, the deaminase is an adenosine deaminase, which catalyzes the hydrolytic deamination of adenine or adenosine. In some embodiments, the deaminase or deaminase domain is an adenosine deaminase, catalyzing the hydrolytic deamination of adenosine or deoxyadenosine to inosine or deoxyinosine, respectively. In some embodiments, the adenosine deaminase catalyzes the hydrolytic deamination of adenine or adenosine in deoxyribonucleic acid (DNA). The adenosine deaminases (e.g. engineered adenosine deaminases, evolved adenosine deaminases) provided herein may be from any organism, such as a bacterium. In some embodiments, the deaminase or deaminase domain is a variant of a naturally-occurring deaminase from an organism, such as a human, chimpanzee, gorilla, monkey, cow, dog, rat, or mouse.

In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. In some embodiments, the adenosine deaminase is from a bacterium, such as, E. coli, S. aureus, S. typhi, S. putrefaciens, H influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. In some embodiments, the deaminase is APOBEC1 or a variant thereof.

The deaminase can be used in conjugation with any of the other CRISPR elements described herein (i.e., as a composition), or the deaminase can be fused to any of the other CRISPR elements (e.g., Cas9 or Cpf1) described herein (i.e., as a complex). In certain embodiments, the deaminase is fused to Cas9, Cpf1, or a variant thereof.

Other Components

The composition can further comprise any other components typically used in nucleic acid or protein delivery formulations. For instance, the composition can further comprise lipids, lipoproteins (e.g., cholesterol and derivatives), phospholipids, polymers or other components of liposomal or micellar delivery vehicles. The composition also can comprise solvent or carrier suitable for administration to cells or hosts, such as a mammal or human.

In some embodiments, the composition further comprises one or more surfactants. The surfactant can be a non-ionic surfactant and/or a zwitterionic surfactant. In some embodiments, the surfactant is a polymer or copolymer of ethylene oxide (EO), propylene oxide (PO), butylene oxide (BO), glycolic acid (GA), lactic acid (LA), or combinations thereof. For example, the surfactant can be polyethylene glycol (PEG), polypropylene glycol, polyglycolic acid (PGA), polylactic acid, or mixtures thereof. A list of exemplary surfactants includes, but is not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-6301NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); polyoxyethylene-9-lauryl ether, and sorbitan esters (commonly known as the Spans), such as sorbitan trioleate (Span 85) and sorbitan monolaurate. In some embodiments, the surfactant is an anticoagulant (e.g., heparin or the like). In some embodiments, the composition further comprises one or more pharmaceutically acceptable carriers and/or excipients.

In some instances, a component (e.g., a nucleic acid component (e.g., a guide nucleic acid, etc.); a protein component (e.g., a Cas9 or Cpf1 polypeptide, a variant Cas9 or Cpf1 polypeptide); and the like) includes a label moiety. The terms “label”, “detectable label”, or “label moiety” as used herein refer to any moiety that provides for signal detection and may vary widely depending on the particular nature of the assay. Label moieties of interest include both directly detectable labels (direct labels)(e.g., a fluorescent label) and indirectly detectable labels (indirect labels)(e.g., a binding pair member). A fluorescent label can be any fluorescent label (e.g., a fluorescent dye (e.g., fluorescein, Texas red, rhodamine, ALEXAFLUOR® labels, and the like), a fluorescent protein (e.g., green fluorescent protein (GFP), enhanced GFP (EGFP), yellow fluorescent protein (YFP), red fluorescent protein (RFP), cyan fluorescent protein (CFP), cherry, tomato, tangerine, and any fluorescent derivative thereof), etc.). Suitable detectable (directly or indirectly) label moieties for use in the methods include any moiety that is detectable by spectroscopic, photochemical, biochemical, immunochemical, electrical, optical, chemical, or other means. For example, suitable indirect labels include biotin (a binding pair member), which can be bound by streptavidin (which can itself be directly or indirectly labeled). Labels can also include: a radiolabel (a direct label)(e.g., ³H, ¹²⁵I, ³⁵S, ^{b 14}C, or ³²P); an enzyme (an indirect label)(e.g., peroxidase, alkaline phosphatase, galactosidase, luciferase, glucose oxidase, and the like); a fluorescent protein (a direct label)(e.g., green fluorescent protein, red fluorescent protein, yellow fluorescent protein, and any convenient derivatives thereof); a metal label (a direct label); a colorimetric label; a binding pair member; and the like. By “partner of a binding pair” or “binding pair member” is meant one of a first and a second moiety, wherein the first and the second moiety have a specific binding affinity for each other. Suitable binding pairs include, but are not limited to: antigen/antibodies (for example, digoxigenin/anti-digoxigenin, dinitrophenyl (DNP)/anti-DNP, dansyl-X-anti-dansyl, fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Any binding pair member can be suitable for use as an indirectly detectable label moiety.

Any given component, or combination of components can be unlabeled, or can be detectably labeled with a label moiety. In some embodiments, when two or more components are labeled, they can be labeled with label moieties that are distinguishable from one another.

Encapsulation and Nanoparticles

In some embodiments of the composition, the polymer combines with the nucleic acid and/or polypeptide and partially or completely encapsulates the nucleic acid and/or polypeptide. The composition can, in some formulations, provide a nanoparticle comprising the polymer and nucleic acid and/or polypeptide.

In some embodiments, the composition can comprise a core nanoparticle in addition to the polymer described herein and the nucleic acid or polypeptide. Any suitable nanoparticle can be used, including metal (e.g., gold) nanoparticles or polymer nanoparticles.

The polymer described herein and the nucleic acid (e.g., guide RNA, donor polynucleotide, or both) or polypeptide can be conjugated directly or indirectly to a nanoparticle surface. For example, the polymer described herein and the nucleic acid (e.g., guide RNA, donor polynucleotide, or both) or polypeptide can be conjugated directly to the surface of a nanoparticle or indirectly through an intervening linker.

Any type of molecule can be used as a linker. For example, a linker can be an aliphatic chain including at least two carbon atoms (e.g., 3, 4, 5, 6, 7, 8, 9, 10 or more carbon atoms), and can be substituted with one or more functional groups including ketone, ether, ester, amide, alcohol, amine, urea, thiourea, sulfoxide, sulfone, sulfonamide, and disulfide functionalities. In embodiments where the nanoparticle includes gold, a linker can be any thiol-containing molecule. Reaction of a thiol group with the gold results in a covalent sulfide (—S—) bond. Linker design and synthesis are well known in the art.

In some embodiments, the nucleic acid conjugated to the nanoparticle is a linker nucleic acid that serves to non-covalently bind one or more elements described herein (e.g., a Cas9 polypeptide, and a guide RNA, a donor polynucleotide, and a Cpf1 polypeptide) to the nanoparticle-nucleic acid conjugate. For instance, the linker nucleic acid can have a sequence that hybridizes to the guide RNA or donor polynucleotide.

The nucleic acid conjugated to the nanoparticle (e.g., a colloidal metal (e.g., gold) nanoparticle; a nanoparticle comprising a biocompatible polymer) can have any suitable length. When the nucleic acid is a guide RNA or donor polynucleotide, the length will be as suitable for such molecules, as discussed herein and known in the art. If the nucleic acid is a linker nucleic acid, it can have any suitable length for a linker, for instance, a length of from 10 nucleotides (nt) to 1000 nt, e.g., from about 1 nt to about 25 nt, from about 25 nt to about 50 nt, from about 50 nt to about 100 nt, from about 100 nt to about 250 nt, from about 250 nt to about 500 nt, or from about 500 nt to about 1000 nt. In some instances, the nucleic acid conjugated to the nanoparticle (e.g., a colloidal metal (e.g., gold) nanoparticle; a nanoparticle comprising a biocompatible polymer) nanoparticle can have a length of greater than 1000 nt.

When the nucleic acid linked (e.g., covalently linked; non-covalently linked) to a nanoparticle comprises a nucleotide sequence that hybridizes to at least a portion of the guide RNA or donor polynucleotide present in a complex of the present disclosure, it has a region with sequence identity to a region of the complement of the guide RNA or donor polynucleotide sequence sufficient to facilitate hybridization. In some embodiments, a nucleic acid linked to a nanoparticle in a complex of the present disclosure has at least 50%, at least 60%, at least 70%, at least 80%, at least 90%, at least 95%, at least 98%, at least 99%, or 100%, nucleotide sequence identity to a complement of from 10 to 50 nucleotides (e.g., from 10 nucleotides (nt) to 15 nt, from 15 nt to 20 nt, from 20 nt to 25 nt, from 25 nt to 30 nt, from 30 nt to 40 nt, or from 40 nt to 50 nt) of a guide RNA or donor polynucleotide present in the complex.

In some embodiments, a nucleic acid linked (e.g., covalently linked; non-covalently linked) to a nanoparticle is a donor polynucleotide, or has the same or substantially the same nucleotide sequence as a donor polynucleotide. In some embodiments, a nucleic acid linked (e.g., covalently linked; non-covalently linked) to a nanoparticle comprises a nucleotide sequence that is complementary to a donor DNA template.

Method of Use

Also provided herein is a method of delivering a nucleic acid and/or polypeptide to a cell, wherein the cell can be in vitro or in vivo. The method comprises administering a composition comprising the polymer and nucleic acid and/or polypeptide, as described herein, to the cell or to a subject containing the cell. The method can be used with respect to any type of cell or subject, but is particularly useful for mammalian cells (e.g., human cells). In some embodiments, the polymer comprises a targeting agent, such that nucleic acid and/or polypeptide is delivered predominantly or exclusively to target cells or tissues (e.g., cells or tissues of the peripheral nervous system, the central nervous system, the eye of the subject, liver, muscle, lung, bone (e.g., hematopoietic cells), or tumor cells or tissues).

When used to deliver a protein or nucleic acid to a cell in a subject (i.e., in vivo), it is desirable that the polymer is stable in serum. Stability in serum can be assessed as a function of the efficiency by which the polymer delivers a protein or nucleic acid payload to a cell in serum (e.g., in vitro or in vivo). Thus, in some embodiments, the polymer delivers a given protein or nucleic acid to a cell in serum with an efficiency greater than pAsp[DET] under the same conditions.

When used with a composition comprising one or more components of a CRISPR system, the method may be employed to edit a target nucleic acid or gene. In some embodiments, a method of modifying a target nucleic acid comprises homology-directed repair (HDR). In some embodiments, use of a complex of the present disclosure to carry out HDR provides an efficiency of HDR of at least 1%, at least 2%, at least 3%, at least 4%, at least 5%, at least 10%, at least 15%, at least 20%, at least 25%, or more than 25%. In some embodiments, a method of modifying a target nucleic acid comprises non-homologous end joining (NHEJ). In some embodiments, use of a complex of the present disclosure to carry out HDR provides an efficiency of NHEJ of at least 1%, at least 2%, at least 3%, at least 4%, at least 5%, at least 10%, at least 15%, at least 20%, at least 25%, or more than 25%.

The following examples further illustrate the invention but, of course, should not be construed as in any way limiting its scope.

### EXAMPLE 1

This example provides guidance for the synthesis of a polymer described herein. The synthesis includes modifying PBLA with an amine and N-(2-aminoethyl)ethane-1,2-diamine (“DET”). An exemplary procedure is as follows.

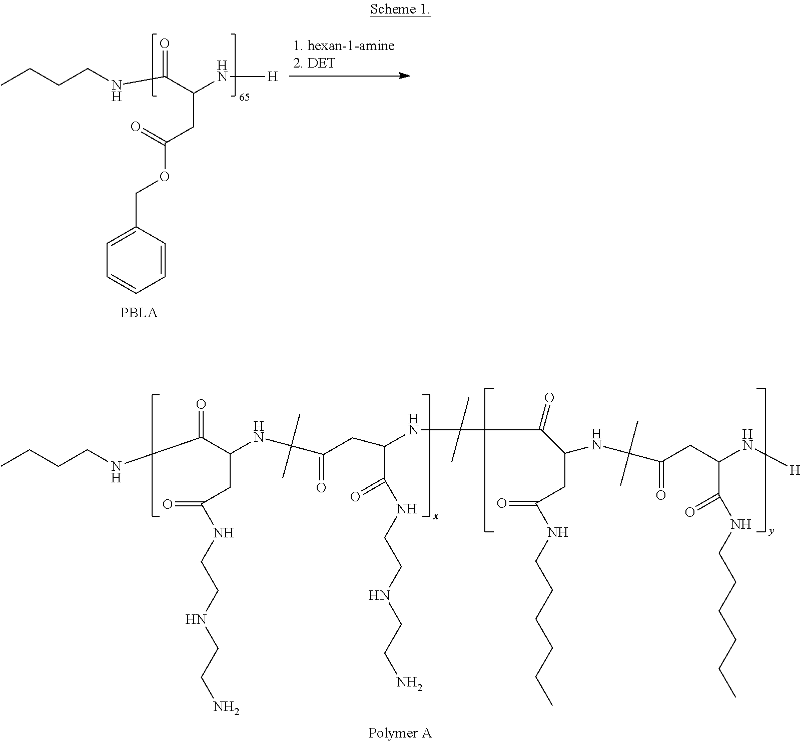

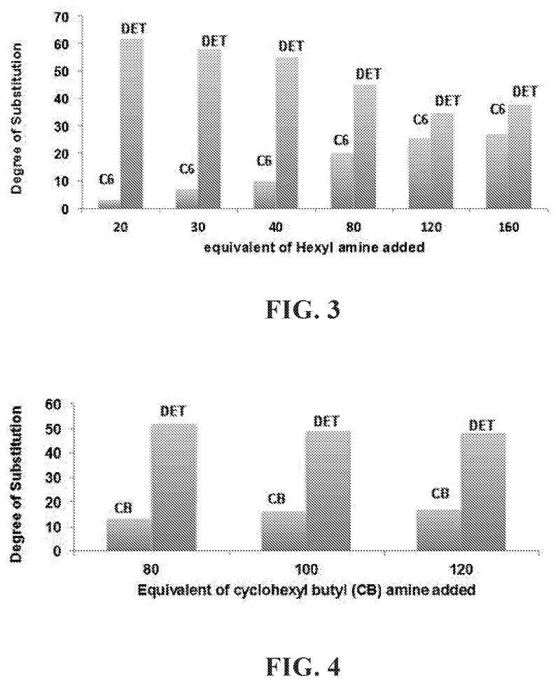

Lyophilized PBLA (50 mg, 0.0037 mMol) was placed into a flask and dissolved in tetrahydrofuran/N-methyl-2-pyrrolidine (1 mL each). To the clear solution was added n-hexylamine (58.8 uL, 0.44 mMol, 120 equivalents), and the clear reaction mixture was stirred for 24 hours at room temperature. After approximately 24 hours, diethylenetriamine (50 equivalents to benzyl group of PBLA segment, 1.0 g) was added to the clear mixture under mild anhydrous conditions. After approximately 18 hours at room temperature, the reaction mixture was precipitated into diethyl ether (10-12× volume, 35 mL). The white precipitate was then centrifuged, and washed twice with diethyl ether. The white polymer was dissolved in 1 M HCl (3 mL) and dialyzed in an excess of deionized water in a 3.5-5 KD cut-off membrane. When the pH of the solution was between 5-6, the dialysis was stopped, and the solution was lyophilized, to give approximately 60 mg of polymer product. A similar procedure was performed using different ratios of n-hexylamine to PBLA to provide polymers A1-A6. The concentration ratios and polymers derived therefrom are set forth in Table 2, with values x and y reported as an average. The degree of substitution was confirmed by ¹H NMR spectroscopy. The results are also plotted in FIG. 3

TABLE 2

| Polymer | Equivalents of hexan-1-amine to PBLA | x | y |
|---|---|---|---|
| Polymer A1 | 20 | 62 | 3 |
| Polymer A2 | 30 | 58 | 7 |
| Polymer A3 | 40 | 55 | 10 |
| Polymer A4 | 80 | 45 | 20 |
| Polymer A5 | 120 | 39.5 | 25.5 |
| Polymer A6 | 160 | 38 | 27 |

As demonstrated by Table 2 and FIG. 3, the degree of substitution of hydrophobic moiety can be controlled by the equivalent of hydrophobic moiety added in the reaction mixture.

### EXAMPLE 2

This example provides guidance for the synthesis of a polymer described herein. The synthesis includes modifying PBLA with an amine and N-(2-aminoethyl)ethane-1,2-diamine (“DET”). An exemplary procedure is as follows.

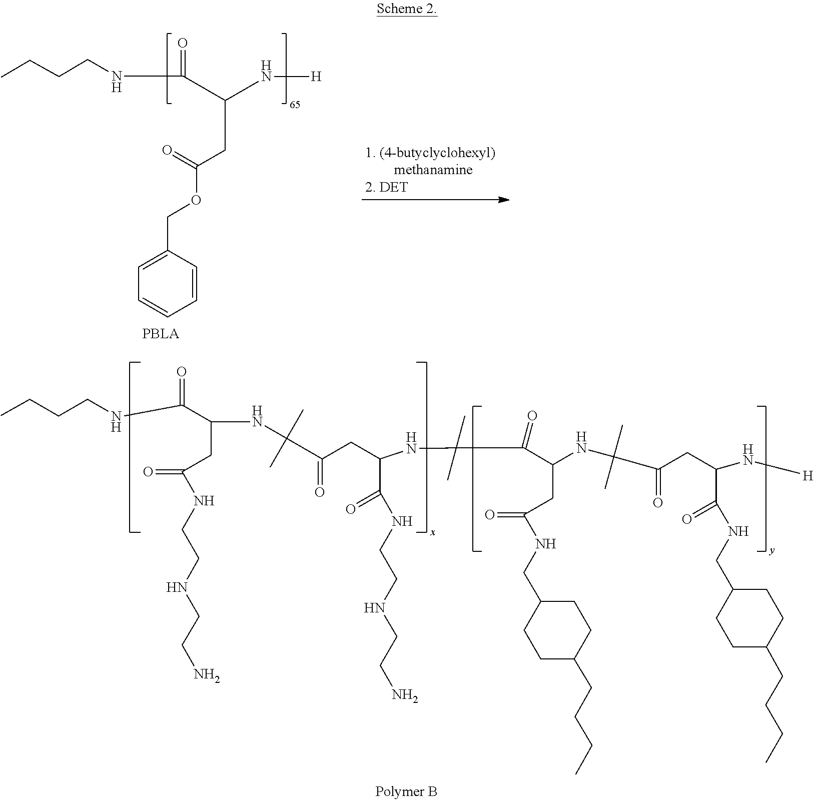

Lyophilized PBLA (50 mg, 0.0037 mMol) was placed into a flask and dissolved in tetrahydrofuran/N-methyl-2-pyrrolidine (1 mL each). To the clear solution was added 1-(4-butylcyclohexyl)methanamine (75 mg, 0.44 mMol, 120 equivalents), and the clear reaction mixture was stirred for 24 hours at room temperature. After approximately 24 hours, diethylenetriamine (50 equivalents to benzyl group of PBLA segment) was added to the clear mixture under mild anhydrous conditions. After approximately 18 hours at room temperature, the reaction mixture was precipitated into diethyl ether (10-12× volume, 35 mL). The white precipitate was then centrifuged, and washed twice with diethyl ether. The white polymer was dissolved in 1 M HCl (3 mL) and dialyzed in an excess of deionized water in a 3.5-5 KD cut-off membrane. When the pH of the solution was between 5-6, the dialysis was stopped, and the solution was lyophilized, to give polymer product. A similar procedure was performed using different ratios of 1-(4-butylcyclohexyl)methanamine to PBLA to provide polymers B1-B3. The concentration ratios, and polymers derived therefrom are set forth in Table 3, with values x and y reported as an average. The degree of substitution was confirmed by ¹H NMR spectroscopy. The results are also plotted in FIG. 4

TABLE 3

| | Equivalents of (4- | | |
|---|---|---|---|
| Polymer | butylcyclohexyl)methanamine to PBLA | X | Y |
| Polymer B1 | 80 | 52 | 13 |
| Polymer B2 | 100 | 49 | 16 |
| Polymer B3 | 120 | 47 | 18 |

As demonstrated by Table 3 and FIG. 4, the degree of substitution of hydrophobic moiety can be controlled by the equivalent of hydrophobic moiety added in the reaction mixture.

### EXAMPLE 3

The following example illustrates the effect of increasing hydrophobic side chain substitution to the polymers described herein on delivery of mRNA to cells.

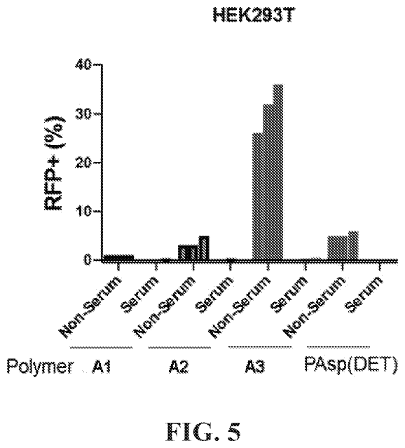

Polymers A1, A2, and A3 of Example 1 were formulated with mRNA encoding Red Fluorescent Protein (RFP) and cultured with HEK293T cells in both serum and non-serum conditions. pAsp[DET] was used as a positive control. The results (FIG. 5) show some transfection in all non-serum samples, with increasing transfection efficiency using polymers that had the higher degree of hexylamine substitution. Much higher transfection was observed using polymer A3.

### EXAMPLE 4

The following example illustrates the use of polymers of the invention to deliver mRNA to various cells.

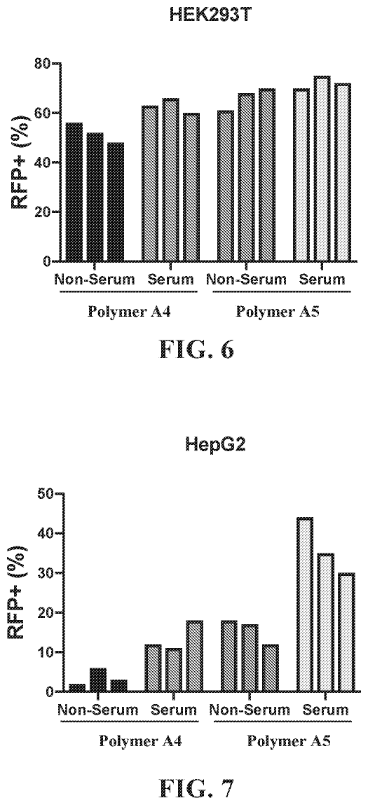

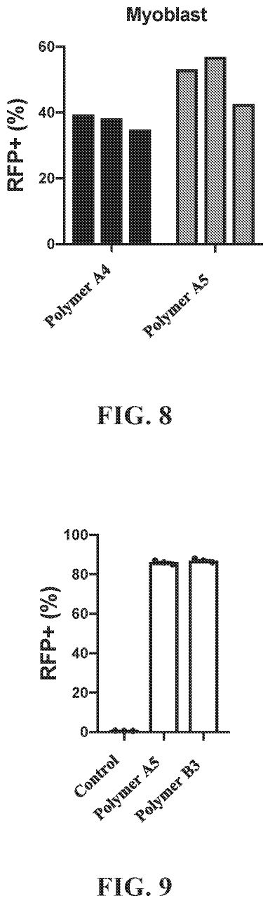

Polymers A4 and A5 of Example 1 were formulated with mRNA encoding mCherry and cultured with HEK293T and HepG2 under both serum and non-serum conditions, and primary myoblasts from Mdx mouse under serum conditions. The results are presented in FIGS. 6-8, which show good transfection in all samples. The highest transfection levels were obtained using polymer A5 having the higher level of hexylamine substitution.

Polymer A5 of Example 1 (hexylamine substitution) and Polymer B3 of Example 2 ((4-butylcyclohexyl)methanamine substitution) were formulated mCherry mRNA and cultured with HEK293T cells. The results are presented in FIG. 9. Both polymers showed excellent transfection efficiency.

### EXAMPLE 5

The following example illustrates the use of polymers of the invention to deliver CRISPR ribonucleoproteins and single guide RNA (sgRNA) to cells.

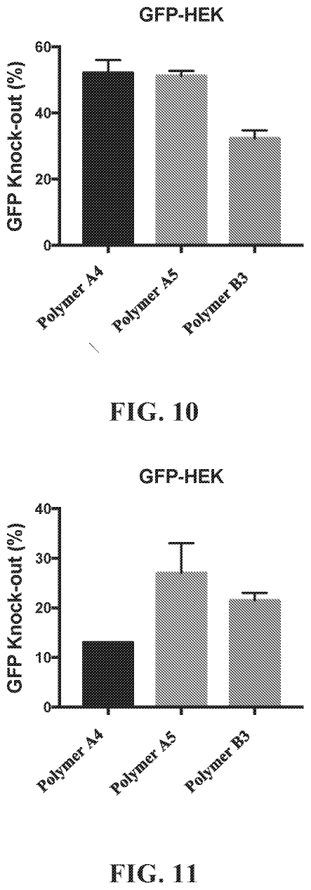

Polymers A4 and A5 of Example 1 (hexylamine substitution) and Polymer B3 of Example 2 ((4-butylcyclohexyl)methanamine substitution) were used for this experiment. Each polymer was mixed with either (a) GFP-targeting sgRNA (600 ng) and Cas9 (3 ug), or (b) GFP-targeting crRNA (300 ng) and Cpf1 (3 ug) to provide loaded polymer nanoparticles. GFP expressing HEK293T cells (GFP-HEK cells) seeded at 20,000 cell density in serum were treated with the loaded polymer nanoparticles and doxycycline induction was conducted two days after the transfection. Flow cytometry was conducted 48 hr after the induction and the percentage cells that were GFP—was quantified. The results are presented in FIG. 10 (Cas9 loaded polymers; n=3 and error bar=SEM) and FIG. 11 (Cpf1 loaded polymers; n=3 and error bar=SEM). All three polymers showed significant GFP knock-out using either Cas9 or Cpf1.

### EXAMPLE 6

The following example illustrates the stability of nanoparticles comprising the polymers of the invention.

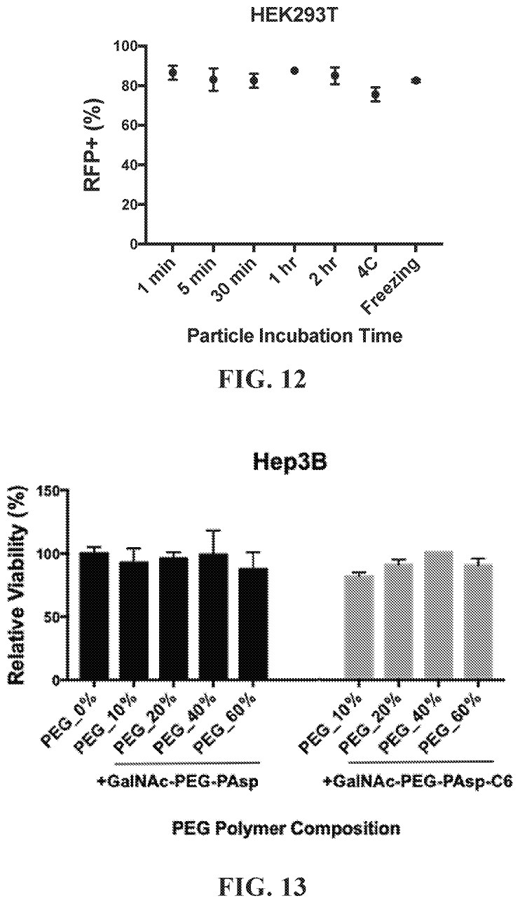

mCherry mRNA was mixed with polymer A5 of Example 1 to provide loaded nanoparticles. One sample of the nanoparticles was incubated from 1 min to 2 hr at room temperature. Another sample was stored at 4 degree Celsius for 2 hr. A third sample was frozen at −80 degree Celsius for 2 hr. The nanoparticles were used to treat HEK293T cells, and mCherry expression was quantified with flow cytometry. The results are presented in FIG. 12 (n=2, error bar=SEM). As shown, the nanoparticles retained almost all transfection efficiency, demonstrating that the nanoparticles were stable under the tested conditions.

### EXAMPLE 7

The following example illustrates the use of the polymers provided herein co-mixed with PEGylated polymers to deliver mRNA to cells.

Polymer A5 of Example 1 was mixed with increasing amounts of GalNAc-PEG-PAsp or GalNAc-PEG-PAsp-C6 (i.e., 10 wt. %, 20 wt. %, 40 wt. % and 60 wt. % of the total composition) and used to deliver mCherry mRNA to Hep3B cells.

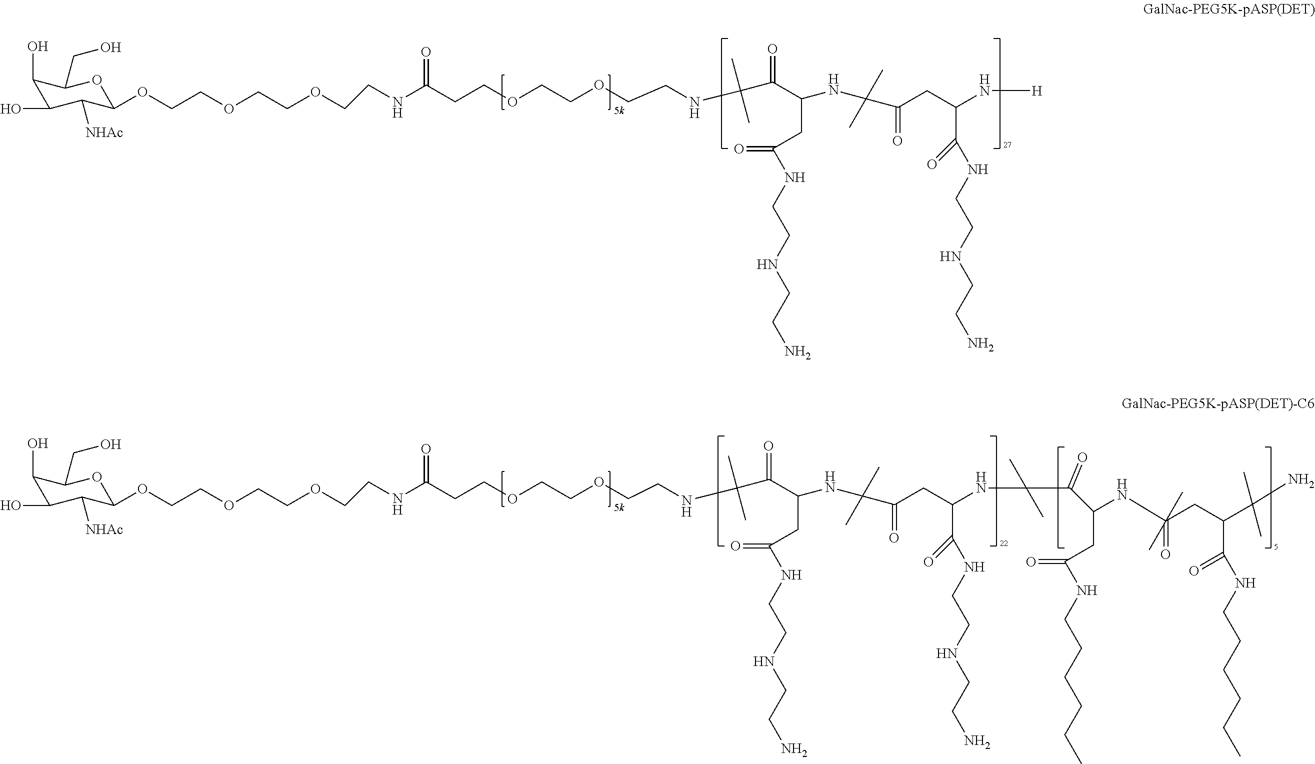

Cell viability was measured by Cell counting kit-8 (CCK-8) assay and RFP+percent was measured with flow cytometry. The results are presented in FIG. 13 (n=2, error bar=SEM). Polymer A5 of Example 1 mixed with either GalNAc-PEG-PAsp or GalNAc-PEG-PAsp-C6 showed very efficient delivery for compositions containing GalNAc-PEG-PAsp or GalNAc-PEG-PAsp-C6 in amounts of 10 wt. %, 20 wt. %, 40 wt. % and 60 wt. % of the total composition. GalNAc-PEG-PAsp and GalNAc-PEG-PAsp-C6 showed a slight reduction of transfection efficiency when transitioning from 40 wt. % to 60 wt. % of the total composition, which was expected. It is known that high composition of PEG can hinder cellular uptake. CCK-8 assay showed that the tested polymers did not cause cell toxicity with the dose that was used. At a dose that yielded more than 60% mRNA transfection, there was no cytotoxicity observed.

Both transfection efficiency and cell viability results show that the polymer provided herein can be co-formulated with PEG-Polymers.

### EXAMPLE 8

Polymer H27N was prepared and used in Examples 9, 10, 12-14, 16-21, 23 provided herein:

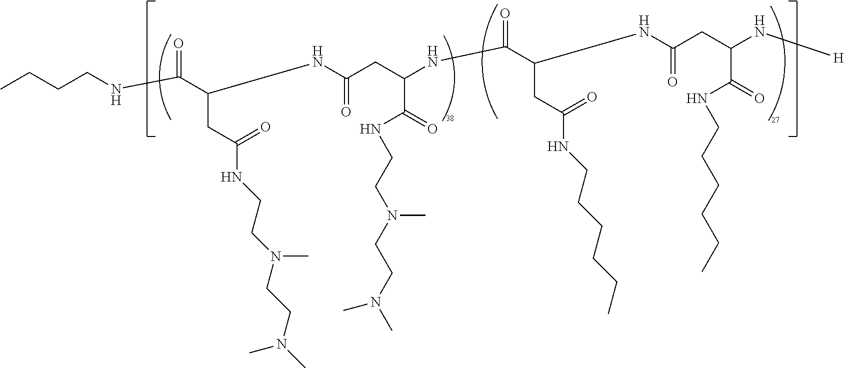

(H27N; bracketing does not imply block copolymer structure) H27N can be prepared by modifying PBLA with N¹-(2-aminoethyl)-N¹,N²,N²-trimethylethane-1,2-diamine and hexylamine. An exemplary procedure is as follows.

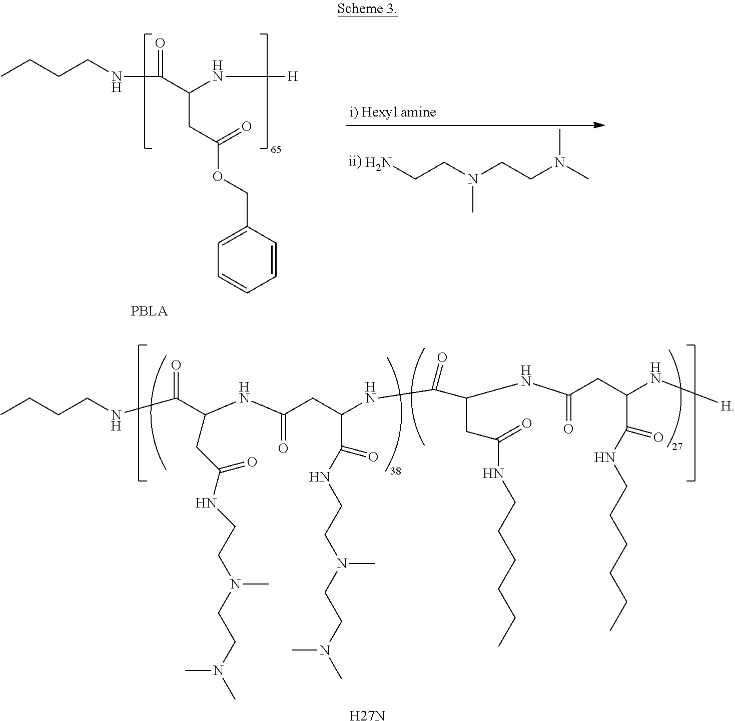

Lyophilized PBLA (50 mg, 0.0037 mmol; degree of polymerization (“DP”) 65) was placed into a flask and dissolved in tetrahydrofuran/N-methyl-2-pyrrolidine (1 mL each). To the clear solution was added n-hexylamine (160 equivalents), and the clear reaction mixture was stirred for 24 hours at room temperature. After approximately 24 hours, N¹-(2-aminoethyl)-N¹,N²,N²-trimethylethane-1,2-diamine (50 equivalents to benzyl group of PBLA segment) was added to the clear mixture under mild anhydrous conditions. After approximately 18 hours at room temperature, the reaction mixture was precipitated into diethyl ether (10-12× volume, 35 mL). The precipitate was then centrifuged, and washed twice with diethyl ether. The polymer was dissolved in 1 M HCl (3 mL) and dialyzed in an excess of deionized water in a 3.5-5 KD cut-off membrane. When the pH of the solution was between 5-6, the dialysis was stopped, and the solution was lyophilized, to give the polymer product.

### EXAMPLE 9

The following example shows the ability of polymer H27N of Example 8 to form a nanoparticle when combined with mCherry mRNA.

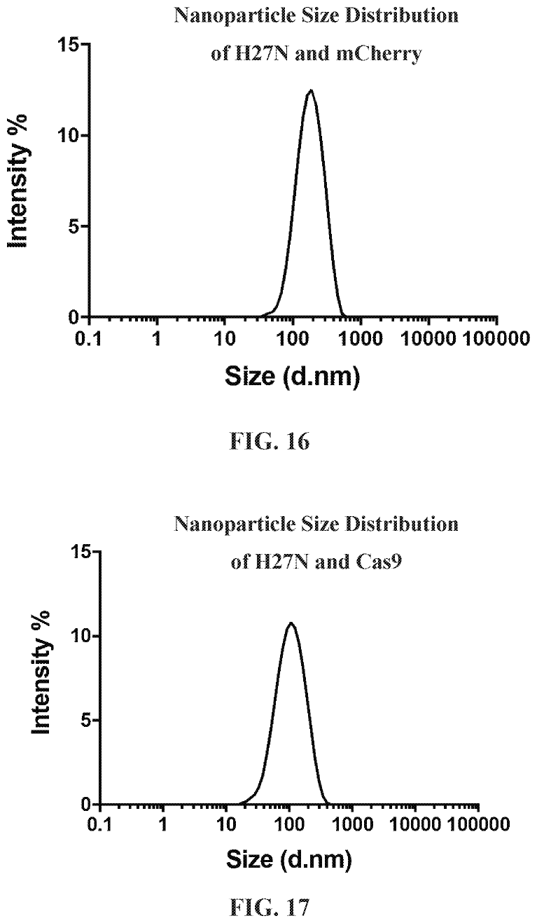

Polymer H27N was combined with mCherry mRNA and the resulting mixture was analyzed using dynamic light scattering. The results are plotted in FIG. 16.

As demonstrated by the dynamic light scattering plot of FIG. 16, the combination of H27N and mCherry mRNA results in clear nanoparticle formation. The resulting nanoparticle had an average diameter of 195 nm and a polydispersity index of 0.16.

### EXAMPLE 10

The following example shows the ability of polymer H27N of Example 8 to form a nanoparticle when combined with Cas9 RNP.

Polymer H27N was combined with Cas9 RNP and the resulting mixture was analyzed using dynamic light scattering. The results are plotted in FIG. 17.

As demonstrated by the dynamic light scattering plot of FIG. 17, the combination of H27N and Cas9 RNP results in clear nanoparticle formation. The resulting nanoparticle had an average diameter of 92 nm and a polydispersity index of 0.21.

### EXAMPLE 11

Four polymers were prepared using a similar synthetic procedure to the one set forth in Example 8. The resulting polymers were used in Examples 12-14 provided herein. As is be apparent from the production method, the bracketing in the below structures does not signify block copolymer structure.

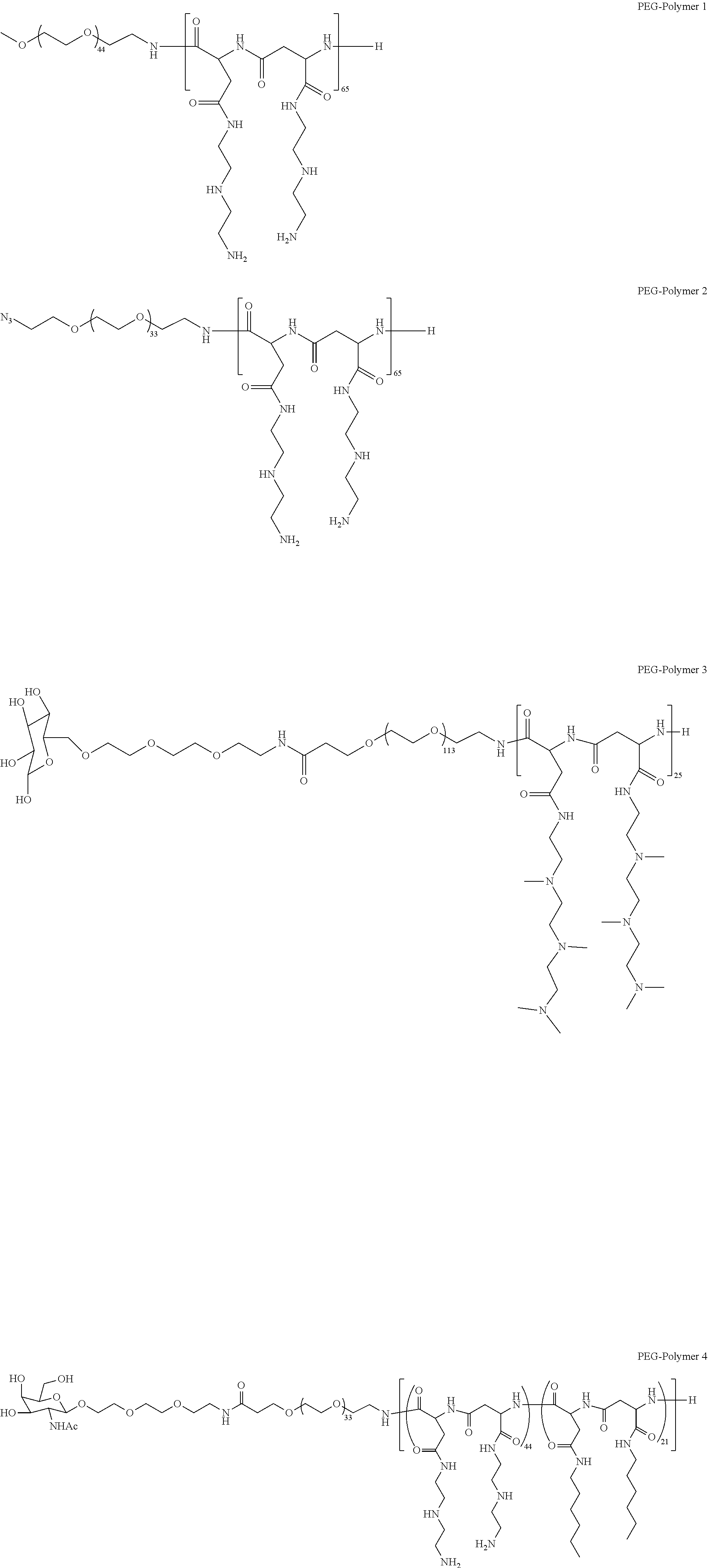

### EXAMPLE 12

The following example illustrates the use of Polymer H27N and PEG-Polymers 2-4 to deliver mRNA to HEK293T cells.

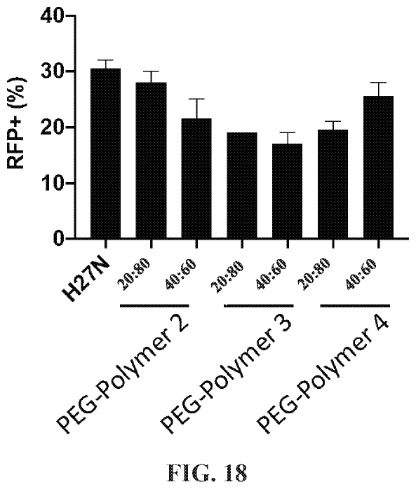

RFP mRNA was delivered with H27N and co-mixtures of H27N and PEG-Polymers 2-4 in a ratio of 20:80 or 40:60 wt. % of PEG-Polymer relative to H27N). Co-mixtures of H27N and PEG-Polymers 2-4 (ratio of 20:80 or 40:60 wt. % of PEG-Polymer relative to H27N) were prepared prior to addition of RFP mRNA. The resulting nanoparticle was treated in HEK293T cells and flow cytometry was used to quantify RFP+cells 24 hours after transfection. The results are plotted in FIG. 18.

As demonstrated by FIG. 18, Polymer H27N was efficient at delivering mRNA in HEK293T cells alone and in combination with PEG-Polymers 2-4, which combinations showed comparable or slightly reduced transfection efficiency in HEK293T cells.

### EXAMPLE 13

The following example illustrates the effect of PEG-Polymers 1-3 on the ability of Polymer H27N to deliver Cas9 RNP to Hep3B cells.

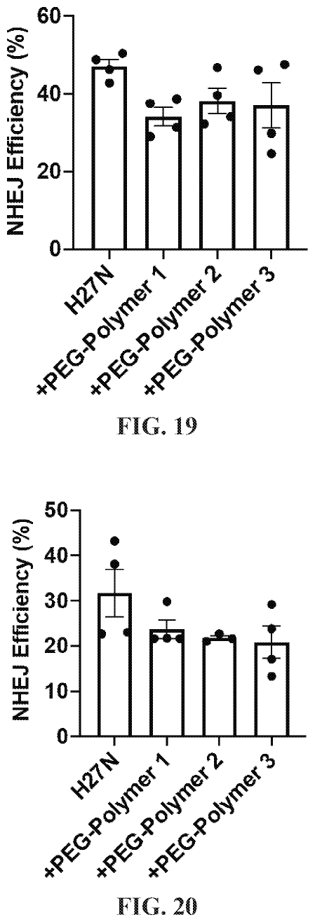

Hep3B cells were seeded 50,000 cells/well in culture medium composed of Dulbecco's Modified Eagle Medium (DMEM) and 10% fetal bovine serum (FBS) to form 40 pmol Cas9 RNP. sgRNA targeting SERPINA1 gene was prepared and Cas9 protein was added slowly and mixed thoroughly by pipetting. Separately, compositions containing H27N polymer and PEG-Polymers 1-3 were prepared using a 1:1 ratio. Nanoparticles were formed by mixing the resulting compositions with sgRNA using a mass ratio of polymer to sgRNA of 4:1. The resulting nanoparticles were treated in Hep3B cells and genomic DNA (gDNA) was extracted using the Qiagen DNeasy Blood and Tissue Protocol 72 hour after the transfection. The experiments were performed in biological duplicate and assay duplicate and ddPCR was used to quantify nonhomologous end joining (NHEJ) efficiency. The results are plotted in FIG. 19.

As demonstrated by FIG. 19, Polymer H27N alone was efficient at gene editing in Hep3B cells. PEG-Polymers 1-3 showed comparable or slightly reduced gene editing efficiency in Hep3B cells.

### EXAMPLE 14

The following example illustrates the effect of PEG-Polymers 1-3 on the ability of Polymer H27N to deliver Cas9 RNP to Hep3B cells.

Hep3B cells were seeded 50,000 cells/well in culture medium composed of Dulbecco's Modified Eagle Medium (DMEM) and 10% fetal bovine serum (FBS) to form 40 pmol Cas9 RNP. sgRNA targeting SERPINA1 gene was prepared and Cas9 protein was added slowly and mixed thoroughly by pipetting. Separately, compositions containing H27N polymer and PEG-Polymers 1-3 were prepared using a 1:1 ratio. Nanoparticles were formed by mixing the resulting compositions with sgRNA using a mass ratio of polymer to sgRNA of 8:1. The resulting nanoparticles were treated in Hep3B cells and genomic DNA (gDNA) was extracted using the Qiagen DNeasy Blood and Tissue Protocol 72 hour after the transfection. The experiments were performed in biological duplicate and assay duplicate and ddPCR was used to quantify nonhomologous end joining (NHEJ) efficiency. The results are plotted in FIG. 20.

As demonstrated by FIG. 20, Polymer H27N alone was efficient at gene editing in Hep3B cells. PEG-Polymers 1-3 showed comparable or slightly reduced gene editing efficiency in Hep3B cells.

### EXAMPLE 15

The following example illustrates the use of polymers of the invention to deliver Cre mRNA to mice as exhibited by Loxp-luciferase mice.

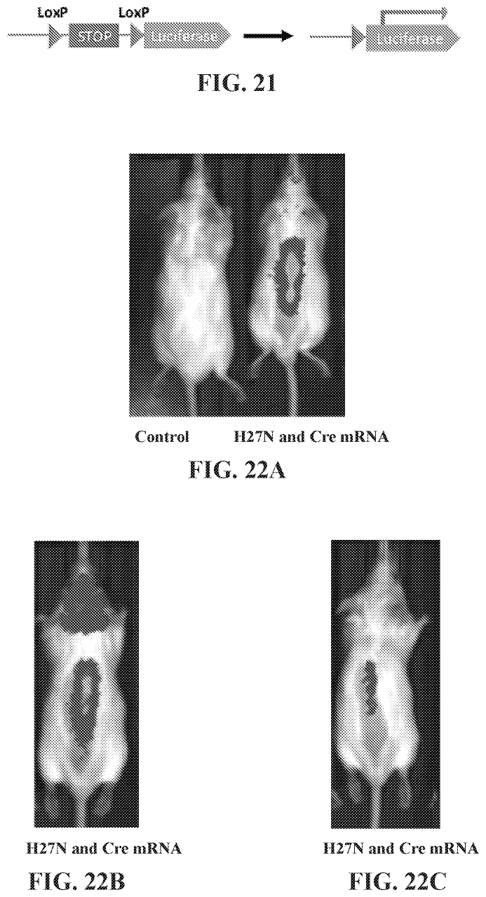

Loxp-luciferase mice having the reporter sequence set forth in FIG. 21 were treated nanoparticles formulated with H27N and Cre mRNA. The control represents an untreated mouse. Administration was via intrathecal (IT) injection. Cre mRNA delivery was assessed via bioluminescence, and the resulting images are set forth in FIGS. 22A-22C.

The mice treated with nanoparticle composition showed significant delivery of Cre mRNA mice .

### EXAMPLE 16

Polymer C can be prepared by modifying PBLA with N¹-(2-aminoethyl)-N¹,N²,N²-trimethylethane-1,2-diamine and 4-methylpentan-l-amine. An exemplary procedure is as follows.

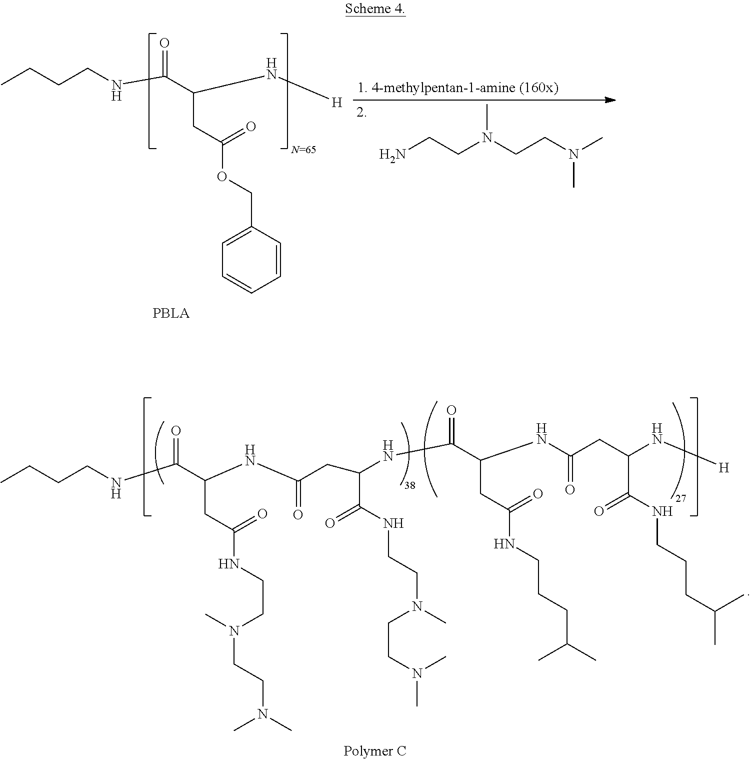

Lyophilized PBLA (50 mg, 0.0037 mMol) was placed into a flask and dissolved in tetrahydrofuran/N-methyl-2-pyrrolidine (1 mL each). To the clear solution was added n-4-methylpentan-1-amine (160 equivalents), and the clear reaction mixture was stirred for 24 hours at room temperature. After approximately 24 hours, N¹-(2-aminoethyl)-N¹,N²,N²-trimethylethane-1,2-diamine (50 equivalents to benzyl group of PBLA segment) was added to the clear mixture under mild anhydrous conditions. After approximately 18 hours at room temperature, the reaction mixture was precipitated into diethyl ether (10-12× volume, 35 mL). The precipitate was then centrifuged, and washed twice with diethyl ether. The polymer was dissolved in 1 M HCl (3 mL) and dialyzed in an excess of deionized water in a 3.5-5 KD cut-off membrane. When the pH of the solution was between 5-6, the dialysis was stopped, and the solution was lyophilized, to give the polymer product.

### EXAMPLE 17

The following example illustrates the use of polymers of the invention to deliver Cre mRNA to mice as exhibited by ai9 mice.

ai9 mice having the same reporter construct illustrated in FIG. 23 were treated with one of two nanoparticle compositions: (i) nanoparticles formulated with a mixture of H27N and PGA-PEG and Cre mRNA with a 4:1 ratio of PGA-PEG:mRNA (“H27N+PGA-PEG (”4:1 PGA-PEG:mRNA ratio“) and (ii) nanoparticles formulated with a mixture of H27N and PGA-PEG and Cre mRNA with a 6:1 ratio of PGA-PEG:mRNA (”H27N+PGA-PEG (“6:1 PGA-PEG:mRNA ratio”). The control represents an untreated mouse. The nanoparticle properties summarized in Table 4.

TABLE 4
PEG-PGA nanoparticle properties.

| | Pre-Lyophilization | | | Post-Lyophilization | | |
|---|---|---|---|---|---|---|
| | Diameter | Zeta- | | Diameter | Zeta- | |
| Particle | (nm) | Potential | PDI | (nm) | Potential | PDI |
| H27N + 4X | 79 nm | −2.5 mV | 0.19 | 92.5 | −2.5 mV | 0.18 |
| PGA-PEG | | | | | | |
| H27N + 6X | 80 nm | −2 mV | 0.29 | 80 nm | −2 mV | Range |
| PGA-PEG | | | | | | |

The resulting nanoparticle formulations were administered to mice via intrathecal (IT) injection. Ten days after treatment, the mice were sacrificed via CO₂ asphyxiation and perfused through the left ventricle with 1% heparinized saline followed by PBS to remove blood. The brain and spinal cord were then harvested. The mouse brains were sectioned at 100 um thickness in the coronal plane and every other section was collected and imaged.

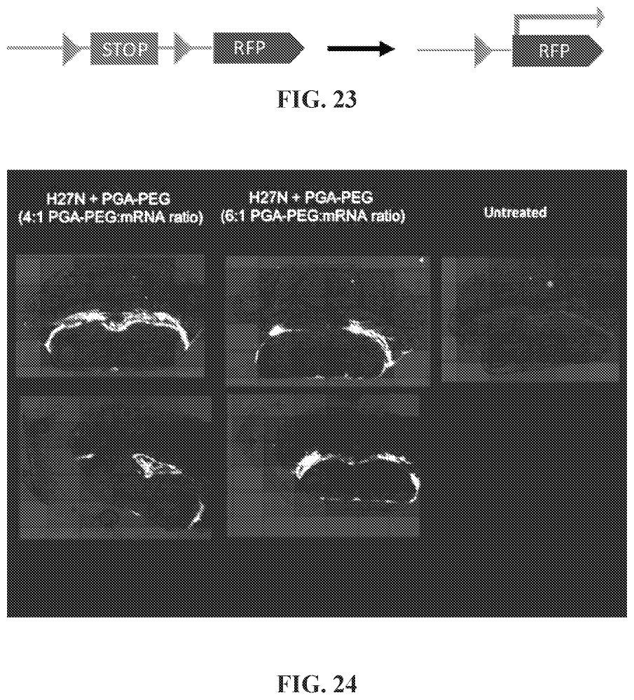

In vivo Cre mRNA delivery was assessed for the rostral and caudal sections of the brain via bioluminescence, and the resulting images are set forth in FIG. 24.

In FIG. 24, each of nanoparticles compositions (i) and (ii) showed increased delivery of Cre mRNA to the caudal sections of the brain stem and cerebellum (i.e., the areas of the brain that are surrounded by cerebrospinal fluid (CSF)), relative to the untreated mice (negative control). In addition, nanoparticle compositions (i) and (ii), containing PGA-PEG qualitatively showed significant visible RFP expression, suggesting that PGA-PEG nanoparticles have an enhanced ability to transfect around the brain stem of caudal region of brain.

### EXAMPLE 18

This example provides guidance for the synthesis of Polymer D described herein. The synthesis includes modifying PBLA with hexyl amine and amine compound 10. An exemplary procedure is as follows.

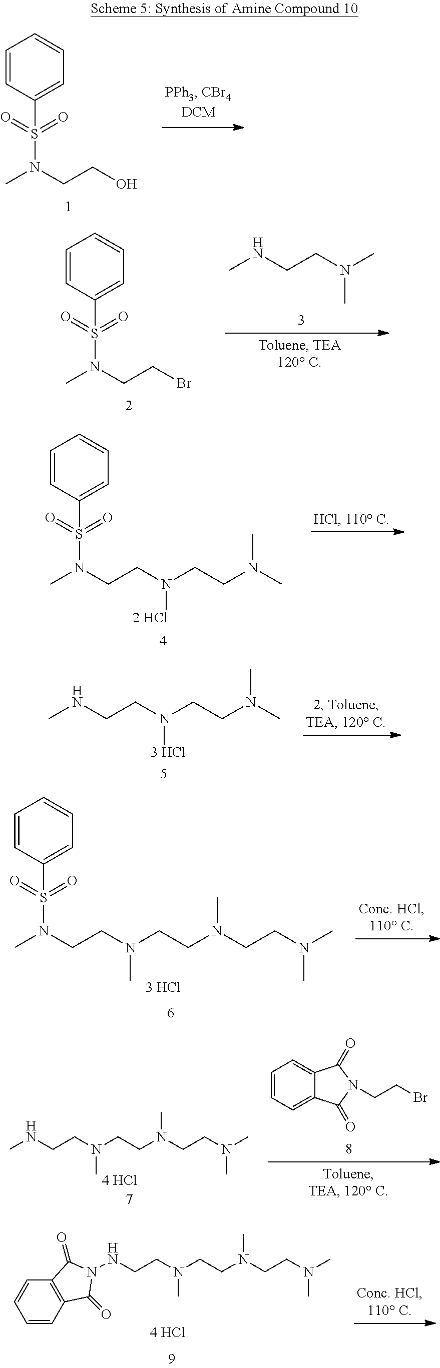

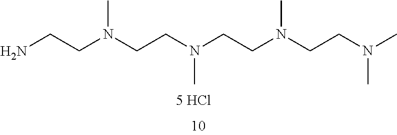 [SMILES: CN(C)CCN(C)CCN(C)CCN(C)CCN]

Amine compound 10 was synthesized using the protocol set forth in Scheme 5.

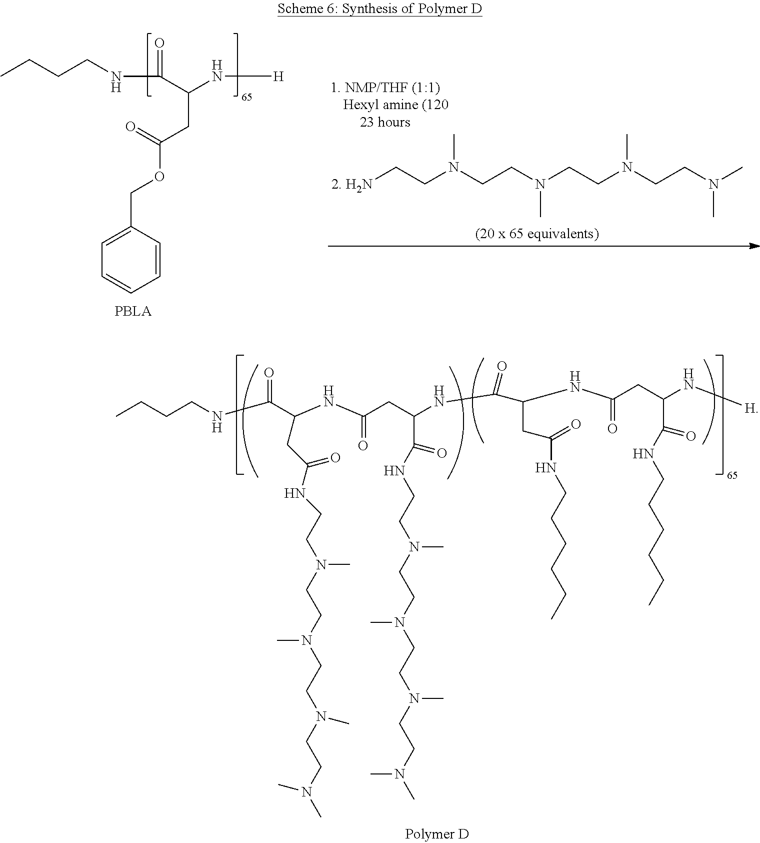

PBLA (25 mg, 0.0018 mmol) was dissolved into the mixture of 500 μL NMP and 500 μL of THF. Then to this reaction mixture was added hexyl amine (21.85 mg, 0.216 mmol) and the reaction mixture was stirred for 23 hours at room temperature. Then to this solution was added amine compound 10 (607 mg, 2.34 mmol) as a free amine form dissolved into 500 μL NMP, 500 μL THF and 500 μL triethylamine. The resulting reaction mixture was stirred at room temperature for 24 hours and the crude reaction mixture was precipitated into diethyl ether (40 mL) to yield crude polymer. Crude polymer was dissolved into 2 mL 1N HCl solution and was dialyzed using 3.5-5 KD cut-off membrane dialysis bag for 48 hours at 4° C. The purified polymer was lyophilized to yield Polymer D (17 mg) as a white solid. 1H NMR (D2O): 4.53 (65 H), 3.63-2.29 (m), 2.17-2.00 (s), 1.41-0.52 (m).

### EXAMPLE 19

This example provides guidance for the synthesis of Polymer E described herein. The synthesis includes modifying PBLA with hexyl amine and amine compound 13. An exemplary procedure is as follows.

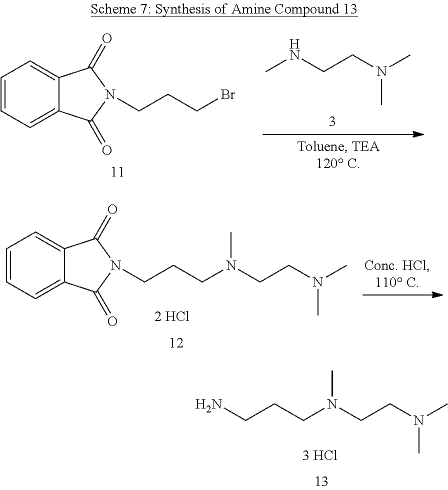

Amine compound 13 was synthesized using the protocol set forth in Scheme 7.

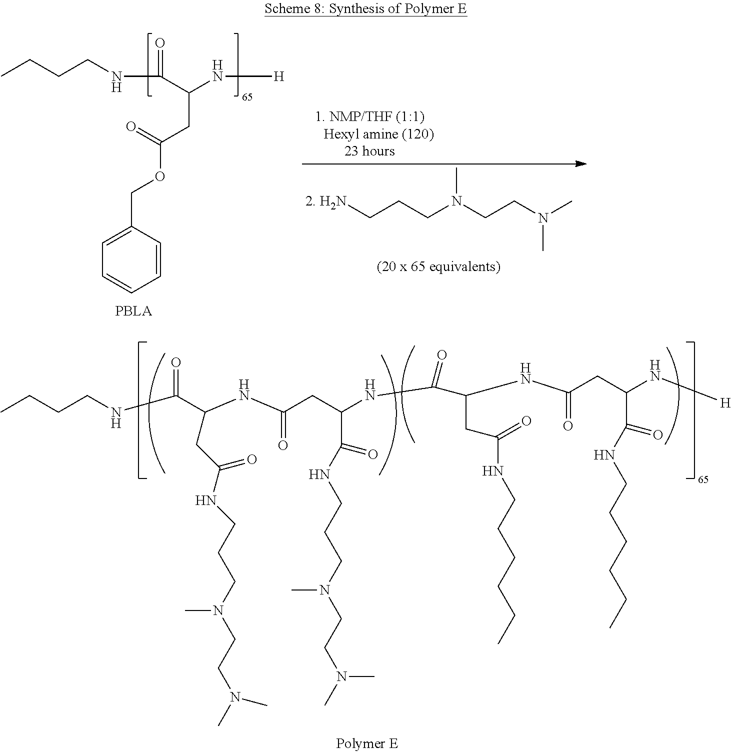

PBLA (25 mg, 0.0018 mmol) was dissolved into the mixture of 500 μL NMP and 500 μL of THF. Then to this reaction mixture was added hexyl amine (21.85 mg, 0.216 mmol) and the reaction mixture was stirred for 23 hours at room temperature. Then to this solution was added amine compound 13 (371.71 mg, 2.34 mmol) as a free amine form dissolved into 500 μL NMP, 500 μL THF and 500 μL triethylamine. The resulting reaction mixture was stirred at room temperature for 24 hours and the crude reaction mixture was precipitated into diethyl ether (40 mL) to yield crude polymer. Crude polymer was dissolved into 2 mL 1N HCl solution and was dialyzed using 3.5-5 KD cut-off membrane dialysis bag for 48 hours at 4° C. The purified polymer was lyophilized to yield Polymer E (20 mg) as a white solid. 1H NMR (D₂O): 4.53 (br s), 3.63-2.29 (m), 2.17-2.00 (s), 1.54 (m), 1.41-0.52 (m).

### EXAMPLE 20

This example provides guidance for the synthesis of Polymer F described herein. The synthesis includes modifying PBLA with cyclohexyl ethyl amine and amine compound 13. An exemplary procedure is as follows.

Amine compound 13 was synthesized using the protocol set forth in Scheme 7 of Example 19.

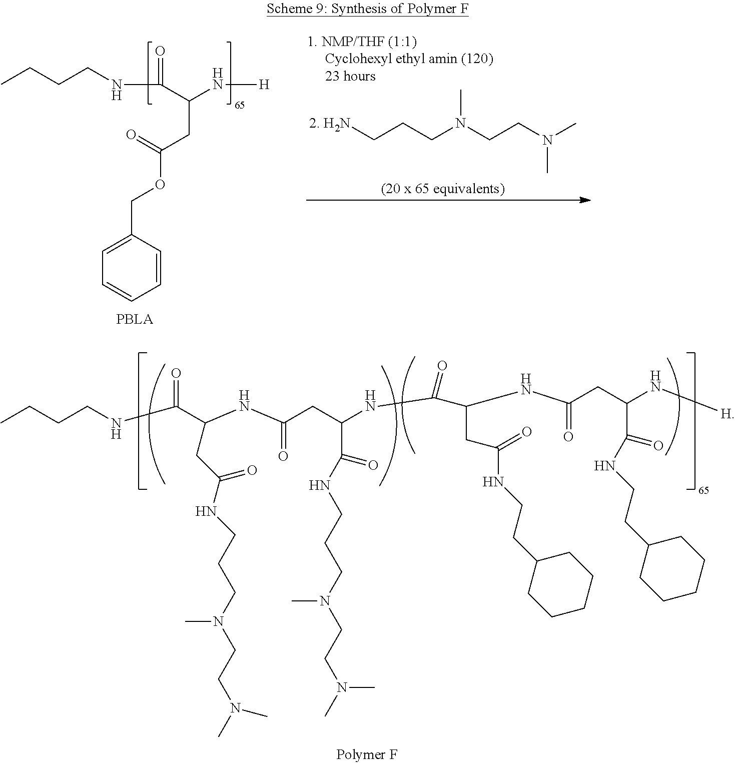

PBLA (25 mg, 0.0018 mmol) was dissolved into the mixture of 500 μL NMP and 500 μL of THF. Then to this reaction mixture was added cyclohexyl ethyl amine (27.48 mg, 0.216 mmol) and the reaction mixture was stirred for 23 hours at room temperature. Then to this solution was added amine compound 13 (371.71 mg, 2.34 mmol) as a free amine form dissolved into 500 μL NMP, 500 μL THF and 500 μL triethylamine. The resulting reaction mixture was stirred at room temperature for 24 hours and the crude reaction mixture was precipitated into diethyl ether (40 mL) to yield crude polymer. Crude polymer was dissolved into 2 mL 1N HCl solution and was dialyzed using 3.5-5 KD cut-off membrane dialysis bag for 48 hours at 4° C. The purified polymer was lyophilized to yield Polymer F (20 mg) as a white solid. 1H NMR (D₂O): 4.53 (br s), 3.63-2.29 (m), 2.17-2.00 (s), 1.55-1.15 (m).

### EXAMPLE 21

This example provides guidance for the synthesis of Polymer G described herein. The synthesis includes modifying PBLA with hexyl amine and amine compound 20. An exemplary procedure is as follows.

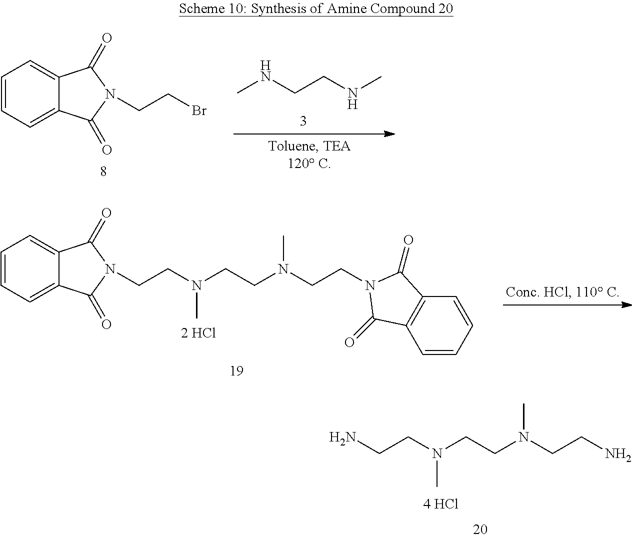

Amine compound 20 was synthesized using the protocol set forth in Scheme 10.

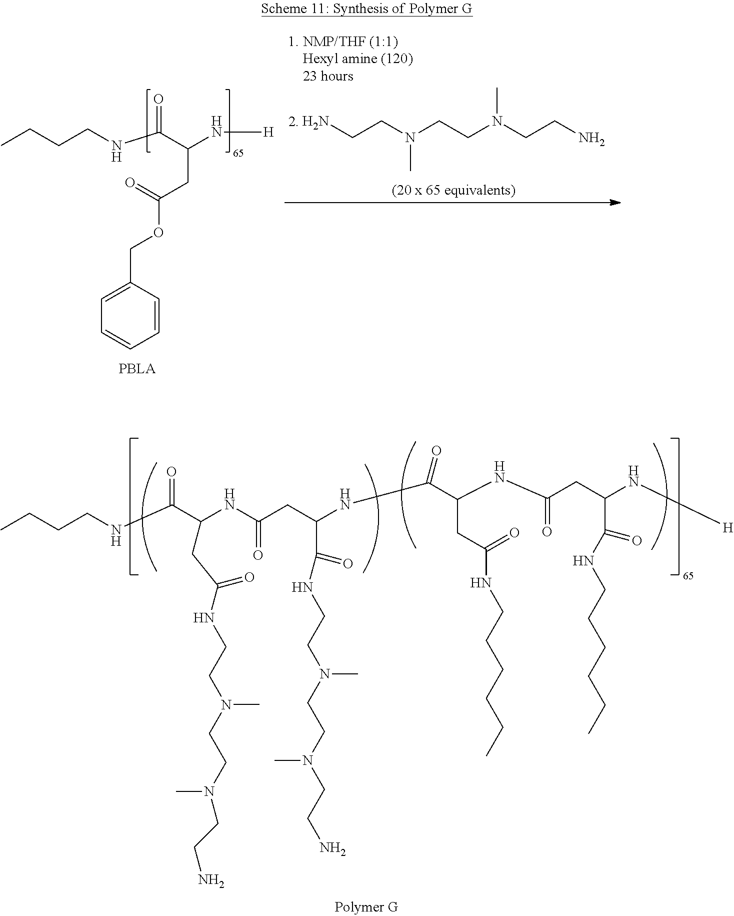

PBLA (25 mg, 0.0018 mmol) was dissolved into the mixture of 500 μL NMP and 500 μL of THF. Then to this reaction mixture was added hexyl amine (21.85 mg, 0.216 mmol) and the reaction mixture was stirred for 23 hours at room temperature. Then to this solution was added amine compound 20 (407.8 mg, 2.34 mmol) as a free amine form dissolved into 500 μL NMP, 500 μL THF and 500 μL triethylamine. The resulting reaction mixture was stirred at room temperature for 24 hours and the crude reaction mixture was precipitated into diethyl ether (40 mL) to yield crude polymer. Crude polymer was dissolved into 2 mL 1N HCl solution and was dialyzed using 3.5-5 KD cut-off membrane dialysis bag for 48 hours at 4° C. The purified polymer was lyophilized to yield Polymer G (20 mg) as a white solid. 1H NMR (D₂O): 4.53 (br s), 3.63-2.29 (m), 2.17-2.00 (s), 1.54 (m), 1.41-0.52 (m).

### EXAMPLE 22

This example provides guidance for the synthesis of Polymer H described herein. The synthesis includes modifying PBLA with hexyl amine and amine compound 22. An exemplary procedure is as follows.

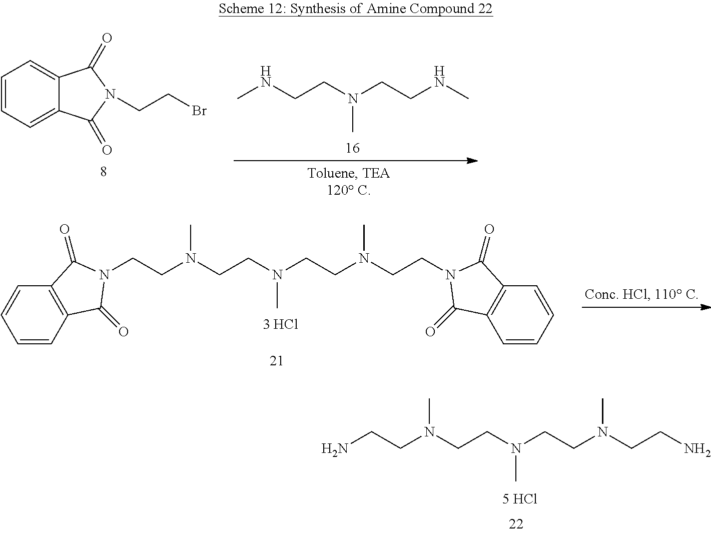

Amine compound 22 was synthesized using the protocol set forth in Scheme 12.

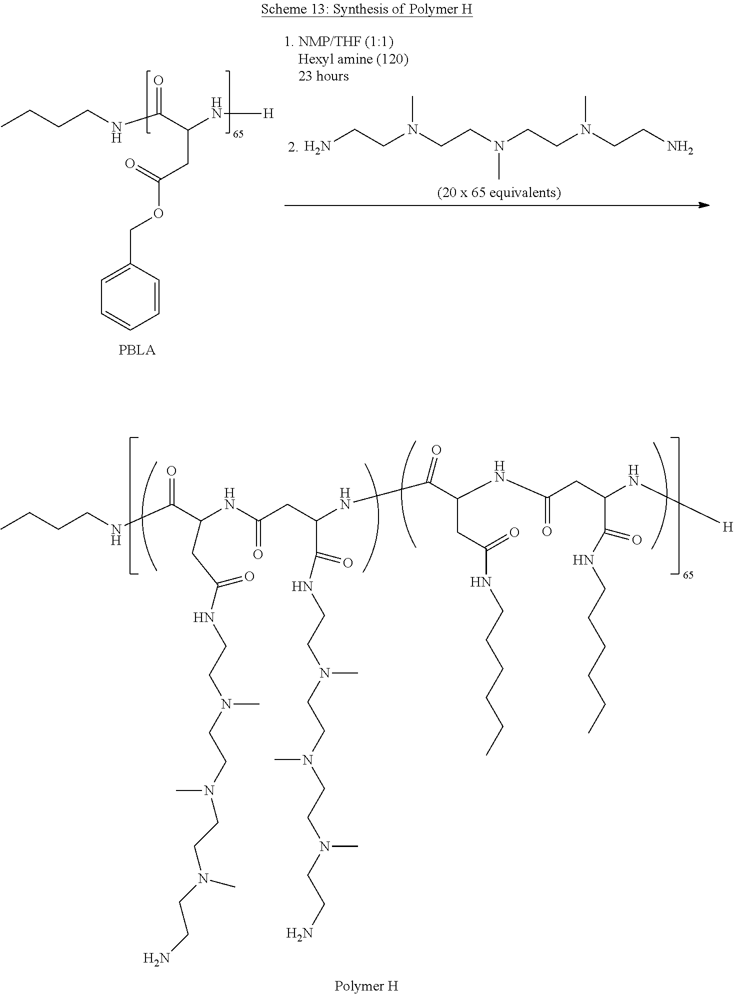

PBLA (25 mg, 0.0018 mmol) was dissolved into the mixture of 500 μL NMP and 500 μL of THF. Then to this reaction mixture was added hexyl amine (21.85 mg, 0.216 mmol) and the reaction mixture was stirred for 23 hours at room temperature. Then to this solution was added amine compound 22 (541.5 mg, 2.34 mmol) as a free amine form dissolved into 500 μL NMP, 500 μL THF and 500 μL triethylamine. The resulting reaction mixture was stirred at room temperature for 24 hours and the crude reaction mixture was precipitated into diethyl ether (40 mL) to yield crude polymer. Crude polymer was dissolved into 2 mL 1N HCl solution and was dialyzed using 3.5-5 KD cut-off membrane dialysis bag for 48 hours at 4° C. The purified polymer was lyophilized to yield Polymer H (20 mg) as a white solid. 1H NMR (D₂O): 4.53 (br s), 3.63-2.29 (m), 2.17-2.00 (s), 1.54 (m), 1.41-0.52 (m).

### EXAMPLE 23

The following example illustrates the ability of nanoparticles comprising the polymers of the invention to deliver mCherry mRNA to to HEK293T cells and Hep3B cells

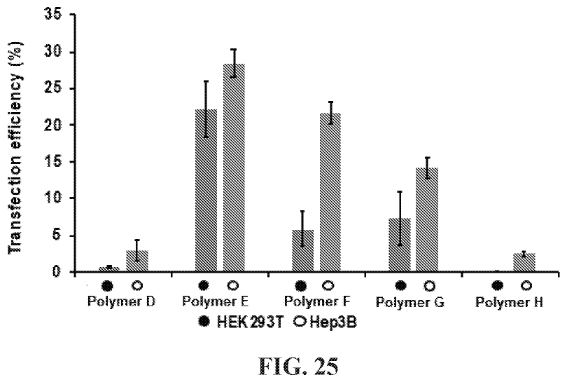

mCherry mRNA was mixed with each of Polymers D-H to provide loaded nanoparticles. The resulting nanoparticles were used to treat HEK293T cells and Hep3B cells, and mCherry expression was quantified with flow cytometry. The results are presented in FIG. 25 (n=2, error bar=SEM).

As demonstrated by FIG. 25, treatment of treat HEK293T cells and Hep3B cells with nanoparticles formed from Polymers E, F, and G resulted in a relatively high transfection efficiency as compared to nanoparticles formed from Polymers D and H.

### EXAMPLE 24

This example provides guidance for the synthesis of Polymer I described herein. The synthesis includes modifying PBLA with hexyl amine and amine compound 15. An exemplary procedure is as follows.

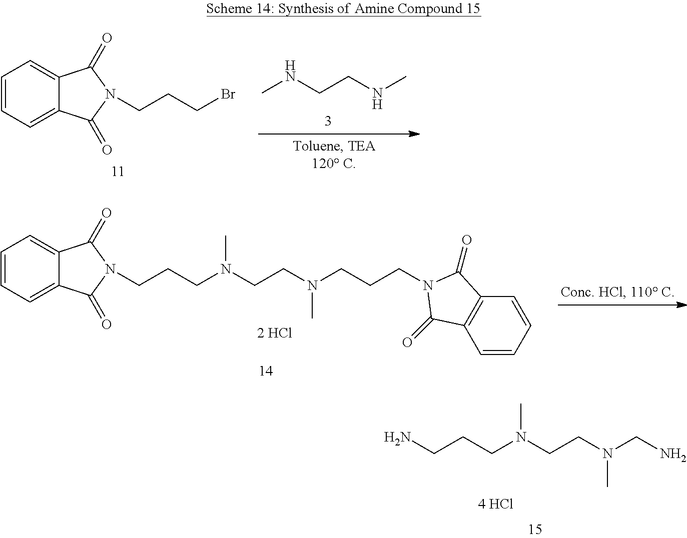

Amine compound 15 was synthesized using the protocol set forth in Scheme 14.

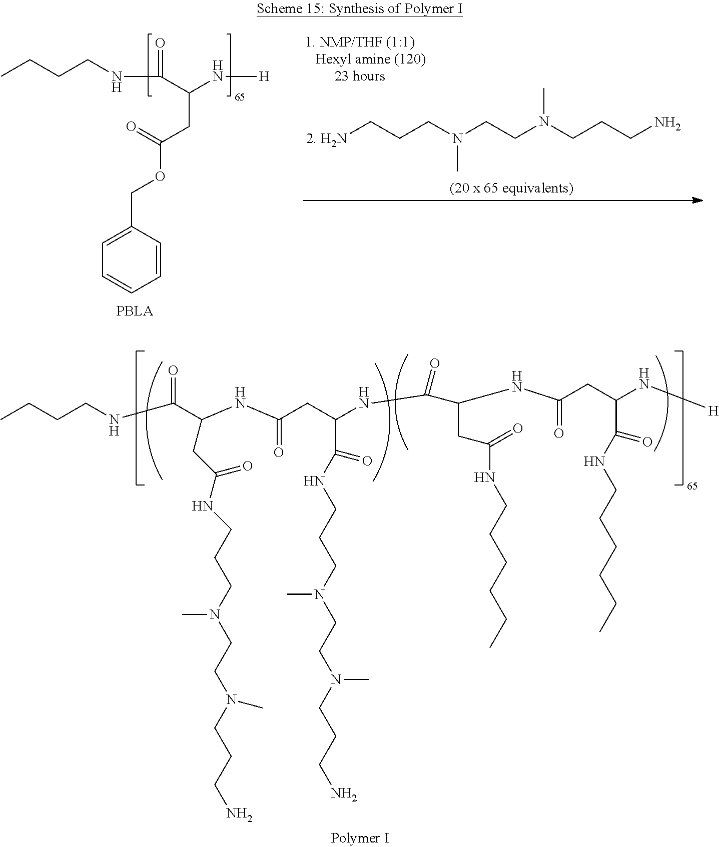

PBLA (25 mg, 0.0018 mmol) was dissolved into the mixture of 500 μL NMP and 500 μL of THF. Then to this reaction mixture was added hexyl amine (21.85 mg, 0.216 mmol) and the reaction mixture was stirred for 23 hours at room temperature. Then to this solution was added amine compound 15 (473.5 mg, 2.34 mmol) as a free amine form dissolved into 500 μL NMP, 500 μL THF and 500 μL triethylamine. The resulting reaction mixture was stirred at room temperature for 24 hours and the crude reaction mixture was precipitated into diethyl ether (40 mL) to yield crude polymer. Crude polymer was dissolved into 2 mL 1N HCl solution and was dialyzed using 3.5-5 KD cut-off membrane dialysis bag for 48 hours at 4° C. The purified polymer was lyophilized to yield Polymer I.

### EXAMPLE 25

This example provides guidance for the synthesis of Polymer J described herein. The synthesis includes modifying PBLA with hexyl amine and amine compound 18. An exemplary procedure is as follows.

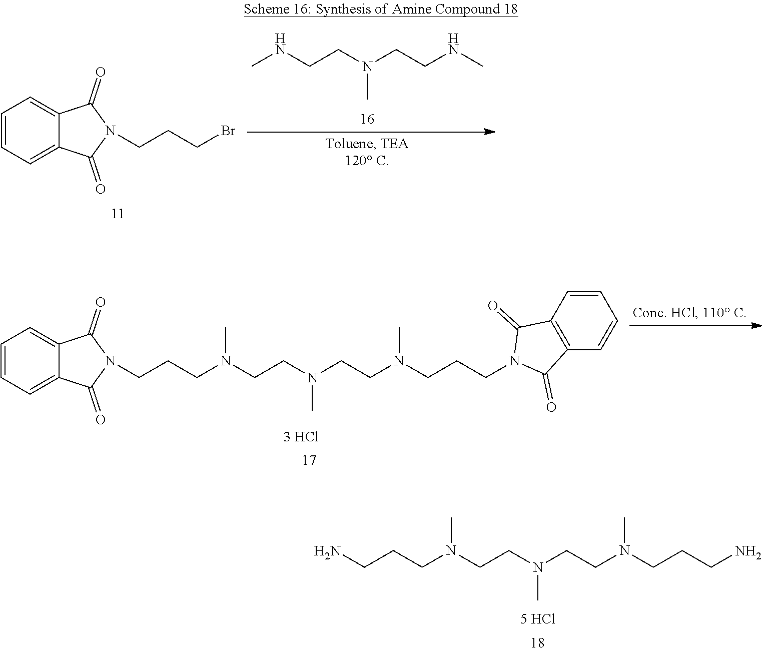

Amine compound 18 was synthesized using the protocol set forth in Scheme 16.

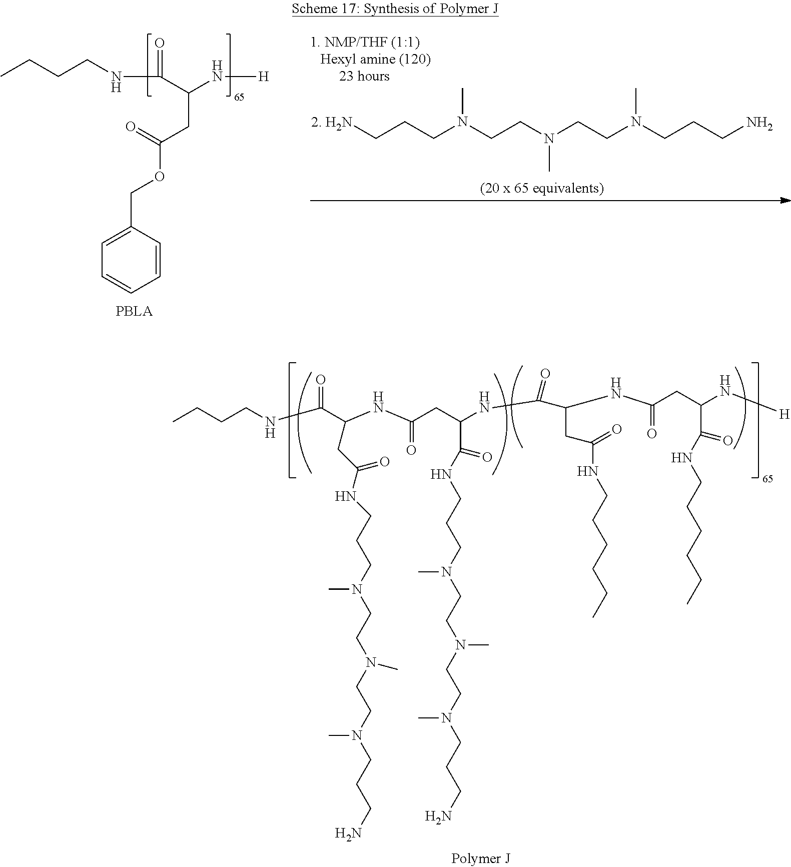

PBLA (25 mg, 0.0018 mmol) was dissolved into the mixture of 500 μL NMP and 500 μL of THF. Then to this reaction mixture was added hexyl amine (21.85 mg, 0.216 mmol) and the reaction mixture was stirred for 23 hours at room temperature. Then to this solution was added amine compound 18 (607 mg, 2.34 mmol) as a free amine form dissolved into 500 μL NMP, 500 μL THF and 500 μL triethylamine. The resulting reaction mixture was stirred at room temperature for 24 hours and the crude reaction mixture was precipitated into diethyl ether (40 mL) to yield crude polymer. Crude polymer was dissolved into 2 mL 1N HCl solution and was dialyzed using 3.5-5 KD cut-off membrane dialysis bag for 48 hours at 4° C. The purified polymer was lyophilized to yield Polymer J.

Preferred embodiments of this invention are described herein, including the best mode known to the inventors for carrying out the invention. Variations of those preferred embodiments can become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a complex” includes a plurality of such complexes and reference to “the Cas9 polypeptide” includes reference to one or more Cas9 polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

1. A polymer comprising a hydrolysable polymer backbone, the polymer backbone comprising:
(i) monomer units with a side chain comprising a hydrophobic group;
(ii) monomer units with a side chain comprising an oligoamine or polyamine; and optionally
(iii) monomer units with a side chain comprising an ionizable group, optionally with a pKa less than 7.

2. The polymer of claim 1, wherein the hydrophobic group comprises an alkyl group, alkenyl group, cycloalkyl group, or cycloalkenyl group.

3. The polymer of claim 1, wherein the hydrophobic group comprises a C₃-C₁₂ linear or branched alkyl group, optionally a C₃-C₆ linear or branched alkyl group.

4. The polymer of claim 1, wherein the oligoamine or polyamine is a group of the formula:
—(CH₂)ₚ₁—[NR²—(CH₂)_{q1}—]ᵣ₁NR²₂;
—(CH₂)ₚ₂—N[—(CH₂)_{q2}—NR²₂]₂;
—(CH₂)ₚ₃—N{[—(CH₂)_{q3}—NR²₂][—(CH₂)_{q4}—NR²—]r₂R²};
—(CH₂)ₚ₄—N{—(CH₂)_{q5}—N[—(CH₂)_{q6}—NR²₂]₂}₂,
—(CH₂)ₚ₁—[NR²—(CH₂)_{q1}—]ᵣ₁NR²—(CH₂)ₛ₁—R⁴—R⁵;
—(CH₂)ₚ₂—N[—(CH₂)_{q2}—NR²—(CH₂)ₛ₂—R⁴—R⁵]₂;
—(CH₂)ₚ₃—N{[—(CH₂)_{q3}—NR²₂][—(CH₂)_{q4}—NR²—]ᵣ₂(CH₂)ₛ₃—R⁴—R⁵};
—(CH₂)ₚ₄—N{—(CH₂)_{q5}—N[—(CH₂)_{q6}—NR²—(CH₂)ₛ₄—R⁴—R⁵]₂}₂;
—(CH₂)ₚ₁ —[NR²—(CH₂)_{q1}—]ᵣ₁NR²—CH₂—CHOH—R⁵;
—(CH₂)ₚ₂—N[—(CH₂)_{q2}—NR²—CH₂—CHOH—R⁵;
—(CH₂)ₚ₃—N{[—(CH₂)_{q3}—NR²₂][—(CH₂)_{q4}—NR²—]ᵣ₂—CH₂—CHOH—R⁵;
—(CH₂)ₚ₄—N{—(CH₂)_{q5}—N[—(CH₂)_{q6}—NR²—CH₂—CHOH—R⁵]₂}₂;
—(CH₂)ₚ₁—[NR²—(CH₂)_{q1}—]ᵣ₁NR²—(CH₂)ₛ₁—R⁵;
—(CH₂)ₚ₂—N[—(CH₂)_{q2}—NR²—(CH₂)ₛ₂—R⁵]₂;
—(CH₂)ₚ₃—N{[—(CH₂)_{q3}—NR²₂][—(CH₂)_{q4}—NR²—]ᵣ₂(CH₂)ₛ₃—R⁵};
—(CH₂)ₚ₄—N{—(CH₂)_{q5}—N[—(CH₂)_{q6}—NR²—(CH₂)ₛ₄—R⁵]₂}₂;
—(CH₂)ₚ₁—[N{(CH₂)ₛ₁—R⁴—R⁵}—(CH₂)_{q1}—]ᵣ₁NR²₂;
—(CH₂)ₚ₁—[N{(CH₂)ₛ₁—R⁵}—(CH₂)_{q1}—]ᵣ₁NR²₂,
—(CH₂)ₚ₁—[NR²—(CH₂)_{q1}—]ᵣ₁NR²—CH(CONH₂)—(CH₂)ₛ₁—R⁵; or
—(CH₂)ₚ₁—[NR²—(CH₂)_{q1}—]ᵣ₁NR²—CH(CONH₂)—(CH₂)ₛ₁—R⁴—R⁵,
wherein p1 to p4, q1 to q6, r1 and r2, and s1 to s4 are each independently an integer of 1 to 5; each instance of R² is independently hydrogen or a C₁-C₁₂ alkyl group, alkenyl group, cycloalkyl group, or cycloalkenyl group, or R² is combined with a second R² so as to form a heterocyclic group; each instance of is independently —C(O)O—, —C(O)NH—, or —S(O)(O)—; and each instance of R⁵ is independently an alkyl group, cycloalkyl group, alkenyl group, cycloalkenyl group, aryl group, heteroalkyl group, heterocyclic group, or combination thereof optionally comprising from 2 to 8 tertiary amines or a substituent comprising a tissue-specific or cell-specific targeting moiety.

5. The polymer of claim 1, wherein the oligoamine or polyamine comprises
—(CH₂)ₚ₁—[NR²—(CH₂)_{q1}—]₁NR²₂;
—(CH₂)ₚ₂—N[—(CH₂)_{q2}—NR²₂]₂;
—(CH₂)ₚ₃—N{[—(CH₂)_{q3}—NR²₂][—(CH₂)_{q4}—NR²—]r₂R²}; or
—(CH₂)ₚ₄—N{—(CH₂)_{q5}—N[—(CH₂)_{q6}—NR²₂]₂}₂;
and each R² is independently hydrogen or a C₁-C₃ alkyl group;
optionally wherein the polyamine comprises
—(CH₂)ₚ₁—[NR²—(CH₂)_{q1}—]ᵣ₁NR²₂.

6. The polymer of claim 1, wherein the hydrolysable polymer backbone comprises about 1 to about 80 mol % of the monomer units having a hydrophobic group, about 1 to about 80 mol % of the monomer units having an oligoamine or polyamine, and 0 to about 80 mol % of the monomer units having an ionizable group.

7. (canceled)

8. (canceled)

9. The polymer of claim 1, wherein the hydrolysable polymer backbone comprises a polyamide.

10. The polymer of claim 1, comprising a structure of Formula 1: 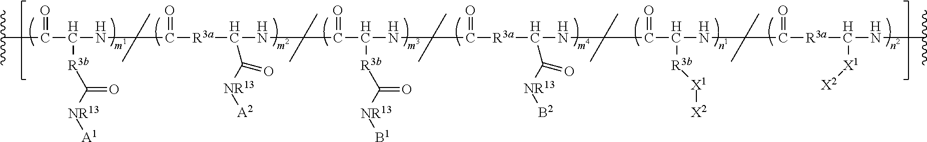
wherein:
each of m¹, m², m³, and m⁴ is an integer from 0 to 1000, provided that the sum of m¹+m²+m³+m⁴ is greater than 5;
each of n¹ and n² is an integer from 0 to 1000, provided that the sum of n¹+n² is greater than 2;
the symbol “/” indicates that the units separated thereby are linked randomly or in any order;
each instance of R^{3a} is independently a methylene or ethylene group;
each instance of R^{3b} is independently a methylene or ethylene group;
each X¹ independently is —C(O)O—, —C(O)NR¹³—, —C(O)—, —S(O)(O)—, or a bond;
each instance of R¹³ is independently hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which can be optionally substituted with one or more substituents;
each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof, any of which can be substituted with one or more substituents;
A¹ and A² are each independently a group of formula
—(CH₂)ₚ₁—[NR²—(CH₂)_{q1}—]ᵣ₁NR²₂;
—(CH₂)ₚ₂—N[—(CH₂)_{q2}—NR²₂]₂;
—(CH₂)ₚ₃—N{[—(CH₂)_{q3}—NR²₂][—(CH₂)_{q4}—NR²—]r₂R²}; or
—(CH₂)ₚ₄—N{—(CH₂)_{q5}—N[—(CH₂)_{q6}—NR²₂]₂}₂,
B¹ and B² are each independently
—(CH₂)ₚ₁—[NR²—(CH₂)_{q1}—]ᵣ₁NR²—(CH₂)ₛ₁—R⁴—R⁵;
—(CH₂)ₚ₂—N[—(CH₂)_{q2}—NR²—(CH₂)ₛ₂—R⁴—R⁵]₂;
—(CH₂)ₚ₃—N{[—(CH₂)_{q3}—NR²₂][—(CH₂)_{q4}—NR²—]ᵣ₂(CH₂)ₛ₃—R⁴—R⁵};
—(CH₂)ₚ₄—N{—(CH₂)_{q5}—N[—(CH₂)_{q6}—NR²—(CH₂)ₛ₄—R⁴—R⁵]₂}₂;
—(CH₂)ₚ₁—[NR²—(CH₂)_{q1}—]ᵣ₁NR²—CH₂—CHOH—R⁵;
—(CH₂)ₚ₂—N[—(CH₂)_{q2}—NR²—CH₂—CHOH—R⁵;
—(CH₂)ₚ₃—N{[—(CH₂)_{q3}—NR²₂][—(CH₂)_{q4}—NR²—]ᵣ₂—CH₂—CHOH—R⁵;
—(CH₂)ₚ₄—N{—(CH₂)_{q5}—N[—(CH₂)_{q6}—NR²—CH₂—CHOH—R⁵]₂}₂;
—(CH₂)ₚ₁—[NR²—(CH₂)_{q1}—]ᵣ₁NR²—(CH₂)ₛ₁—R⁵;
—(CH₂)ₚ₂—N[—(CH₂)_{q2}—NR²—(CH₂)ₛ₂—R⁵]₂;
—(CH₂)ₚ₃—N{[—(CH₂)_{q3}—NR²₂][—(CH₂)_{q4}—NR²—]ᵣ₂(CH₂)ₛ₃—R⁵};
—(CH₂)ₚ₄—N{—(CH₂)_{q5}—N[—(CH₂)_{q6}—NR²—(CH₂)ₛ₄—R⁵]₂}₂;
—(CH₂)ₚ₁—[N{(CH₂)ₛ₁—R⁴—R⁵}—(CH₂)_{q1}—]ᵣ₁NR²₂;
—(CH₂)ₚ₁—[N{(CH₂)ₛ₁—R⁵}—(CH₂)_{q1}—]ᵣ₁NR²₂,
—(CH₂)ₚ₁—[NR²—(CH₂)_{q1}—]ᵣ₁NR²—CH(CONH₂)—(CH₂)ₛ₁—R⁵; or
—(CH₂)ₚ₁—[NR²—(CH₂)_{q1}—]ᵣ₁NR²—CH(CONH₂)—(CH₂)ₛ₁—R⁴—R⁵,
wherein p1 to p4, q1 to q6, r1 and r2, and s1 to s4 are each independently an integer of 1 to 5;
each instance of R² is independently hydrogen or a C₁-C₁₂ alkyl group, C₂-C₁₂ alkenyl group, C₃-C₁₂cycloalkyl group, or C₃-C₁cycloalkenyl group, or R² is combined with a second R² so as to form a heterocyclic group; each instance of R⁴ is independently —C(O)O—, —C(O)NH—, —O—C(O)O—, or —S(O)(O)—; and each instance of R⁵ is independently an alkyl group, cycloalkyl group, alkenyl group, cycloalkenyl group, aryl group, heteroalkyl group, heterocyclic group, or combination thereof optionally comprising from 2 to 8 tertiary amines or a substituent comprising a tissue-specific or cell-specific targeting moiety.

11. (canceled)

12. The polymer of claim 10, wherein each of B¹ and B² is a group of formula —(CH₂)₂—NH—(CH₂)₂—NH—(CH₂)₂—R⁴—R⁵.

13. (canceled)

14. The polymer of claim 10, wherein R⁴ is —C(O)—O—.

15. The polymer of claim 10, wherein R² is a C₁-C₃ alkyl

16. (canceled)

17. (canceled)

18. The polymer of claim 10 having the structure of Formula 4: 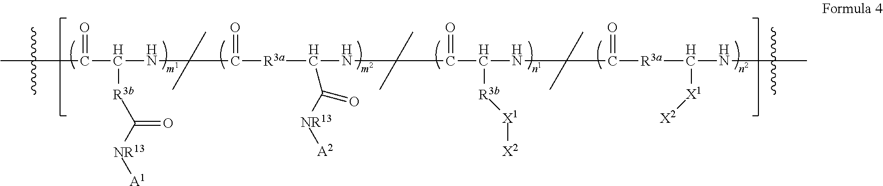
wherein m¹, m², n¹, n², R^{3a}, R^{3b}, R¹³, X¹, X², A¹, and A² are as defined in claim 10.

19.-20. (canceled)

21. The polymer of claim 10, having the formula: 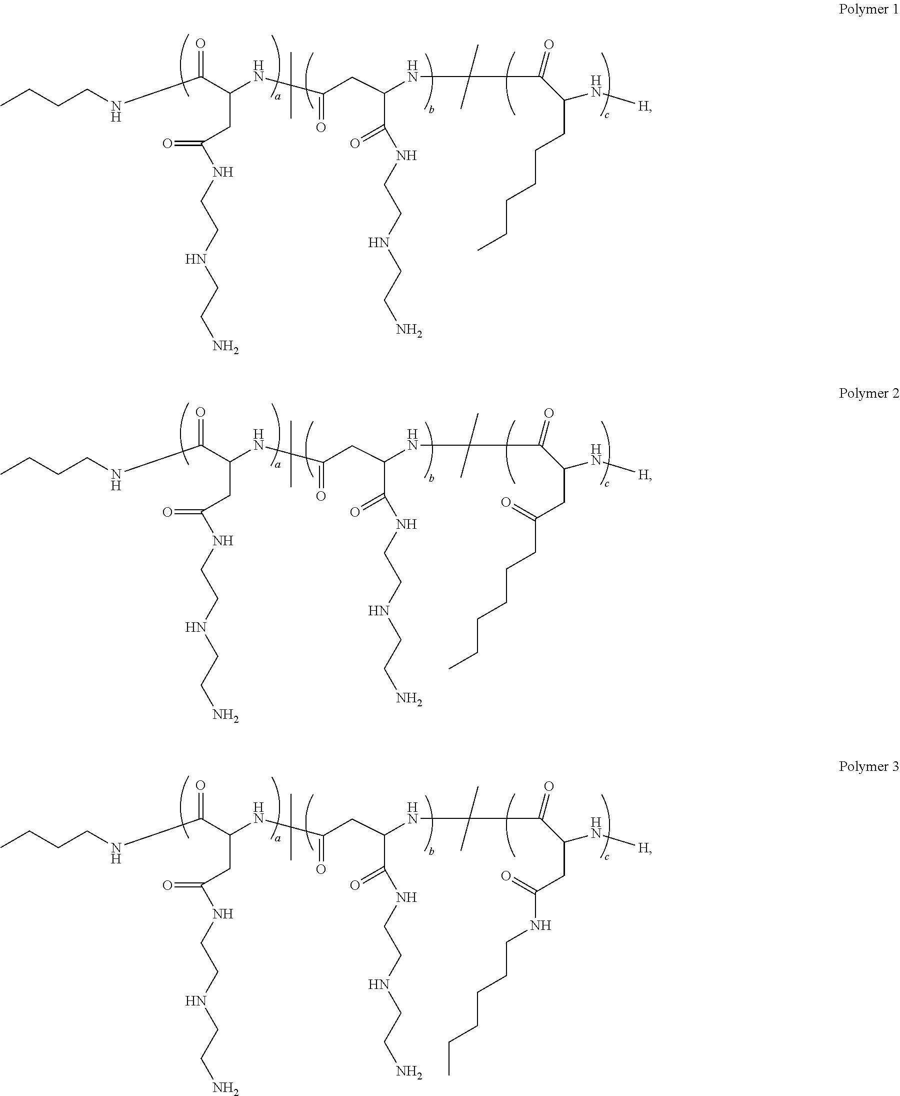 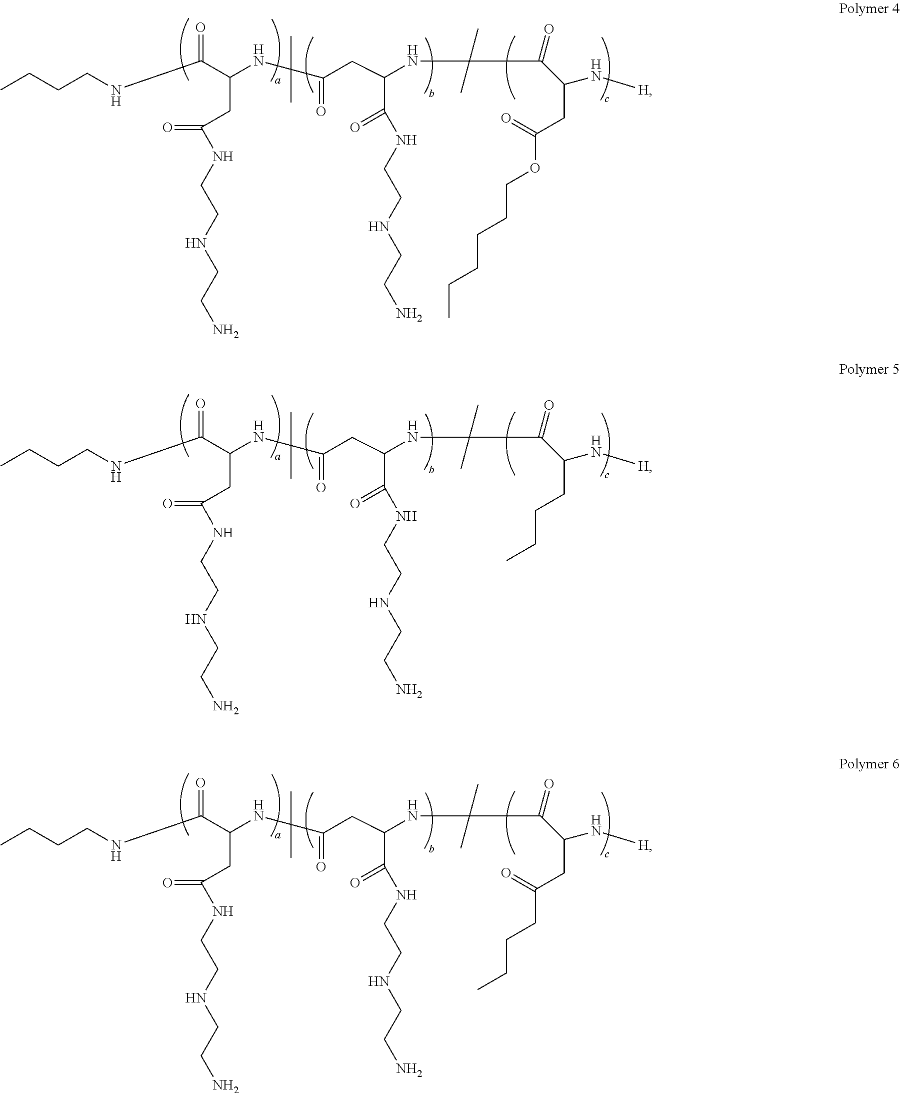 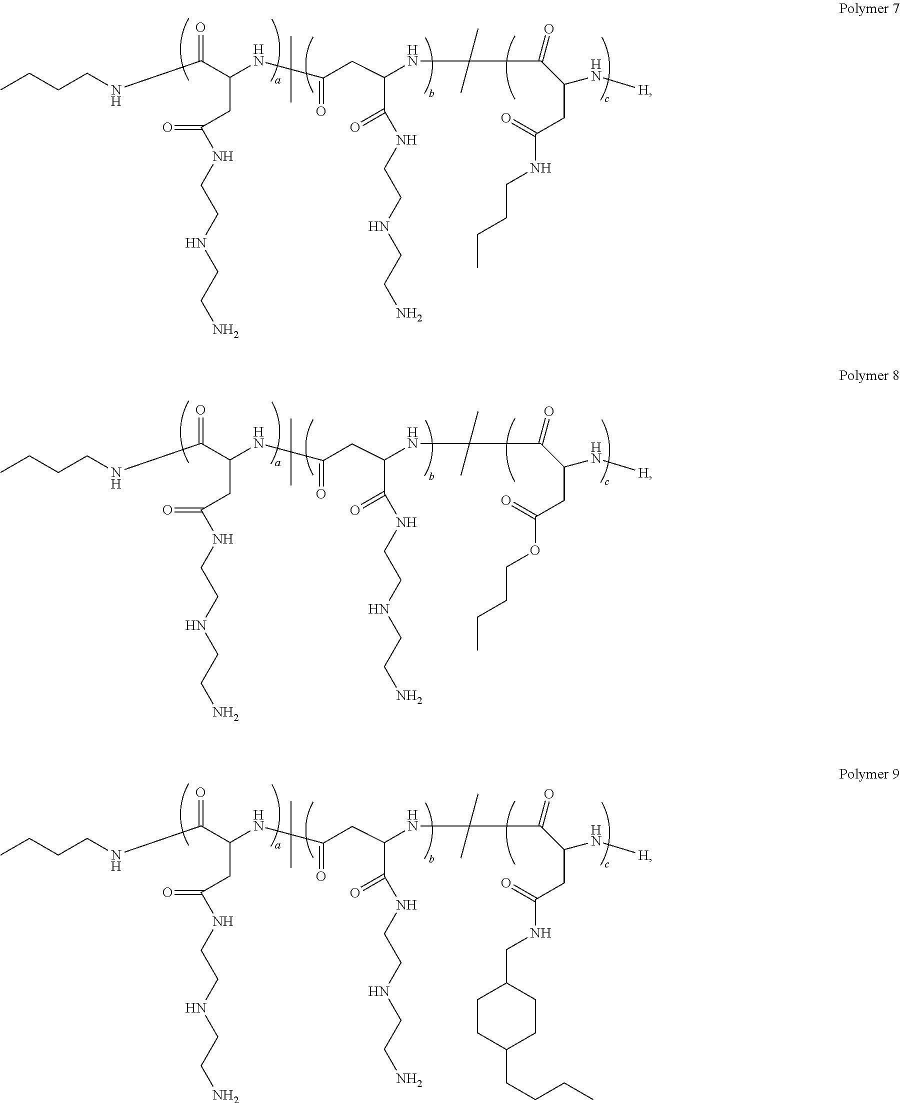 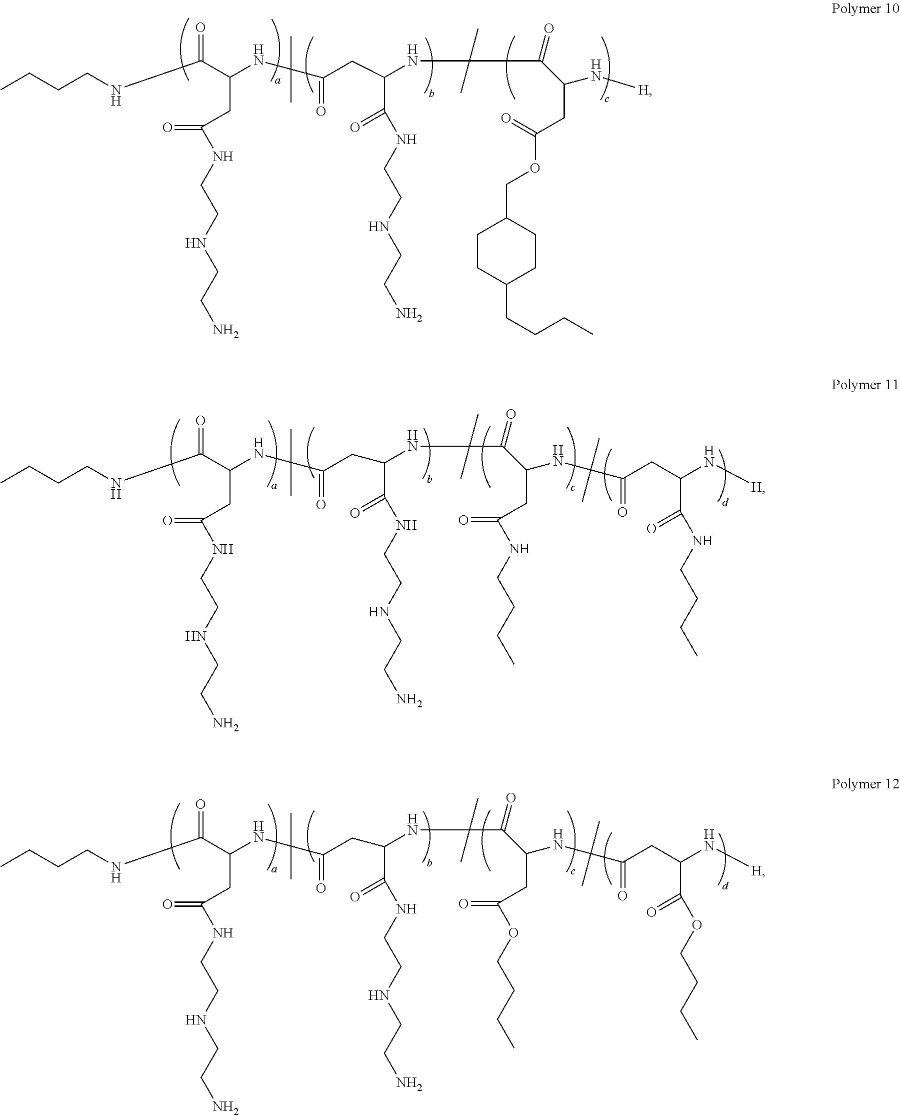 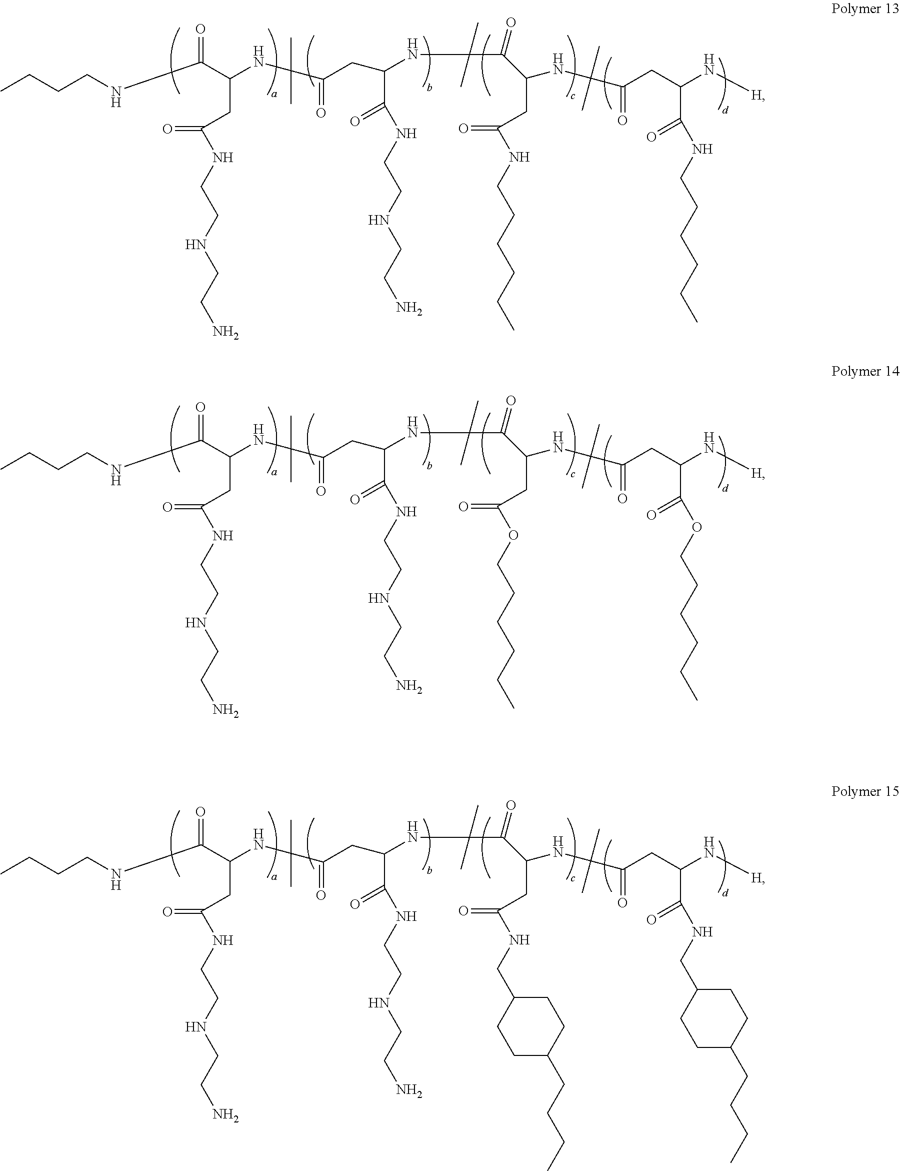 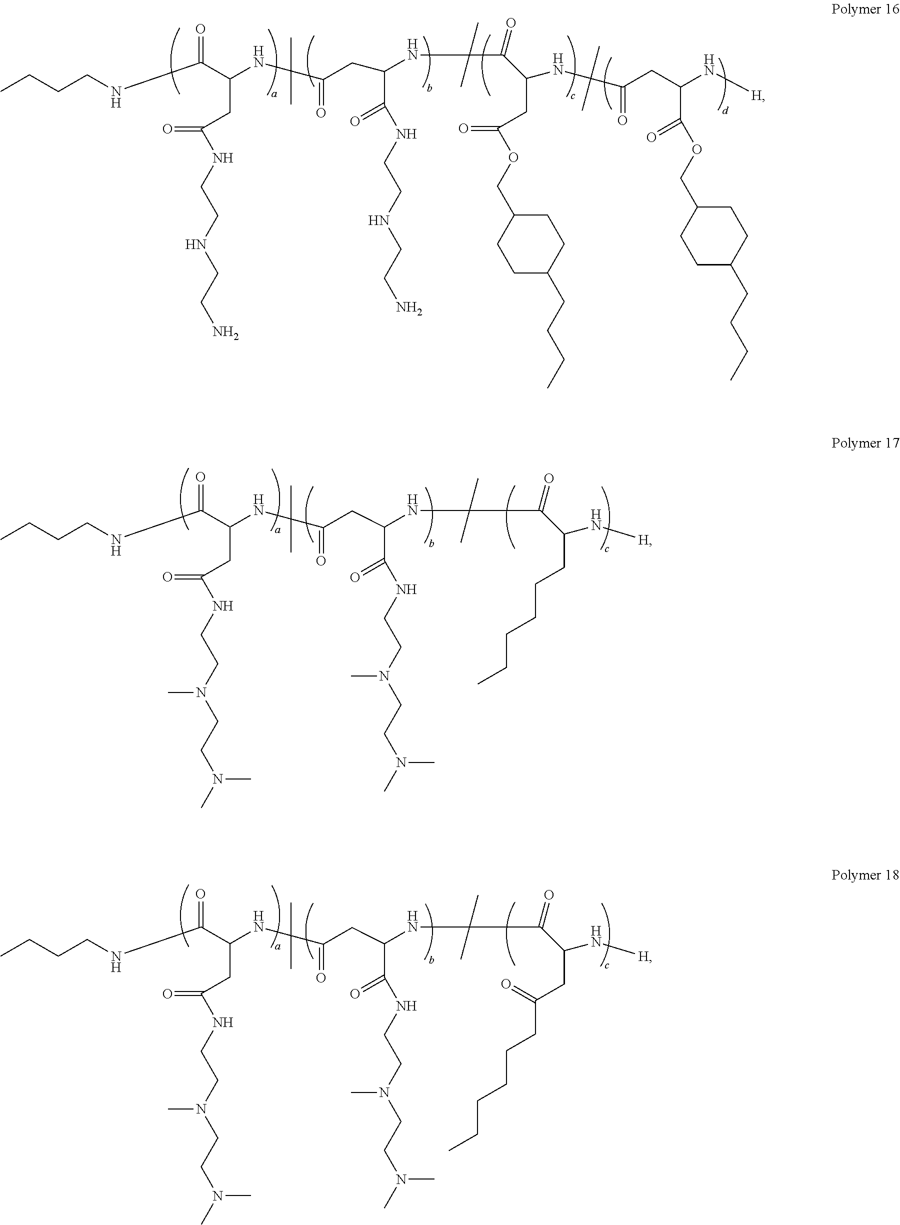 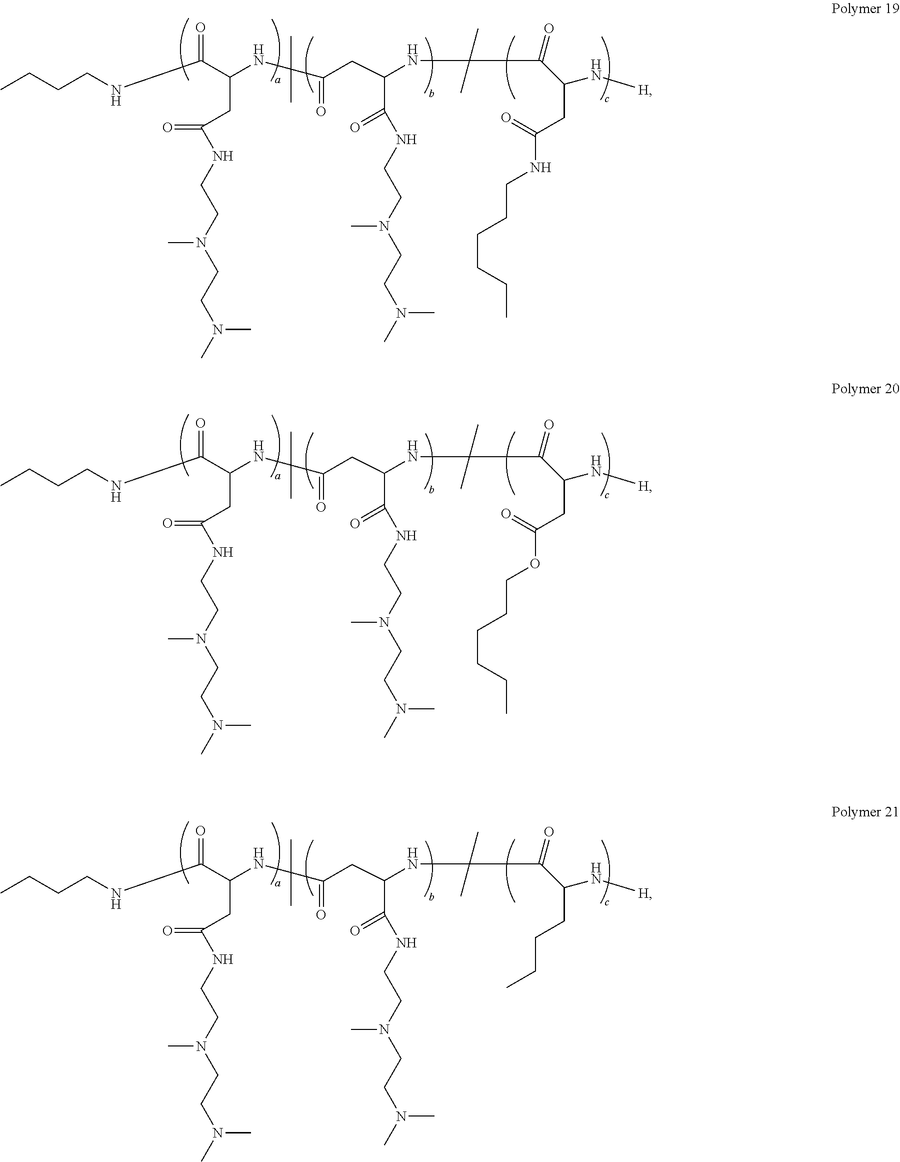 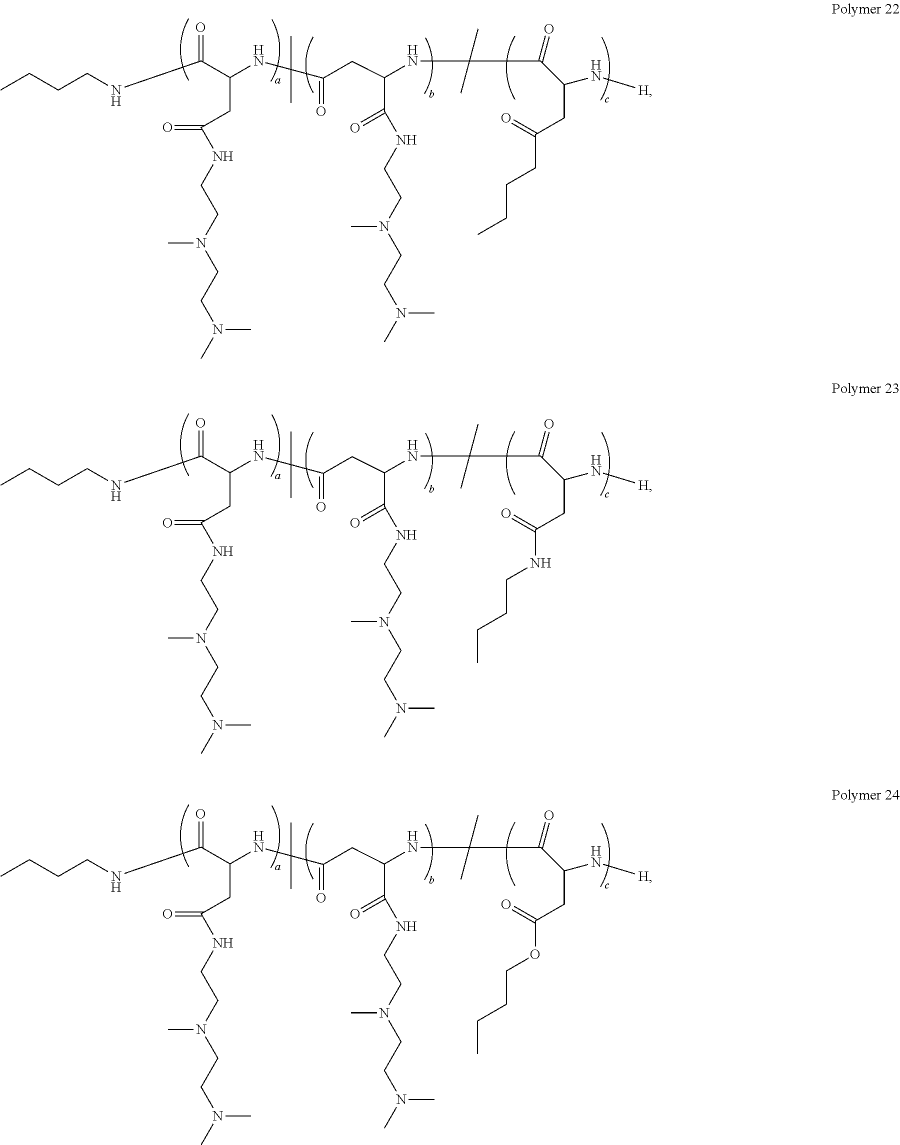 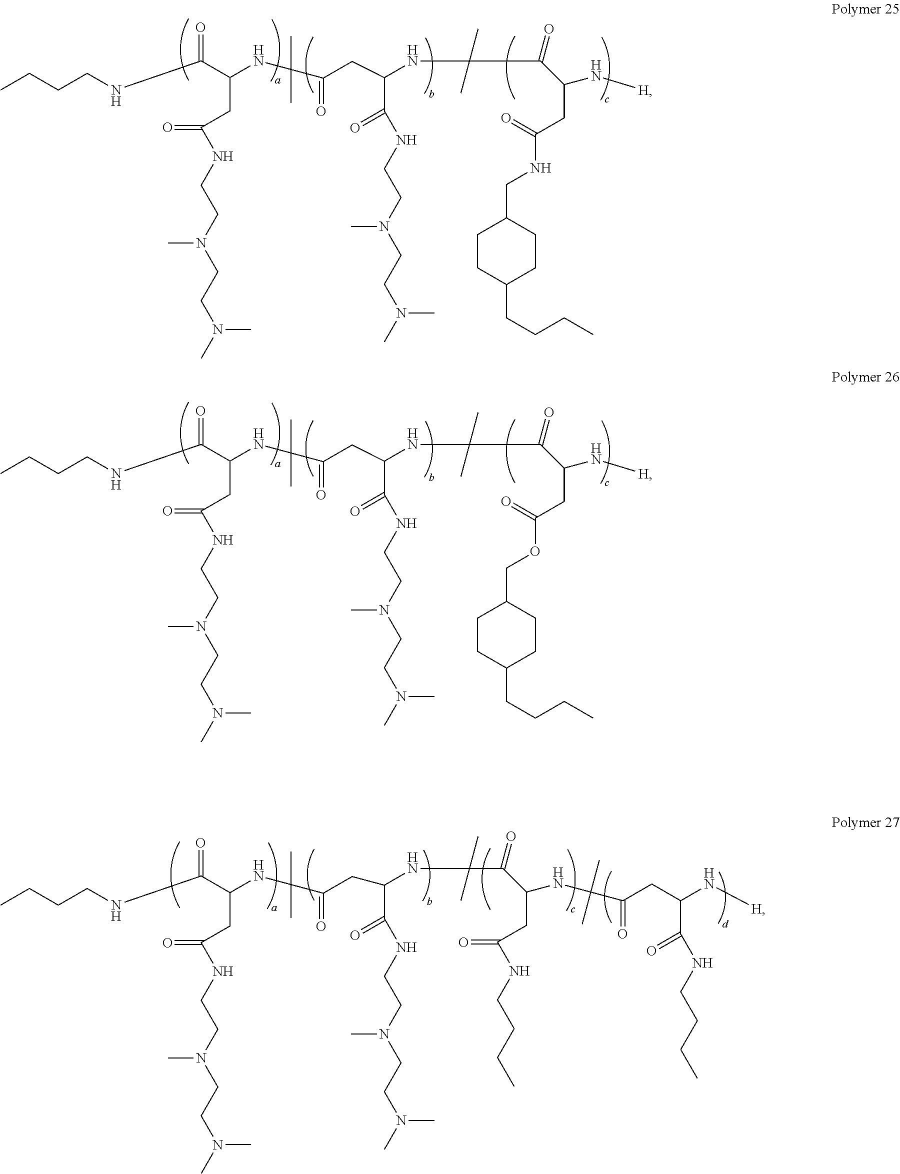 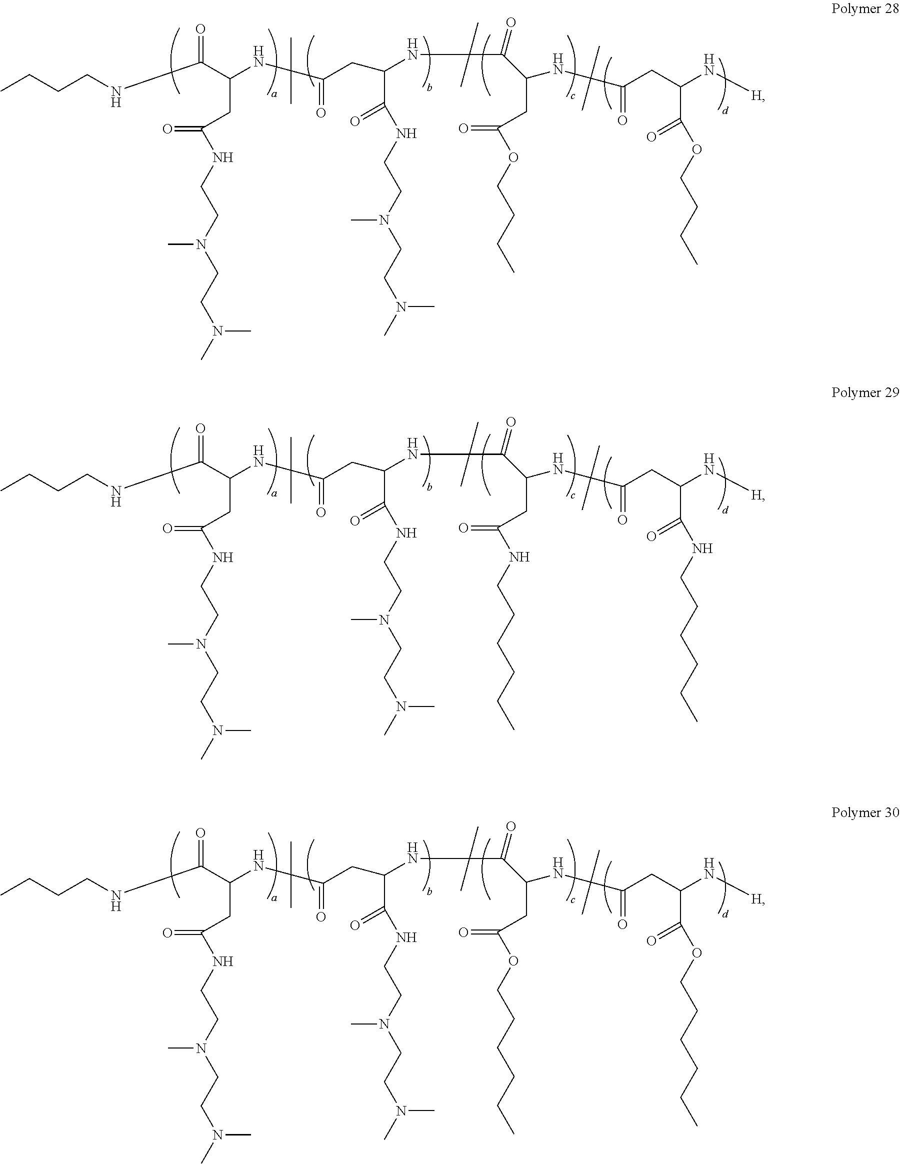 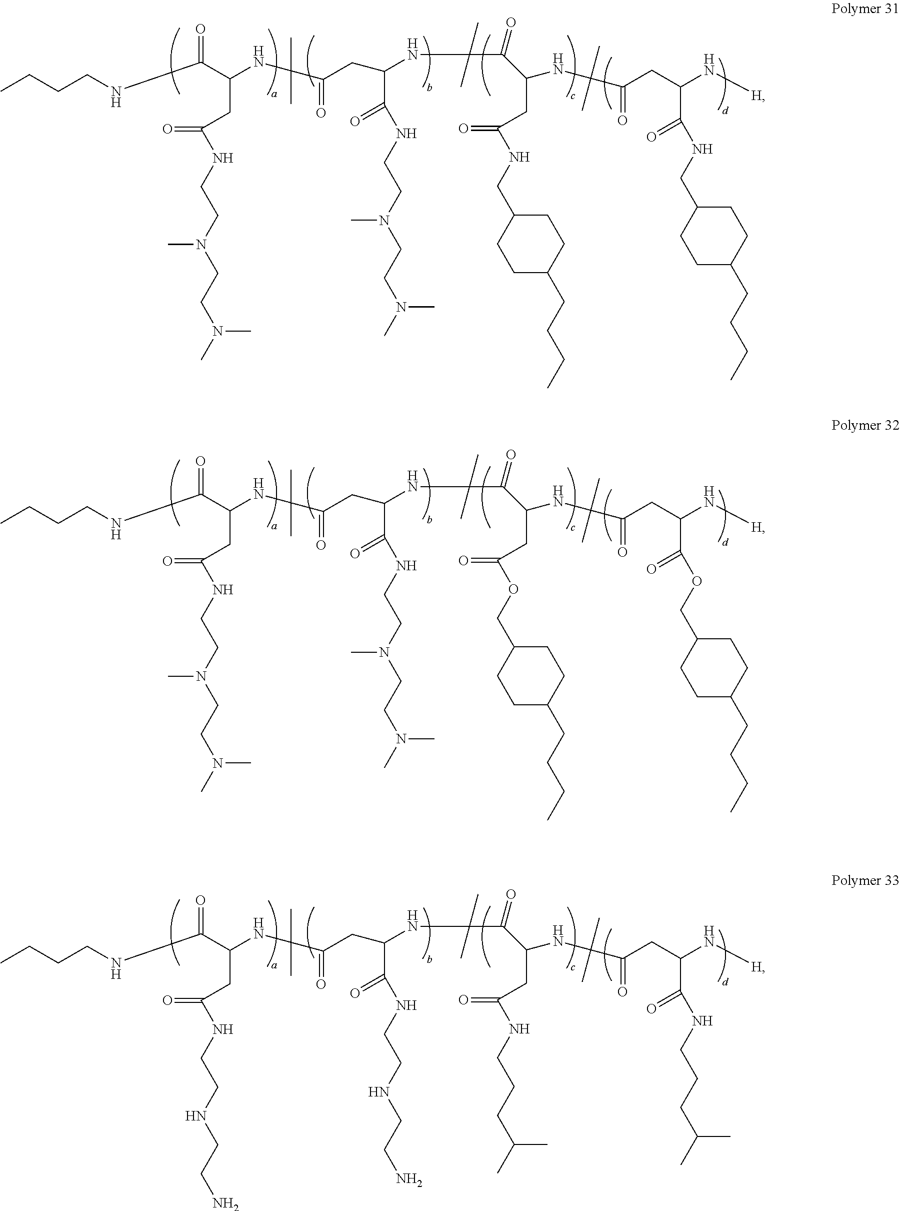 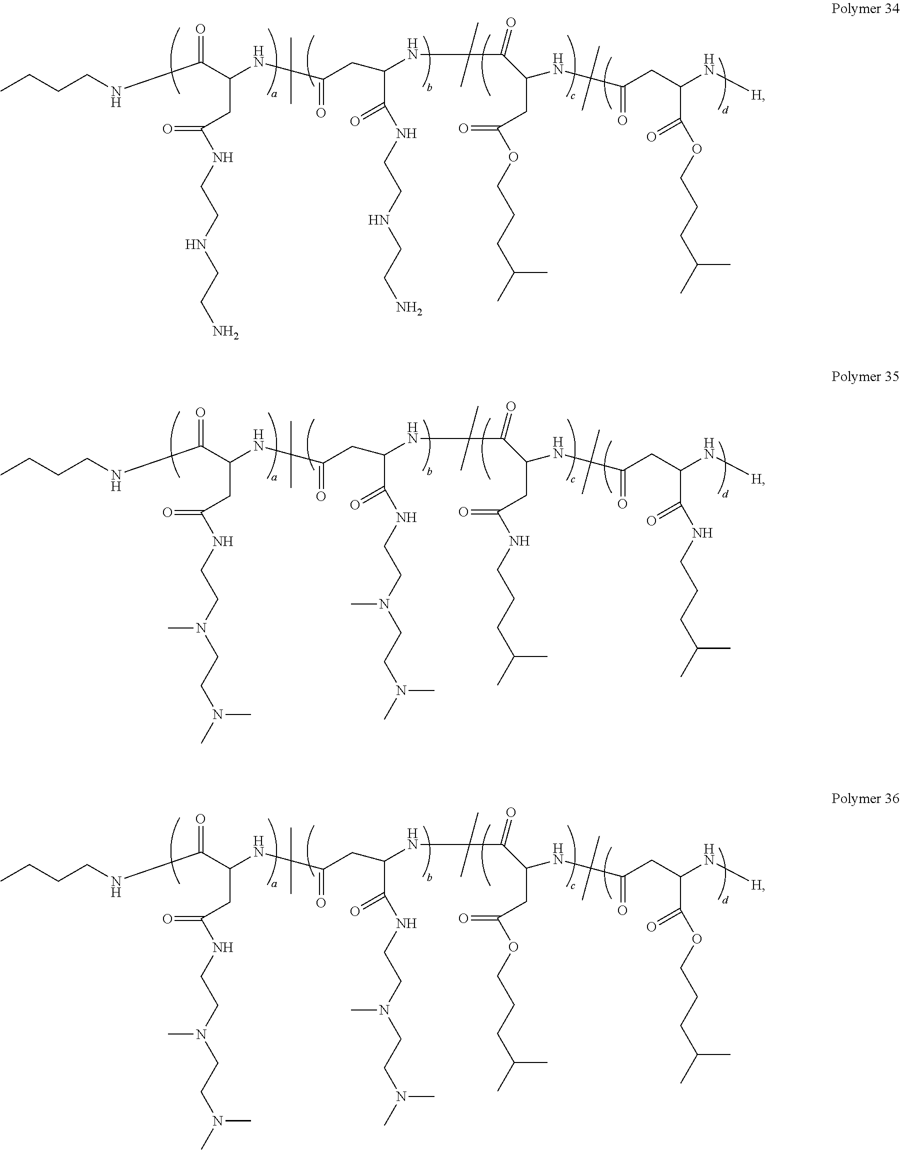 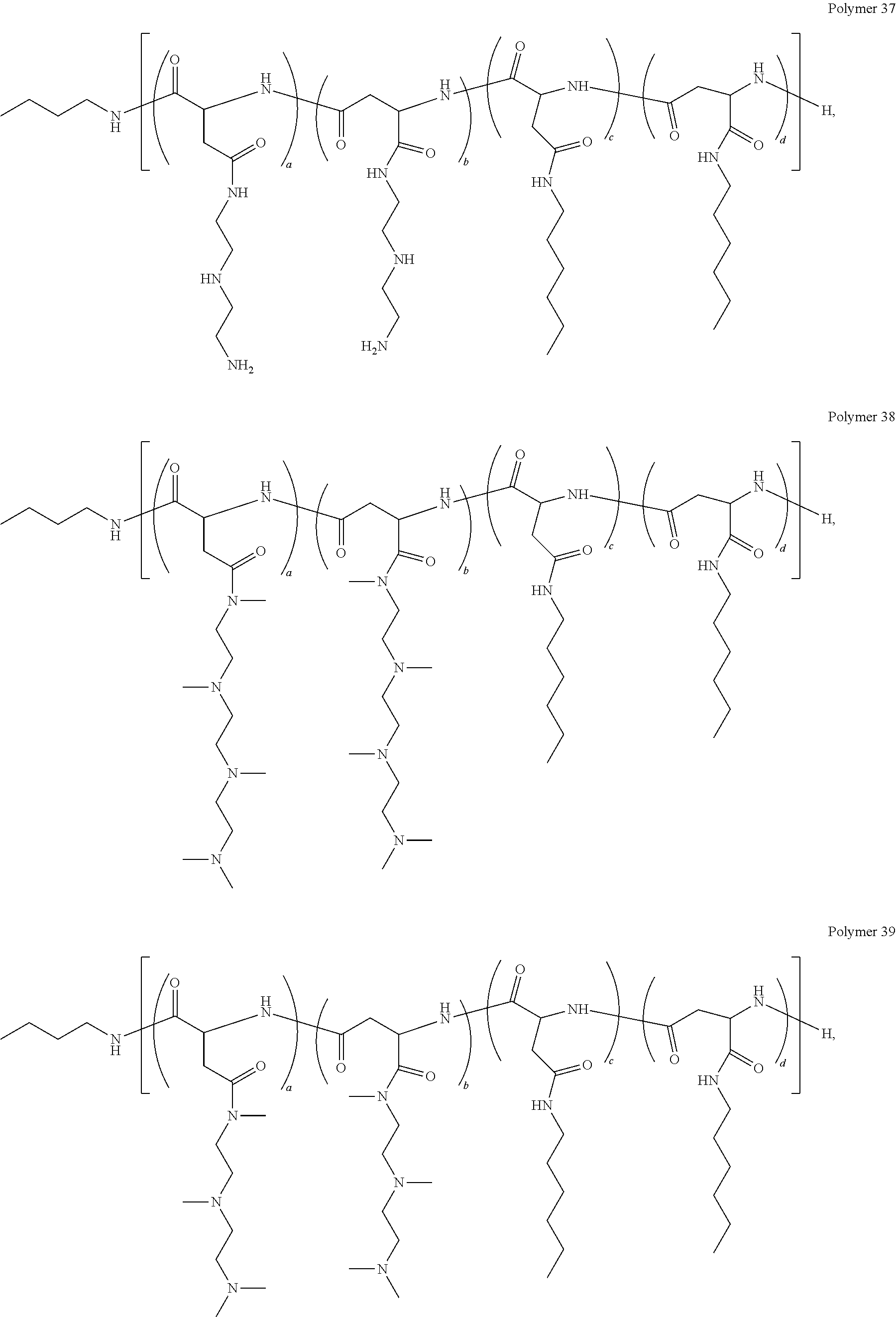 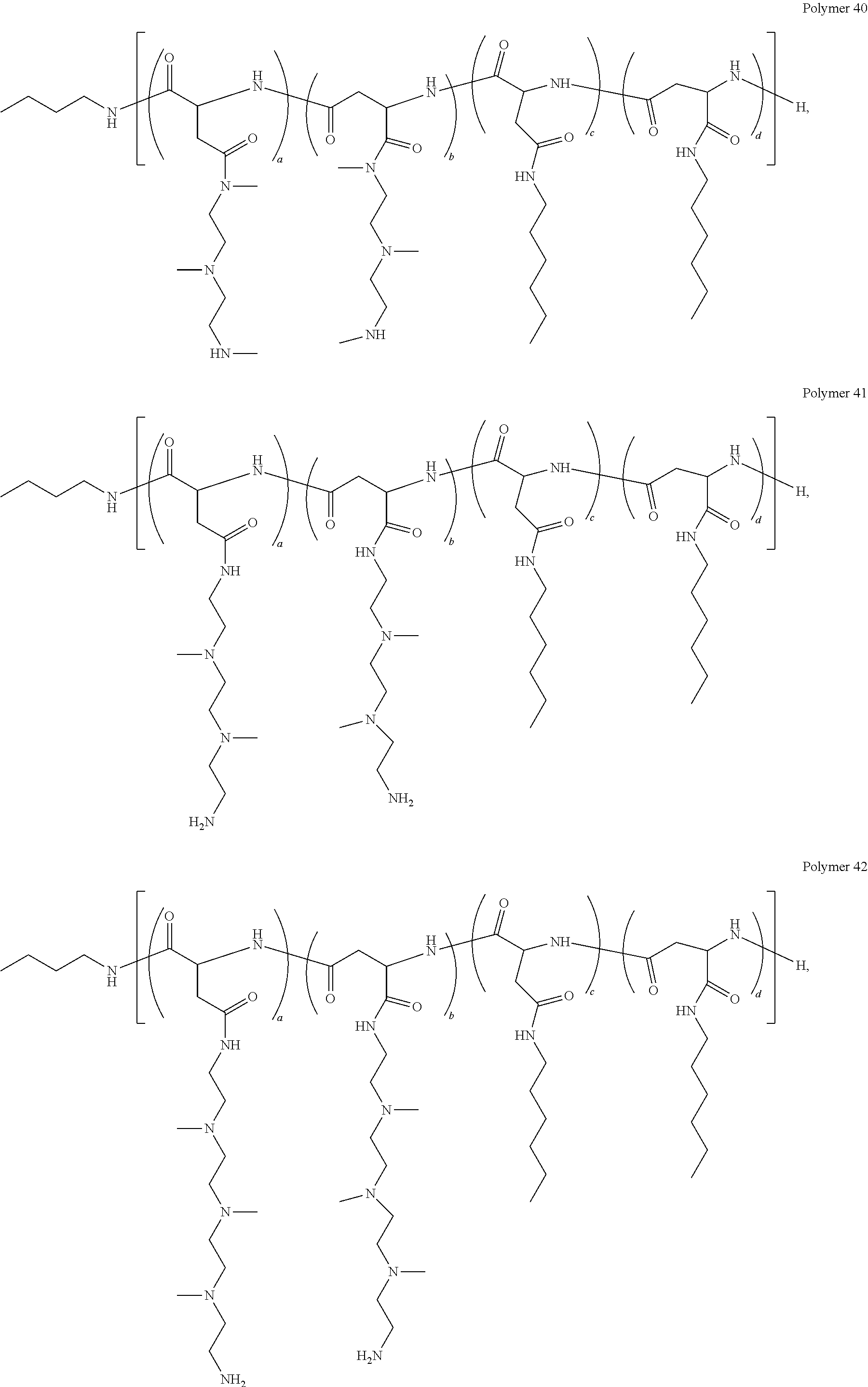 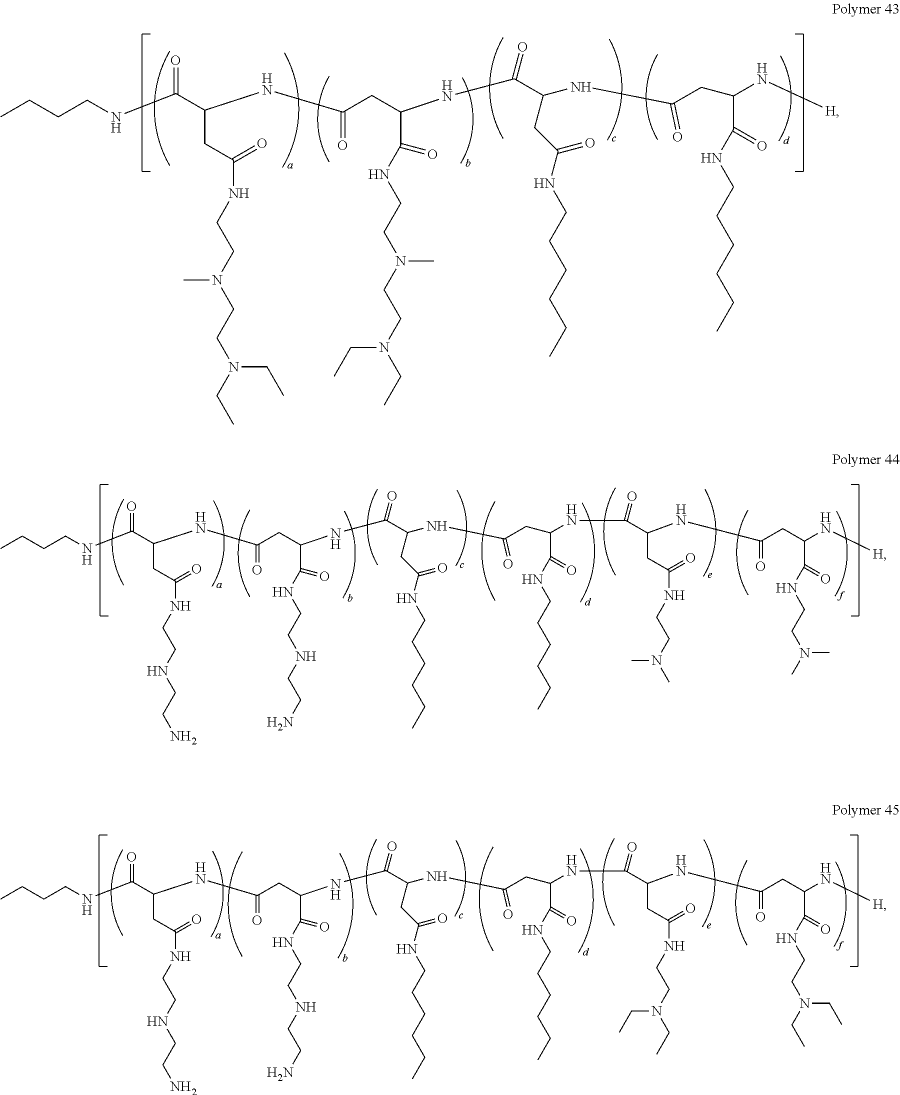 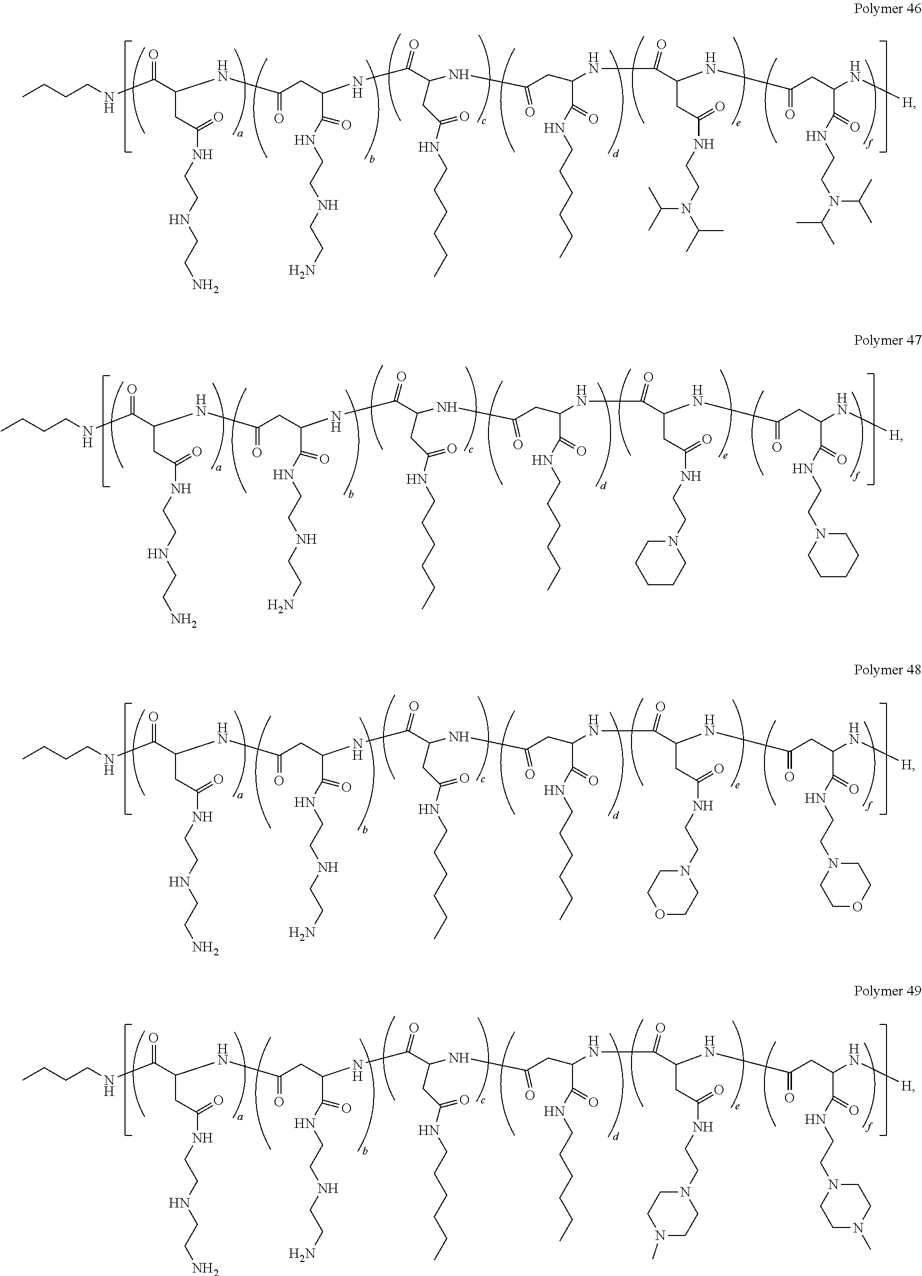 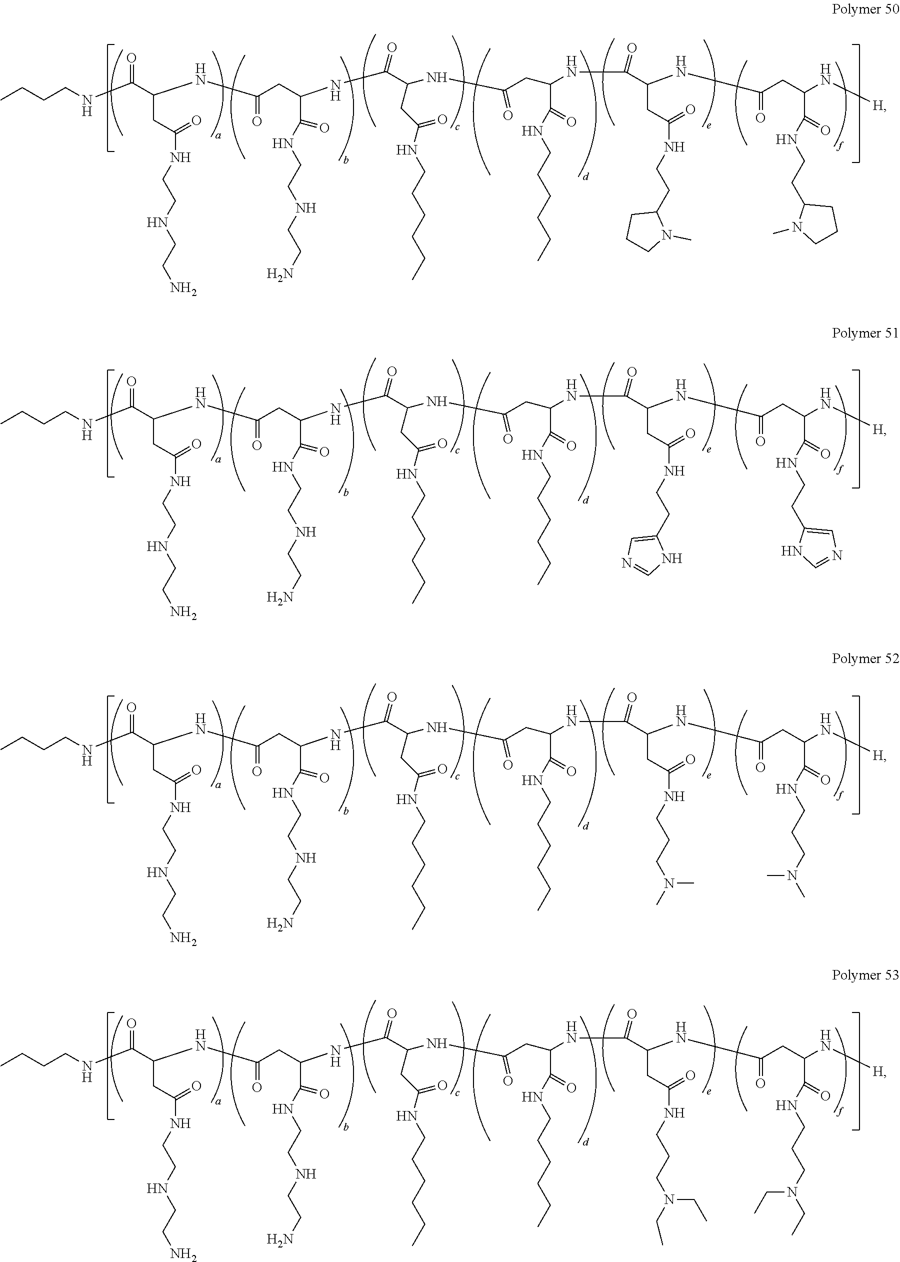 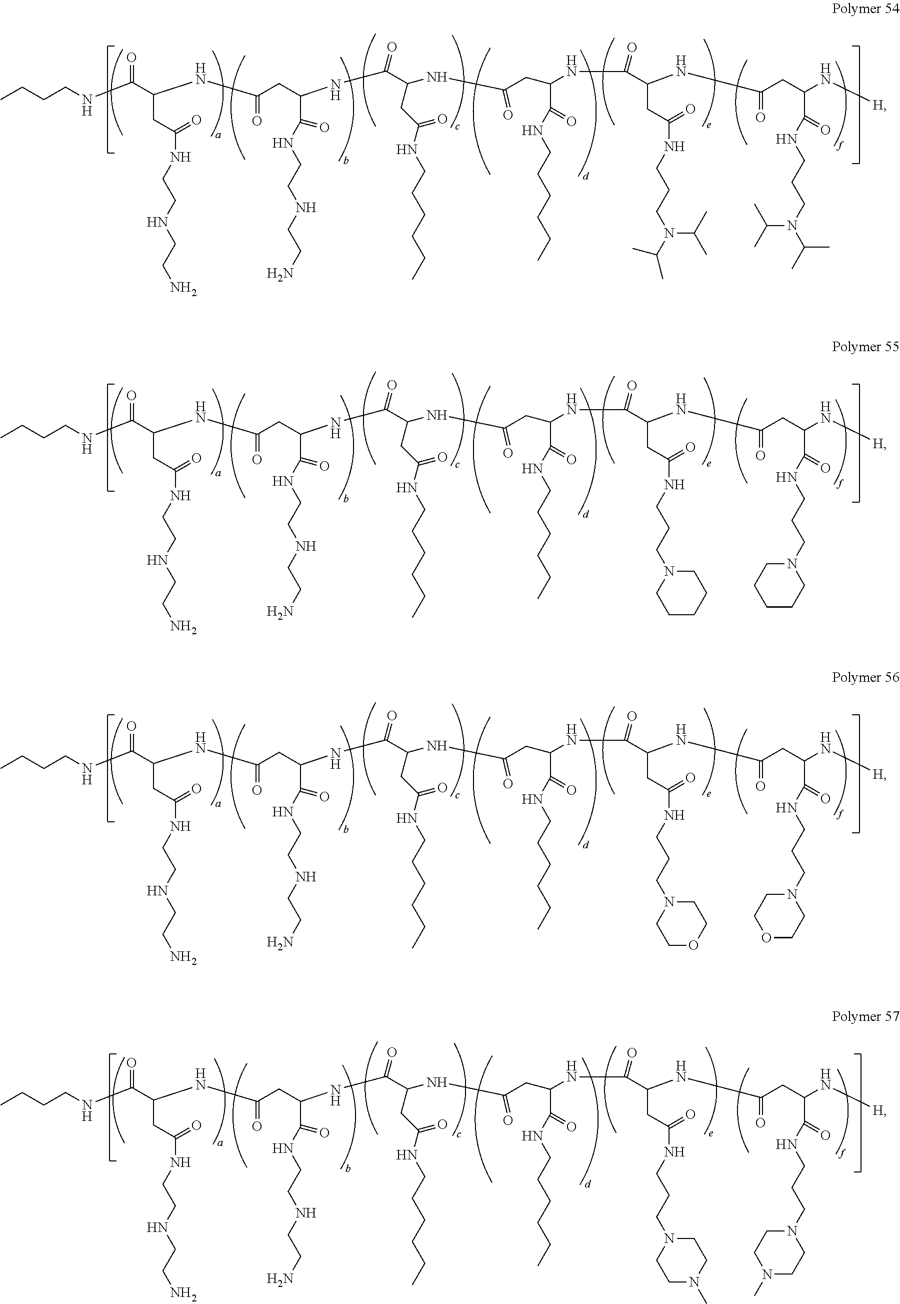 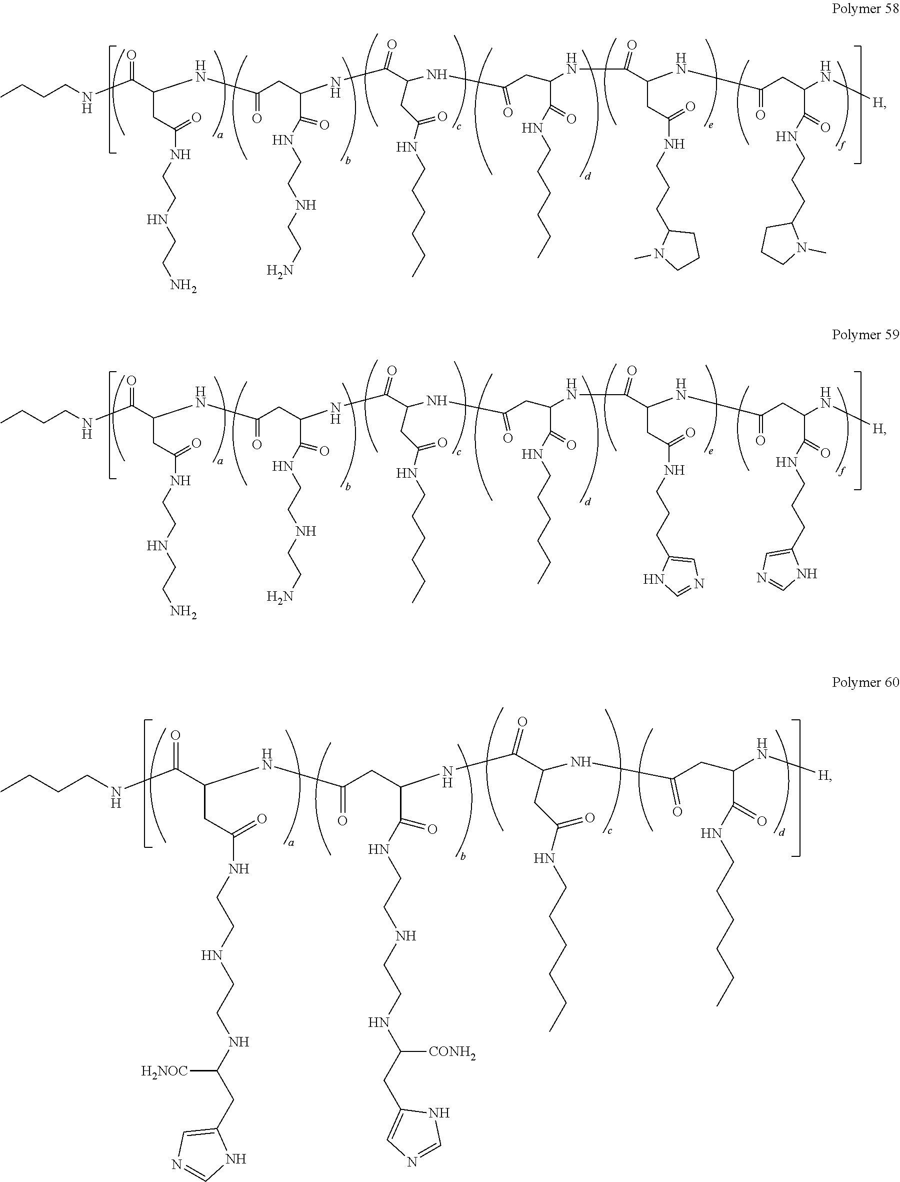 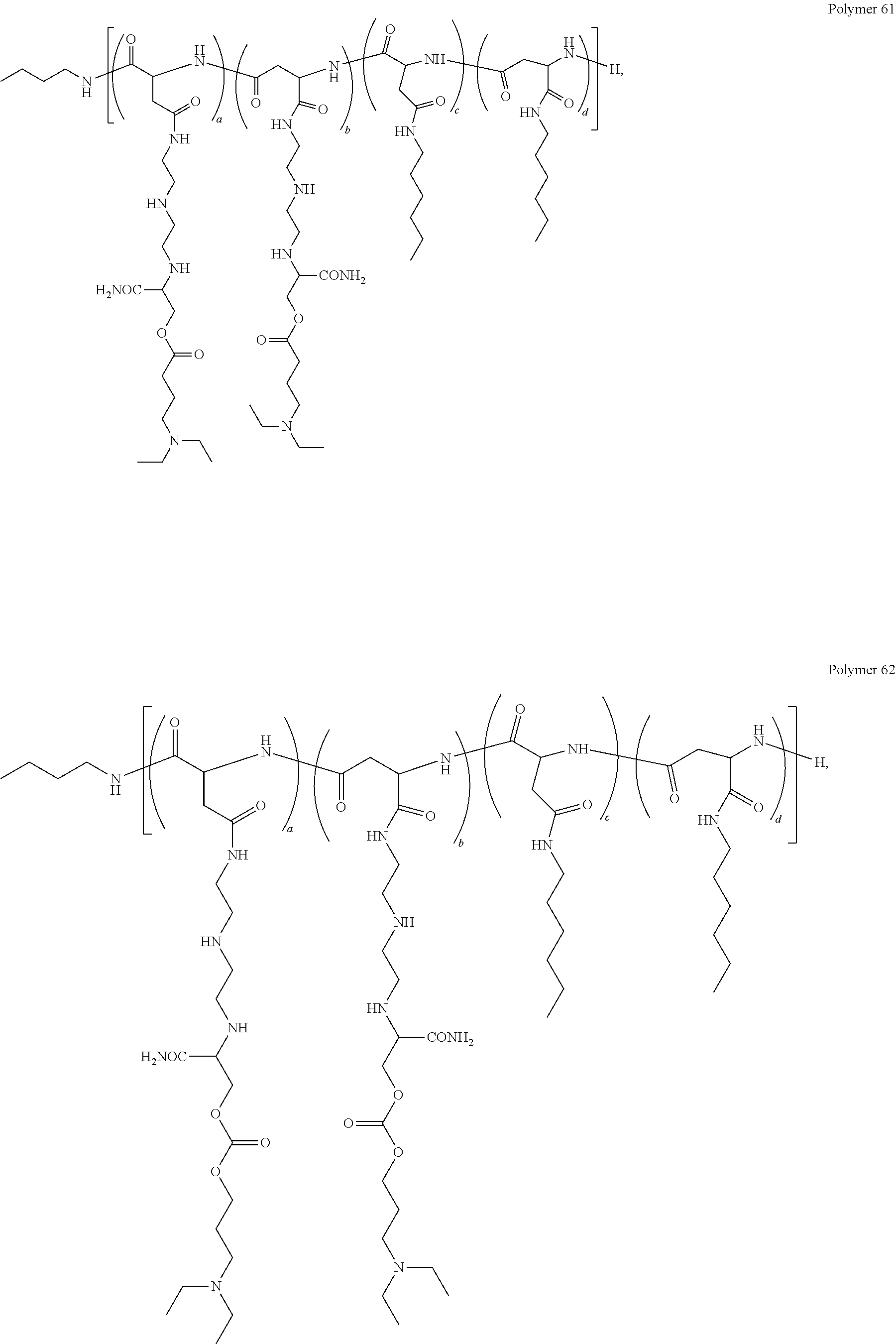 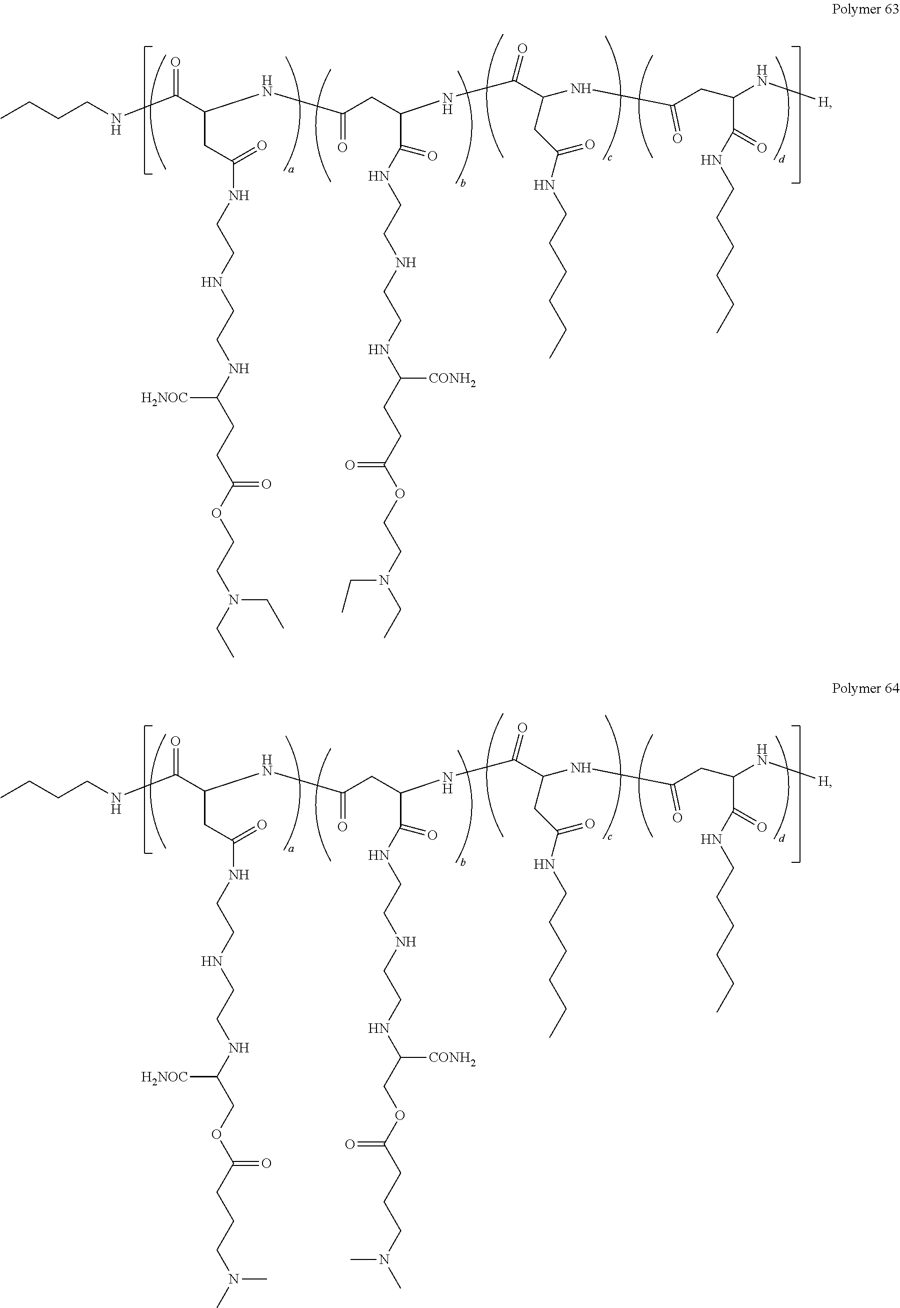 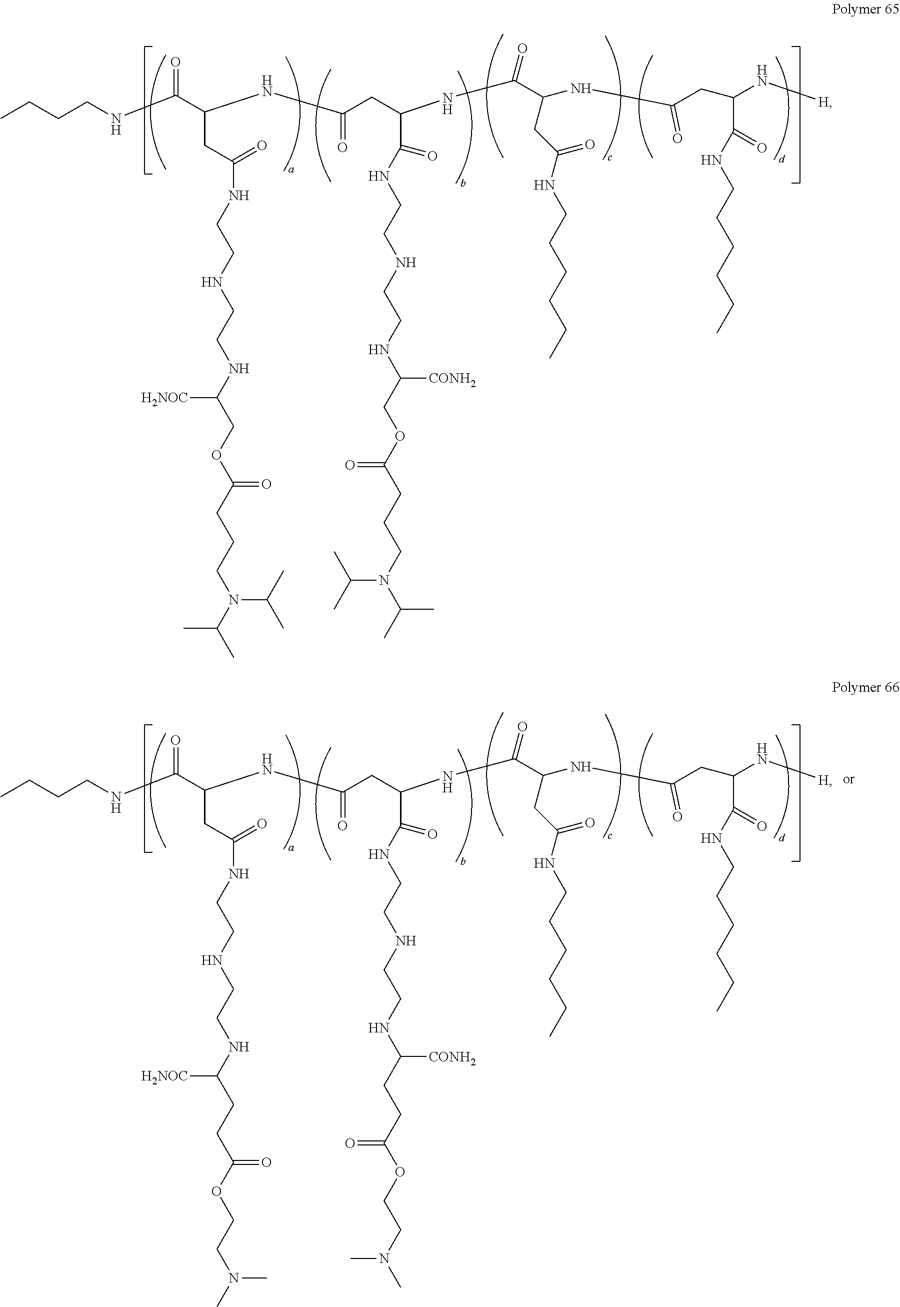 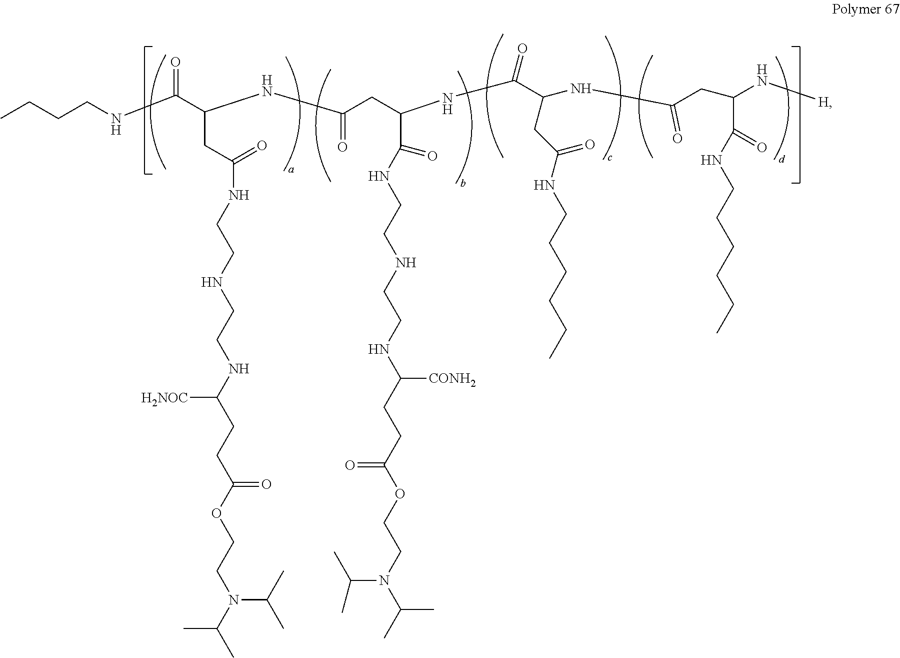
wherein (a+b) is from about 5 to about 65, (c+d) is from about 2 to about 60, and (e+f) is from about 2 to about 60; or 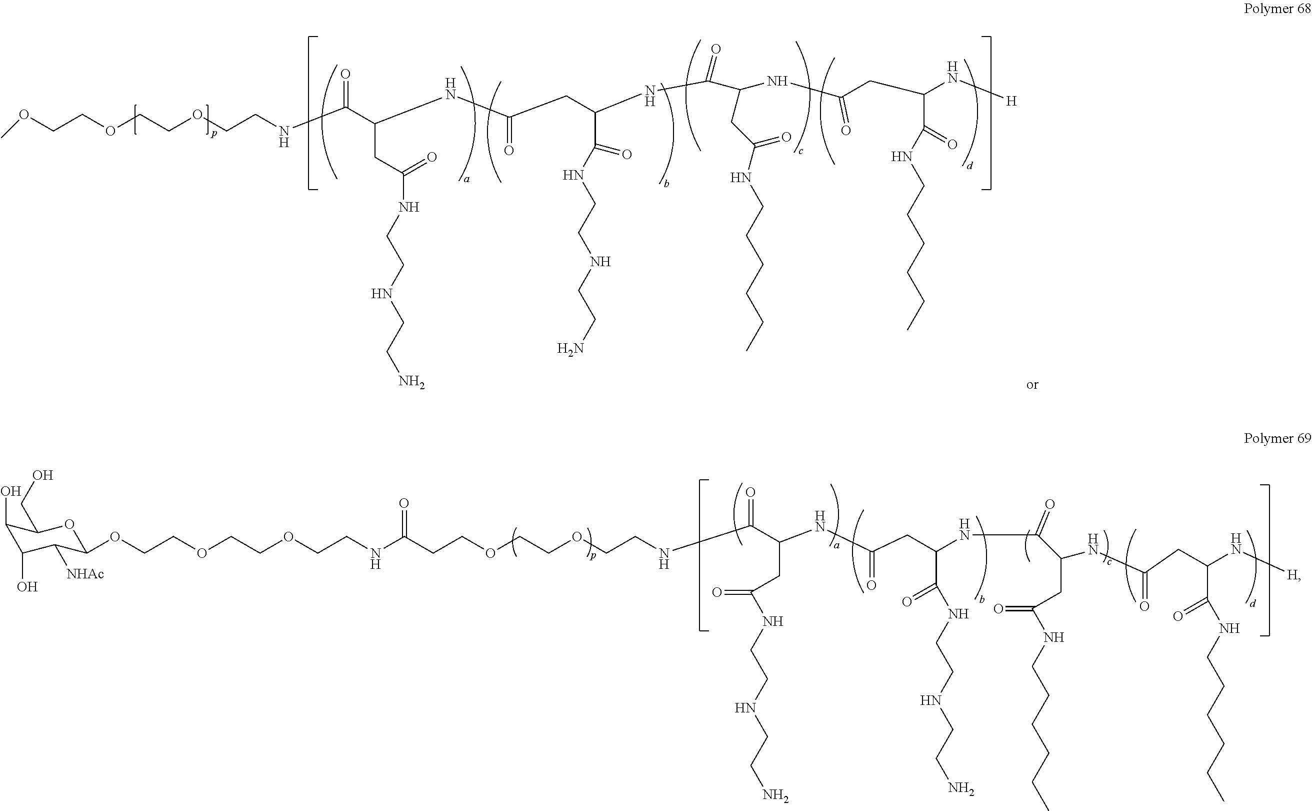
wherein (a+b) is from about 5 to about 65, (c+d) is from about 2 to about 60, and each instance of p is independently an integer from 2 to 200.

22.-23. (canceled)

24. A method of preparing a polymer of Formula 1 according to claim 10, the method comprising:
(a) providing a polymer of Formula 4: 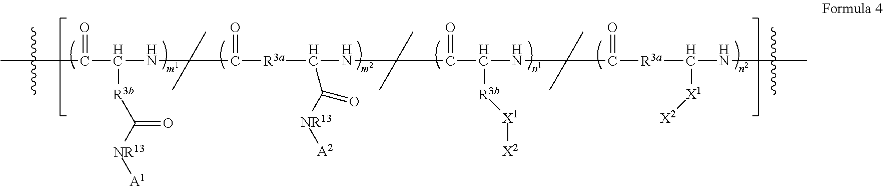
and
(b) modifying a portion of groups A¹ and/or A² of the polymer of Formula 4 to provide the polymer of Formula 1: 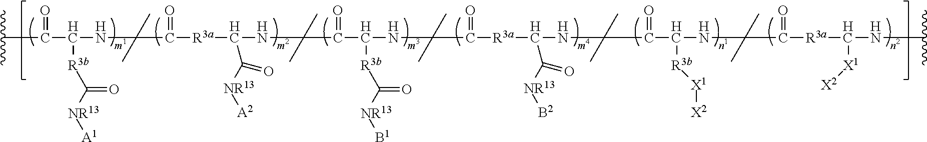
wherein m¹, m², m³, m⁴, n¹, n² R^{3a}, R^{3b}, R¹³, X¹, X², A¹, A², B¹, and B² of Formulas 1 and 4 are as defined in claim 10;
optionally wherein:
modifying a portion of groups A¹ and/or A² of the polymer of Formula 4 comprises reacting a portion of groups with a compound having the structure: 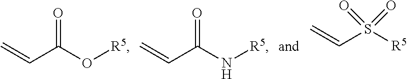
to provide the polymer of Formula 1;
wherein A¹ and A² are each independently a group of formula
—(CH₂)ₚ₁—[NR²—(CH₂)_{q1}—]ᵣ₁NR²₂;
—(CH₂)ₚ₂—N[—(CH₂)_{q2}—NR²₂]₂;
—(CH₂)ₚ₃—N{[—(CH₂)_{q3}—NR²₂][—(CH₂)_{q4}—NR²—]ᵣ₂R²}; or
—(CH₂)ₚ₄—N{—(CH₂)_{q5}—N[—(CH₂)_{q6}—NR²₂]₂}₂,
in which B¹ and B² are:
—(CH₂)ₚ₁—[NR²—(CH₂)_{q1}—]ᵣ₁NR²—(CH₂)ₛ₁—R⁴—R⁵;
—(CH₂)ₚ₂—N[—(CH₂)_{q2}—NR^{2—(CH}₂)ₛ₂—R⁴—R⁵]₂;
—(CH₂)ₚ₃—N{[—(CH₂)_{q3}—NR²₂][—(CH₂)_{q4}—NR²—]ᵣ₂(CH₂)ₛ₃—R⁴—R⁵};
—(CH₂)ₚ₄—N{—(CH₂)_{q5}—N[—(CH₂)_{q6}—NR²—(CH₂)ₛ₄—R⁴—R⁵]₂}₂;
—(CH₂)ₚ₁—[N{(CH₂)ₛ₁—R⁴—R⁵}—(CH₂)_{q1}—]ᵣ₁NR²₂;
—(CH₂)ₚ₁—[NR²—(CH₂)_{q1}—]ᵣ₁NR^{2—CH(CONH}₂)—(CH₂)ₛ₁—R⁴—R⁵.

25. (canceled)

26. The method of claim 24, wherein wherein A¹ and A² are both:
—(CH₂)ₚ₁—[NR²—(CH₂)_{q1}—]ᵣ₁NR²₂;
and B¹ and B² are both:
—(CH₂)ₚ₁—[NR²—(CH₂)_{q1}—]ᵣ₁NR²—(CH₂)ₛ₁—R⁴—R⁵.

27. A method of preparing a polymer of Formula 4 according to claim 18: 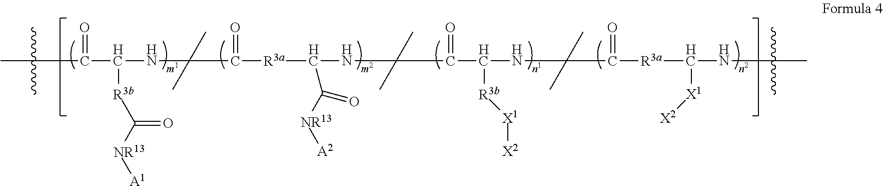
the method comprising:
(I) reacting a polymer of Formula 2: 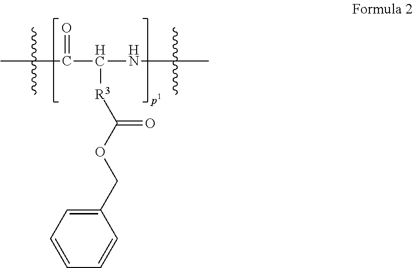
with (a) a compound of the formula HNR¹³A¹ and/or HNR¹³A²; and (b) a compound of formula H₂NX² or HOX², simultaneously or in any sequential order; or
(II) reacting a polymer of Formula 3 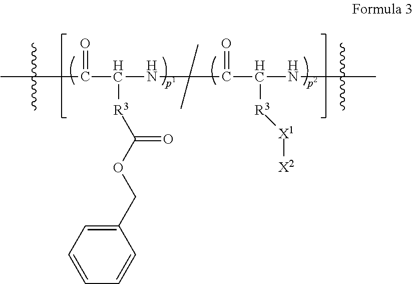
with a compound of the formula HNR¹³A¹ and/or HNR¹³A²;
wherein,
p¹ is an integer from 1 to 2000;
p² is an integer from 1 to 2000;
each R³ is independently a methylene or ethylene group;
and m¹, m², n¹, n², R^{3a}, R^{3b}, R¹³ X¹ , X², A¹, and A² are as defined in claim 10.

28.-30. (canceled)

31. A composition comprising a polymer of claim 1 and a nucleic acid and/or polypeptide; optionally wherein:
the composition comprises a guide nucleic acid and/or donor nucleic acid;
the composition comprises an RNA-guided endonuclease or nucleic acid encoding same
the composition comprises a DNA recombinase;
the composition comprises a zinc finger nuclease;
the composition comprises a transcription activator-like effector nuclease.

32.-39. (canceled)

40. The composition of claim 31, wherein the composition comprises a nanoparticle comprising the polymer and the nucleic acid or polypeptide.

41. The composition of claim 31, wherein the composition comprises a second polymer that comprises polyethylene oxide.

42. A method of delivering a nucleic acid and/or polypeptide to a cell, the method comprising administering the composition of claim 31 to the cell; optionally wherein:
the cell is in a subject and the composition is administered to the subject
the polymer comprises a tissue-specific targeting moiety that localizes the polymer to tissues of the peripheral nervous system, the central nervous system, liver, muscle, lung, bone, or the eye of the subject;
the polymer comprises a targeting moiety that preferentially binds to tumor cells; or
the composition comprises one or more of an RNA guided endonuclease or nucleic acid encoding same, a guide nucleic acid, and a donor nucleic acid, and the composition facilitates editing of a target.

43.-47. (canceled)


SEQUENCE LISTING (20 sequences)

SEQ ID NO: 1 - protein, 1368 aa, Streptococcus pyogenes
  MISC_FEATURE (1)..(20): AKP81606.1
  MISC_FEATURE (1)..(1368): AKP81606.1
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNSLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 2560 aa, Francisella tularensis
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNSLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEMSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 3 - protein, 15 aa, Artificial Sequence
  Synthetic
IGLDIGTNSVGWAVI

SEQ ID NO: 4 - protein, 8 aa, Artificial Sequence
  Synthetic Sequence
IVIEMARE

SEQ ID NO: 5 - protein, 27 aa, Artificial Sequence
  Synthetic Sequence
DVDHIVPQSFLKDDSIDNKVLTRSDKN

SEQ ID NO: 6 - protein, 8 aa, Artificial Sequence
  Synthetic Sequence
HHAHDAYL

SEQ ID NO: 7 - protein, 11 aa, Artificial Sequence
  Synthetic Sequence
YGRKKRRQRRR

SEQ ID NO: 8 - protein, 12 aa, Artificial Sequence
  Synthetic Sequence
RRQRRTSKLMKR

SEQ ID NO: 9 - protein, 27 aa, Artificial Sequence
  Synthetic Sequence
GWTLNSAGYLLGKINLKALAALAKKIL

SEQ ID NO: 10 - protein, 33 aa, Artificial Sequence
  Synthetic Sequence
KALAWEAKLAKALAKALAKHLAKALAKALKCEA

SEQ ID NO: 11 - protein, 16 aa, Artificial Sequence
  Synthetic Sequence
RQIKIWFQNRRMKWKK

SEQ ID NO: 12 - protein, 11 aa, Artificial Sequence
  Synthetic Sequence
YGRKKRRQRRR

SEQ ID NO: 13 - protein, 9 aa, Artificial Sequence
  Synthetic Sequence
RKKRRQRRR

SEQ ID NO: 14 - protein, 11 aa, Artificial Sequence
  Synthetic Sequence
YGRKKRRQRRR

SEQ ID NO: 15 - protein, 8 aa, Artificial Sequence
  Synthetic Sequence
RKKRRQRR

SEQ ID NO: 16 - protein, 11 aa, Artificial Sequence
  Synthetic Sequence
YARAAARQARA

SEQ ID NO: 17 - protein, 11 aa, Artificial Sequence
  Synthetic Sequence
THRLPRRRRRR

SEQ ID NO: 18 - protein, 11 aa, Artificial Sequence
  Synthetic Sequence
GGRRARRRRRR

SEQ ID NO: 19 - protein, 1307 aa, Artificial Sequence
  Synthetic Sequence
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 20 - protein, 1228 aa, Artificial Sequence
  Synthetic
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK